SEQUENCE LISTING (270 sequences)

SEQ ID NO: 1 - protein, 42 aa, synthetic construct
KRGRKKLLYIFKQPFMRPVQTTQEEDGCSCRFPEEEEGGCEL

SEQ ID NO: 2 - protein, 42 aa, synthetic construct; identical to SEQ ID NO: 1

SEQ ID NO: 3 - protein, 30 aa, synthetic construct
QPFMRPVQTTQEEDGCSCRFPEEEEGGCEL

SEQ ID NO: 4 - protein, 37 aa, synthetic construct
RRDQRLPPDAHKPPGGGSFRTPIQEEQADAHSTLAKI

SEQ ID NO: 5 - protein, 36 aa, synthetic construct
RKAWRLPNTPKPCWGNSFRTPIQEEHTDAHFTLAKI

SEQ ID NO: 6 - protein, 21 aa, synthetic construct
GGGSFRTPIQEEQADAHSTLA

SEQ ID NO: 7 - protein, 48 aa, synthetic construct
QRRKYRSNKGESPVEPAEPCHYSCPREEEGSTIPIQEDYRKPEPACSP

SEQ ID NO: 8 - protein, 58 aa, synthetic construct
QLGLHIWQLRSQCMWPRETQLLLEVPPSTEDARSCQFPEEERGERSAEEKGRLGDLWV

SEQ ID NO: 9 - protein, 67 aa, synthetic construct
SQLCCQLKFWLPIGCAAFVVVCILGCILICWLTKKKYSSSVHDPNGEYMFMRAVNTAKKSRLTDVTL

SEQ ID NO: 10 - protein, 76 aa, synthetic construct
LFPGPSKPFWVLVVVGGVLACYSLLVTVAFIIFWVRSKRSRLLHSDYMNMTPRRPGPTRKHYQPYAPPRDFAAYRS

SEQ ID NO: 11 - protein, 18 aa, synthetic construct
TPRRPGPTRKHYQPYAPP

SEQ ID NO: 12 - protein, 85 aa, synthetic construct
SQLCCQLKFWLPIGCAAFVVVCILGCILICWLTKKKYSSSVHDPNGEYMFMTPRRPGPTRKHYQPYAPPRAVNTAKKSRLTDVTL

SEQ ID NO: 13 - protein, 109 aa, synthetic construct
SQLCCQLKFWLPIGCAAFVVVCILGCILICWLTKKKYSSSVHDPNGEYMFMRAVNTAKKSRLTDVTLKRGRKKLLYIFKQPFMRPVQTTQEEDGCSCRFPEEEEGGCEL

SEQ ID NO: 14 - protein, 97 aa, synthetic construct
SQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCEL

SEQ ID NO: 15 - protein, 88 aa, synthetic construct
SQLCCQLKFWLPIGCAAFVVVCILGCILICWLTKKKYSSSVHDPNGEYMFMRAVNTAKKSRLTDVTLGGGSFRTPIQEEQADAHSTLA

SEQ ID NO: 16 - protein, 115 aa, synthetic construct
SQLCCQLKFWLPIGCAAFVVVCILGCILICWLTKKKYSSSVHDPNGEYMFMTPRRPGPTRKHYQPYAPPRAVNTAKKSRLTDVTLQPFMRPVQTTQEEDGCSCRFPEEEEGGCEL

SEQ ID NO: 17 - protein, 106 aa, synthetic construct
SQLCCQLKFWLPIGCAAFVVVCILGCILICWLTKKKYSSSVHDPNGEYMFMTPRRPGPTRKHYQPYAPPRAVNTAKKSRLTDVTLGGGSFRTPIQEEQADAHSTLA

SEQ ID NO: 18 - skipped

SEQ ID NO: 19 - protein, 288 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 20 - protein, 194 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSR

SEQ ID NO: 21 - protein, 195 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSR

SEQ ID NO: 22 - protein, 163 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPA

SEQ ID NO: 23 - protein, 163 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRP

SEQ ID NO: 24 - protein, 240 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRS

SEQ ID NO: 25 - protein, 231 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTL

SEQ ID NO: 26 - protein, 273 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLKRGRKKLLYIFKQPFMRPVQTTQEEDGCSCRFPEEEEGGCEL

SEQ ID NO: 27 - protein, 261 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCEL

SEQ ID NO: 28 - protein, 252 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLGGGSFRTPIQEEQADAHSTLA

SEQ ID NO: 29 - protein, 279 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMTPRRPGPTRKHYQPYAPPRAVNTAKKSRLTDVTLQPFMRPVQTTQEEDGCSCRFPEEEEGGCEL

SEQ ID NO: 30 - protein, 270 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMTPRRPGPTRKHYQPYAPPRAVNTAKKSRLTDVTLGGGSFRTPIQEEQADAHSTLA

SEQ ID NO: 31 - protein, 140 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPA

SEQ ID NO: 32 - protein, 216 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRS

SEQ ID NO: 33 - protein, 258 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 34 - protein, 246 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSQPFMRPVQTTQEEDGCSCRFPEEEEGGCEL

SEQ ID NO: 35 - protein, 237 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSGGGSFRTPIQEEQADAHSTLA

SEQ ID NO: 36 - protein, 207 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTL

SEQ ID NO: 37 - protein, 249 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLKRGRKKLLYIFKQPFMRPVQTTQEEDGCSCRFPEEEEGGCEL

SEQ ID NO: 38 - protein, 237 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCEL

SEQ ID NO: 39 - protein, 228 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLGGGSFRTPIQEEQADAHSTLA

SEQ ID NO: 40 - protein, 255 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMTPRRPGPTRKHYQPYAPPRAVNTAKKSRLTDVTLQPFMRPVQTTQEEDGCSCRFPEEEEGGCEL

SEQ ID NO: 41 - protein, 246 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMTPRRPGPTRKHYQPYAPPRAVNTAKKSRLTDVTLGGGSFRTPIQEEQADAHSTLA

SEQ ID NO: 42 - protein, 315 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCELQNPATSQHPPPPPGHRSQAPSHRPPPPGHRVQHQPQKRPPAPSGTQVHQQKGPPLPRPRVQPKPPHGAAENSLSPSSN

SEQ ID NO: 43 - protein, 352 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVSYLRQWVVIPPPLSSPGPQAS

SEQ ID NO: 44 - protein, 430 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCELQNPATSQHPPPPPGHRSQAPSHRPPPPGHRVQHQPQKRPPAPSGTQVHQQKGPPLPRPRVQPKPPHGAAENSLSPSSNNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVSYLRQWVVIPPPLSSPGPQAS

SEQ ID NO: 45 - protein, 273 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLALYLLRRDQRLPPDAHKPPGGGSFRTPIQEEQADAHSTLAKI

SEQ ID NO: 46 - protein, 293 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLKKVAKKPTNKAPHPKQEPQEINFPDDLPGSNTAAPVQETLHGCQPVTQEDGKESRISVQERQ

SEQ ID NO: 47 - protein, 279 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLQRRKYRSNKGESPVEPAEPCHYSCPREEEGSTIPIQEDYRKPEPACSP

SEQ ID NO: 48 - protein, 273 aa, synthetic construct; identical to SEQ ID NO: 26

SEQ ID NO: 49 - protein, 78 aa, synthetic construct
QNPATSQHPPPPPGHRSQAPSHRPPPPGHRVQHQPQKRPPAPSGTQVHQQKGPPLPRPRVQPKPPHGAAENSLSPSSN

SEQ ID NO: 50 - protein, 115 aa, synthetic construct
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVSYLRQWVVIPPPLSSPGPQAS

SEQ ID NO: 51 - protein, 307 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFAC

SEQ ID NO: 52 - protein, 261 aa, synthetic construct; identical to SEQ ID NO: 27

SEQ ID NO: 53 - protein, 252 aa, synthetic construct; identical to SEQ ID NO: 28

SEQ ID NO: 54 - protein, 283 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLAPHPKQEPQEINFPDDLPGSNTAAPVQETLHGCQPVTQEDGKESRISVQERQ

SEQ ID NO: 55 - protein, 270 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLGESPVEPAEPCHYSCPREEEGSTIPIQEDYRKPEPACSP

SEQ ID NO: 56 - protein, 282 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSALYLLRRDQRLPPDAHKPPGGGSFRTPIQEEQADAHSTLAKI

SEQ ID NO: 57 - protein, 302 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 58 - protein, 288 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSQRRKYRSNKGESPVEPAEPCHYSCPREEEGSTIPIQEDYRKPEPACSP

SEQ ID NO: 59 - protein, 282 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 60 - protein, 270 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSQPFMRPVQTTQEEDGCSCRFPEEEEGGCEL

SEQ ID NO: 61 - protein, 261 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSGGGSFRTPIQEEQADAHSTLA

SEQ ID NO: 62 - protein, 292 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSAPHPKQEPQEINFPDDLPGSNTAAPVQETLHGCQPVTQEDGKESRISVQERQ

SEQ ID NO: 63 - protein, 279 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSGESPVEPAEPCHYSCPREEEGSTIPIQEDYRKPEPACSP

SEQ ID NO: 64 - protein, 282 aa, synthetic construct; identical to SEQ ID NO: 59

SEQ ID NO: 65 - protein, 227 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAVAAILGLGLVLGLLGPLAILLALYLLRRDQRLPPDAHKPPGGGSFRTPIQEEQADAHSTLAKI

SEQ ID NO: 66 - protein, 233 aa, synthetic construct
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAIISFFLALTSTALLFLLFFLTLRFSVVKRGRKKLLYIFKQPFMRPVQTTQEEDGCSCRFPEEEEGGCEL

SEQ ID NO: 67-69 - skipped (3 entries)

SEQ ID NO: 70 - DNA, 234 bp, synthetic construct
  source 1..234: mol_type = other DNA
cagaatcctgccacctctcagcaccctccacctccacctggacacagatctcaggccccatctcacagacctccaccacctggtcatcgggtgcagcatcagccccagaaaagacctcctgctcctagcggaacacaggtgcaccagcaaaagggacctccactgcctagacctagagtgcagcctaagcctcctcatggcgctgccgagaatagcctgtctcctagcagcaac

SEQ ID NO: 71 - DNA, 345 bp, synthetic construct
  source 1..345: mol_type = other DNA
aattgcagaaacacaggcccctggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctgcccctgaataccgacgcttacctgagcctgcaagagctgcaaggacaggaccctacacacctggtgtcctacctgagacagtgggtcgtgatccctccacctctctctagtcctggacctcaggcctct

SEQ ID NO: 72 - DNA, 864 bp, synthetic construct
  source 1..864: mol_type = other DNA
atgcagattcctcaagctccttggcctgtcgtgtgggccgttctgcaacttggatggcggcctggctggttcctggactctcctgacagaccctggaatcctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagttccagacactggtcgtgggagttgttggcggactgctgggatctctggtgctgcttgtttgggtgctcgccgtgatctgtagcagagccgccagaggaacaatcggcgccagaaggacaggccagcctctgaaagaggatccctctgctgtccccgtgttcagcgtggactatggcgagctggatttccagtggcgggaaaagacacccgagcctccagtgccttgtgtgcctgagcagacagagtacgccaccatcgtgttccctagcggcatgggcacatctagccctgccagaagaggatctgccgacggacctagatctgcccagcctctcagacctgaggatggccactgttcttggcctctt

SEQ ID NO: 73 - DNA, 420 bp, synthetic construct
  source 1..420: mol_type = other DNA
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgct

SEQ ID NO: 74 - DNA, 648 bp, synthetic construct
  source 1..648: mol_type = other DNA
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgctctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatct

SEQ ID NO: 75 - DNA, 774 bp, synthetic construct
  source 1..774: mol_type = other DNA
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgctctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatccaagcggggcagaaagaagctgctgtacatcttcaagcagcccttcatgcggcccgtgcagaccacacaagaggaagatggctgctcctgtcggttccccgaggaagaagaaggcggttgcgaactg

SEQ ID NO: 76 - DNA, 738 bp, synthetic construct
  source 1..738: mol_type = other DNA
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgctctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatcccagcctttcatgaggcctgtgcagaccacacaagaagaggacggctgctcctgtcggttccccgaggaagaggaaggcggttgcgaactt

SEQ ID NO: 77 - DNA, 711 bp, synthetic construct
  source 1..711: mol_type = other DNA
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgctctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatctggcggcggaagcttcagaacccctatccaagaggaacaggccgacgctcactctacactggct

SEQ ID NO: 78 - DNA, 621 bp, synthetic construct
  source 1..621: mol_type = other DNA
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacactt

SEQ ID NO: 79 - DNA, 747 bp, synthetic construct
  source 1..747: mol_type = other DNA
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctgaagcggggcagaaagaaactgctgtacatcttcaagcagcccttcatgcggcccgtgcagaccacacaagaggaagatggctgctcctgcagattccccgaggaagaagaaggcggctgcgaactt

SEQ ID NO: 80 - DNA, 711 bp, synthetic construct
  source 1..711: mol_type = other DNA
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttcagcctttcatgaggcccgtgcagaccacacaagaagaggacggctgctcctgcagattccccgaggaagaggaaggcggttgcgaactt

SEQ ID NO: 81 - DNA, 684 bp, synthetic construct
  source 1..684: mol_type = other DNA
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttggcggcggaagctttagaacccctatccaagaggaacaggccgacgctcactctacactggct

SEQ ID NO: 82 - DNA, 765 bp, synthetic construct
  source 1..765: mol_type = other DNA
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgacccctagaaggcctggacctaccagaaagcactaccagccttacgctcctcctagagccgtgaacaccgccaagaagtccagactgaccgacgtgacacttcagcctttcatgaggcccgtgcagaccacacaagaagaggacggctgctcctgcagattccccgaggaagaggaaggcggttgcgaactt

SEQ ID NO: 83 - DNA, 738 bp, synthetic construct
  source 1..738: mol_type = other DNA
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgacccctagaaggcctggacctaccagaaagcactaccagccttacgctcctcctagagccgtgaacaccgccaagaagtccagactgaccgacgtgacacttggcggcggaagctttagaacccctatccaagaggaacaggccgacgctcactctacactggct

SEQ ID NO: 84 - DNA, 945 bp, synthetic construct
  source 1..945: mol_type = other DNA
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctccagcctttcatgaggcctgtgcagaccacacaagaagaggacggctgctcctgtcggttccccgaggaagaggaaggcggttgcgaactccagaatcctgccacctctcagcaccctccacctccacctggacacagatctcaggccccatctcacagacctccaccacctggtcatcgggtgcagcatcagccccagaaaagacctcctgctcctagcggaacacaggtgcaccagcaaaagggacctccactgcctagacctagagtgcagcctaagcctcctcatggcgctgccgagaatagcctgtctcctagcagcaac

SEQ ID NO: 85 - DNA, 1056 bp, synthetic construct
  source 1..1056: mol_type = other DNA
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctccagcctttcatgaggcctgtgcagaccacacaagaagaggacggctgctcctgtcggttccccgaggaagaggaaggcggctgcgaactgaattgcagaaacacaggcccctggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctgcccctgaataccgacgcttacctgagcctgcaagagctgcaaggacaggaccctacacacctggtgtcctacctgagacagtgggtcgtgatccctccacctctctctagtcctggacctcaggcctct

SEQ ID NO: 86 - DNA, 1290 bp, synthetic construct
  source 1..1290: mol_type = other DNA
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctccagcctttcatgaggcctgtgcagaccacacaagaagaggacggctgctcctgtcggttccccgaggaagaggaaggcggttgcgaactccagaatcctgccacctctcagcaccctccacctccacctggacacagatctcaggccccatctcacagacctccaccacctggtcatcgggtgcagcatcagccccagaaaagacctcctgctcctagcggaacacaggtgcaccagcaaaagggacctccactgcctagacctagagtgcagcctaagcctcctcatggcgctgccgagaatagcctgtctcctagcagcaacaactgccgcaacacaggcccctggctgaagaaagtgctgaagtgcaacacccctgatccgagcaagttctttagccagctgagcagcgagcatggcggcgacgttcagaaatggctgtctagcccatttccaagcagcagcttcagccctggtggactggcccctgagattagccctctggaagtgctggaacgggacaaagtgacccagctgctgcccctgaataccgacgcttacctgagcctgcaagagctgcaaggacaggaccctacacacctggtgtcctacctgagacagtgggtcgtgatcccacctcctttgagcagtccaggacctcaggcctct

SEQ ID NO: 87-90 - skipped (4 entries)

SEQ ID NO: 91 - DNA, 921 bp, synthetic construct
  source 1..921: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgc

SEQ ID NO: 92-108 - skipped (17 entries)

SEQ ID NO: 109 - protein, 56 aa, synthetic construct
CWLTKKKYSSSVHDPNGEYMFMTPRRPGPTRKHYQPYAPPRAVNTAKKSRLTDVTL

SEQ ID NO: 110-116 - skipped (7 entries)

SEQ ID NO: 117 - protein, 307 aa, synthetic construct; identical to SEQ ID NO: 51

SEQ ID NO: 118 - protein, 307 aa, synthetic construct; identical to SEQ ID NO: 51

SEQ ID NO: 119 - skipped

SEQ ID NO: 120 - protein, 118 aa, synthetic construct
LFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 121 - protein, 106 aa, synthetic construct
LFPGPSKPFWVLVVVGGVLACYSLLVTVAFIIFWVRSKRSRLLHSDYMNMTPRRPGPTRKHYQPYAPPRDFAAYRSQPFMRPVQTTQEEDGCSCRFPEEEEGGCEL

SEQ ID NO: 122 - protein, 97 aa, synthetic construct
LFPGPSKPFWVLVVVGGVLACYSLLVTVAFIIFWVRSKRSRLLHSDYMNMTPRRPGPTRKHYQPYAPPRDFAAYRSGGGSFRTPIQEEQADAHSTLA

SEQ ID NO: 123 - protein, 109 aa, synthetic construct
SQLCCQLKFWLPIGCAAFVVVCILGCILICWLTKKKYSSSVHDPNGEYMFMRAVNTAKKSRLTDVTLAAGAAALLYIFKQPFMRPVQTTQEEDGCSCRFPEEEEGGCEL

SEQ ID NO: 124 - protein, 109 aa, synthetic construct
SQLCCQLKFWLPIGCAAFVVVCILGCILICWLTKKKYSSSVHDPNGEYMFMRAVNTAKKSRLTDVTLKRGRKKLLYIFAQPFMRPVQTTQEEDGCSCRFPEEEEGGCEL

SEQ ID NO: 125 - protein, 109 aa, synthetic construct
SQLCCQLKFWLPIGCAAFVVVCILGCILICWLTKKKYSSSVHDPNGEYMFMRAVNTAKKSRLTDVTLAAGAAALLYIFAQPFMRPVQTTQEEDGCSCRFPEEEEGGCEL

SEQ ID NO: 126 - protein, 104 aa, synthetic construct
SQLCCQLKFWLPIGCAAFVVVCILGCILICWLTKKKYSSSVHDPNGEYMFMRAVNTAKKSRLTDVTLRRDQRLPPDAHKPPGGGSFRTPIQEEQADAHSTLAKI

SEQ ID NO: 127 - protein, 104 aa, synthetic construct
SQLCCQLKFWLPIGCAAFVVVCILGCILICWLTKKKYSSSVHDPNGEYMFMRAVNTAKKSRLTDVTLAADQALPPDAHKPPGGGSFRTPIQEEQADAHSTLAKI

SEQ ID NO: 128 - protein, 104 aa, synthetic construct
SQLCCQLKFWLPIGCAAFVVVCILGCILICWLTKKKYSSSVHDPNGEYMFMRAVNTAKKSRLTDVTLRRDQRLPPDAHAPPGGGSFRTPIQEEQADAHSTLAAI

SEQ ID NO: 129 - protein, 104 aa, synthetic construct
SQLCCQLKFWLPIGCAAFVVVCILGCILICWLTKKKYSSSVHDPNGEYMFMRAVNTAKKSRLTDVTLAADQALPPDAHAPPGGGSFRTPIQEEQADAHSTLAAI

SEQ ID NO: 130 - protein, 282 aa, synthetic construct; identical to SEQ ID NO: 59

SEQ ID NO: 131 - protein, 270 aa, synthetic construct; identical to SEQ ID NO: 60

SEQ ID NO: 132 - protein, 261 aa, synthetic construct; identical to SEQ ID NO: 61

SEQ ID NO: 133 - protein, 491 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 134 - protein, 380 aa, synthetic construct
MALPVTALLLPLALLLHAARPEVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 135 - protein, 510 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSGGGGSGGGGSGGGGSGGGGSAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 136 - protein, 499 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSGGGGSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 137 - protein, 185 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFAC

SEQ ID NO: 138-146 - skipped (9 entries)

SEQ ID NO: 147 - protein, 38 aa, synthetic construct
CWLTKKKYSSSVHDPNGEYMFMRAVNTAKKSRLTDVTL

SEQ ID NO: 148 - protein, 9 aa, synthetic construct
SLLMWITQV

SEQ ID NO: 149-150 - skipped (2 entries)

SEQ ID NO: 151 - DNA, 354 bp, synthetic construct
  source 1..354: mol_type = other DNA
ctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatccaagcggggcagaaagaagctgctgtacatcttcaagcagcccttcatgcggcccgtgcagaccacacaagaggaagatggctgctcctgtcggttccccgaggaagaagaaggcggttgcgaactg

SEQ ID NO: 152 - DNA, 318 bp, synthetic construct
  source 1..318: mol_type = other DNA
ctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatcccagcctttcatgaggcctgtgcagaccacacaagaagaggacggctgctcctgtcggttccccgaggaagaggaaggcggttgcgaactt

SEQ ID NO: 153 - DNA, 291 bp, synthetic construct
  source 1..291: mol_type = other DNA
ctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatctggcggcggaagcttcagaacccctatccaagaggaacaggccgacgctcactctacactggct

SEQ ID NO: 154 - DNA, 201 bp, synthetic construct
  source 1..201: mol_type = other DNA
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacactt

SEQ ID NO: 155 - DNA, 327 bp, synthetic construct
  source 1..327: mol_type = other DNA
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctgaagcggggcagaaagaaactgctgtacatcttcaagcagcccttcatgcggcccgtgcagaccacacaagaggaagatggctgctcctgcagattccccgaggaagaagaaggcggctgcgaactt

SEQ ID NO: 156 - DNA, 291 bp, synthetic construct
  source 1..291: mol_type = other DNA
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttcagcctttcatgaggcccgtgcagaccacacaagaagaggacggctgctcctgcagattccccgaggaagaggaaggcggttgcgaactt

SEQ ID NO: 157 - DNA, 264 bp, synthetic construct
  source 1..264: mol_type = other DNA
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttggcggcggaagctttagaacccctatccaagaggaacaggccgacgctcactctacactggct

SEQ ID NO: 158 - DNA, 345 bp, synthetic construct
  source 1..345: mol_type = other DNA
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgacccctagaaggcctggacctaccagaaagcactaccagccttacgctcctcctagagccgtgaacaccgccaagaagtccagactgaccgacgtgacacttcagcctttcatgaggcccgtgcagaccacacaagaagaggacggctgctcctgcagattccccgaggaagaggaaggcggttgcgaactt

SEQ ID NO: 159 - DNA, 318 bp, synthetic construct
  source 1..318: mol_type = other DNA
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgacccctagaaggcctggacctaccagaaagcactaccagccttacgctcctcctagagccgtgaacaccgccaagaagtccagactgaccgacgtgacacttggcggcggaagctttagaacccctatccaagaggaacaggccgacgctcactctacactggct

SEQ ID NO: 160 - DNA, 327 bp, synthetic construct
  source 1..327: mol_type = other DNA
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctggctgccggcgctgcagctctgctgtacatcttcaagcagcccttcatgcggcccgtgcagaccacacaagaggaagatggctgctcctgcagattccccgaggaagaagaaggcggctgcgaactt

SEQ ID NO: 161 - DNA, 327 bp, synthetic construct
  source 1..327: mol_type = other DNA
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctgaagcggggcagaaagaaactgctgtacatcttcgcacagcccttcatgcggcccgtgcagaccacacaagaggaagatggctgctcctgcagattccccgaggaagaagaaggcggctgcgaactt

SEQ ID NO: 162 - DNA, 327 bp, synthetic construct
  source 1..327: mol_type = other DNA
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctggctgccggcgctgcagctctgctgtacatcttcgcacagcccttcatgcggcccgtgcagaccacacaagaggaagatggctgctcctgcagattccccgaggaagaagaaggcggctgcgaactt

SEQ ID NO: 163 - DNA, 312 bp, synthetic construct
  source 1..312: mol_type = other DNA
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttaggagagaccagcgtctgccaccagatgcacataagccacctggcggcggaagctttagaacccctatccaagaggaacaggccgacgctcactctacactggctaaaatc

SEQ ID NO: 164 - DNA, 312 bp, synthetic construct
  source 1..312: mol_type = other DNA
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttgccgcggaccaggccctgccaccagatgcacataagccacctggcggcggaagctttagaacccctatccaagaggaacaggccgacgctcactctacactggctaaaatc

SEQ ID NO: 165 - DNA, 312 bp, synthetic construct
  source 1..312: mol_type = other DNA
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttaggagagaccagcgtctgccaccagatgcacatgcaccacctggcggcggaagctttagaacccctatccaagaggaacaggccgacgctcactctacactggctgcaatc

SEQ ID NO: 166 - DNA, 312 bp, synthetic construct
  source 1..312: mol_type = other DNA
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttgccgcggaccaggccctgccaccagatgcacatgcaccacctggcggcggaagctttagaacccctatccaagaggaacaggccgacgctcactctacactggctgcaatc

SEQ ID NO: 167 - DNA, 420 bp, synthetic construct
  source 1..420: mol_type = other DNA
ttcctggactctcctgacagaccctggaatcctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgcc

SEQ ID NO: 168 - DNA, 585 bp, synthetic construct
  source 1..585: mol_type = other DNA
atggctgtgatggctcctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagttccagacactggtcgtgggagttgttggcggcctgctgggatctctggttctgcttgtttgggtgctcgccgtgatctgctctaga

SEQ ID NO: 169 - DNA, 720 bp, synthetic construct
  source 1..720: mol_type = other DNA
atggctgtgatggctcctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgctctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatct

SEQ ID NO: 170 - DNA, 846 bp, synthetic construct
  source 1..846: mol_type = other DNA
atggctgtgatggcccctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgctctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatccaagcggggcagaaagaagctgctgtacatcttcaagcagcccttcatgcggcccgtgcagaccacacaagaggaagatggctgctcctgtcggttccccgaggaagaagaaggcggttgcgaactg

SEQ ID NO: 171 - DNA, 810 bp, synthetic construct
  source 1..810: mol_type = other DNA
atggctgtgatggcccctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgctctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatcccagcctttcatgaggcctgtgcagaccacacaagaagaggacggctgctcctgtcggttccccgaggaagaggaaggcggttgcgaactt

SEQ ID NO: 172 - DNA, 783 bp, synthetic construct
  source 1..783: mol_type = other DNA
atggctgtgatggcccctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgctctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatctggcggcggaagcttcagaacccctatccaagaggaacaggccgacgctcactctacactggct

SEQ ID NO: 173 - DNA, 693 bp, synthetic construct
  source 1..693: mol_type = other DNA
atggctgtgatggctcctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacactt

SEQ ID NO: 174 - DNA, 819 bp, synthetic construct
  source 1..819: mol_type = other DNA
atggctgtgatggctcctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctgaagcggggcagaaagaaactgctgtacatcttcaagcagcccttcatgcggcccgtgcagaccacacaagaggaagatggctgctcctgcagattccccgaggaagaagaaggcggctgcgaactt

SEQ ID NO: 175 - DNA, 783 bp, synthetic construct
  source 1..783: mol_type = other DNA
atggctgtgatggctcctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttcagcctttcatgaggcccgtgcagaccacacaagaagaggacggctgctcctgcagattccccgaggaagaggaaggcggttgcgaactt

SEQ ID NO: 176 - DNA, 756 bp, synthetic construct
  source 1..756: mol_type = other DNA
atggctgtgatggctcctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttggcggcggaagctttagaacccctatccaagaggaacaggccgacgctcactctacactggct

SEQ ID NO: 177 - DNA, 837 bp, synthetic construct
  source 1..837: mol_type = other DNA
atggctgtgatggctcctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgacccctagaaggcctggacctaccagaaagcactaccagccttacgctcctcctagagccgtgaacaccgccaagaagtccagactgaccgacgtgacacttcagcctttcatgaggcccgtgcagaccacacaagaagaggacggctgctcctgcagattccccgaggaagaggaaggcggttgcgaactt

SEQ ID NO: 178 - DNA, 810 bp, synthetic construct
  source 1..810: mol_type = other DNA
atggctgtgatggctcctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgacccctagaaggcctggacctaccagaaagcactaccagccttacgctcctcctagagccgtgaacaccgccaagaagtccagactgaccgacgtgacacttggcggcggaagctttagaacccctatccaagaggaacaggccgacgctcactctacactggct

SEQ ID NO: 179 - DNA, 1473 bp, synthetic construct
  source 1..1473: mol_type = other DNA
atggctctgcctgtgacagctctgctgctgcctctggctctgcttctgcatgccgccagacctgacatcgtgctgacacagtctccacctagcctggccatgagcctgggaaagagagccaccatcagctgtagagccagcgagagcgtgacaatcctgggctctcacctgatccactggtatcagcagaagcccggccagcctcctacactgctgattcagctggcctccaatgtgcagacaggcgtgccagccagattttctggcagcggcagcagaaccgacttcaccctgacaatcgaccccgtggaagaggacgatgtggccgtgtactactgcctccagagccggacaatccccagaacatttggcggaggcaccaagctggaaatcaagggcagcacaagcggctctggcaagcctggatctggcgagggatctaccaagggacagatccagctggtgcagtctggccccgagctgaagaaacctggcgagacagtgaagatcagctgcaaggccagcggctacaccttcaccgactacagcatcaactgggtcaagagagcccctggcaagggcctgaaatggatgggctggatcaacaccgaaaccagagagcccgcctacgcctacgacttcagaggcagattcgccttcagcctggaaaccagcgccagcacagcctacctccagatcaacaacctgaagtacgaggacaccgccacctacttttgcgccctggattacagctacgccatggactattggggccagggcacaagcgtgaccgtgtcctctacaacaacccctgctcctcggcctccaacaccagctcctacaattgcctctcagcccctgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtactgcaagcggggcagaaagaagctgctgtacatcttcaagcagcccttcatgcggcccgtgcagaccacacaagaggaagatggctgctcctgtcggttccctgaggaagaagaaggcggctgcgagctgagagtgaagttcagcagatccgccgacgctcctgcctatcagcagggacagaatcagctctacaacgagctgaacctggggcgcagagaagagtacgacgtgctggacaagagaagaggcagggaccctgagatgggcggaaagccccagagaagaaagaaccctcaagagggcctgtacaatgagctgcaaaaggacaagatggccgaggcctacagcgagatcggaatgaagggcgaacgcagaagaggaaagggccacgacggactgtatcagggcctgagcacagccaccaaggacacctatgatgccctgcacatgcaggccctgcctccaaga

SEQ ID NO: 180 - DNA, 1140 bp, synthetic construct
  source 1..1140: mol_type = other DNA
atggctctgcctgtgacagctctgctgctgcctctggctctgcttctgcatgccgctagacctgaggtgcagctgcttgaatctggcggaggactggttcagcctggcggatctctgagactgtcttgtgccgccagcggcttcacctttagcagctacgccatgagctgggtccgacaggctcctggcaaaggccttgaatgggtgtccagcatcagcggcagcggcgactacatctactacgccgatagcgtgaagggcagattcaccatcagccgggacatcagcaagaacaccctgtacctccagatgaacagcctgagagccgaggacaccgccgtgtactactgtgccaaagaaggcaccggcgccaatagcagcctggccgattatagaggccagggcacactggtcaccgtgtccagtttcgtgcctgtgttcctgcctgccaagcctaccacaacacccgctcctagacctccaacaccagctccaacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgagcctggtcatcacactgtactgcaaccaccggaacaagcggggcagaaagaagctgctgtacatctttaagcagcccttcatgcggcccgtgcagaccacacaagaggaagatggctgctcctgtcggttccccgaggaagaagaaggcggctgcgagctgagagtgaagttcagcagatccgcagacgcccctgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 181 - DNA, 1530 bp, synthetic construct
  source 1..1530: mol_type = other DNA
atggctctgcctgtgacagctctgctgctgcctctggctctgcttctgcatgccgctagaccccaagtgaagctggaagagtctggcggaggactggtgcaggctggcagatctctgagactgtcttgtgccgccagcgagcacacctttagctctcacgtgatgggctggttcagacaggcccctggcaaagaaagggaaagcgtggccgttatcggctggcgggatatcagcacaagctacgccgatagcgtgaagggcagattcaccatcagccgggacaacgccaagaaaaccctgtacctccagatgaacagcctgaagcctgaggacaccgccgtgtactactgcgccgccagaagaattgacgccgccgactttgattcttggggccagggaacccaagtgaccgtttctagcggaggcggtggaagtggcggcggtggatcaggtggtggtggatctggtggcggaggaagcggcggaggcggatctgctgttcagcttgttgaatcaggtggcggcctggttcaggccggggattctcttagactgacctgcacagccagcggcagagccttcagcacctacttcatggcctggtttcggcaggctcccggaaaagaacgggaatttgtggccggaatcgcttggagcggaggctctacagcctatgccgattccgtgaaaggccggtttaccatcagcagagataatgccaaaaacacggtgtacctgcaaatgaactctctgaagtccgaggatacggccgtctattactgtgccagcagaggcatcgaggtggaagagtttggagcctggggacagggcacacaagtcacagtgtctagcaccagcaccaccacaccagctcctagacctccaactcctgctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgagcctggtcatcaccctgtactgcaagcggggcagaaagaagctgctgtacatcttcaagcagcccttcatgcggcccgtgcagaccacacaagaggaagatggctgctcctgtcggttccccgaggaagaagaaggcggctgcgagctgagagtgaagttcagcagaagtgccgacgctcccgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcaaaaggacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 182 - DNA, 1497 bp, synthetic construct
  source 1..1497: mol_type = other DNA
atggctctgcctgtgacagctctgctgctgcctctggctctgcttctgcatgccgctagaccccaagtgaagctggaagagtctggcggaggactggtgcaggctggcagatctctgagactgtcttgtgccgccagcgagcacacctttagctctcacgtgatgggctggttcagacaggcccctggcaaagaaagggaaagcgtggccgttatcggctggcgggatatcagcacaagctacgccgatagcgtgaagggcagattcaccatcagccgggacaacgccaagaaaaccctgtacctccagatgaacagcctgaagcctgaggacaccgccgtgtactactgcgccgccagaagaattgacgccgccgactttgattcttggggccagggaacccaagtgaccgtttctagcggaggcggtggaagtggcggcggtggatcaggtggtggtggatctgaagtgcagctggtggaatcaggcggcggtcttgttcaagccggtggttcactgagactgagctgtgccgcttccggcagaacctttaccatgggatggtttaggcaggctccagggaaagaacgcgagttcgtggccgccatttctctgtctccaacactggcctactacgccgagtccgtgaaaggccggttcacaatctccagagataatgccaagaacaccgtggtgctgcaaatgaactccctgaagccagaagatacagccctgtattactgtgccgccgaccggaagtccgtgatgagcatcagacctgactactggggacagggcacacaagtcacagtgtccagcaccagcaccacaacacccgctcctagacctccaacaccagctccaacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgagcctggtcatcaccctgtactgcaagcggggcagaaagaagctgctgtacatcttcaagcagcccttcatgcggcccgtgcagaccacacaagaggaagatggctgctcctgtcggttccccgaggaagaagaaggcggctgcgagctgagagtgaagttcagcagatccgccgacgctcctgcctatcagcagggacagaaccagctgtacaacgagctgaacctggggagaagagaagagtacgacgtgctggataagcggagaggcagagatcctgagatgggcggaaagccccagcggagaaagaatcctcaagagggcctgtataatgagctgcaaaaggacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 183 - DNA, 555 bp, synthetic construct
  source 1..555: mol_type = other DNA
atggctctgcctgtgacagctctgctgctgcctctggctctgcttctgcatgccgctagacctgaagtgcagttgcaggcttctggcggaggacttgctcaacctggcggaagcctgagactgtcttgtgccgcctctggcagaaccttcagcacctacttcatggcctggttcagacagcctcctggcaaaggcctggaatacgttggcggaatccgttggagtgatggcgtgccacactacgccgatagcgtgaagggcagattcaccatcagccgggacaacgccaagaacaccgtgtacctccagatgaacagcctgagagccgaggataccgccgtgtacttctgtgccagcagaggaatcgccgacggcagcgattttggctcttatggccagggcacccaagtgaccgtgtccagcacaacaacccctgctcctagacctcctacaccagctcctacaatcgccagccagcctctgtctctgaggccagaggcttgtagacctgctgctggcggagccgtgcatacaagaggactggatttcgcctgc

SEQ ID NO: 184-192 - skipped (9 entries)

SEQ ID NO: 193 - protein, 41 aa, synthetic construct
RSKRSRLLHSDYMNMTPRRPGPTRKHYQPYAPPRDFAAYRS

SEQ ID NO: 194 - DNA, 228 bp, synthetic construct
  source 1..228: mol_type = other DNA
ctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatct

SEQ ID NO: 195 - DNA, 126 bp, synthetic construct
  source 1..126: mol_type = other DNA
aagcggggcagaaagaaactgctgtacatcttcaagcagcccttcatgcggcccgtgcagaccacacaagaggaagatggctgctcctgcagattccccgaggaagaagaaggcggctgcgaactt

SEQ ID NO: 196 - DNA, 111 bp, synthetic construct
  source 1..111: mol_type = other DNA
aggagagaccagcgtctgccaccagatgcacataagccacctggcggcggaagctttagaacccctatccaagaggaacaggccgacgctcactctacactggctaaaatc

SEQ ID NO: 197 - DNA, 90 bp, synthetic construct
  source 1..90: mol_type = other DNA
cagcctttcatgaggcccgtgcagaccacacaagaagaggacggctgctcctgcagattccccgaggaagaggaaggcggttgcgaactt

SEQ ID NO: 198 - DNA, 63 bp, synthetic construct
  source 1..63: mol_type = other DNA
ggcggcggaagctttagaacccctatccaagaggaacaggccgacgctcactctacactggct

SEQ ID NO: 199 - skipped

SEQ ID NO: 200 - protein, 332 aa, synthetic construct
MLGIWTLLPLVLTSVARLSSKSWAQVTDINSKGLELRKTVTTVETQNLEGLHHDGQFCHKPCPPGERKARDCTWGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGWEAKIDEIKNDNVQDTAEQKVQLLRNWHQLHGKKEAYDTLIKDLKKANLCTLAEKIQTIILKDITSDSENSNFRNEIQSLV

SEQ ID NO: 201 - DNA, 1005 bp, synthetic construct
  source 1..1005: mol_type = other DNA
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtc

SEQ ID NO: 202 - DNA, 750 bp, synthetic construct
  source 1..750: mol_type = other DNA
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggttccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgctcaaggacattactagtgactcagaaaattcaaacttcagaaatgaaatccaaagcttggtc

SEQ ID NO: 203 - protein, 332 aa, synthetic construct
MLGIWTLLPLVLTSVARLSSKSWAQVTDINSKGLELRKTVTTVETQNLEGLHHDGQFCHKPCPPGERKARDCTWGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGWEAKIVEIKNDNVQDTAEQKVQLLRNWHQLHGKKEAYDTLIKDLKKANLCTLAEKIQTIILKDITSDSENSNFRNEIQSLV

SEQ ID NO: 204 - DNA, 1005 bp, synthetic construct
  source 1..1005: mol_type = other DNA
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagttgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtc

SEQ ID NO: 205 - protein, 357 aa, synthetic construct
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 206 - protein, 335 aa, synthetic construct
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 207 - DNA, 1005 bp, synthetic construct
  source 1..1005: mol_type = other DNA
agaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggaccctcaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgtcggaatgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgttcatg

SEQ ID NO: 208 - DNA, 384 bp, synthetic construct
  source 1..384: mol_type = other DNA
ggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcc

SEQ ID NO: 209 - DNA, 325 bp, synthetic construct
  source 1..325: mol_type = other DNA
ggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaag

SEQ ID NO: 210 - DNA, 598 bp, synthetic construct
  source 1..598: mol_type = other DNA
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 211 - protein, 22 aa, synthetic construct
GSGATNFSLLKQAGDVEENPGP

SEQ ID NO: 212 - DNA, 66 bp, synthetic construct
  source 1..66: mol_type = other DNA
ggatccggcgccaccaatttcagcctgctgaaacaggctggcgacgtggaagagaaccctggacct

SEQ ID NO: 213 - protein, 21 aa, synthetic construct
GSGEGRGSLLTCGDVEENPGP

SEQ ID NO: 214 - DNA, 63 bp, synthetic construct
  source 1..63: mol_type = other DNA
gggtctggggaaggacggggttctcttttaacgtgtggtgacgtggaggaaaatccgggacca

SEQ ID NO: 215 - protein, 67 aa, synthetic construct; identical to SEQ ID NO: 9

SEQ ID NO: 216 - protein, 24 aa, synthetic construct
MAVMAPRTLVLLLSGALALTQTWA

SEQ ID NO: 217 - DNA, 72 bp, synthetic construct
  source 1..72: mol_type = other DNA
atggctgtgatggcccctagaacactggtgctgctgctgtctggtgccctggctctgactcagacatgggcc

SEQ ID NO: 218 - protein, 22 aa, synthetic construct
MLLLVTSLLLCELPHPAFLLIP

SEQ ID NO: 219 - DNA, 66 bp, synthetic construct
  source 1..66: mol_type = other DNA
atgctgctgctggttacatctctgctgctgtgcgagctgccccatcctgcctttctgctgatcccc

SEQ ID NO: 220 - protein, 21 aa, synthetic construct
MALPVTALLLPLALLLHAARP

SEQ ID NO: 221 - DNA, 63 bp, synthetic construct
  source 1..63: mol_type = other DNA
atggctctgcctgtgacagctctgctgctgcctctggctctgcttctgcatgccgctagacct

SEQ ID NO: 222 - protein, 23 aa, synthetic construct
MQIPQAPWPVVWAVLQLGWRPGW

SEQ ID NO: 223 - DNA, 69 bp, synthetic construct
  source 1..69: mol_type = other DNA
atgcagattcctcaagctccttggcctgtcgtgtgggccgttctgcaacttggatggcggcctggctgg

SEQ ID NO: 224 - protein, 25 aa, synthetic construct
MLGIWTLLPLVLTSVARLSSKSVNA

SEQ ID NO: 225 - DNA, 75 bp, synthetic construct
  source 1..75: mol_type = other DNA
atgttgggcatatggaccctactacccttggtcctgacttcggtggcgcggctatcgagcaaatctgtcaacgcg

SEQ ID NO: 226 - protein, 39 aa, synthetic construct
GESPVEPAEPCHYSCPREEEGSTIPIQEDYRKPEPACSP

SEQ ID NO: 227 - protein, 42 aa, synthetic construct
ALYLLRRDQRLPPDAHKPPGGGSFRTPIQEEQADAHSTLAKI

SEQ ID NO: 228 - protein, 62 aa, synthetic construct
KKVAKKPTNKAPHPKQEPQEINFPDDLPGSNTAAPVQETLHGCQPVTQEDGKESRISVQERQ

SEQ ID NO: 229 - protein, 265 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 230 - protein, 335 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 231 - protein, 148 aa, synthetic construct
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSN

SEQ ID NO: 232 - protein, 17 aa, synthetic construct
LGWLCLLLLPIPLIVWV

SEQ ID NO: 233 - protein, 145 aa, synthetic construct
KRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 234 - protein, 85 aa, synthetic construct
SKYITTIAGVMTLSQVKGFVRKNGVNEAKIDEIKNDNVQDTAEQKVQLLRNWHQLHGKKEAYDTLIKDLKKANLCTLAEKIQTII

SEQ ID NO: 235 - protein, 197 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQK

SEQ ID NO: 236 - protein, 172 aa, synthetic construct
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQK

SEQ ID NO: 237 - protein, 250 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLLKDITSDSENSNFRNEIQSLV

SEQ ID NO: 238 - protein, 335 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVQKNGVNEAKIVEIKNDNVQDTAEQKVQLLRNWHQLHGKKEAYDTLIKDLKKANLCTLAEKIQTIILKDITSDSENSNFRNEIQSLV

SEQ ID NO: 239 - protein, 52 aa, synthetic construct
APHPKQEPQEINFPDDLPGSNTAAPVQETLHGCQPVTQEDGKESRISVQERQ

SEQ ID NO: 240 - protein, 63 aa, synthetic construct
VAAILGLGLVLGLLGPLAILLALYLLRRDQRLPPDAHKPPGGGSFRTPIQEEQADAHSTLAKI

SEQ ID NO: 241 - protein, 84 aa, synthetic construct
ALVVIPIIFGILFAILLVLVFIKKVAKKPTNKAPHPKQEPQEINFPDDLPGSNTAAPVQETLHGCQPVTQEDGKESRISVQERQ

SEQ ID NO: 242 - protein, 69 aa, synthetic construct
ILVIFSGMFLVFTLAGALFLHQRRKYRSNKGESPVEPAEPCHYSCPREEEGSTIPIQEDYRKPEPACSP

SEQ ID NO: 243 - protein, 69 aa, synthetic construct
IISFFLALTSTALLFLLFFLTLRFSVVKRGRKKLLYIFKQPFMRPVQTTQEEDGCSCRFPEEEEGGCEL

SEQ ID NO: 244 - protein, 277 aa, synthetic construct
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 245 - protein, 277 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 246 - protein, 260 aa, synthetic construct
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 247 - protein, 255 aa, synthetic construct
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 248 - skipped

SEQ ID NO: 249 - DNA, 660 bp, synthetic construct
  source 1..660: mol_type = other DNA
atgttgcgattgcttttggccttgaatttgttcccctcgattcaggttaccggaaataagatacttgtaaagcagtcgccaatgctcgtggcgtatgataatgctgtgaacttatcgtgtaagtactcttacaaccttttttcgagggaatttcgcgctagtctacacaagggtctagactcggctgtggaggtgtgcgtggtctatggtaattattcgcagcagcttcaggtttacagcaagactggcttcaactgtgacggtaaactaggaaacgagtcagtgaccttttacctccaaaacctatatgtaaaccaaacagacatttatttttgcaagatagaggtgatgtaccctcctccctatctagacaacgagaagtccaatggaacaataatccacgttaaggggaaacacctttgcccctcgccactgttcccaggtccctcaaagcccttttgggtcctagttgtcgtggggggggtattggcatgctattcgttgttggtcacagtcgcctttataatcttttgggttagaagtaagcgcagtcgtctactccacagcgattatatgaatatgacaccaaggcgtcccggccctacgcgaaaacattaccagccctacgcgccgcctcgagatttcgcggcttatcgaagc

SEQ ID NO: 250 - DNA, 597 bp, synthetic construct
  source 1..597: mol_type = other DNA
atgaagagcggtctatggtacttcttcctattctgtctgagaatcaaagtgttaaccggagaaattaatggatcggccaattatgagatgtttattttccataacggcggagtacaaatactctgtaagtaccctgacatagtacagcagtttaagatgcagcttttgaaagggggccaaattctctgcgacttaaccaagacaaagggatctggaaacactgtatcgataaagtccctcaagttctgtcattcccaattatcgaataacagcgtatctttcttcttatacaatctggaccactcccacgccaattattacttctgtaacctcagtattttcgacccgccacctttcaaggttactttgacaggaggttatctgcatatctacgagtcgcagctttgttgtcagctcaagttctggctgcccataggttgcgccgcgtttgtagtagtttgtattttgggatgtatcttgatttgttggcttaccaagaagaagtacagctcgtcggtccatgatcctaatggcgagtacatgtttatgagggccgtgaatacagccaaaaaatcccgcttaacggacgttacctta

SEQ ID NO: 251 - DNA, 228 bp, synthetic construct
  source 1..228: mol_type = other DNA
ctgttcccaggtccctcaaagcccttttgggtcctagttgtcgtggggggggtattggcatgctattcgttgttggtcacagtcgcctttataatcttttgggttagaagtaagcgcagtcgtctactccacagcgattatatgaatatgacaccaaggcgtcccggccctacgcgaaaacattaccagccctacgcgccgcctcgagatttcgcggcttatcgaagc

SEQ ID NO: 252 - DNA, 201 bp, synthetic construct
  source 1..201: mol_type = other DNA
tcgcagctttgttgtcagctcaagttctggctgcccataggttgcgccgcgtttgtagtagtttgtattttgggatgtatcttgatttgttggcttaccaagaagaagtacagctcgtcggtccatgatcctaatggcgagtacatgtttatgagggccgtgaatacagccaaaaaatcccgcttaacggacgttacctta

SEQ ID NO: 253 - DNA, 831 bp, synthetic construct
  source 1..831: mol_type = other DNA
atgtgcgttggggcccgtcgccttggccgcggtccgtgtgcagctttattgctacttggcctagggttgtcgacagtgaccggcctgcactgtgtgggggacacctatccatcgaatgaccggtgttgccatgaatgcaggccgggaaatggcatggtgtcgcggtgtagccgctcccagaatactgtgtgtcgtccttgcggccccggcttctataatgacgtagtatcgagtaaaccttgtaagccgtgcacgtggtgcaatcttaggtccggaagtgaacgtaagcaattatgcacagctactcaggacaccgtgtgtcgttgccgagcagggacacagcccctcgattcatataaacctggagttgattgcgcaccgtgtcccccagggcatttttcacctggcgacaatcaggcttgcaaaccatggactaactgtactctagcagggaagcatacgctccaaccggcttctaatagctctgatgcgatatgcgaagatcgtgatccccccgcaacccagccacaggagacacagggaccccccgcccgtccgatcaccgtgcagccgaccgaagcatggccgcgtacttcgcaggggccttcgactcgacccgtcgaggttcccggtggtagagcagttgcagctattttaggcctgggtcttgtgcttgggttgttgggtcccctcgccatactactagctttgtatttgttgcgccgcgatcaacgcttgcctcccgatgcgcacaagccacctggaggcgggtctttccggacacctatacaagaagaacaggcggacgcccatagcacactcgctaagatt

SEQ ID NO: 254 - DNA, 189 bp, synthetic construct
  source 1..189: mol_type = other DNA
gttgcagctattttaggcctgggtcttgtgcttgggttgttgggtcccctcgccatactactagctttgtatttgttgcgccgcgatcaacgcttgcctcccgatgcgcacaagccacctggaggcgggtctttccggacacctatacaagaagaacaggcggacgcccatagcacactcgctaagatt

SEQ ID NO: 255 - DNA, 126 bp, synthetic construct
  source 1..126: mol_type = other DNA
gctttgtatttgttgcgccgcgatcaacgcttgcctcccgatgcgcacaagccacctggaggcgggtctttccggacacctatacaagaagaacaggcggacgcccatagcacactcgctaagatt

SEQ ID NO: 256 - DNA, 63 bp, synthetic construct
  source 1..63: mol_type = other DNA
ggaggcgggtctttccggacacctatacaagaagaacaggcggacgcccatagcacactcgct

SEQ ID NO: 257 - DNA, 831 bp, synthetic construct
  source 1..831: mol_type = other DNA
atggtgcgactcccgctacaatgtgtgttgtgggggtgcttactaacggcggtccatccagaaccacctacagcctgtcgggagaagcagtacctaattaatagtcagtgctgcagtctatgtcagccgggacagaaactagtatccgactgtaccgaatttaccgagaccgagtgcctaccgtgtggtgaatcagaattcctcgatacgtggaatagagagacccactgtcaccaacacaagtattgcgaccctaacttaggactgagggtgcagcaaaaggggacatcggagacggatacaatttgtacatgcgaggaagggtggcattgtacatccgaagcatgtgagagttgcgttcttcaccgtagctgtagtccgggatttggggtgaaacaaattgctactggcgtctcggatacgatctgcgaaccctgtcctgtaggatttttctcaaatgtttcttcagcgttcgagaaatgtcatccatggacctcgtgcgagacgaaggatttagtagtgcagcaagctggaacgaataaaaccgatgtagtctgtgggccgcaagatcgcttacgagcgttggttgttattcctatcatcttcggaatcctattcgcaattctcctggtattggttttcataaaaaaggtagcgaagaaaccgacaaacaaggccccccacccgaaacaggagccacaggagattaatttccctgacgatcttccgggcagtaacactgctgcaccggtgcaggaaactttgcacggatgtcaaccggtcacgcaagaagatggaaaagaaagtcgaatctccgtgcaagaacgccag

SEQ ID NO: 258 - DNA, 252 bp, synthetic construct
  source 1..252: mol_type = other DNA
gcgttggttgttattcctatcatcttcggaatcctattcgcaattctcctggtattggttttcataaaaaaggtagcgaagaaaccgacaaacaaggccccccacccgaaacaggagccacaggagattaatttccctgacgatcttccgggcagtaacactgctgcaccggtgcaggaaactttgcacggatgtcaaccggtcacgcaagaagatggaaaagaaagtcgaatctccgtgcaagaacgccag

SEQ ID NO: 259 - DNA, 186 bp, synthetic construct
  source 1..186: mol_type = other DNA
aaaaaggtagcgaagaaaccgacaaacaaggccccccacccgaaacaggagccacaggagattaatttccctgacgatcttccgggcagtaacactgctgcaccggtgcaggaaactttgcacggatgtcaaccggtcacgcaagaagatggaaaagaaagtcgaatctccgtgcaagaacgccag

SEQ ID NO: 260 - DNA, 156 bp, synthetic construct
  source 1..156: mol_type = other DNA
gccccccacccgaaacaggagccacaggagattaatttccctgacgatcttccgggcagtaacactgctgcaccggtgcaggaaactttgcacggatgtcaaccggtcacgcaagaagatggaaaagaaagtcgaatctccgtgcaagaacgccag

SEQ ID NO: 261 - DNA, 780 bp, synthetic construct
  source 1..780: mol_type = other DNA
atggcgcgcccccacccttggtggctttgtgtgctggggacactggtgggactgtccgcaacgcccgctccaaagagttgccccgaacgacattattgggcacaaggtaaactctgttgtcaaatgtgtgagcctggaacgtttctcgtcaaggattgcgaccaacatagaaaagcggctcagtgcgatccgtgtattcctggagtaagtttcagcccggatcaccatacaagacctcactgcgagtcttgtcgtcattgtaactcagggttgttggtgagaaactgcacaattacagccaacgctgaatgcgcatgcagaaacggttggcaatgccgagacaaagaatgcacggagtgcgatcccctcccgaatccttctttgacggcccgttcctcccaagcactaagtccacatccccaaccaacgcaccttccttacgtatctgaaatgttagaggcacgtacggctgggcatatgcaaacccttgccgattttcgacagttacccgctaggactttgtcaacacactggccgccccagagatcgctgtgctcgtcggattttattaggatcctggtcattttttctgggatgtttctcgtgtttaccttagcgggagcattattcctacatcaacgcaggaagtatcgctccaacaagggcgaaagtccagttgaacctgccgaaccatgccactattcgtgcccccgagaggaggagggaagtaccatacctatccaagaagactatcgcaagcccgagccagcatgcagccct

SEQ ID NO: 262 - DNA, 207 bp, synthetic construct
  source 1..207: mol_type = other DNA
atcctggtcattttttctgggatgtttctcgtgtttaccttagcgggagcattattcctacatcaacgcaggaagtatcgctccaacaagggcgaaagtccagttgaacctgccgaaccatgccactattcgtgcccccgagaggaggagggaagtaccatacctatccaagaagactatcgcaagcccgagccagcatgcagccct

SEQ ID NO: 263 - DNA, 144 bp, synthetic construct
  source 1..144: mol_type = other DNA
caacgcaggaagtatcgctccaacaagggcgaaagtccagttgaacctgccgaaccatgccactattcgtgcccccgagaggaggagggaagtaccatacctatccaagaagactatcgcaagcccgagccagcatgcagccct

SEQ ID NO: 264 - DNA, 117 bp, synthetic construct
  source 1..117: mol_type = other DNA
ggcgagagccccgtggaacctgccgagccttgtcactacagctgccccagagaggaagagggcagcacaatccccatccaagaggactacagaaagcccgagcctgcctgctctcct

SEQ ID NO: 265 - DNA, 765 bp, synthetic construct
  source 1..765: mol_type = other DNA
atggggaacagttgctacaacatagtcgcgacgctactactagtgttgaattttgagcgtaccagatcgctgcaggatccgtgttcgaattgccctgcaggaacgttttgtgacaacaacagaaatcaaatatgttccccatgcccgccgaactccttttcttcggccggaggccagcgaacttgcgacatctgccgccaatgcaagggagtcttccggactcggaaagaatgctcctccacgagtaacgccgaatgcgattgcacccctgggtttcactgcctgggcgcagggtgcagcatgtgcgagcaggattgcaagcaaggtcaggagctgacgaaaaaaggatgcaaagactgttgttttggaacgttcaatgatcaaaaacgcggtatctgtaggccttggacaaactgcagtctagacggaaaatcggtgctcgttaatggcacgaaggaacgcgatgtcgtctgcggtcccagcccggctgatctatcaccaggtgcatcatccgtaacaccccccgctcctgcaagagaaccgggccactcccctcaaattataagtttcttcttggccttgacttcgacggcgctattgtttttgctcttctttcttacgttacggttttccgttgtcaaaagagggcgaaaaaagttgctttatattttcaaacaaccatttatgcgaccggttcaaaccacacaagaggaagatggatgctcctgccggttcccagaagaggaggagggcggttgcgagctc

SEQ ID NO: 266 - DNA, 207 bp, synthetic construct
  source 1..207: mol_type = other DNA
attataagtttcttcttggccttgacttcgacggcgctattgtttttgctcttctttcttacgttacggttttccgttgtcaaaagagggcgaaaaaagttgctttatattttcaaacaaccatttatgcgaccggttcaaaccacacaagaggaagatggatgctcctgccggttcccagaagaggaggagggcggttgcgagctc

SEQ ID NO: 267 - DNA, 90 bp, synthetic construct
  source 1..90: mol_type = other DNA
cagcctttcatgaggcctgtgcagaccacacaagaagaggacggctgctcctgtcggttccccgaggaagaggaaggcggttgcgaactt

SEQ ID NO: 268 - DNA, 1005 bp, synthetic construct
  source 1..1005: mol_type = other DNA
atgttgggcatatggaccctactacccttggtcctgacttcggtggcgcggctatcgagcaaatctgtcaacgcgcaagtgactgacattaactccaagggtctcgagctacgaaagacggtcactaccgttgaaacgcagaatcttgaaggcctccaccacgacggtcaattttgccacaaaccctgtccgcccggtgagcgcaaggcccgtgattgcacagttaacggtgacgaacctgactgcgttccatgccaagaggggaaggaatacacagacaaggcccatttctctagcaaatgtaggcggtgccgtctctgtgacgagggacatggtcttgaggttgaaataaactgtactcggacacaaaacaccaagtgcaggtgtaagccgaattttttttgtaactctaccgtgtgtgaacattgtgatccttgtacgaaatgtgagcacggaattataaaggaatgtactttaacttccaacacaaagtgcaaggaagagggctctcggagcaatctaggatggttatgcttgttactccttccgattccacttattgtctgggttaagcgtaaagaagtgcagaagacatgtcgaaagcataggaaagagaaccaaggtagccatgagtcccctaccctcaatcccgaaaccgtggccattaatctgtccgatgtggacttgtcgaaatatataaccactattgcaggcgtcatgacgttaagtcaagtgaaaggattcgttcgcaaaaacggggtgaacgaagcgaagatcgatgagatcaagaacgataatgtccaagacacggctgaacaaaaggtgcagcttctcaggaactggcatcaattacatggtaaaaaggaagcgtacgacacgttaattaaggacctcaaaaaagccaatttgtgcaccctagcggagaaaatccaaaccattatactcaaagatataacttcagactcggaaaactctaacttccgtaacgagattcagtcgctggtt

SEQ ID NO: 269 - DNA, 795 bp, synthetic construct
  source 1..795: mol_type = other DNA
ttcctggactctcctgacagaccctggaatcctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagttccagacactggtcgtgggagttgttggcggactgctgggatctctggtgctgcttgtttgggtgctcgccgtgatctgtagcagagccgccagaggaacaatcggcgccagaaggacaggccagcctctgaaagaggatccctctgctgtccccgtgttcagcgtggactatggcgagctggatttccagtggcgggaaaagacacccgagcctccagtgccttgtgtgcctgagcagacagagtacgccaccatcgtgttccctagcggcatgggcacatctagccctgccagaagaggatctgccgacggacctagatctgcccagcctctcagacctgaggatggccactgttcttggcctctt

SEQ ID NO: 270 - DNA, 1071 bp, synthetic construct
  source 1..1071: mol_type = other DNA
atgctgctgctggttacatctctgctgctgtgcgagctgccccatcctgcctttctgctgatccccagaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggaccctcaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgtcggaatgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgttcatg

(19) United States
(12) Patent Application Publication
(10) Pub. No.: US 2025/0352581 A1
(43) Pub. Date: Nov. 20, 2025

(54) COMBINATION THERAPIES COMPRISING FUNCTIONAL COMPONENTS OF PD-1 SWITCH RECEPTOR AND FAS DOMINANT NEGATIVE RECEPTOR

(71) Applicant: Neomics Pharmaceutials LLC, Rockville, MD (US)

(72) Inventors: William A. COMRIE, Silver Spring, MD (US); Mohammed H. SOFI, Rockville, MD (US); Wenshan HAO, Parsippany, NJ (US)

(21) Appl. No.: 19/213,097

(22) Filed: May 20, 2025

Related U.S. Application Data
(60) Provisional application No. 63/649,724, filed on May 20, 2024.

(51) Int. Cl. A61K 35/17; A61K 40/11; A61K 40/31; A61K 40/42; A61P 35/00; C07K 14/705; C07K 14/71; C07K 16/28

(52) U.S. Cl. CPC A61K 35/17; A61K 40/11; A61K 40/31; A61K 40/421; A61P 35/00; C07K 14/70575; C07K 14/70578; C07K 14/71; C07K 16/2827; A61K 2239/13; A61K 2239/21; A61K 2239/22; A61K 2239/23

(57) ABSTRACT

The present application relates to PD-1 switch receptors, e.g., chimeric PD-1 switch receptors, in combination with a FAS dominant negative receptor, optionally in combination with a safety switch, e.g., truncated EGFR, which can be used in adoptive cell therapy to treat human diseases and disorders.

### CROSS-REFERENCE TO RELATED APPLICATIONS

This application claims priority to and the benefit of U.S. Provisional Application No. 63/649,724, filed on May 20, 2024, the contents of which are hereby incorporated by reference in their entirety.

### REFERENCE TO AN ELECTRONIC SEQUENCE LISTING

The contents of the electronic sequence listing (NEOM-006-001US-ST26.xml, Size: 268,646 bytes; and Date of Creation: May 16, 2025) are herein incorporated by reference in their entirety.

### BACKGROUND

While adoptive cell therapies (ACT) show efficacy in cancer treatment, various suppressive mechanisms within the tumor microenvironment (TME) can decrease the maximal efficacy of adoptively transferred cells. Addressing tumor inhibition of adoptively transferred cells using genetic engineering thus holds the potential to greatly increase the efficacy of T Cell ACT.

A well-defined inhibitory mechanism exploited by tumor cells to limit T cell tumor reactivity is the activation of immune checkpoint receptors on T cells, such as CTLA-4 and PD-1. Activation of these receptors can suppress the anti-tumor immune response, leading to immune escape and tumor growth. Systemic administration of immune checkpoint inhibitors (ICIs), led by anti PD-1 and PD-L1 antibodies, has produced durable therapeutic effect with significant extension in survival of cancer patients of various types, proving the essential role of the PD-1 receptor in regulating T cell activity in the TME and its pharmacological value as a key drug target for cancer immunotherapy. Inhibition of the PD-1/PD-L1 pathway in ACT can also be alternatively achieved by engineering T cells to be refractory to the presence of PD-L1/2 on tumor cells through genetic engineering. Approaches under ongoing bench and clinical evaluation include PD-1 knockout, overexpression of PD-1 decoy receptors, and T cell secretion of anti-PD-1/PD-L1 antibodies. The cell-intrinsic anti-PD-1 strategy may lead to the development of new immunotherapies with improved efficacy and safety.

In addition to the negative regulatory signals upregulated in the TME, tumor-reactive T cells face the additional roadblock of sub-optimal expression of costimulatory molecules, such as CD80/86 in the TME, and low abundance of optimal T cell antigens. These factors converge to prevent productive T Cell Receptor signaling and cellular activation. A desired genetic engineering strategy is therefore to compensate T cells for insufficient costimulatory signaling. Preferential activation of the engineered costimulatory signaling in the tumor microenvironment can neutralize different suppressive mechanisms from tumor cells and promote full-fledged T cell activation and tumor-directed lytic activity.

Activated T cells commonly express tumor necrosis factor receptor superfamily member 6 (CD95/FAS), a pro-apoptotic molecule involved in control of peripheral T cell homeostasis and inhibition of overt antigen-dependent T cell proliferation. Interaction of FAS with FASL on neighboring cells induces apoptosis, representing a critical negative regulatory pathway that controls T cell peripheral numbers. Importantly, reports have demonstrated that FASL can be upregulated in the TME, both on tumor cells and accessory cells, and can target tumor-reactive T cells for apoptosis. Consequently, significant loss of activated T cells due to FAS/FASL interactions in the TME can pose an obstacle to the persistence of tumor-reactive T cells, thereby reducing ACT efficacy. Disruption of the FAS pathway can protect T cells from apoptosis to enhance the expansion and persistence of adoptively transferred T cells.

Adopted transfer of genetically engineered T cells may cause acute or chronic side effects. Precise and swift removal of engineered cells from systematic circulation can be an effective way to control adverse effects of ACT. The functionally inert, non-immunogenic cell surface marker derived from human epidermal growth factor receptor (huEGFRt) can serve as a safety switch that efficiently eliminates transgene-positive cells with the clinically approved drug cetuximab.

Importantly, the above-mentioned pathways, as well as other ways of immune evasion adopted by tumor cells do not act independently, but in concert with each other, to limit the anti-tumor immune response. It may therefore be necessary to protect adoptively transferred T cells from multiple negative-regulatory pathways to achieve maximal effect. To concomitantly alter multiple facets of T cell physiology for the development of effective and safe ACT, new approaches of multicomponent genetic engineering are needed.

The present application addresses this need through the invention of a synthetic Immune Checkpoint Switch (ICS) transgene cassette that can simultaneously express a PD-L1 targeting third-generation chimeric costimulatory receptor, a FAS dominant negative receptor, and a safety switch (truncated EGFR) in T cells.

### SUMMARY

The present disclosure provides, in some aspects, an isolated cell comprising: a) a chimeric PD-1 switch receptor comprising: (i) an extracellular domain that binds to PD-L1 or PD-L2; (ii) a transmembrane domain; and (iii) an intracellular domain; and b) a FAS dominant negative receptor (FASDD).

In some aspects, the present disclosure provides a composition comprising one or more isolated nucleic acids, wherein the one or more isolated nucleic acids encode: a) at least one chimeric PD-1 switch receptor, the chimeric PD-1 switch receptor comprising: (i) an extracellular domain that binds to PD-L1 or PD-L2; (ii) a transmembrane domain; and (iii) an intracellular domain; and b) a FAS dominant negative receptor (FASDD).

In some aspects, the present disclosure provides an isolated nucleic acid encoding: a) a chimeric PD-1 switch receptor comprising: (i) an extracellular domain that binds to PD-L1 or PD-L2; (ii) a transmembrane domain; and (iii) an intracellular domain; and b) a FAS dominant negative receptor (FASDD).

In some aspects, the present disclosure provides a composition comprising one or more isolated oligopeptides, wherein the one or more isolated oligopeptides comprise: a) at least one chimeric PD-1 switch receptor, the chimeric PD-1 switch receptor comprising: (i) an extracellular domain that binds to PD-L1 or PD-L2; (ii) a transmembrane domain; and (iii) an intracellular domain; and b) a FAS dominant negative receptor (FASDD).

Methods of using such cells, compositions, and nucleic acids, such as for treatment of a disease or disorder, are also provided herein.

While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

### DESCRIPTION OF THE DRAWINGS

FIGS. 1A-1C are schematic depicting the modular components of an Immune Checkpoint Switch (“ICS”) for cell-intrinsic checkpoint blockade. FIG. 1A depicts that the ICS gene is a multi-component synthetic gene. FIG. 1B depicts that the modular components of the ICS gene encode for three polypeptides of PD-1 chimeric co-stimulatory receptors (“CCR”) (PD-1 CCR or PD-1 switch receptor), FAS dominant negative receptor (FASDD), and truncated EGFR (huEGFRt). FIG. 1C depicts that the polypeptides are post-translationally processed in T cells into three trans-membrane membrane proteins.

FIG. 2 are flow cytometry plots showing the ICS component expression in transduced T cells. Expression of PD-1, FAS, and huEGFRt is shown with the indicated construct, in third generation and truncated third-generation PD-1 switch receptors with or without FAS dominant negative (“FASDD”) expression.

FIGS. 3A-3D are line graphs depicting the ability of 2^{nd} generation ICS transgenes or individual ICS components to kill PD-L1 expressing target cells. FIG. 3A and FIG. 3C (showing A375 cells) and FIG. 3B and FIG. 3D (showing A375-PDL1 cells) depict target cell killing in response to T cells transduced with the indicated second generation ICS or ICS component gene constructs.

FIGS. 4A-4D are line graphs depicting that transgenes containing various ICS or ICS components enhance killing of cancer cells by transduced T cells in an HLA-mismatch assay and in the presence of increasing PD-1 ligand. FIG. 4A and FIG. 4C demonstrate killing of low PD-L1 A375 cancer cells; FIG. 4B and FIG. 4D demonstrate killing of high PD-L1 A375 cells.

FIGS. 5A-5D are line graphs depicting that transgenes containing various ICS transgenes or individual ICS components enhance T cell proliferation in an HLA-mismatch assay and in the presence of increasing PD-1 ligands. FIG. 5A and FIG. 5C demonstrate the number of dividing CD8 T cells after a 120-hour co-incubation with low PD-L1 A375 targets. FIG. 5B and FIG. 5D demonstrate the number of dividing CD8 T cells after a 120-hour co-incubation with high-PD-L1 A375 targets.

FIGS. 6A-6D are line graphs demonstrating that transgenes containing various ICS constructs or individual ICS components enhance T cell cytotoxicity in the presence of PD-1 ligands. FIG. 6A and FIG. 6C depict K562 target cell killing after a 96-hour co-incubation with transduced T cells and the indicated dose of anti-CD3. FIG. 6B and FIG. 6D depict K562-PDL1 target cell killing after a 96-hour co-incubation with transduced T cells and the indicated dose of anti-CD3.

FIGS. 7A-7D are line graphs depicting that transgenes containing various ICS constructs or ICS transgene components enhance T cell proliferation in the presence of PD-1 ligands. FIG. 7A and FIG. 7C demonstrate the number of transduced T cells after a 96-hour co-incubation with K562 targets and the indicated anti-CD3 concentration. FIG. 7B and FIG. 7D demonstrate the number of transduced T cells after a 96-hour co-incubation with K562-PDL1 targets and the indicated anti-CD3 concentration.

FIGS. 8A-8D are line graphs demonstrating transgenes containing various ICS constructs or ICS transgene components increase cytokine secretion in the presence of PD-L1:IFNγ (FIG. 8A) and TNF (FIG. 8C) production by transduced T cells stimulated in the presence of K562 cells and the indicated amount of anti-CD3 for 18 hours, and IFNγ (FIG. 8B) and TNF (FIG. 8D) production by transduced T cells stimulated in the presence of K562-PDL1 cells and the indicated amount of anti-CD3 for 18 hours.

FIGS. 9A-9B depict that the ICS transgene containing PD-1-28-TNR9 (41BBt)_FASDD_huEGFRt functional components (V308) increases T cell alloreactivity. FIG. 9A are bar graphs demonstrating that the number of dividing T cells in co-culture following six days with the indicated cell line with or without the addition of pembrolizumab or ICS transduction. FIG. 9B is a quantitation of fold increase in dividing T cells by ICS gene over T cells or T cells with pembrolizumab.

FIGS. 10A-10B are bar graphs depicting that the ICS transgene containing PD-1-28-TNR9 (41BBt)_FASDD_huEGFRt functional components (V308) enhances T cell alloreactivity under increasing PD-L1 expression on tumor cells. FIG. 10A shows the number of dividing T cells in co-culture following six days with 50,000 cells of the indicated cell line with or without the addition of pembrolizumab or ICS transgene transduction. FIG. 10B shows the number of dividing T cells in co-culture following six days with 100,000 cells of the indicated cell line with or without the addition of pembrolizumab or ICS transduction.

FIG. 11 depicts differential cancer cell killing by T cells transduced with a high affinity NY-ESO-1 TCR along with transgenes expressing various ICS components as indicated.

FIGS. 12A-12C are line graphs depicting that the truncated third-generation PD-1-28-TNR9 (41BBt) switch receptor of the ICS transgene enhances the efficacy of T cells engineered to express both low and high-affinity TCRs. FIGS. 12A, 12B, and 12C depict killing of H1755, A375, or H522 target cells by T cells engineered to express either the 1G4 NY-ESO1/HLA-A2-specific TCR or one of three high affinity versions with or without concurrent expression of the PD-1-28-TNR9 (41BBt) PD-1 switch receptor.

FIG. 13 depicts that inclusion of the FAS-dominant negative polypeptide component of the ICS transgene protects transduced cells from FAS-mediated apoptosis, as demonstrated by T cell loss following an 18-hour incubation with the indicated concentration of recombinant FAS ligand.

FIGS. 14A-14C are line graphs depicting the ICS transgene combining PD-1 switch receptor and FAS-DN functional components enhances antigen-specific T Cell expansion. FIG. 14A depicts 1G4 low affinity NY-ESO-1 TCR-bearing T Cell number. FIG. 14B depicts GSK-794 high affinity NY-ESO-1 TCR-bearing T Cell number, and FIG. 14C shows NPB2007 high affinity NY-ESO-1 TCR-bearing T Cell number after 4 days in culture with A549 and A549-PDL1 cells incubated with the indicated amount of NY-ESO-1 agonist peptide as analyzed by flow cytometry.

FIGS. 15A-15C are line graphs depicting the ICS transgene PD-1-28-TNR9 (41BBt)_FASDD results in increased T cell-mediated killing of cancer cells than PD-1 blockade alone with increasing presence of PD-L1. FIG. 15A depicts 1G4 low affinity NY-ESO-1 TCR-bearing T Cell number, FIG. 15B depicts GSK-794 high affinity NY-ESO-1 TCR-bearing T Cell number, and FIG. 15C depicts NPB2007 high affinity NY-ESO-1 TCR-bearing Target Cell number after 4 days in culture with A375 and A375-PDL1 cells which express both NY-ESO-1 and HLA-A2 endogenously.

FIGS. 16A-16C are line graphs depicting the ICS transgene PD-1-28-TNR9 (41BBt)_FASDD results in increased T cell proliferation in response to A375 and A375-PDL1 target cells expressing endogenous pMHC complex, above PD-1 blockade alone. FIG. 16A depicts 1G4 low affinity NY-ESO-1 TCR-bearing T cell number. FIG. 16B depicts GSK-794 high affinity NY-ESO-1 TCR-bearing T cell number. FIG. 16C depicts NPB2007 high affinity NY-ESO-1 TCR-bearing T cell number after 4 days in culture with A375 and A375-PDL1 cells which express both NY-ESO-1 and HLA-A2 endogenously.

FIGS. 17A-17D are graphs depicting the ICS transgene PD1-28-TNR9 (41BBt)_FASDD_huEGFRt (V308) potentiates T cells for in vivo tumor killing in a HCC827 murine model. FIG. 17A and FIG. 17B depict Kaplan-Meier plots for the paired T cell doses (5×10⁵ and 1.6×10⁵) T cells, respectively. FIG. 17C and FIG. 17D depict tumor volume over time following adoptive transfer of the indicated T cell population, respectively. Arrows indicate pembrolizumab injection, blue arrow indicates T cell injection.

FIGS. 18A-18C are graphs depicting the ICS transgene PD-1-28-TNR9 (41BBt)_FASDD_huEGFRt (V308) potentiates T Cells for in vivo tumor killing (NCI-H292 model). FIG. 18A and FIG. 18B demonstrate tumor volume in mice following a single infusion of untransduced cells or ICS-T Cells, respectively on day 0. FIG. 18C is a Kaplan Meier plot of mouse survival following adoptive transfer of T cells.

FIG. 19A and FIG. 19B are graphs depicting that the ICS transgene PD-1-28 TNR9 (41BBt)_FASDD_huEGFRt (V308) does not induce tonic signaling or alter cytokine/TCR dependent growth. FIG. 19A demonstrates Tag-It violet dilution of untransduced or V308 transduced cells by flow cytometry. FIG. 19B depicts total cell number after 96 hours of proliferation with the indicated stimulation conditions.

FIGS. 20A-20H depict that the ICS transgene PD-1-28-TNR9 (41BBt)_FASDD_huEGFRt (V308) enhances expansion and function of Tumor Infiltrating Lymphocytes. FIG. 20A demonstrates the percentage of EGFR positive cells following pre-REP and REP of TIL expansion. FIG. 20B depicts the total fold expansion of TILs with or without V308 transduction. FIG. 20C depicts CD8 positive T cells following TIL expansion with or without V308 transduction. FIG. 20D shows killing of K562, K562-PDL1, and K562-PDL1/FAS-L target cells following a 96-hour co-culture with the indicated TIL population. FIG. 20E depicts T cell number following a 96-hour co-culture of TILS with or without V308 transduction and the indicated target cell. Production of cytokines IFNγ (FIG. 20F), IL2 (FIG. 20G), and TNF (FIG. 20H) was determined following an 18-hour co-culture of TILS with or without V308 transduction and the indicated target cell.

### DETAILED DESCRIPTION

In some aspects, provided herein are chimeric co-stimulatory intracellular domains in combination with FAS dominant-negative receptors, or functional components thereof. In some aspects, provided herein are third-generation chimeric co-stimulatory intracellular domains in combination with FAS dominant-negative receptors. The third-generation chimeric co-stimulatory intracellular domains provided herein comprise: (a) a first signaling domain that is based on the intracellular signaling domain of a CD28 family protein; and (b) at least a second signaling domain that comprises a mutant intracellular signaling domain of a TNFR family protein. Such combinations may be useful, for example, in protecting transduced cells from FAS-mediated apoptosis.

In some aspects, provided herein is an isolated cell comprising: a) a chimeric PD-1 switch receptor comprising: (i) an extracellular domain that binds to PD-L1 or PD-L2; (ii) a transmembrane domain; and (iii) an intracellular domain; and b) a FAS dominant negative receptor (FASDD).

In some embodiments, the FASDD comprises a dominant negative FAS mutant.

In some embodiments, the dominant-negative FAS mutant comprises at least one modification (e.g., one, two, three, four, five or more modifications) in the cytoplasmic death domain of FAS or in the extracellular ligand binding domain.

In some embodiments, the FASDD comprises a deletion of amino acid residues 230-314 of SEQ ID NO:200 or a portion thereof. In some embodiments, the FASDD comprises SEQ ID NO:201. In some embodiments, the FASDD comprises a point mutation at position 260 of SEQ ID NO:200. In some embodiments, the point mutation is D260V. In some embodiments, the FASDD comprises SEQ ID NO:203. In some embodiments, the FASDD comprises one or more mutations selected from Thr13fs; Val15_Ala16insTer; Thr28Ala; Lys33Glu; Leu37Ter; His54Arg; Cys59Phe; His60fs; Pro62Arg; Cys73Gly; Asp78fs; Cys82Arg; Val83Met; Pro84Leu; His96Arg; Asp108Gly; Glu116Gly; Arg121Trp; Thr122Ile; Cys135Phe; Thr138Ile; Val139fs; Asp144Glu; Thr160Ala; Thr163Ile; Gly169Val; Gly175Glu; Leu177Arg; Cys178Tyr; Leu179Arg; Ile184Val; Val188fs; Lys193Arg; Glu194Lys; Asn206fs; His210Tyr; Ser212Thr; Thr214Ile, Thr214Asn; Val220fs, Val220Met; Ile222Met; Asn223His; Lys231fs; Tyr232Cys, Tyr232His; Thr234Ala; Thr235del; Thr241Lys, Thr241Pro; Gly247Ala; Val249Leu; Arg250Pro, Arg250Gln; Gly253 Asp, Gly253Ser, Gly253Val; Val254Ala; Ala257Asp; Ile259Arg, Ile259Thr; Asp260Gly; Asp260Val, Asp260Tyr; Ile262Ser; Thr270Ile, Thr270Lys; Glu272Gly, Glu272Lys; Gln273Ter, Gln273 Arg, Glu289Asp; Ala290Glu; Leu294fs; Ala301Thr; Thr305Ile; Ile310Ser; Leu315Phe; Thr319Ile; Ser320Gly; Asp321Asn; Asn326Asp; Glu330fs; or a combination thereof, wherein the one or more mutations are relative to SEQ ID NO: 200.

In some embodiments, the expression of the endogenous FAS gene in the cell is reduced or inhibited. Such reduced or inhibited expression of the endogenous FAS gene can be using any suitable methodology known in the art, e.g., CRISPR, TALEN, or RNA-inhibition.

It is contemplated that the reduction or inhibition of the endogenous FAS gene may be alone or in combination with the expression of the FASDD.

In some embodiments, the extracellular domain of the PD-1 switch receptor is derived from PD-1. In some embodiments, the extracellular domain comprises SEQ ID NO:9.

In some embodiments, the PD-1 switch receptor comprises a transmembrane domain derived from CD8, PD-1, CD28, ICOS, or IgG.

In some embodiments, the PD-1 switch receptor comprises an intracellular domain connected to the C-terminus of the transmembrane domain. In some embodiments, the intracellular domain comprises a first and at least a second signal transduction domain, wherein the first and the at least second signal transduction domains are non-identical. In some embodiments, the first signal transduction domain is derived from CD28, CD28H, ICOS, or a combination thereof and the at least second domain is derived from CD137 (4-1BB), CD134 (OX-40), CD40 (TNFRSF5), or CD27 (TNFRSF7).

In some embodiments, the at least second signal transduction domain comprises a mutant CD137 (4-1BB) intracellular domain, a mutant CD134 (OX-40) intracellular domain, a mutant TNFRSF5 intracellular domain, or a mutant TNFRSF7 intracellular domain.

In some embodiments, the mutant CD137 intracellular domain comprises a truncated CD137 intracellular domain. In some embodiments, the truncated CD137 intracellular domain comprises an amino acid sequence according to amino acid position 13 to amino acid position 42 of the CD137 intracellular domain; a deletion of a continuous stretch of one, two, three, four, five, six, seven, eight, nine, ten or more amino acids from the N-terminus the CD137 intracellular domain; or a deletion of one, two, three, four, five, six, seven, eight, nine, ten or more amino acids from amino acid position 1 to amino acid position 12 of the N-terminus of the CD137 intracellular domain. In some embodiments, the truncated CD137 intracellular domain comprises an amino acid sequence according to SEQ ID NO:3; a deletion of one, two, three or four lysine residue(s) from amino acid position 1 to amino acid position 12 of the N-terminus of the CD137 intracellular domain; one or more lysine mutation(s) from amino acid position 1 to amino acid position 12 of the N-terminus of the CD137 intracellular domain, e.g., one or more lysine mutation(s) at amino acid positions selected from amino acid positions 1, 5, 6 and 12 of the N-terminus of the CD137 intracellular domain; a deletion of one or more proximal basic amino acids from amino acid position 1 to amino acid position 12 of the N-terminus of the CD137 intracellular domain; and/or one or more proximal basic amino acid mutation(s) from amino acid position 1 to amino acid position 12 of the N-terminus of the CD137 intracellular domain, e.g., one or more proximal basic amino acid mutation(s) at amino acid positions selected from amino acid positions 1, 2, 3, 4, 5 and 6 of the N-terminus of the CD137 intracellular domain, e.g., a lysine mutation at amino acid position 12 of the N-terminus of the CD137 intracellular domain.

In some embodiments, the mutant CD134 intracellular domain comprises a truncated CD134 intracellular domain. In some embodiments, the truncated CD134 intracellular domain comprises an amino acid sequence according to amino acid position 15 to amino acid position 37 of the CD134 intracellular domain; a deletion of a continuous stretch of one, two, three, four, five, six, seven, eight, nine, ten, eleven, twelve, thirteen or more amino acids from the N-terminus of the CD134 intracellular domain; or a deletion of one, two, three, four, five, six, seven, eight, nine, ten, eleven, twelve, thirteen or more amino acids from amino acid position 1 to amino acid position 14 of the N-terminus of the CD134 intracellular domain. In some embodiments, the truncated CD134 intracellular domain comprises an amino acid sequence according to SEQ ID NO:6; a deletion of a lysine residue from amino acid position 1 to amino acid position 14 of the N-terminus of the CD134 intracellular domain; a lysine mutation at amino acid position 12 of the N-terminus of the CD134 intracellular domain; a deletion of one or more proximal basic amino acids from amino acid position 1 to amino acid position 14 of the N-terminus of the CD134 intracellular domain; and/or one or more proximal basic amino acid mutation(s) from amino acid position 1 to amino acid position 14 of the N-terminus of the CD134 intracellular domain. In some embodiments, the one or more proximal basic amino acid mutation(s) at amino acid positions are selected from amino acid positions 1, 2, and 5 of the N-terminus of the CD134 intracellular domain. In some embodiments, the mutant CD134 intracellular domain further comprises a lysine mutation at amino acid position 12 of the N-terminus of the CD134 intracellular domain.

In some embodiments, the mutant CD40 intracellular domain comprises a truncated CD40 intracellular domain. In some embodiments, the truncated CD40 intracellular domain comprises an amino acid sequence according to amino acid position 11 to amino acid position 62 of the CD40 intracellular domain. In some embodiments, the truncated CD40 intracellular domain comprises a deletion of a continuous stretch of one, two, three, four, five, six, seven, eight, nine, ten, or more amino acids from the N-terminus of the CD40 intracellular domain. In some embodiments, the truncated CD40 intracellular domain comprises a deletion of one or more proximal basic amino acids from amino acid position 1 to amino acid position 10 of the N-terminus of the CD40 intracellular domain. In some embodiments, the truncated CD40 intracellular domain comprises an amino acid sequence according to SEQ ID NO:239. In some embodiments, the truncated CD40 intracellular domain comprises a deletion of a lysine residue from amino acid position 1 to amino acid position 10 of the N-terminus of the CD40 intracellular domain. In some embodiments, the truncated CD40 intracellular domain comprises a lysine mutation at amino acid position 10 of the N-terminus of the CD40 intracellular domain. In some embodiments, the truncated CD40 intracellular domain comprises a deletion of one or more proximal basic amino acids from amino acid position 1 to amino acid position 10 of the N-terminus of the CD40 intracellular domain. In some embodiments, the truncated CD40 intracellular domain comprises or more proximal basic amino acid mutation(s) from amino acid position 1 to amino acid position 10 of the N-terminus of the CD40 intracellular domain, optionally one or more proximal basic amino acid mutation(s) at amino acid positions selected from amino acid positions 1, 2, 5, 6, and 10 of the N-terminus of the CD40 intracellular domain.

In some embodiments, the mutant CD27 intracellular domain comprises a truncated CD27 intracellular domain. In some embodiments, the truncated CD27 intracellular domain comprises an amino acid sequence according to amino acid position 9 to amino acid position 48 of the CD27 intracellular domain. In some embodiments, the truncated CD27 intracellular domain comprises a deletion of a continuous stretch of one, two, three, four, five, six, seven, eight, nine, or more amino acids from the N-terminus of the CD27 intracellular domain. In some embodiments, the truncated CD27 intracellular domain comprises a deletion of one or more proximal basic amino acids from amino acid position 1 to amino acid position 9 of the N-terminus of the CD27 intracellular domain. In some embodiments, the truncated CD27 intracellular domain comprises an amino acid sequence according to SEQ ID NO:226. In some embodiments, the truncated CD27 intracellular domain comprises a deletion of a lysine residue from amino acid position 1 to amino acid position 9 of the N-terminus of the CD27 intracellular domain. In some embodiments, the truncated CD27 intracellular domain comprises a lysine mutation at amino acid position 9 of the N-terminus of the CD27 intracellular domain. In some embodiments, the truncated CD27 intracellular domain comprises a deletion of one or more proximal basic amino acids from amino acid position 1 to amino acid position 9 of the N-terminus of the CD27 intracellular domain. In some embodiments, the truncated CD27 intracellular domain comprises one or more proximal basic amino acid mutation(s) from amino acid position 1 to amino acid position 9 of the N-terminus of the CD27 intracellular domain. In some embodiments, the truncated CD27 intracellular domain comprises one or more proximal basic amino acid mutation(s) at amino acid positions selected from amino acid positions 4 and 9 of the N-terminus of the CD27 intracellular domain.

In some embodiments, the transmembrane and intracellular domain comprise any one of SEQ ID NOs: 147, 193, and 240-243.

In some embodiments, the intracellular domain further comprises a third signal transduction domain, optionally wherein the third signal transduction domain is derived from a CD2 signaling domain, a MYD88 signaling domain, an interleukin 2 receptor binding (IL-2RB) protein signaling domain, or a combination thereof.

In some embodiments, the intracellular domain further comprises a fourth signal transduction domain, optionally wherein the fourth signal transduction domain is derived from a CD2 signaling domain, a MYD88 signaling domain, or an interleukin 2 receptor binding (IL-2RB) protein signaling domain or a combination thereof, and wherein the third and the fourth signal transduction domain are not identical.

In some embodiments, the cell further comprises a safety switch, optionally wherein the safety switch is selected from a truncated EGFR, an RQR8 protein, and an inducible Caspase-9.

In some embodiments, the truncated EGFR comprises SEQ ID NO:205 or 206.

In some embodiments, the cell is an immunomodulatory cell. In some embodiments, the immunomodulatory cell is a T cell, a natural killer T cell (NK-T cell), or a tumor infiltrating lymphocyte (TIL). In some embodiments, the T cell is an allogenic T cell. In some embodiments, the T cell is an autologous T cell. In some embodiments, the T cell is a naïve T cell, an early memory T cell, a stem cell-like T cell, a stem memory T cell (T_{SCM}), a central memory T cell (T_{CM}), or a regulatory T cell (T_{reg}).

In some embodiments, the cell further comprises a sequence encoding an artificial antigen receptor, a therapeutic polypeptide, or an immune cell modulatory protein, or a combination thereof. In some embodiments, the artificial antigen receptor comprises a chimeric antigen receptor (CAR) or an exogenous T cell receptor (TCR).

In some aspects, provided herein is a population of cells of any one of the above aspects or embodiments.

In some embodiments the population of cells is enriched. In some embodiments, the population is enriched for CD137-expressing cells from peripheral blood. In some embodiments, when the population comprises TILs, the population is enriched after stimulation with one or more tumor antigens.

In some aspects, provided herein is a composition comprising one or more isolated nucleic acids, wherein the one or more isolated nucleic acids encode: a) at least one chimeric PD-1 switch receptor, the chimeric PD-1 switch receptor comprising: (i) an extracellular domain that binds to PD-L1 or PD-L2; (ii) a transmembrane domain; and (iii) an intracellular domain; and b) a FAS dominant negative receptor (FASDD).

In some aspects, provided herein is a composition comprising one or more isolated oligopeptides, wherein the one or more isolated oligopeptides comprise: a) at least one chimeric PD-1 switch receptor, the chimeric PD-1 switch receptor comprising: (i) an extracellular domain that binds to PD-L1 or PD-L2; (ii) a transmembrane domain; and (iii) an intracellular domain; and b) a FAS dominant negative receptor (FASDD).

In some aspects, provided herein is an isolated nucleic acid encoding: a) a chimeric PD-1 switch receptor comprising: (i) an extracellular domain that binds to PD-L1 or PD-L2; (ii) a transmembrane domain; and (iii) an intracellular domain; and b) a FAS dominant negative receptor (FASDD).

In some embodiments, the isolated nucleic acid is a single nucleic acid. In some embodiments, the isolated nucleic acids comprise more than one nucleic acid, e.g., 2, 3, 4, or 5 or more nucleic acids.

In some embodiments, the isolated oligopeptide is an oligopeptide. In some embodiments, the isolated oligopeptides comprise more than one oligopeptide, e.g., 2, 3, 4, or 5 or more oligopeptides.

In some embodiments, the dominant negative FAS mutant comprises at least one modification (e.g., one, two, three, four, five or more modifications) in the cytoplasmic death domain or ligand binding domain of FAS.

In some embodiments, the FASDD comprises a deletion of amino acid residues 230-314 of SEQ ID NO:200 or a portion thereof. In some embodiments, the FASDD comprises SEQ ID NO:201. In some embodiments, the FASDD comprises a point mutation at position 260 of SEQ ID NO:50. In some embodiments, the point mutation is D260V. In some embodiments, the FASDD comprises SEQ ID NO:203. In some embodiments, the FASDD comprises one or more mutations selected from Thr13fs; Val15_Ala16insTer; Thr28Ala; Lys33Glu; Leu37Ter; His54Arg; Cys59Phe; His60fs; Pro62Arg; Cys73Gly; Asp78fs; Cys82Arg; Val83Met; Pro84Leu; His96Arg; Asp108Gly; Glu116Gly; Arg121Trp; Thr122Ile; Cys135Phe; Thr138Ile; Val139fs; Asp144Glu; Thr160Ala; Thr163Ile; Gly169Val; Gly175Glu; Leu177Arg; Cys178Tyr; Leu179Arg; Ile184Val; Val188fs; Lys193Arg; Glu194Lys; Asn206fs; His210Tyr; Ser212Thr; Thr214Ile, Thr214Asn; Val220fs, Val220Met; Ile222Met; Asn223His; Lys231fs; Tyr232Cys, Tyr232His; Thr234Ala; Thr235del; Thr241Lys, Thr241Pro; Gly247Ala; Val249Leu; Arg250Pro, Arg250Gln; Gly253Asp, Gly253Ser, Gly253Val; Val254Ala; Ala257Asp; Ile259Arg, Ile259Thr; Asp260Gly; Asp260Val, Asp260Tyr; Ile262Ser; Thr270Ile, Thr270Lys; Glu272Gly, Glu272Lys; Gln273Ter, Gln273 Arg, Glu289Asp; Ala290Glu; Leu294fs; Ala301Thr; Thr305Ile; Ile310Ser; Leu315Phe; Thr319Ile; Ser320Gly; Asp321Asn; Asn326Asp; Glu330fs; or a combination thereof, wherein the one or more mutations are relative to SEQ ID NO: 200.

In some embodiments, the extracellular domain is derived from PD-1. In some embodiments, the extracellular domain comprises SEQ ID NO:9.

In some embodiments, the transmembrane domain is derived from CD8, PD-1, CD28, ICOS, or IgG.

In some embodiments, the intracellular domain of PD-1 switch receptor is connected to the C-terminus of the transmembrane domain. In some embodiments, the intracellular domain comprises a first and at least a second signal transduction domain, wherein the first and the at least second signal transduction domains are non-identical. In some embodiments, the first signal transduction domain is derived from CD28, CD28H, ICOS, or a combination thereof and the at least second domain is derived from CD137 (4-1BB), CD134 (OX-40), CD40 (TNFRSF5), or CD27 (TNFRSF7).

In some embodiments, the at least second signal transduction domain comprises a mutant CD137 (4-1BB) intracellular domain or a mutant CD134 (OX-40) intracellular domain. In some embodiments, the mutant CD137 intracellular domain comprises a truncated CD137 intracellular domain. In some embodiments, the truncated CD137 intracellular domain comprises an amino acid sequence according to amino acid position 13 to amino acid position 42 of the CD137 intracellular domain; a deletion of a continuous stretch of one, two, three, four, five, six, seven, eight, nine, ten or more amino acids from the N-terminus the CD137 intracellular domain; or a deletion of one, two, three, four, five, six, seven, eight, nine, ten or more amino acids from amino acid position 1 to amino acid position 12 of the N-terminus of the CD137 intracellular domain. In some embodiments, the truncated CD137 intracellular domain comprises an amino acid sequence according to SEQ ID NO:3; a deletion of one, two, three or four lysine residue(s) from amino acid position 1 to amino acid position 12 of the N-terminus of the CD137 intracellular domain; one or more lysine mutation(s) from amino acid position 1 to amino acid position 12 of the N-terminus of the CD137 intracellular domain, e.g., one or more lysine mutation(s) at amino acid positions selected from amino acid positions 1, 5, 6 and 12 of the N-terminus of the CD137 intracellular domain; a deletion of one or more proximal basic amino acids from amino acid position 1 to amino acid position 12 of the N-terminus of the CD137 intracellular domain; and/or one or more proximal basic amino acid mutation(s) from amino acid position 1 to amino acid position 12 of the N-terminus of the CD137 intracellular domain, e.g., one or more proximal basic amino acid mutation(s) at amino acid positions selected from amino acid positions 1, 2, 3, 4, 5 and 6 of the N-terminus of the CD137 intracellular domain, e.g., a lysine mutation at amino acid position 12 of the N-terminus of the CD137 intracellular domain.

In some embodiments, the mutant CD134 intracellular domain comprises a truncated CD134 intracellular domain. In some embodiments, the truncated CD134 intracellular domain comprises an amino acid sequence according to amino acid position 15 to amino acid position 37 of the CD134 intracellular domain; a deletion of a continuous stretch of one, two, three, four, five, six, seven, eight, nine, ten, eleven, twelve, thirteen or more amino acids from the N-terminus of the CD134 intracellular domain; or a deletion of one, two, three, four, five, six, seven, eight, nine, ten, eleven, twelve, thirteen or more amino acids from amino acid position 1 to amino acid position 14 of the N-terminus of the CD134 intracellular domain. In some embodiments, the truncated CD134 intracellular domain comprises an amino acid sequence according to SEQ ID NO:6; a deletion of a lysine residue from amino acid position 1 to amino acid position 14 of the N-terminus of the CD134 intracellular domain; a lysine mutation at amino acid position 12 of the N-terminus of the CD134 intracellular domain; a deletion of one or more proximal basic amino acids from amino acid position 1 to amino acid position 14 of the N-terminus of the CD134 intracellular domain; and/or one or more proximal basic amino acid mutation(s) from amino acid position 1 to amino acid position 14 of the N-terminus of the CD134 intracellular domain. In some embodiments, the one or more proximal basic amino acid mutation(s) at amino acid positions are selected from amino acid positions 1, 2, and 5 of the N-terminus of the CD134 intracellular domain. In some embodiments, the mutant CD134 intracellular domain further comprises a lysine mutation at amino acid position 12 of the N-terminus of the CD134 intracellular domain.

In some embodiments, the mutant CD40 intracellular domain comprises a truncated CD40 intracellular domain. In some embodiments, the truncated CD40 intracellular domain comprises an amino acid sequence according to amino acid position 11 to amino acid position 62 of the CD40 intracellular domain. In some embodiments, the truncated CD40 intracellular domain comprises a deletion of a continuous stretch of one, two, three, four, five, six, seven, eight, nine, ten, or more amino acids from the N-terminus of the CD40 intracellular domain. In some embodiments, the truncated CD40 intracellular domain comprises a deletion of one or more proximal basic amino acids from amino acid position 1 to amino acid position 10 of the N-terminus of the CD40 intracellular domain. In some embodiments, the truncated CD40 intracellular domain comprises an amino acid sequence according to SEQ ID NO:239. In some embodiments, the truncated CD40 intracellular domain comprises a deletion of a lysine residue from amino acid position 1 to amino acid position 10 of the N-terminus of the CD40 intracellular domain. In some embodiments, the truncated CD40 intracellular domain comprises a lysine mutation at amino acid position 10 of the N-terminus of the CD40 intracellular domain. In some embodiments, the truncated CD40 intracellular domain comprises a deletion of one or more proximal basic amino acids from amino acid position 1 to amino acid position 10 of the N-terminus of the CD40 intracellular domain. In some embodiments, the truncated CD40 intracellular domain comprises or more proximal basic amino acid mutation(s) from amino acid position 1 to amino acid position 10 of the N-terminus of the CD40 intracellular domain, optionally one or more proximal basic amino acid mutation(s) at amino acid positions selected from amino acid positions 1, 2, 5, 6, and 10 of the N-terminus of the CD40 intracellular domain.

In some embodiments, the mutant CD27 intracellular domain comprises a truncated CD27 intracellular domain. In some embodiments, the truncated CD27 intracellular domain comprises an amino acid sequence according to amino acid position 9 to amino acid position 48 of the CD27 intracellular domain. In some embodiments, the truncated CD27 intracellular domain comprises a deletion of a continuous stretch of one, two, three, four, five, six, seven, eight, nine, or more amino acids from the N-terminus of the CD27 intracellular domain. In some embodiments, the truncated CD27 intracellular domain comprises a deletion of one or more proximal basic amino acids from amino acid position 1 to amino acid position 9 of the N-terminus of the CD27 intracellular domain. In some embodiments, the truncated CD27 intracellular domain comprises an amino acid sequence according to SEQ ID NO:226. In some embodiments, the truncated CD27 intracellular domain comprises a deletion of a lysine residue from amino acid position 1 to amino acid position 9 of the N-terminus of the CD27 intracellular domain. In some embodiments, the truncated CD27 intracellular domain comprises a lysine mutation at amino acid position 9 of the N-terminus of the CD27 intracellular domain. In some embodiments, the truncated CD27 intracellular domain comprises a deletion of one or more proximal basic amino acids from amino acid position 1 to amino acid position 9 of the N-terminus of the CD27 intracellular domain. In some embodiments, the truncated CD27 intracellular domain comprises one or more proximal basic amino acid mutation(s) from amino acid position 1 to amino acid position 9 of the N-terminus of the CD27 intracellular domain. In some embodiments, the truncated CD27 intracellular domain comprises one or more proximal basic amino acid mutation(s) at amino acid positions selected from amino acid positions 4 and 9 of the N-terminus of the CD27 intracellular domain.

In some embodiments, the transmembrane and intracellular domain comprise any one of SEQ ID NOs: 147, 193, and 240-243.

In some embodiments, the intracellular domain further comprises a third signal transduction domain. In some embodiments, the third signal transduction domain is derived from a CD2 signaling domain, a MYD88 signaling domain, an interleukin 2 receptor binding (IL-2RB) protein signaling domain, or a combination thereof.

In some embodiments, the intracellular domain further comprises a fourth signal transduction domain. In some embodiments, the fourth signal transduction domain is derived from a CD2 signaling domain, a MYD88 signaling domain or an interleukin 2 receptor binding (IL-2RB) protein signaling domain or a combination thereof. In some embodiments, the third and the fourth signal transduction domains are not identical.

In some embodiments, the cell or composition further comprises a safety switch. In some embodiments, the safety switch is selected from a truncated EGFR, an RQR8 protein, and an inducible Caspase-9. In some embodiments, the truncated EGFR comprises SEQ ID NO: 205 or SEQ ID NO:206.

In some embodiments, the PD-1 switch receptor and FASDD are within a single transgene. In some embodiments, the single transgene is post-translationally processed into two proteins.

In some embodiments, the PD-1 switch receptor, FASDD, and safety switch are within a single transgene. In some embodiments, the single transgene is post-translationally processed into three proteins.

In some aspects, provided herein is an isolated cell comprising the composition or the isolated nucleic acid of any one of the above aspects or embodiments.

In some embodiments, the cell is an immunomodulatory cell. In some embodiments, the immunomodulatory cell is a T cell, a natural killer T cell (NK-T cell), or a tumor infiltrating lymphocyte (TIL).

In some embodiments, the T cell is an allogenic T cell. In some embodiments, the T cell is a naïve T cell, an early memory T cell, a stem cell-like T cell, a stem memory T cell (T_{SCM}), a central memory T cell (T_{CM}), or a regulatory T cell (T_{reg}).

In some embodiments, the T cell is an autologous T cell. In some embodiments, the T cell is a naïve T cell, an early memory T cell, a stem cell-like T cell, a stem memory T cell (T_{SCM}), a central memory T cell (T_{CM}), or a regulatory T cell (T_{reg}).

In some embodiments, the cell further comprises a sequence encoding an artificial antigen receptor, a therapeutic polypeptide, or an immune cell modulatory protein, or a combination thereof.

In some embodiments, the artificial antigen receptor comprises a chimeric antigen receptor (CAR) or an exogenous T cell receptor (TCR).

In some aspects, provided herein is a population of cells of any one of the above aspects or embodiments.

In some embodiments, the population of cells is enriched. In some embodiments, the population is enriched for CD137-expressing cells from peripheral blood. In some embodiments, when the population comprises TILs, the population is enriched after stimulation with one or more tumor antigens.

In some aspects, provided herein is a pharmaceutical composition comprising an effective amount of the isolated cell of any one of the above aspects or embodiments, the population of cells of any one of the above aspects or embodiments, the composition of any one of the above aspects or embodiments, or the isolated nucleic acid of any one of the above aspects or embodiments, and a pharmaceutically acceptable carrier or excipient.

In some aspects, provided herein is a method of treating a disease or disorder in a subject in need thereof, the method comprising administering an effective amount of the isolated cell of any one of the above aspects or embodiments, the population of cells of any one of the above aspects or embodiments, the composition of any one of the above aspects or embodiments, the isolated nucleic acid of any one of the above aspects or embodiments, or the pharmaceutical composition of the above aspect to the subject.

In some embodiments, the disease or disorder is a cancer, an autoimmune disease or disorder, an infectious disease, an inflammatory disease, a renal disease or disorder, a lung disease or disorder, a liver disease or disorder, a cardiovascular system disease or disorder, a neurodegenerative disease or disorder, or a metabolic disease or disorder. In some embodiments, the cancer is a solid tumor or a hematologic cancer. In some embodiments, the solid cancer is a sarcoma, a carcinoma, or a melanoma. In some embodiments, the hematological cancer is a leukemia, a lymphoma, or a myeloma. In some embodiments, the cancer is selected from acute leukemia, acute lymphoblastic leukemia (ALL), acute lymphocytic leukemia, B cell, T cell or FAB ALL, acute myeloid leukemia (AML), acute myelogenous leukemia, chronic myelocytic leukemia (CML), chronic lymphocytic leukemia (CLL), hairy cell leukemia, myelodysplastic syndrome (MDS), Hodgkin's lymphoma, Hodgkin's disease, non-Hodgkin's lymphoma, multiple myeloma, colorectal carcinoma, pancreatic carcinoma, nasopharyngeal carcinoma, malignant histiocytosis, paraneoplastic syndrome/hypercalcemia of malignancy, bladder cancer, breast cancer, colorectal cancer, endometrial cancer, head cancer, neck cancer, hereditary nonpolyposis cancer, liver cancer, lung cancer, non-small cell lung cancer, ovarian cancer, pancreatic cancer, prostate cancer, renal cell carcinoma, testicular cancer, adenocarcinomas, sarcomas, malignant melanoma, and hemangioma. In some embodiments, the infectious disease is caused by a bacterium, a virus, a fungus, a protozoan, or a parasite. In some embodiments, the neurodegenerative disorder is Alzheimer's disease (AD) and other dementias, Parkinson's disease (PD) and PD-related disorders, prion disease, Motor neuron diseases (MND), Huntington's disease (HD), Spinocerebellar ataxia (SCA), or Spinal muscular atrophy (SMA). In some embodiments, the inflammatory disease is systemic lupus erythematosus, rheumatoid arthritis, inflammatory bowel disease, or multiple sclerosis.

### Definitions

Unless indicated otherwise, the terms “co-stimulatory molecule”, “costimulatory molecule”, “co stimulatory molecule”, “co-stimulatory protein”, “costimulatory protein”, “co stimulatory protein”, “co-stimulatory receptor”, “costimulatory receptor” “co stimulatory receptor” and “switch receptor” are used interchangeably, to refer to the recombinant T cell co-stimulatory receptors (RTCRs) comprising the chimeric co-stimulatory intracellular domains of the present application. These terms may be used in combination with terms such as “recombinant T cell”, “recombinant”, “chimeric T cell”, and “chimeric”, to refer to the RTCRs of the present application.

As described herein, “a recombinant T cell co-stimulatory receptor” or “switch receptor” of the present disclosure is a “costimulatory molecule” “co-stimulatory receptor” or “co-stimulatory protein” generated by operably linking an extracellular domain to an intracellular chimeric intracellular protein of the present disclosure.

As used herein, a “safety switch” refers to a molecule, e.g., a polypeptide, that selectively depletes cells expressing the ICS transgene cassette of the present disclosure. In some embodiments, a safety switch is a truncated EGFR.

“CD137” as described herein is a member of the tumor necrosis factor (TNF) receptor family, and also referred to as 4-1BB, CD137, tumor necrosis factor receptor superfamily member 9 (TNFRSF9) and induced by lymphocyte activation (ILA). As described herein, the terms “CD137”, “4-1BB”, “4-1BB wt”, “4-1BB wild type”, “BB”, “BB wt” and “BB wild type” are used interchangeably throughout, for example, when describing constructs or co-stimulatory molecules of the present application, unless otherwise indicated.

“CD134” as described herein is a member of the tumor necrosis factor (TNF) receptor family, and also referred to as OX-40, ACT35, IMD16, TXGPIL and tumor necrosis factor receptor superfamily member 4 (TNFRSF4). As described herein, the terms “CD134”, “OX-40”, “OX40”, “OX-40 wild type”, “OX-40 wt”, “OX40 wild type”, “OX40 wt”, “40”, “40 wild type” and “40 wt” are used interchangeably throughout, for example, when describing constructs or co-stimulatory molecules of the present application, unless otherwise indicated.

“CD27” as described herein is a member of the tumor necrosis factor (TNF) receptor family, and also referred to as S152, Tp55, T14, and tumor necrosis factor receptor superfamily member 7 (TNFRSF7). As described herein, the terms “CD27”, “S152”, “TNFRSF7”, “CD27 wild type”, “CD27 wt”, “CD27 wild type”, “27” and “CD27 wt” are used interchangeably throughout, for example, when describing constructs or co-stimulatory molecules of the present application, unless otherwise indicated.

“CD40” as described herein is a member of the tumor necrosis factor (TNF) receptor family, and also referred to as Bp50, P50, CDW40, B Cell-Associated Molecule, and tumor necrosis factor receptor superfamily member 5 (TNFRSF5). As described herein, the terms “CD40”, “Bp50”, “P50”, “CDW40”, “B Cell-Associated Molecule”, “CD40 wild type”, “CD40 wt”, “CD40 wild type”, “40” and “CD405 wt” are used interchangeably throughout, for example, when describing constructs or co-stimulatory molecules of the present application, unless otherwise indicated.

An “increased” or “enhanced” amount (e.g., with respect to cancer cell proliferation or expansion, antitumor response, cancer cell metastasis) refers to an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

A “decreased” or “reduced” or “lesser” amount (e.g., with respect to tumor size, cancer cell proliferation or growth) refers to a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein. In various embodiments, tumor burden is determined using linear dimensional methods (e.g., Response Evaluation Criteria in Solid Tumors (RECIST) v1.1 (Eisenhauer, et al., Eur J Cancer. (2009) 45 (2): 228-47). In various embodiments, tumor burden is determined using volumetric analysis (e.g., positron emission tomography (PET)/computed tomography (CT) scan). See, e.g., Paydary, et al., Mol Imaging Biol. (2019) 21 (1): 1-10; Li, et al., AJR Am J Roentgenol. (2021) 217 (6): 1433-1443; and Kerner, et al., EJNMMI Res. (2016) December; 6 (1): 33.

An “anti-tumor effect” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence or recurrence of a tumor, e.g., a relapse after remission.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound described in the present application which, upon administration to a subject, is capable of providing a compound described in this application or an active metabolite or residue thereof. As is known to those of skill in the art, “salts” of the compounds described in the present application may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, though not in themselves pharmaceutically acceptable, may be employed in the preparation of salts useful as intermediates in obtaining the compounds described in the application and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW4+, wherein W is C1-4 alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds described in the present application compounded with a suitable cation such as Nat, NH4+, and NW4+ (wherein W is a C1-4 alkyl group), and the like.

As used herein, the terms “specific binding,” “specifically binds to,” “specific for,” “selectively binds,” and “selective for,” with regard to the binding of a protein or antigen-binding fragment as described herein to a target molecule, a particular antigen (e.g., a polypeptide target), or an epitope on a particular antigen, mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the protein or antigen-binding fragment to the target molecule is competitively inhibited by the control molecule. A protein or antigen-binding fragment as described herein that “specifically binds to” or is “specific for” a particular polypeptide or an epitope on a particular polypeptide is one that binds to that particular polypeptide or epitope on a particular polypeptide without substantially binding to any other polypeptide or polypeptide epitope. In some instances, the protein or antigen-binding fragment as described herein specifically binds to an antigen, e.g., a polypeptide target, with dissociation constant (K_{D}) as described herein, for example, in the form of an scFv, Fab, or other form of a protein measured at a temperature of about 4° C., 25° C., 37° C., or 42° C. Affinities of a protein or antigen-binding fragment as described herein can be readily determined using conventional techniques, for example, those described by Scatchard et al., Ann. N. Y. Acad. Sci. USA, 51:660 (1949), ELISA assays, biolayer interferometry (BLI) assays, and surface plasmon resonance (SPR) assays. Binding properties of a protein or antigen-binding fragment as described herein to antigens, cells, or tissues thereof may generally be determined and assessed using immunodetection methods including, for example, immunofluorescence-based assays, such as immuno-histochemistry (IHC) and/or fluorescence-activated cell sorting (FACS). Generally, but not necessarily, reference to “binding” means “specific binding.”

Throughout the specification the term “signal domain”, “signaling domain”, and “signal transduction domain”, are used interchangeably, unless the context dictates otherwise.

The term “about” or “approximately” can be understood as within 10%, 9%, 8%, 7%, 6%, 5%, 4%, 3%, 2%, 1%, 0.5%, 0.1%, 0.05%, or 0.01% of the stated value. In some embodiments, “about” or “approximately” can be understood as within 5%, 4%, 3%, 2%, 1%, 0.5%, 0.1%, 0.05%, or 0.01% of the stated value. In some embodiments, “about” or “approximately” can be understood as within 2%, 1%, 0.5%, 0.1%, 0.05%, or 0.01% of the stated value. In some embodiments, “about” or “approximately” can be understood as within 1%, 0.5%, 0.1%, 0.05%, or 0.01% of the stated value.

Throughout the description, where compositions are described as having, including, or comprising specific components, or where processes and methods are described as having, including, or comprising specific steps, it is contemplated that, additionally, there are compositions described in the present application that consist essentially of, or consist of, the recited components, and that there are processes and methods according to the present application that consist essentially of, or consist of, the recited processing steps.

As a general matter, compositions specifying a percentage are by weight unless otherwise specified. Further, if a variable is not accompanied by a definition, then the previous definition of the variable controls.

The CD28 family proteins have a single extracellular immunoglobulin variable-like (IgV) domain followed by a short cytoplasmic tail. Members of the CD28 family proteins include CD28, CD28H, inducible costimulator (ICOS), cytotoxic T lymphocyte-associated antigen 4 (CTLA-4, CD152), program death-1 (PD-1), and B- and T-lymphocyte attenuator (BTLA). CD28, CD28H and ICOS are co-stimulatory proteins that are expressed on T cells that promote activation, high levels of cytokine/chemokine expression, resistance to apoptosis, and proliferation of T cells.

The Tumor Necrosis Factor Receptor (TNFR) family proteins includes TNFR1 (tumor necrosis factor receptor 1/TNFRSF1A), TNFR2 (tumor necrosis factor receptor 2/TNFRSF1B), lymphotoxin β receptor/TNFRSF3, OX40/TNFRSF4, CD40/TNFRSF5, Fas/TNFRSF6, decoy receptor 3/TNFRSF6B, CD27/TNFRSF7, CD30/TNFRSF8, 4-1BB/TNFRSF9, DR4 (death receptor 4/TNFRSF10A), DR5 (death receptor 5/TNFRSF10B), decoy receptor 1/TNFRSF10C, decoy receptor 2/TNFRSF10D, RANK (receptor activator of NF-kappa B/TNFRSF11A), OPG (osteoprotegerin/TNFRSF11B), DR3 (death receptor 3/TNFRSF25), TWEAK receptor/TNFRSF12A, TACI/TNFRSF13B, BAFF-R (BAFF receptor/TNFRSF13C), HVEM (herpes virus entry mediator/TNFRSF14), nerve growth factor receptor/TNFRSF16, BCMA (B cell maturation antigen/TNFRSF17, GITR (glucocorticoid-induced TNF receptor/TNFRSF18), TAJ (toxicity and JNK inducer/TNFRSF19), RELT/TNFRSF19L, DR6 (death receptor 6/TNFRSF21), TNFRSF22, TNFRSF23, ectodysplasin A2 isoform receptor/TNFRS27 and ectodysplasin 1-anhidrotic receptor. Interactions between tumor necrosis factor superfamily (TNFSF) ligands and TNF receptor superfamily (TNFRSF) receptors provide the co-stimulatory signals that control the survival, proliferation, differentiation, and effector function of immune cells. Depending upon the specific intracellular signal induced by TNFRSF members, they can be categorized into three groups-death domain (DD)-containing receptors, decoy receptors, and TNF receptor-associated factor (TRAF)-binding receptors. Some TNFRSFs such as TNFR-1, Fas, DR3, DR4, DR5, and DR6, contain their own DDs and/or interact with other cytoplasmic DD-containing adaptor molecules. Some other TNFRSFs, such as TNFR-2, CD27, CD30, CD40, glucocorticoid-induced TNFR family-related gene (GITR), Fn1, lymphotoxin beta-receptor (LTBR), OX40, receptor activator of NF-κB (RANK), and XEDAR, lack a DD and contain motifs with four to six amino acids called TRAF-interacting motifs (TIMs) which recruits TRAF proteins. TRAF proteins are adaptor molecules that activate multiple downstream signaling pathways such as NF-κB, Janus kinase (JNK), ERK, p38MAPK, and PI3K that help in cell survival, proliferation, and cytokine production. In some embodiments, the first signaling domain that is based on the intracellular signaling domain of a CD28 family protein is selected from a CD28 protein, ICOS protein or a combination thereof. In some embodiments, the at least second signaling domain is based on a mutant of the intracellular signaling domain of a TNFR family protein is selected from CD137 (4-1BB), CD134 (OX-40), CD40, and CD27.

In some aspects, provided herein are chimeric co-stimulatory intracellular domains based on the third-generation co-stimulatory domains of the present application. Reduced surface expression is a major hindrance in the development of chimeric co-stimulatory proteins for therapeutic purposes. The present disclosure provides chimeric co-stimulatory intracellular domains generated through mutations in the third-generation co-stimulatory domains of the present application that are both highly expressed and highly functional compared to the current second-generation and third-generation chimeric receptors that are effective in inducing costimulation. The chimeric co-stimulatory intracellular domains provided herein comprise: (a) a first signaling domain that is based on the intracellular signaling domain of a CD28 protein, ICOS protein or a combination thereof; and (b) at least a second signaling domain that is a mutant CD137 (4-1BB) intracellular domain, a wild type or mutant CD134 (OX-40) intracellular domain, a wild type or mutant CD40 intracellular domain, or a wild type or mutant CD27 intracellular domain. In some embodiments, the mutant CD137 (4-1BB) intracellular domain, the mutant CD134 (OX-40) intracellular domain, the mutant CD40 intracellular domain, or the mutant CD27 intracellular domain comprises a deletion, an insertion or a substitution of one or more amino acids in the membrane proximal portion of the CD137, CD134, CD40, or CD27 intracellular domain. In some embodiments, the one or more amino acids in the membrane proximal portion are ubiquitination sites involved in the ubiquitination and degradation of the CD137, CD134, CD40, or CD27 protein. In some embodiments, the mutant CD137 (4-1BB) intracellular domain, the mutant CD134 (OX-40) intracellular domain, the mutant CD40 intracellular domain, or the mutant CD27 intracellular domain comprises substitution or deletion of one or more lysine residues in the membrane proximal portion of the CD137, CD134, CD40, or CD27 intracellular domain. In some embodiments, the lysine residues are ubiquitination sites involved in the ubiquitination and degradation of the CD137, CD134, CD40, or CD27 protein. In some embodiments, the chimeric co-stimulatory intracellular domains provided herein further comprise a third signaling domain.

In some embodiments, the co-stimulatory intracellular domain of the present application can be combined or fused in frame with the extracellular domain of any known co-stimulatory protein, a cell intrinsic immune checkpoint inhibitor, a chimeric antigen receptor, an antibody or a portion thereof, a ligand or a receptor thereof, a cytokine or a receptor thereof, a chemokine or a receptor thereof or a complement receptor, to form a functional recombinant T cell co-stimulatory receptor (RTCR). In some embodiments, the RTCR can be expressed in a cell in combination with another T cell receptor (TCR), chimeric antigen receptor or co-stimulatory protein. A RTCR comprising the co-stimulatory intracellular domain disclosed herein, when co-expressed with a TCR in a T cell, significantly increases the cell surface expression of the RTCR, and/or cell proliferation, activation, persistence, cytokine production and/or effector function of the T cell, as compared to a second-generation co-stimulatory receptor.

A highly efficacious adoptive cell therapeutic targeting a shared and safe tumor associated antigen and comprising a cell-intrinsic inhibitor of T cell exhaustion able to withstand the suppressive tumor microenvironment is described in the present application. An exemplary chimeric molecule expressing the extracellular domain of PD-1 and a functionally optimized chimeric intracellular co-stimulatory domain are disclosed herein. Modified T cells expressing the chimeric molecule of the present disclosure are generated to show the efficacy of the chimeric molecule in enhancing T cell stimulation, activation and proliferation. Both molecules are expressed on the same T cell, creating a TCR-T product that responds robustly to tumor cells expressing both the cognate MHC/peptide complex and high levels of PD-L1/PD-L2.

For exemplification, a cell-intrinsic inhibitor of T cell exhaustion is developed by co-expression of third generation chimeric PD-1 receptors combined with T cell receptors targeting tumor associated or specific antigens to enhance the efficacy of T cell mediated killing of tumor cells. The 3^{rd} generation chimeric receptors disclosed herein can be used in combination with any endogenous or modified T cell receptors as well as with chimeric artificial receptors (CARs). The 3^{rd}-generation co-stimulatory molecules disclosed herein produce T cells with high physiological avidity and persistent proliferative potential, while negating negative signaling by PD-1, delivering instead co-stimulatory signals in a PD-L1 rich environment. The co-stimulatory molecule can be co-expressed with a tumor associated antigen (TAA) specific TCR and used to target PD-L1/PD-L2 and the TAA expressing tumors. This demonstrates that the synergistic effect between the TCR activation and co-stimulatory molecule significantly increases the therapeutic window and generates a potentially more effective candidate for clinical investigation. In the disclosure described below, the design of the third-generation chimeric proteins is systematically optimized, to further validate in vitro the improved anti-cancer effectiveness, and to investigate the in vivo anti-tumor efficacy.

Co-stimulatory molecules incorporating the extracellular domains of PD-1 with the intracellular domains of CD28, ICOS, CD27, CD40, CD134, and CD137 alone and in various combinations are generated. These sequences are optimized for surface expression and functionality by incorporating key mutations/deletions within the signaling domain of the chimeric receptors, focusing on the junction between CD28 and TNF-receptor family signaling domains. The functionality of these receptors is tested based on surface expression, in-vitro signaling, in-vitro T cell conjugation, cytokine production, proliferation, and cytotoxicity using a combination of soluble and plate-bound antibody stimulations and K562 target cells expressing PD-L1 or A375 tumor cells.

The disclosure herein provides an approach in which the TCR-T product co-expresses a chimeric co-stimulatory molecule alongside a recombinant TAA-specific TCR or an endogenous TCR. This approach allows for the targeting of the tumor associated antigen with simultaneous antagonization of checkpoint inhibition and delivery of co-stimulatory signals to the transfused T cell product. This approach not only results in a much-improved product, but also helps to develop a universal function-boosting platform for additional TCR-T products.

The key technical challenge hindering the clinical adoption of 3^{rd}-generation CARs combining the two domains has been the abnormally low expression of chimeric proteins at the cell surface and associated diminished functionality. This has held true in co-stimulatory molecules where the combination of the CD28 and CD137 signaling domains resulted in a poorly expressed and non-functional receptor (Ankri, 2013). Disclosed herein is a switch receptor/co-stimulatory molecule based on the ICOS/CD137 signaling domain and optimized for surface expression that is a significant improvement over past trials using the CD28 signaling domain alone, mediating both increased effector function and persistence of adoptively transferred cells. Results described herein show that the 3^{rd} generation co-stimulatory molecule disclosed herein produces T cells with high physiological avidity and persistent proliferation potential, while negating negative signaling by PD-1, delivering instead a co-stimulatory signal in a PD-L1 rich environment. The switch receptor/co-stimulatory molecule disclosed herein can be co-expressed with an endogenous TCR or a TAA specific TCR and used to target PD-L1/PD-L2 expressing tumors. This demonstrates that the synergistic effect between the TCR activation and co-stimulatory molecule significantly increases the therapeutic window for a potentially more effective candidate for clinical investigation.

Tumor associated antigens (“TAA”) and tumor specific antigens allow for the immunological targeting of the tumor with relatively minimal risk of off-tumor, on-target side effects. Tumor cells can upregulate these antigens which can then be targeted by the human immune response or ACT. Tumor infiltrating lymphocytes (“TILs”) may be a source of TAA-specific cells. The disclosure herein combines a co-stimulatory molecule based on 3^{rd}-generation CARs that exhibits superior functionality to CD28-based receptors with a new affinity enhanced TCR targeting TAAs to generate a TCR-T product that resists the suppressive function of the TME, and a FAS dominant negative receptor.

The present disclosure provides a recombinant T cell co-stimulatory receptor (RTCR), comprising: (a) an extracellular domain; (b) a transmembrane domain; and (c) a chimeric intracellular domain comprising a first and at least a second signal transduction domains, wherein the first and the at least second signal transduction domains are non-identical; and wherein the at least second signal transduction domain comprises a mutant intracellular signaling domain of a tumor necrosis factor receptor (TNFR) family protein.

In some embodiments, the mutant intracellular signaling domain of a TNFR family protein is any one of a mutant CD137 (4-1BB) intracellular domain, a WT or mutant CD134 (OX-40) intracellular domain, a WT or mutant CD40 intracellular domain, or a WT or mutant CD27 intracellular domain.

The present disclosure provides a recombinant T cell co-stimulatory receptor (RTCR), comprising: (a) an extracellular domain; (b) a transmembrane domain; and (c) a chimeric intracellular domain comprising a first and at least a second signal transduction domains, wherein the first and the at least second signal transduction domains are non-identical; and wherein the at least second signal transduction domain comprises a mutant CD137 (4-1BB) intracellular domain or a WT or mutant CD134 (OX-40) intracellular domain, a WT or mutant CD40 intracellular domain, or a WT or mutant CD27 intracellular domain.

### Compositions Comprising PD-1 Switch Receptor and FAS Dominant Negative Receptor

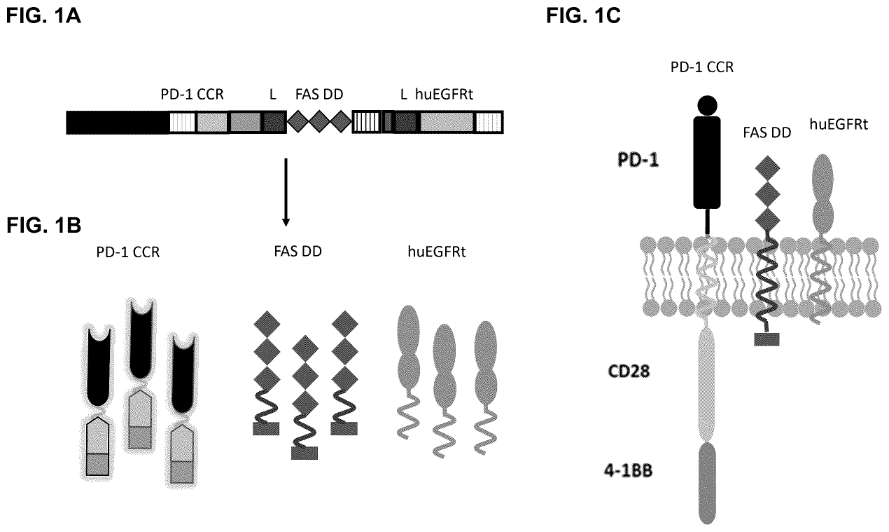

In some aspects, the present disclosure provides a synthetic Immune Checkpoint Switch (ICS) transgene cassette that can simultaneously express a PD-L1 targeting third-generation chimeric costimulatory receptor (i.e., a PD-1 switch receptor), a FAS dominant-negative receptor, and a safety switch (e.g., truncated EGFR) in T cells, referred to as “ICS transgene.” ICS transgene is a multi-component synthetic gene (see FIG. 1A). The ICS gene encodes a single polypeptide (FIG. 1B) that is co-translationally processed via ribosomal skipping at incorporated sites into multiple transmembrane proteins PD-1CCR and FASDD. In some embodiments, the single polypeptide further comprises a safety switch, e.g., huEGFRt.

In a non-limiting example, the third generation (3G) PD-1 Chimeric Co-stimulatory Receptor (also called chimeric PD-1 switch receptor, 3G PD-1CCR or PD-1 CCR, FIG. 1A-1C), is a chimeric protein made from the extracellular domain of human PD-1 protein, a transmembrane domain from human CD28 protein, and signaling domains from human CD28 and 4-1BB in cis configuration. The PD-1 CCR acts as a T cell co-stimulatory receptor that changes PD-L1 from an inhibitory ligand into a co-stimulatory agonist. In the tumor microenvironment (“TME”) expressing PD-L1, or PD-L2, PD-1 CCR binds to PD-L1/2, competitively subduing its activation of endogenous PD-1 while simultaneously stimulating PI3K and TRAF activities to increase effector functions and further overcome intracellular checkpoint signaling. Overall, T cells are lifted from inhibition of PD-1 and other checkpoint inhibitors in the TME and are directed to deliver potent tumor killing.

The FAS Dominant Negative Receptor, or FASDD (FIGS. 1A-1C), has a truncated intracellular death domain, thereby preventing it from initiating the downstream pro-apoptotic signaling cascade. The FASDD gets incorporated into the FAS trimer, alongside endogenous FAS, thereby preventing the function of the endogenous protein and acting as a dominant negative protein. Expression of FASDD can increase T cell survival by preventing FASL and/or TCR reactivation-induced cell apoptosis. As the FAS-mediated apoptotic pathway is active at higher levels of TCR signaling, the FASDD plays an important role in preventing the apoptosis of high-affinity T cell clones and apoptosis that may result in PD-1 rich environments due to signaling from the PD-1 CCR. Prevention of the FAS-mediated apoptosis leads to increased T cell proliferation and improved tumor-killing efficacy, while potentially limiting the need for large numbers of cells during adoptive transfer, as the potential of each cell is increased.

Truncated human EGFR, (“EGFRt” or “huEGFRt”), has a deletion of both extracellular and intracellular domains of wild-type EGFR to eliminate ligand binding and activation of intracellular signaling. Administration of anti-EGFR antibody drug cetuximab targets therapeutic T cells expressing huEGFRt and removes them from systematic circulation. Inclusion of huEGFRt in the ICS gene cassette can be used as a safety measure to eliminate or reduce adoptively transferred T cells if intolerable adverse events occur.

### PD-1 Switch Receptor

The present disclosure provides for a chimeric PD-1 switch receptor comprising: (i) an extracellular domain that binds to PD-L1 or PD-L2; (ii) a transmembrane domain; and (iii) an intracellular domain.

An extracellular domain that binds to PD-L1 or PD-L2 can comprise an extracellular domain from endogenous PD-1, an antigen-binding domain (e.g., an anti-PD-L1 or anti-PD-L2 antibody or antigen-binding fragment thereof), or an engineered binding domain. The extracellular domain that binds to PD-L1 or PD-L2 activates one or more intracellular costimulatory domain(s) when bound.

In some embodiments, the intracellular domain is selected from a CD137 intracellular domain, a CD134 intracellular domain, a CD27 intracellular domain, and a CD40 intracellular domain.

In some embodiments, the CD137 intracellular domain can be from a mammalian CD137. In some embodiments, the mammalian CD137 can be a human CD137, a mouse CD137, a rat CD137 or a monkey CD137. In some embodiments, the CD137 intracellular domain can be from a human CD137, or an isoform or a variant thereof, comprising an amino acid sequence identical to any one of the human CD137 amino acid sequence according to GenBank Accession Nos: U03397, AAA62478, NP_001552, Q07011, AAH06196 and XP 006710681. In some embodiments, the CD137 intracellular domain can be from a mouse CD137, or an isoform or a variant thereof, comprising an amino acid sequence identical to any one of the mouse CD137 amino acid sequence according to GenBank Accession Nos: NP_001070977.1, NP_001070976.1, NP_035742.1, NP_033430.1, P20334.1, XP 011248530.1, XP_011248530.1, ABI30213.1, BAE32724.1 and AAH28507.1. In some embodiments, the CD137 intracellular domain can be from a rat CD137, or an isoform or a variant thereof, comprising an amino acid sequence identical to any one of the rat CD137 amino acid sequence according to GenBank Accession Nos: NP_852049.1, NP_001020944.1, BAD99404.1, XP_008762504.1, XP_006239534.1, EDL81196.1, AAH97483.1, EHB16663.1, EHB16663.1, KFO38282.1, XP_010618177.1, XP_029414155.1, XP_029414154.1, XP_021099219.1 and XP_012888584.1. In some embodiments, the CD137 intracellular domain can be from a monkey CD137, or an isoform or a variant thereof, comprising an amino acid sequence identical to any one of the monkey CD137 amino acid sequence according to GenBank Accession Nos: ABY47575.1, ABI30212.1, ABY47577.1, ABY47576.1 and ABY47578.1.

In some embodiments, the CD137 intracellular domain, as described herein, comprises an amino acid sequence starting from the amino acid position 214 to the last amino acid at the C-terminal end of the amino acid sequence of the human CD137 protein, described herein. In some embodiments, the CD137 intracellular domain, as described herein, comprises an amino acid sequence starting from the amino acid position 215 to the last amino acid at the C-terminal end of the amino acid sequence of the mouse CD137 protein, described herein.

In some embodiments, the mutant CD137 intracellular domain described herein is from any one of the CD137 proteins as described herein, comprising one or more mutation(s), wherein the mutation can be addition/insertion, deletion/truncation or substitution/replacement of one or more amino acids within the amino acid sequence of the CD137 protein. In some embodiments, the mutant CD137 intracellular domain described herein is any one of the CD137 intracellular domain sequences, as described herein, comprising one or more mutation(s), wherein the mutation can be addition/insertion, deletion/truncation or substitution/replacement of one or more amino acids within the amino acid sequence of the CD137 intracellular domain. In some embodiments, the mutant CD137 intracellular domain described herein is a CD137 intracellular domain as described herein, comprising a deletion or substitution of one or more amino acids within the amino acid sequence of the CD137 intracellular domain that can be targets for ubiquitination. In some embodiments, the mutant CD137 intracellular domain described herein is a CD137 protein as described herein, comprising a deletion or substitution, of one or more lysine residues within the amino acid sequence of the CD137 intracellular domain that can be targets for ubiquitination. In some embodiments, the mutant CD137 intracellular domain described herein is a CD137 protein as described herein, comprising a deletion or substitution, of one, two, three or four lysine residues within the amino acid sequence of the CD137 intracellular domain that can be targets for ubiquitination. In some embodiments, the lysine residues within the amino acid sequence of the CD137 intracellular domain described herein, that can be deleted or substituted are at amino acid positions 214, 218, 219 and/or 225 of the CD137 intracellular domain.

In some embodiments, the mutant CD137 intracellular domain can be a truncated CD137 intracellular domain. A truncated CD137 intracellular domain as described herein can be any one of the CD137 proteins described herein, in which a continuous stretch of more than one, two, three, four, five, six, seven, eight, nine, ten, eleven, twelve, thirteen, fourteen, fifteen, twenty, twenty-five, fifty, hundred, two hundred or more amino acids are deleted from the N-terminus the CD137 protein as described herein. A truncated CD137 intracellular domain as described herein can be any one of the CD137 intracellular domain sequences described herein, in which a continuous stretch of more than one, two, three, four, five, six, seven, eight, nine, ten or more amino acids are deleted from the N-terminus the CD137 intracellular domain as described herein. In some embodiments, the amino acids deleted from the N-terminus the CD137 intracellular domain include one or more proximal polybasic amino acids of the CD137 intracellular domain.

In some embodiments, the mutant CD137 intracellular domain can be a truncated CD137 intracellular domain. In some embodiments, the truncated CD137 intracellular domain comprises an amino acid sequence according to amino acid position 13 to amino acid position 42 of the CD137 intracellular domain, of the present disclosure. In some embodiments, the truncated CD137 intracellular domain comprises a deletion of a continuous stretch of one, two, three, four, five, six, seven, eight, nine, ten or more amino acids from the N-terminus of the CD137 intracellular domain, of the present disclosure. In some embodiments, the truncated CD137 intracellular domain comprises a deletion of one, two, three, four, five, six, seven, eight, nine, ten or more amino acids from amino acid position 1 to amino acid position 12 of the N-terminus of the CD137 intracellular domain, of the present disclosure. In some embodiments, the truncated CD137 intracellular domain comprises a deletion of amino acid position 1 to amino acid position 12 of the N-terminus of the CD137 intracellular domain, of the present disclosure. In some embodiments, the CD137 intracellular domain comprises an amino acid sequence according to SEQ ID NO: 1.

In some embodiments, the truncated CD137 intracellular domain comprises an amino acid sequence according to SEQ ID NO: 3. In some embodiments, the truncated CD137 intracellular domain comprises an amino acid sequence having at least 50%, 55%, 60%, 65%, 70%, 75%, 80%, 85%, 90%, 95%, 97% or 99% identity to SEQ ID NO: 3.

A truncated CD137 intracellular domain as described herein, is referred to as “truncated CD137”, “CD137t”, “truncated 4-1BB”, “4-1BBt”, “truncated BB” or “BBt” interchangeably throughout, for example, when describing constructs or co-stimulatory molecules of the present application, unless otherwise indicated. In some embodiments, the mutant CD137 intracellular domain comprises a deletion of one, two, three or four lysine residue(s) from amino acid position 1 to amino acid position 12 of the N-terminus of the CD137 intracellular domain, of the present disclosure. In some embodiments, the mutant CD137 intracellular domain comprises one or more lysine mutation(s) from amino acid position 1 to amino acid position 12 of the N-terminus of the CD137 intracellular domain, of the present disclosure. In some embodiments, the mutant CD137 intracellular domain comprises one or more lysine mutation(s) at amino acid positions selected from amino acid positions 1, 5, 6 and 12 of the N-terminus of the CD137 intracellular domain, of the present disclosure. In some embodiments, the one or more lysine mutation(s) are lysine to alanine mutations. In some embodiments, the CD137 intracellular domain comprises an amino acid sequence according to SEQ ID NO: 1.

In some embodiments, the mutant CD137 intracellular domain comprises a deletion of one or more proximal basic amino acids from amino acid position 1 to amino acid position 12 of the N-terminus of the CD137 intracellular domain, of the present disclosure. In some embodiments, the mutant CD137 intracellular domain comprises one or more proximal basic amino acid mutation(s) from amino acid position 1 to amino acid position 12 of the N-terminus of the CD137 intracellular domain, of the present disclosure. In some embodiments, the mutant CD137 intracellular domain comprises one or more proximal basic amino acid mutation(s) at amino acid positions selected from amino acid positions 1, 2, 3, 4, 5 and 6 of the N-terminus of the CD137 intracellular domain, of the present disclosure. In some embodiments, the mutant CD137 intracellular domain comprising one or more proximal basic amino acid mutation(s), of the present disclosure, further comprises a lysine mutation at amino acid position 12 of the N-terminus of the CD137 intracellular domain, of the present disclosure. In some embodiments, the lysine mutation is a lysine to alanine mutation. In some embodiments, the CD137 intracellular domain comprises an amino acid sequence according to SEQ ID NO: 1.

In some embodiments, the CD134 intracellular domain can be from a mammalian CD134. In some embodiments, the mammalian CD134 can be a human CD134, a mouse CD134, a rat CD134 or a monkey CD134. In some embodiments, the CD134 intracellular domain can be from a human CD134, or an isoform or a variant thereof, comprising an amino acid sequence identical to any one of the human CD134 amino acid sequence according to GenBank Accession Nos: NP_003318, AAI05071, AAI05073, XP_016857721.1, XP 016857720.1, XP_011540377.1, XP_011540379.1, XP_011540378.1, XP_011540376.1, P43489.1, NP_001284491.1, NP_003317.1, EAW56278.1 and CAB96543.1. In some embodiments, the CD134 intracellular domain can be from a mouse CD134, or an isoform or a variant thereof, comprising an amino acid sequence identical to any one of the mouse CD134 amino acid sequence according to GenBank Accession Nos: NP_035789.1, AAI39267.1, AAI39240.1, NP_033478.1, XP_006538787.3, P47741.1, EDL15067.1, CAA79772.1, CAA59476.1, XP_021017102.2, and XP_021056714.1. In some embodiments, the CD134 intracellular domain can be from a rat CD134, or an isoform or a variant thereof, comprising an amino acid sequence identical to any one of the rat CD134 amino acid sequence according to GenBank Accession Nos: NP_035789.1, NP_037181.1, P15725.1, EDL81353.1, CAB96543.1, and CAA34897.1. In some embodiments, the CD134 intracellular domain can be from a monkey CD134, or an isoform or a variant thereof, comprising an amino acid sequence identical to any one of the monkey CD134 amino acid sequence according to GenBank Accession Nos: XP_010375483.1, XP_001090870.1, XP 021523144.1, XP_017750744.1, XP_003939714.1, XP_026313229.1, XP_026313228.1, XP 003890998.2, XP 025242473.1, XP 011768627.1, XP_005545179.1, XP_011886513.1, XP 011886512.1, XP_011857387.1 and XP_011811769.1.

In some embodiments, the CD134 intracellular domain, as described herein, comprises an amino acid sequence starting from amino acid position 241 to the last amino acid at the C-terminal end of the amino acid sequence of any one of the human CD134 protein, described herein. In some embodiments, the CD134 intracellular domain, as described herein, comprises an amino acid sequence starting from the amino acid position 236 to the last amino acid at the C-terminal of the amino acid sequence of the mouse CD134 protein, described herein.

In some embodiments, the mutant CD134 intracellular domain described herein is from any one of the CD134 proteins as described herein, comprising one or more mutation(s), wherein the mutation can be addition/insertion, deletion/truncation or substitution/replacement of one or more amino acids within the amino acid sequence of the CD134 protein. In some embodiments, the mutant CD134 intracellular domain described herein, is any one of the CD134 intracellular domain sequences as described herein, comprising one or more mutation(s), wherein the mutation can be addition/insertion, deletion/truncation or substitution/replacement of one or more amino acids within the amino acid sequence of the CD134 intracellular domain. In some embodiments, the mutant CD134 intracellular domain described herein is a CD134 intracellular domain as described herein, comprising a deletion or substitution of one or more amino acids within the amino acid sequence of the CD134 intracellular domain that can be targets for ubiquitination. In some embodiments, the mutant CD134 intracellular domain described herein is a CD134 protein as described herein, comprising a deletion or substitution, of one or more lysine residues within the amino acid sequence of the CD134 intracellular domain that can be targets for ubiquitination. In some embodiments, the mutant CD134 intracellular domain described herein is a CD134 protein as described herein, comprising a deletion or substitution, of one or two lysine residues within the amino acid sequence of the CD134 intracellular domain that can be targets for ubiquitination. In some embodiments, the lysine residues within the amino acid sequence of the CD134 intracellular domain described herein, that can be deleted or substituted are at amino acid positions 252 and/or 276 of the CD134 intracellular domain.

In some embodiments, the mutant CD134 intracellular domain can be a truncated CD134 intracellular domain. A truncated CD134 intracellular domain as described herein can be any one of the CD134 proteins described herein, in which a continuous stretch of more than one, two, three, four, five, six, seven, eight, nine, ten, eleven, twelve, thirteen, fourteen, fifteen, twenty, twenty-five, fifty, hundred, two hundred or more amino acids are deleted from the N-terminus the CD137 protein as described herein. A truncated CD134 intracellular domain as described herein can be any one of the CD134 intracellular domain sequences described herein, in which a continuous stretch of more than one, two, three, four, five, six, seven, eight, nine, ten, eleven, twelve, thirteen or more amino acids are deleted from the N-terminus the CD134 intracellular domain as described herein. In some embodiments, the amino acids deleted from the N-terminus the CD134 intracellular domain include one or more proximal polybasic amino acids of the CD134 intracellular domain.

In some embodiments, the truncated CD134 intracellular domain comprises an amino acid sequence according to amino acid position 15 to amino acid position 37 of a CD134 intracellular domain, of the present disclosure. In some embodiments, the truncated CD134 intracellular domain comprises a deletion of a continuous stretch of one, two, three, four, five, six, seven, eight, nine, ten, eleven, twelve, thirteen or more amino acids from the N-terminus of the CD134 intracellular domain, of the present disclosure. In some embodiments, the truncated CD134 intracellular domain comprises a deletion of one, two, three, four, five, six, seven, eight, nine, ten, eleven, twelve, thirteen or more amino acids from amino acid position 1 to amino acid position 14 of the N-terminus of the CD134 intracellular domain, of the present disclosure. In some embodiments, the truncated CD134 intracellular domain comprises a deletion of amino acid position 1 to amino acid position 14 of the N-terminus of the CD134 intracellular domain, of the present disclosure. In some embodiments, the CD134 intracellular domain comprises an amino acid sequence according to SEQ ID NO: 4.

In some embodiments, the mutant CD134 intracellular domain comprises an amino acid sequence according to SEQ ID NO: 6. In some embodiments, the mutant CD134 intracellular domain comprises an amino acid sequence having at least 50%, 55%, 60%, 65%, 70%, 75%, 80%, 85%, 90%, 95%, 97% or 99% identity to SEQ ID NO: 6.

A truncated CD134 intracellular domain as described herein, is referred to as “truncated CD134”, “CD134t”, “truncated OX-40”, “truncated OX40”, “OX-40t”, “OX40t” and “40t” are used interchangeably throughout, for example, when describing constructs or co-stimulatory molecules of the present application, unless otherwise indicated.

In some embodiments, the mutant CD134 intracellular domain comprises a deletion of a lysine residue from amino acid position 1 to amino acid position 14 of the N-terminus of the CD134 intracellular domain, of the present disclosure. In some embodiments, the mutant CD134 intracellular domain comprises a lysine mutation at amino acid position 12 of the N-terminus of the CD134 intracellular domain, of the present disclosure. In some embodiments, the lysine mutation is a lysine to alanine mutation. In some embodiments, the CD134 intracellular domain comprises an amino acid sequence according to SEQ ID NO: 4.

In some embodiments, the mutant CD134 intracellular domain comprises a deletion of one or more proximal basic amino acids from amino acid position 1 to amino acid position 14 of the N-terminus of the CD134 intracellular domain, of the present disclosure. In some embodiments, the mutant CD134 intracellular domain comprises one or more proximal basic amino acid mutation(s) from amino acid position 1 to amino acid position 14 of the N-terminus of the CD134 intracellular domain, of the present disclosure. In some embodiments, the mutant CD134 intracellular domain comprises one or more proximal basic amino acid mutation(s) at amino acid positions selected from amino acid positions 1, 2, and 5 of the N-terminus of the CD134 intracellular domain, of the present disclosure. In some embodiments, the mutant CD137 intracellular domain further comprises a lysine mutation at amino acid position 12 of the N-terminus of the CD137 intracellular domain, of the present disclosure. In some embodiments, the CD134 intracellular domain comprises an amino acid sequence according to SEQ ID NO: 4.

In some embodiments, the CD27 intracellular domain can be from a mammalian CD27. In some embodiments, the mammalian CD27 can be a human CD27, a mouse CD27, a rat CD27 or a monkey CD27. In some embodiments, the CD27 intracellular domain can be from a human CD27, or an isoform or a variant thereof, comprising an amino acid sequence identical to any one of the human CD27 amino acid sequence according to GenBank Accession Nos: NP_001233.2, NP_001400192.1, NP_001400193.1, NP_001400194.1, NP_001400195.1, NP_001400196.1, NP_001400197.1, XP_016875723.1, XP_047285856.1, and XP_011519344.1. In some embodiments, the CD27 intracellular domain can be from a mouse CD27, or an isoform or a variant thereof, comprising an amino acid sequence identical to any one of the mouse CD27 amino acid sequence according to GenBank Accession Nos: NP_001028298.1, NP_001036029.1, NP_001273682.1, XP_006505964.1, XP_006505963.1, XP 011239590.1, and XP_006505961.1. In some embodiments, the CD27 intracellular domain can be from a rat CD27, or an isoform or a variant thereof, comprising an amino acid sequence identical to any one of the rat CD27 amino acid sequence according to GenBank Accession Nos: NP 001019506.1 or XP_038964140.1. In some embodiments, the CD27 intracellular domain can be from a monkey CD27, or an isoform or a variant thereof, optionally comprising an amino acid sequence identical to any one of the monkey CD27 amino acid sequence according to GenBank Accession Nos: XP_508952.2, XP_063641489.1, and XP_054519965.1.

In some embodiments, the mutant CD27 intracellular domain described herein is from any one of the CD27 proteins as described herein, comprising one or more mutation(s), wherein the mutation can be addition/insertion, deletion/truncation or substitution/replacement of one or more amino acids within the amino acid sequence of the CD27 protein. In some embodiments, the mutant CD27 intracellular domain described herein, is any one of the CD27 intracellular domain sequences as described herein, comprising one or more mutation(s), wherein the mutation can be addition/insertion, deletion/truncation or substitution/replacement of one or more amino acids within the amino acid sequence of the CD27 intracellular domain. In some embodiments, the mutant CD27 intracellular domain described herein is a CD27 intracellular domain as described herein, comprising a deletion or substitution of one or more amino acids within the amino acid sequence of the CD27 intracellular domain that can be targets for ubiquitination. In some embodiments, the mutant CD27 intracellular domain described herein is a CD27 protein as described herein, comprising a deletion or substitution, of one or more lysine residues within the amino acid sequence of the CD27 intracellular domain that can be targets for ubiquitination. In some embodiments, the mutant CD27 intracellular domain described herein is a CD27 protein as described herein, comprising a deletion or substitution, of one or two lysine residues within the amino acid sequence of the CD27 intracellular domain that can be targets for ubiquitination.

In some embodiments, the mutant CD27 intracellular domain can be a truncated CD27 intracellular domain. A truncated CD27 intracellular domain as described herein can be any one of the CD27 proteins described herein, in which a continuous stretch of more than one, two, three, four, five, six, seven, eight, nine, ten, eleven, twelve, thirteen, fourteen, fifteen, twenty, twenty-five, or more amino acids are deleted from the N-terminus the CD27 protein as described herein. A truncated CD27 intracellular domain as described herein can be any one of the CD27 intracellular domain sequences described herein, in which a continuous stretch of more than one, two, three, four, five, six, seven, eight, nine, ten, eleven, twelve, thirteen or more amino acids are deleted from the N-terminus the CD27 intracellular domain as described herein. In some embodiments, the amino acids deleted from the N-terminus the CD27 intracellular domain includes one or more proximal polybasic amino acids of the CD27 intracellular domain.

In some embodiments, the truncated CD27 intracellular domain comprises a deletion of a continuous stretch of one, two, three, four, five, six, seven, eight, nine, ten, eleven, twelve, thirteen or more amino acids from the N-terminus of the CD27 intracellular domain, of the present disclosure.

In some embodiments, the mutant CD27 intracellular domain comprises an amino acid sequence according to SEQ ID NO: 226. In some embodiments, the mutant CD27 intracellular domain comprises an amino acid sequence having at least 50%, 55%, 60%, 65%, 70%, 75%, 80%, 85%, 90%, 95%, 97% or 99% identity to SEQ ID NO: 226.

A truncated CD27 intracellular domain as described herein, is referred to as “truncated CD27”, “CD27t”, “truncated TNFRSF7”, “truncated TNR7”, and “TNR7t” are used interchangeably throughout, for example, when describing constructs or co-stimulatory molecules of the present application, unless otherwise indicated.

In some embodiments, the CD40 intracellular domain can be from a mammalian CD40. In some embodiments, the mammalian CD40 can be a human CD40, a mouse CD40, a rat CD40 or a monkey CD40. In some embodiments, the CD40 intracellular domain can be from a human CD40, or an isoform or a variant thereof, comprising an amino acid sequence identical to any one of the human CD40 amino acid sequence according to GenBank Accession Nos: NP_001241.1, NP_001289682.1, NP_001309350.1, NP_001309351.1, NP 001349687.1, NP_001411268.1, NP_690593.1, XP_016883624.1, XP_016883625.1, XP 011527411.1, XP_047296557.1, XP_054180217.1, XP_054180218.1, XP_054180221.1, and XP 054180220.1. In some embodiments, the CD40 intracellular domain can be from a mouse CD40, or an isoform or a variant thereof, comprising an amino acid sequence identical to any one of the mouse CD40 amino acid sequence according to GenBank Accession Nos: NP_035741.2, NP_733803.2, NP_733804.1, NP_733805.1, XP_006499218.1, and XP 006499217.1. In some embodiments, the CD40 intracellular domain can be from a rat CD40, or an isoform or a variant thereof, comprising an amino acid sequence identical to any one of the rat CD40 amino acid sequence according to GenBank Accession Nos: NP_599187.1, XP_038960127.1, XP_008760685.1, XP_006235573.1, XP_038960128.1, XP 038960129.1, XP_008760686.1, XP_008760687.1, and XP_063139113.1. In some embodiments, the CD40 intracellular domain can be from a monkey CD40, or an isoform or a variant thereof, optionally comprising an amino acid sequence identical to any one of the monkey CD40 amino acid sequence according to GenBank Accession Nos: NP_001252791.1, XP_015004305.1, XP_028683146.1, XP_028683148.1, and XP 028683147.1.

In some embodiments, the mutant CD40 intracellular domain described herein is from any one of the CD40 proteins as described herein, comprising one or more mutation(s), wherein the mutation can be addition/insertion, deletion/truncation or substitution/replacement of one or more amino acids within the amino acid sequence of the CD40 protein. In some embodiments, the mutant CD40 intracellular domain described herein, is any one of the CD40 intracellular domain sequences as described herein, comprising one or more mutation(s), wherein the mutation can be addition/insertion, deletion/truncation or substitution/replacement of one or more amino acids within the amino acid sequence of the CD40 intracellular domain. In some embodiments, the mutant CD40 intracellular domain described herein is a CD40 intracellular domain as described herein, comprising a deletion or substitution of one or more amino acids within the amino acid sequence of the CD40 intracellular domain that can be targets for ubiquitination. In some embodiments, the mutant CD40 intracellular domain described herein is a CD40 protein as described herein, comprising a deletion or substitution, of one or more lysine residues within the amino acid sequence of the CD40 intracellular domain that can be targets for ubiquitination. In some embodiments, the mutant CD40 intracellular domain described herein is a CD40 protein as described herein, comprising a deletion or substitution, of one or two lysine residues within the amino acid sequence of the CD40 intracellular domain that can be targets for ubiquitination.

In some embodiments, the mutant CD40 intracellular domain can be a truncated CD40 intracellular domain. A truncated CD40 intracellular domain as described herein can be any one of the CD40 proteins described herein, in which a continuous stretch of more than one, two, three, four, five, six, seven, eight, nine, ten, eleven, twelve, thirteen, fourteen, fifteen, twenty, twenty-five, fifty, or more amino acids are deleted from the N-terminus the CD40 protein as described herein. A truncated CD40 intracellular domain as described herein can be any one of the CD40 intracellular domain sequences described herein, in which a continuous stretch of more than one, two, three, four, five, six, seven, eight, nine, ten, eleven, twelve, thirteen or more amino acids are deleted from the N-terminus the CD40 intracellular domain as described herein. In some embodiments, the amino acids deleted from the N-terminus the CD40 intracellular domain include one or more proximal polybasic amino acids of the CD40 intracellular domain.

In some embodiments, the truncated CD40 intracellular domain comprises a deletion of a continuous stretch of one, two, three, four, five, six, seven, eight, nine, ten, eleven, twelve, thirteen or more amino acids from the N-terminus of the CD40 intracellular domain, of the present disclosure.

In some embodiments, the mutant CD40 intracellular domain comprises an amino acid sequence according to SEQ ID NO: 239. In some embodiments, the mutant CD40 intracellular domain comprises an amino acid sequence having at least 50%, 55%, 60%, 65%, 70%, 75%, 80%, 85%, 90%, 95%, 97% or 99% identity to SEQ ID NO: 239.

A truncated CD40 intracellular domain as described herein, is referred to as “truncated CD40”, “CD40t”, “truncated TNFRSF5”, “truncated TNR5”, and “TNR5t” are used interchangeably throughout, for example, when describing constructs or co-stimulatory molecules of the present application, unless otherwise indicated.

TABLE 1
Amino acid sequences of second signal transduction domains of RTCR

| | |
|---|---|
| Human CD137/4-1BB | KRGRKKLLYIFKQPFMRPVQTTQEEDGCSCRFPEEEEGGCEL |
| (SEQ ID NO: 1) | |
| Mouse CD137/4-1BB | RKKFPHIFKQPFKKTTGAAQEEDACSCRCPQEEEGGGGGYEL |
| (SEQ ID NO: 2) | |
| truncated/mutated | QPFMRPVQTTQEEDGCSCRFPEEEEGGCEL |
| CD137/4-1BB (SEQ ID | |
| NO: 3) | |
| Human CD134/OX-40 | RRDQRLPPDAHKPPGGGSFRTPIQEEQADAHSTLAKI |
| (SEQ ID NO: 4) | |
| Mouse CD134 | RKAWRLPNTPKPCWGNSFRTPIQEEHTDAHFTLAKI |
| (SEQ ID NO: 5) | |
| truncated/mutated | GGGSFRTPIQEEQADAHSTLA |
| CD134/OX-40 (SEQ ID | |
| NO: 6) | |
| Human CD27 | QRRKYRSNKGESPVEPAEPCHYSCPREEEGSTIPIQEDYRKPEP |
| (SEQ ID NO: 7) | ACSP |
| Truncated CD27 | GESPVEPAEPCHYSCPREEEGSTIPIQEDYRKPEPACSP |
| (SEQ ID NO: 226) | |
| Human GITR | QLGLHIWQLRSQCMWPRETQLLLEVPPSTEDARSCQFPEEERGE |
| (SEQ ID NO: 8) | RSAEEKGRLGDLWV |
| TNFRSF4/OX40/CD134 | ALYLLRRDQRLPPDAHKPPGGGSFRTPIQEEQADAHSTLAKI |
| Signaling Domain | |
| (SEQ ID NO: 227) | |
| TNFRSF5 | KKVAKKPTNKAPHPKQEPQEINFPDDLPGSNTAAPVQETLHGCQ |
| (SEQ ID NO: 228) | PVTQEDGKESRISVQERQ |
| Truncated TNFRSF5 | APHPKQEPQEINFPDDLPGSNTAAPVQETLHGCQPVTQEDGKES |
| (SEQ ID NO: 239) | RISVQERQ |

Where applicable, membrane-proximal poly-basic regions are italicized.
Potential PI3K binding sites are bold and underlined. TRAF1/2 binding motifs,
major motif Px(Q/E)E and minor motifs Px(Q/E)x, are underlined. Potential
ubiquitination sites are in bold.

In some embodiments, the chimeric intracellular domain comprises a first signal transduction domain derived from a protein of the CD28 family. In some embodiments, the first signal transduction domain derived from any one of CD28, CD28H, ICOS or a combination thereof.

In some embodiments, the chimeric intracellular domain comprises a first signal transduction domain derived from ICOS protein.

The “ICOS protein” as described herein is an inducible T cell co-stimulatory protein, also referred to as AILIM, CD278, CCLP, CRP-1, H4, Ly115 and CVID1. In some embodiments, the ICOS intracellular domain can be from a mammalian ICOS. In some embodiments, the mammalian ICOS can be a human ICOS, a mouse ICOS, a rat ICOS or a monkey ICOS. In some embodiments, the ICOS intracellular domain can be from a human ICOS, or an isoform or a variant thereof, comprising an amino acid sequence identical to any one of the human ICOS amino acid sequence according to GenBank Accession Nos: AAH28006.1, NP_036224.1, AIC51287.1, AIC60036.1, NP_036224.1, Q9Y6W8.1, EAW70357.1, EAW70356.1, EAW70355.1, AAL40934.1, AAL40933.1, CAC06612.1, AAX93073.1, AAM00909.1, AAH28210.1 and CAD59742.1. In some embodiments, the ICOS intracellular domain can be from a mouse ICOS, or an isoform or a variant thereof, comprising an amino acid sequence identical to any one of the mouse ICOS amino acid sequence according to GenBank Accession Nos: NP_059508.2, Q9WVS0.2, EDL00161.1, CAM13242.1, CAM13241.1, CAB71153.1, AAG48732.1, AAH34852.1, XP_006496203.1, XP 006496202.1, XP 006496201.1, ACX50464.1, ACX50463.1, AAH28006.1, XP 021052880.1, XP_029334968.1 and XP_021030282.1. In some embodiments, the ICOS intracellular domain can be from a rat ICOS, or an isoform or a variant thereof, comprising an amino acid sequence identical to any one of the rat ICOS amino acid sequence according to GenBank Accession Nos: NP_072132.1, Q9R1T7.1, XP_008765358.1, XP_006245100.1, XP_006245099.1, EDL98922.1, EDL98921.1, XP_038940099.1, XP_032755449.1, XP 017457364.1, XP 006256324.1, XP_006256323.1, XP_006256322.1, XP_029425757.1, XP 029425757.1, XP 021119236.1, XP 012929934.1, XP 012867370.1 and XP 012867363.1. In some embodiments, the ICOS intracellular domain can be from a monkey ICOS, or an isoform or a variant thereof, comprising an amino acid sequence identical to any one of the monkey ICOS amino acid sequence according to GenBank Accession Nos: XP_007964137.1, NP_001253918.1, XP_010350939.1, XP_012301785.1, XP_012301784.1, XP_017739861.1, XP_010334714.1, XP_003925677.1, AFH29328.1, XP_008997520.1, XP_023075107.1, XP_023075099.1, XP_021779593.1, XP_003907887.1, XP_025260988.1, XP 025260987.1, XP_025260986.1, XP_011716287.1, XP_011716285.1, XP_005574075.1, XP 011903009.1, XP 011805288.1, XP_011805287.1, XP_011847867.1, XP_011847866.1, XP 017392362.1, XP_033086489.1, XP_032134414.1, XP_032134413.1, and XP 017802331.1.

In some embodiments, the human ICOS intracellular domain as described herein, comprises an amino acid sequence from amino acid position 133 to the last amino acid at the C-terminus of the amino acid sequence of the human ICOS protein, described herein. In some embodiments, the human ICOS intracellular domain as described herein, comprises an amino acid sequence from an amino acid position at one, two, three, four, five, six, seven, eight, nine, ten or more amino acids N-terminus to the amino acid position 133, to the last amino acid at the C-terminus of the amino acid sequence of the human ICOS protein, described herein. In some embodiments, the human ICOS intracellular domain as described herein, comprises an amino acid sequence from amino acid position 133 to an amino acid position at one, two, three, four, five, six, seven, eight, nine, ten or more amino acids N-terminus to the last amino acid at the C-terminus of the amino acid sequence of the human ICOS protein, described herein. In some embodiments, the human ICOS intracellular domain as described herein, comprises an amino acid sequence from an amino acid position at one, two, three, four, five, six, seven, eight, nine, ten or more amino acids N-terminus to the amino acid position 133, to an amino acid position at one, two, three, four, five, six, seven, eight, nine, ten or more amino acids N-terminus to the last amino acid at the C-terminus of the amino acid sequence of the human ICOS protein, described herein.

In some embodiments, the human ICOS (28) intracellular domain as described herein, comprises a portion of the ICOS domain amino acid sequence from amino acid position 133 to amino acid position 183, and a portion of the ICOS domain amino acid sequence from amino acid position 184 to the last amino acid at the C-terminus of the amino acid sequence of the human ICOS protein, described herein. In some embodiments, the human ICOS (28) intracellular domain as described herein, comprises a portion of the ICOS domain amino acid sequence from an amino acid position at one, two, three, four, five, six, seven, eight, nine, ten or more amino acids N-terminus to the amino acid position 133, to amino acid position 183 of the human ICOS protein, described herein. In some embodiments, the human ICOS (28) intracellular domain as described herein, comprises a portion of the ICOS domain amino acid sequence from amino acid position 133, to an amino acid position at one, two, three, four, five, six, seven, eight, nine, ten or more amino acids C-terminus to the amino acid position 183 of the human ICOS protein, described herein. In some embodiments, the human ICOS (28) intracellular domain as described herein, comprises a portion of the ICOS domain amino acid sequence from an amino acid position at one, two, three, four, five, six, seven, eight, nine, ten or more amino acids N-terminus to the amino acid position 133, to an amino acid position at one, two, three, four, five, six, seven, eight, nine, ten or more amino acids C-terminus to the amino acid position 183 of the human ICOS protein, described herein.

The “CD28 protein”, also referred to as Tp44, is a constitutively expressed receptor for CD80 (B7.1) and CD86 (B7.2) proteins on naïve T cells and is important for T cell activation. In some embodiments, the CD28 intracellular domain can be from a mammalian CD28. In some embodiments, the mammalian CD28 can be a human CD28, a mouse CD28, a rat CD28 or a monkey CD28. In some embodiments, the CD28 intracellular domain can be from a human CD28, or an isoform or a variant thereof, comprising an amino acid sequence identical to any one of the human CD28 amino acid sequence according to GenBank Accession Nos: P10747.1, NP_001230007.1, NP_001230006.1, NP_006130.1, EAW70350.1, EAW70349.1, EAW70348.1, EAW70347.1, AIC48451.1, CAC29237.1, AAA51945.1, AAA51944.1, AAL40931.1, AAF33794.1, AAF33793.1, AAF33792.1, XP_011510499.1, XP_011510497.1, XP 011510496.1, AAI12086.1, AAH93698.1, ABK41938.1, AAY24123.1, CAD57003.1 and AAA60581. In some embodiments, the CD28 intracellular domain can be from a mouse CD28, or an isoform or a variant thereof, comprising an amino acid sequence identical to any one of the mouse CD28 amino acid sequence according to GenBank Accession Nos: AAA37396.1, NP_031668.3, P31041.2, AAH64058.1, EDL00156.1, CAM13249.1, XP_036012281.1, XP 021054806.1, XP_021027481.1, XP_036015651.1, and XP_030104805. In some embodiments, the CD28 intracellular domain can be from a rat CD28, or an isoform or a variant thereof, comprising an amino acid sequence identical to any one of the rat CD28 amino acid sequence according to GenBank Accession Nos: CAA39003.1, NP_037253.2, P31042.1, XP_008765300.1, EDL98926.1, XP_032755445.1, XP_034354910.1, XP_019061859.2, XP_008844474.1, XP 004851403.1 and XP 012865504.1. In some embodiments, the CD28 intracellular domain can be from a monkey CD28, or an isoform or a variant thereof, comprising an amino acid sequence identical to any one of the monkey CD28 amino acid sequence according to GenBank Accession Nos: ABH06891.1, ABH08508.1, ABH06892.1, ABH08509.1, ABQ09493.1, NP 001274262.1, NP_001036106.2, ABG77998.1, ABG77997.1 and XP_014966207.1.

The “CD28H protein”, also referred to CD28 homolog, transmembrane and immunoglobulin domain-containing protein 2, has co-stimulatory activity in T cells by binding to B7H7. CD28H was initially described as a molecule involved in cell-cell interaction, cell migration, and angiogenesis of epithelial and endothelial cells (7, 8). CD28H has a single extracellular immunoglobulin domain followed by a transmembrane domain and a 110 amino acid-long cytoplasmic region. In some embodiments, the CD28 intracellular domain can be from a mammalian CD28H. In some embodiments, the mammalian CD28 can be a human CD28H, a mouse CD28H, a rat CD28H or a monkey CD28H. In some embodiments, the CD28H intracellular domain can be from a human CD28H, or an isoform or a variant thereof, comprising an amino acid sequence identical to any one of the human CD28H amino acid sequence according to GenBank Accession Nos: NP_001295161.1, NP_001162597.1, Q96BF3.2, XP_024307127.1 and XP 016881773.1.

In some embodiments, the human CD28 intracellular domain as described herein, comprises an amino acid sequence from amino acid position 145 to the last amino acid at the C-terminus of the amino acid sequence of the human CD28 protein, described herein. In some embodiments, a portion of the human CD28 intracellular domain as described herein, can comprise an amino acid sequence from about amino acid position 195 to about amino acid position 212 of the amino acid sequence of the human CD28 protein, described herein. In some embodiments, a portion of the human CD28 intracellular domain as described herein, can comprises an amino acid sequence from one, two, three, four, five, six, seven, eight, nine or 10 or more amino acid amino acid position N-terminus to amino acid position 195 to one, two, three, four, five, six, seven, eight, nine or 10 or more amino acid amino acid position C-terminus amino acid position 220 of the amino acid sequence of the human CD28 protein, described herein.

In some embodiments, the first signal transduction domain derived from ICOS comprises an amino acid sequence according to SEQ ID NO: 9. In some embodiments, the first signal transduction domain derived from ICOS comprises an amino acid sequence having at least 50%, 55%, 60%, 65%, 70%, 75%, 80%, 85%, 90%, 95%, 97% or 99% identity to SEQ ID NO: 9.

In some embodiments, the chimeric intracellular domain comprises a first signal transduction domain comprising a portion of a CD28 intracellular domain combined with an ICOS protein (ICOS (28) domain) according to SEQ ID NO: 9. In some embodiments, the ICOS (28) domain comprises the portion of CD28 intracellular domain inserted N-terminal to the PI-3K binding site of the ICOS protein according to SEQ ID NO: 9. In some embodiments, the ICOS (28) domain comprises the portion of CD28 inserted at 1, 2, 3, 4 or 5 amino acid position N-terminal to the PI-3K binding site of the ICOS protein according to SEQ ID NO: 9. In some embodiments, the ICOS (28) domain comprises the portion of CD28 inserted C-terminal to the PI-3K binding site of the ICOS protein according to SEQ ID NO: 9. In some embodiments, the ICOS (28) domain comprises the portion of CD28 inserted at 1, 2, 3, 4 or 5 amino acid position C-terminal to the PI-3K binding site of the ICOS protein according to SEQ ID NO: 9.

In some embodiments, the portion of CD28 is inserted at any amino acid position before amino acid position 48 within an ICOS protein of amino acid sequence according to SEQ ID NO: 9. In some embodiments, the portion of CD28 is inserted at any amino acid position between amino acid position 1 and amino acid position 48, within an ICOS protein of amino acid sequence according to SEQ ID NO: 9. In some embodiments, the portion of CD28 is inserted between the amino acid position 47 and amino acid position 48 of an ICOS protein with the amino acid sequence according to SEQ ID NO: 9. In some embodiments, the portion of CD28 is inserted between the amino acid position 46 and amino acid position 47 of an ICOS protein with the amino acid sequence according to SEQ ID NO: 9. In some embodiments, the portion of CD28 is inserted between the amino acid position 45 and amino acid position 46 of an ICOS protein with the amino acid sequence according to SEQ ID NO: 9. In some embodiments, the portion of CD28 is inserted between the amino acid position 44 and amino acid position 45 of an ICOS protein with the amino acid sequence according to SEQ ID NO: 9. In some embodiments, the portion of CD28 is inserted between the amino acid position 43 and amino acid position 44 of an ICOS protein with the amino acid sequence according to SEQ ID NO: 9.

In some embodiments, the portion of CD28 is inserted at any position after amino acid position 51 within an ICOS protein of amino acid sequence according to SEQ ID NO: 9. In some embodiments, the portion of CD28 is inserted at any amino acid position between amino acid position 51 and amino acid position 67, within an ICOS protein of amino acid sequence according to SEQ ID NO: 9. In some embodiments, the portion of CD28 is inserted between the amino acid position 51 and amino acid position 52 of an ICOS protein with the amino acid sequence according to SEQ ID NO: 9. In some embodiments, the portion of CD28 is inserted between the amino acid position 53 and amino acid position 54 of an ICOS protein with the amino acid sequence according to SEQ ID NO: 9. In some embodiments, the portion of CD28 is inserted between the amino acid position 54 and amino acid position 55 of an ICOS protein with the amino acid sequence according to SEQ ID NO: 9. In some embodiments, the portion of CD28 is inserted between the amino acid position 56 and amino acid position 57 of an ICOS protein with the amino acid sequence according to SEQ ID NO: 9. In some embodiments, the portion of CD28 is inserted between the amino acid position 57 and amino acid position 58 of an ICOS protein with the amino acid sequence according to SEQ ID NO: 9.

In some embodiments, the portion of CD28 of the ICOS (28) domain disclosed herein comprises an amino acid sequence according to amino acid position 51 to amino acid position 68 of a CD28 signaling domain according to SEQ ID NO: 10. In some embodiments, the portion of CD28 of the ICOS (28) domain disclosed herein comprises an amino acid sequence according to amino acid position 51 to amino acid position 76 of a full length CD28 signaling domain according to SEQ ID NO: 10. In some embodiments, the portion of CD28 of the ICOS (28) domain disclosed herein comprises an amino acid sequence according to amino acid position 45 to amino acid position 68 of a CD28 signaling domain according to SEQ ID NO: 10. In some embodiments, the portion of CD28 inserted within the ICOS (28) domain comprises a PRRP motif. In some embodiments, the portion of CD28 inserted within the ICOS (28) domain comprises an amino acid sequence according to SEQ ID NO: 11. In some embodiments, the portion of CD28 inserted within the ICOS (28) domain comprises an amino acid sequence having at least 50%, 55%, 60%, 65%, 70%, 75%, 80%, 85%, 90%, 95%, 97% or 99% identity to SEQ ID NO: 11.

In some embodiments, the ICOS (28) domain comprises an amino acid sequence according to SEQ ID NO: 12. In some embodiments, the ICOS (28) comprises an amino acid sequence having at least 50%, 55%, 60%, 65%, 70%, 75%, 80%, 85%, 90%, 95%, 97% or 99% identity to SEQ ID NO: 12.

In some embodiments, the chimeric intracellular domain comprises a first signal transduction domain derived from CD28. In some embodiments, the first signal transduction domain derived from CD28 comprises an amino acid sequence according to SEQ ID NO: 10. In some embodiments, the first signal transduction domain derived from CD28 comprises an amino acid sequence having at least 50%, 55%, 60%, 65%, 70%, 75%, 80%, 85%, 90%, 95%, 97% or 99% identity to SEQ ID NO: 10.

A signal transduction domain derived from CD28 as described herein, is referred to as “CD28” or “28”, interchangeably throughout.

```
TABLE 2
Amino acid sequences of first intracellular signaling domains.
ICOS signaling domain (Other name: ICOS): Stalk (underlined),
TM (regular font), intracellular domain IC)(bold) and PI-3K
binding site (bold and underlined)
                                                                                                          (SEQ ID NO: 9)
SQLCCQLKFWLPIGCAAFVVVCILGCILICWLTKKKYSSSVHDPNGEYMFMRAVNTAKKSRLTDVTL
CD28 Transmembrane_CD28 Signaling Domain (Other names: CD28 or 28
CD28 Stalk (underlined), TM (regular font), intracellular domain
(IC)(bold), PI3K regulatory subunit binding, GRB2, GADS association
domain (bold and dotted-underlined), ITK interaction site (bold and
double-underlined,GRB2, GADS, LCK interaction site (bold and dash-
underlined)

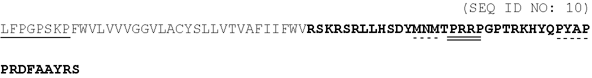

CD28 fragment (Other names: mini-CD28 or (28)) PRRP motif in bold
      (SEQ ID NO: 11)
TPRRPGPTRKHYQPYAPP
ICOS (28) domain (Other name: ICOS (mini-CD28)) intracellular
domain (italicized), TBK1 binding site (TRAF-like motif)
(italicized and dotted-underlined), PI-3K regulatory subunit
binding site (italicized and underlined), CD28 portion (bold),
distal domain (italicized and dash-underlined)

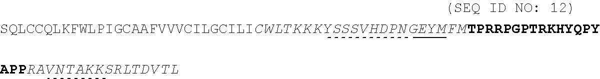

```

In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 13. In some embodiments, the chimeric intracellular domain comprises an amino acid sequence according to any one of SEQ ID NOs: 14-17. In some embodiments, the chimeric intracellular domain comprises an amino acid sequence having at least 50%, 55%, 60%, 65%, 70%, 75%, 80%, 85%, 90%, 95%, 97% or 99% identity to any one of SEQ ID NOs: 14-17. In some embodiments, the chimeric intracellular domain comprises an amino acid sequence according to SEQ ID NO: 14. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 14. In some embodiments, the chimeric intracellular domain comprises an amino acid sequence according to SEQ ID NO: 15. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 15. In some embodiments, the chimeric intracellular domain comprises an amino acid sequence according to SEQ ID NO: 16. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 16. In some embodiments, the chimeric intracellular domain comprises an amino acid sequence according to SEQ ID NO: 17. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 17.

In some embodiments, the chimeric intracellular domain comprises an amino acid sequence according to any one of SEQ ID NOs: 120-129. In some embodiments, the chimeric intracellular domain comprises an amino acid sequence having at least 50%, 55%, 60%, 65%, 70%, 75%, 80%, 85%, 90%, 95%, 97% or 99% identity to any one of SEQ ID NOs: 120-129. In some embodiments, the chimeric intracellular domain comprises an amino acid sequence according to SEQ ID NO: 120. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 120. In some embodiments, the chimeric intracellular domain comprises an amino acid sequence according to SEQ ID NO: 121. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 121. In some embodiments, the chimeric intracellular domain comprises an amino acid sequence according to SEQ ID NO: 122. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 122.

In some embodiments, the chimeric intracellular domain comprises an amino acid sequence according to SEQ ID NO: 123. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 123. In some embodiments, the chimeric intracellular domain comprises an amino acid sequence according to SEQ ID NO: 124. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 124. In some embodiments, the chimeric intracellular domain comprises an amino acid sequence according to SEQ ID NO: 125. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 125. In some embodiments, the chimeric intracellular domain comprises an amino acid sequence according to SEQ ID NO: 126. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 126. In some embodiments, the chimeric intracellular domain comprises an amino acid sequence according to SEQ ID NO: 127. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 127. In some embodiments, the chimeric intracellular domain comprises an amino acid sequence according to SEQ ID NO: 128. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 128. In some embodiments, the chimeric intracellular domain comprises an amino acid sequence according to SEQ ID NO: 129. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 129.

TABLE 3
Amino acid sequences of chimeric intracellular domains of RTCR.

ICOS Transmembrane_ICOS Signaling Domain_4-1BB Signaling Domain (other names:
ICOS-4-1BB (CD137) intracellular domain, ICOS-137; ICOS_137; ICOS137; ICOS_BB;
ICOS-BB; ICOSBB; ICOSBBwt; ICOS_BBwt or ICOS_BB wild type) (SEQ ID NO: 13):
ICOS sequence underlined and 4-1BB (BB) domain in normal font
SQLCCQLKFWLPIGCAAFVVVCILGCILICWLTKKKYSSSVHDPNGEYMFMRAVNTAKKSR
LTDVTLKRGRKKLLYIFKQPFMRPVQTTQEEDGCSCRFPEEEEGGCEL
ICOS Transmembrane_ICOS Signaling Domain_Truncated 4-1BB Signaling Domain
(other names: ICOS-truncated 4-1BB (CD137) intracellular domain; ICOS_137t;
ICOS137t; ICOS-137t; ICOS_BBt; ICOSBBt or ICOS-BBt) (SEQ ID NO: 14): ICOS
sequence underlined and mutated/truncated 4-1BB (BBt) domain in normal font
SQLCCQLKFWLPIGCAAFVVVCILGCILICWLTKKKYSSSVHDPNGEYMFMRAVNTAKKSR
LTDVTLQPFMRPVQTTQEEDGCSCRFPEEEEGGCEL
ICOS Transmembrane_ICOS Signaling Domain_Truncated OX-40 Signaling Domain
(other names: ICOS-truncated OX-40 (CD134) intracellular domain, ICOS_OX40t; ICOS-
OX40t; ICOS_40t; ICOS40t or ICOS-40t (SEQ ID NO: 15): ICOS sequence underlined
and mutated/truncated OX-40 (OX40t, 40t) domain in normal font
SQLCCQLKFWLPIGCAAFVVVCILGCILICWLTKKKYSSSVHDPNGEYMFMRAVNTAKKSR
LTDVTLGGGSFRTPIQEEQADAHSTLA
ICOS Transmembrane_ICOS Signaling Domain (mini-CD28)_Truncated 4-1BB Signaling
Domain (other names: ICOS(28)-truncated 4-1BB (BBt) intracellular domain,
ICOS(28)_BBt; ICOS(28)BBt; ICOS(28)-BBt; ICOS(28)_4-1BBt; ICOS(28)4-1BBt or
ICOS(28)-4-1BBt) (SEQ ID NO: 16): ICOS sequence underlined, CD28 portion in bold
and BBt domain in normal font
SQLCCQLKFWLPIGCAAFVVVCILGCILICWLTKKKYSSSVHDPNGEYMFMTPRRPGPTRK
HYQPYAPPRAVNTAKKSRLTDVTLQPFMRPVQTTQEEDGCSCRFPEEEEGGCEL
ICOS Transmembrane_ICOS Signaling Domain (mini-CD28)_Truncated OX-40 Signaling
Domain (other names: ICOS(28)-truncated OX-40; ICOS(28)-OX40t; ICOS(28)_OX40t;
ICOS(28)OX40t; ICOS (28)-40t; ICOS (28)_40t or ICOS(28)40t) (CD134) intracellular
domain (SEQ ID NO: 17): ICOS sequence underlined, CD28 portion in bold and OX40
(OX-40t, 40t) domain in normal font
SQLCCQLKFWLPIGCAAFVVVCILGCILICWLTKKKYSSSVHDPNGEYMFMTPRRPGPTRK
HYQPYAPPRAVNTAKKSRLTDVTLGGGSFRTPIQEEQADAHSTLA
ICOS(28) (shortened ICOS(28)) (SEQ ID NO: 109): ICOS Transmembrane_ICOS
Signaling Domain (mini-CD28)
CWLTKKKYSSSVHDPNGEYMFMTPRRPGPTRKHYQPYAPPRAVNTAKKSRLTDV
TL
CD28 Transmembrane_CD28 Signaling Domain_4-1BB Signaling Domain (other names:
CD28_BBwt signaling domain; CD28_BB; CD28BB; CD28-BB; 28_BBwt; 28BB; 28_BB
or 28-BB) (SEQ NO: 120): CD28 Transmembrane domain_CD28 Signaling Domain_4-
1BB Signaling Domain
LFPGPSKPFWVLVVVGGVLACYSLLVTVAFIIFWVRSKRSRLLHSDYMNMTPRRPGPTRKH
YOPYAPPRDFAAYRSKRGRKKLLYIFKQPFMRPVQTTQEEDGCSCRFPEEEEGGCEL
CD28 Transmembrane_CD28 Signaling Domain_truncated 4-1BB Signaling Domain
(other names: CD28_BBt signaling domain; CD28BBt; CD28-BBt; 28_BBt; 28BB; or 28-
BBt) (SEQ ID NO: 121):
LFPGPSKPFWVLVVVGGVLACYSLLVTVAFIIFWVRSKRSRLLHSDYMNMTPRRPGPTRKH
YQPYAPPRDFAAYRSQPFMRPVQTTQEEDGCSCRFPEEEEGGCEL
CD28 Transmembrane_CD28 Signaling Domain_truncated OX-40 Signaling Domain
(other names: CD28_OX40t signaling domain; 28-OX40t; 28-40t; 28_OX40t; 28_40t;
280X40t or 2840t) (SEQ ID NO: 122):
LFPGPSKPFWVLVVVGGVLACYSLLVTVAFIIFWVRSKRSRLLHSDYMNMTPRRPGPTRKH
YQPYAPPRDFAAYRSGGGSFRTPIQEEQADAHSTLA
Transmembrane_ICOS Signaling Domain_4-1BB Signaling Domain with mutated
polybasic region (other names: ICOS_BB(xPB); ICOSBB(xPB) or ICOS-BB(xPB)) (SEQ
ID NO: 123): ICOS
SQLCCQLKFWLPIGCAAFVVVCILGCILICWLTKKKYSSSVHDPNGEYMFMRAVNTAKKSR
LTDVTLAAGAAALLYIFKQPFMRPVQTTQEEDGCSCRFPEEEEGGCEL
ICOS Transmembrane_ICOS Signaling Domain_4-1BB Signaling Domain with mutated
lysines (Other names: ICOS_BB(xUB); ICOS-BB(xUB) or ICOSBB(xUB)) ((SEQ ID
NO: 124):
SQLCCQLKFWLPIGCAAFVVVCILGCILICWLTKKKYSSSVHDPNGEYMFMRAVNTAKKSR
LTDVTLKRGRKKLLYIFAQPFMRPVQTTQEEDGCSCRFPEEEEGGCEL
ICOS Transmembrane_ICOS Signaling Domain_4-1BB Signaling Domain with mutated
lysines and polybasic regions (Other names: ICOS_BB(xPB xUB); ICOS-BB(xPB xUB) or
ICOSBB(xPB xUB)) (SEQ ID NO: 125):
SQLCCQLKFWLPIGCAAFVVVCILGCILICWLTKKKYSSSVHDPNGEYMFMRAVNTAKKSR
LTDVTLAAGAAALLYIFAQPFMRPVQTTQEEDGCSCRFPEEEEGGCEL
ICOS Transmembrane_ICOS Signaling Domain_OX-40 Signaling Domain (Other names:
ICOS_OX40wt; ICOS-40; ICOS_40 wild type; ICOS_40wt; ICOS_40wt; ICOS-40wt;
ICOS-OX40wt; ICOSOX40wt; ICOS40 or ICOS40wt) (SEQ ID NO: 126):
SQLCCQLKFWLPIGCAAFVVVCILGCILICWLTKKKYSSSVHDPNGEYMFMRAVNTAKKSR
LTDVTLRRDQRLPPDAHKPPGGGSFRTPIQEEQADAHSTLAKI
ICOS Transmembrane_ICOS Signaling Domain_OX-40 Signaling Domain with mutated
polybasic region (other names: ICOS_OX40(xPB); ICOS_40(xPB); ICOS-40(xPB);
ICOS40(xPB); ICOS-OX40(xPB); or ICOSOX40(xPB)) (SEQ ID NO: 127):
SQLCCQLKFWLPIGCAAFVVVCILGCILICWLTKKKYSSSVHDPNGEYMFMRAVNTAKKSR
LTDVTLAADQALPPDAHKPPGGGSFRTPIQEEQADAHSTLAKI
ICOS Transmembrane_ICOS Signaling Domain_OX-40 Signaling Domain with mutated
lysine residues (other names: ICOS_OX40(xUB) ICOS_40(xUB); ICOS-40(xUB);
ICOS40(xUB); ICOS-OX40(xUB); or ICOSOX40(xUB)) (SEQ ID NO: 128):
SQLCCQLKFWLPIGCAAFVVVCILGCILICWLTKKKYSSSVHDPNGEYMFMRAVNTAKKSR
LTDVTLRRDQRLPPDAHAPPGGGSFRTPIQEEQADAHSTLAAI
ICOS Transmembrane_ICOS Signaling Domain_OX-40 Signaling Domain with mutated
lysine residues and polybasic regions (other names: ICOS_OX40(xPBxUB)
ICOS_40(xPBxUB); ICOS-40(xPBxUB); ICOS40(xPBxUB); ICOS-OX40(xPBxUB); or
ICOSOX40(xPBxUB)) (SEQ ID NO: 129):
SQLCCQLKFWLPIGCAAFVVVCILGCILICWLTKKKYSSSVHDPNGEYMFMRAVNTAKKSR
LTDVTLAADQALPPDAHAPPGGGSFRTPIQEEQADAHSTLAAI
ICOS stalk, transmembrane, and signaling domain (SEQ ID NO: 215)
SQLCCQLKFWLPIGCAAFVVVCILGCILICWLTKKKYSSSVHDPNGEYMFMRAVNTAKKSR
LTDVTL
ICOS intracellular domain (SEQ ID NO: 147)
CWLTKKKYSSSVHDPNGEYMFMRAVNTAKKSRLTDVTL
CD28 intracellular domain (SEQ ID NO: 193)
RSKRSRLLHSDYMNMTPRRPGPTRKHYQPYAPPRDFAAYRS

In some embodiments, the chimeric intracellular domain further comprises a third signal transduction domain. In some embodiments, the third signal transduction domain is derived from any one of a CD2 signaling domain, a MYD88 signaling domain, or an interleukin 2 receptor binding (IL-2RB) protein signaling domain.

In some embodiments, the third signal transduction domain of the chimeric intracellular domain is a mutant CD2 signaling domain. In some embodiments, the third signal transduction domain of the chimeric intracellular domain is a truncated CD2 signaling domain. In some embodiments, the third signal transduction domain of the chimeric intracellular domain is a truncated CD2 signaling domain comprising an amino acid sequence according to SEQ ID NO: 49. In some embodiments, the third signal transduction domain of the chimeric intracellular domain is a truncated CD2 signaling domain comprising an amino acid sequence having at least 50%, 55%, 60%, 65%, 70%, 75%, 80%, 85%, 90%, 95%, 97% or 99% identity to SEQ ID NO: 49.

In some embodiments, the third signal transduction domain of the chimeric intracellular domain is an IL-2RB protein signaling domain comprising an amino acid sequence according to SEQ ID NO: 50. In some embodiments, the third signal transduction domain of the chimeric intracellular domain is an IL-2RB protein signaling domain comprising an amino acid sequence having at least 50%, 55%, 60%, 65%, 70%, 75%, 80%, 85%, 90%, 95%, 97% or 99% identity to SEQ ID NO: 50.

In some embodiments, the chimeric intracellular domain further comprises a fourth signal transduction domain. In some embodiments, the fourth signal transduction domain is derived from any one of a CD2 signaling domain or an interleukin 2 receptor binding (IL-2RB) protein signaling domain or a combination thereof, wherein the third and the fourth signal transduction domain are not identical.

In some embodiments, the fourth signal transduction domain of the chimeric intracellular domain is a mutant CD2 signaling domain. In some embodiments, the fourth signal transduction domain of the chimeric intracellular domain is a truncated CD2 signaling domain. In some embodiments, the fourth signal transduction domain of the chimeric intracellular domain is a truncated CD2 signaling domain comprising an amino acid sequence according to SEQ ID NO: 49. In some embodiments, the fourth signal transduction domain of the chimeric intracellular domain is a truncated CD2 signaling domain comprising an amino acid sequence having at least 50%, 55%, 60%, 65%, 70%, 75%, 80%, 85%, 90%, 95%, 97% or 99% identity to SEQ ID NO: 49.

In some embodiments, the fourth signal transduction domain of the chimeric intracellular domain is an IL-2RB protein signaling domain comprising an amino acid sequence according to SEQ ID NO: 50. In some embodiments, the fourth signal transduction domain of the chimeric intracellular domain is an IL-2RB protein signaling domain comprising an amino acid sequence having at least 50%, 55%, 60%, 65%, 70%, 75%, 80%, 85%, 90%, 95%, 97% or 99% identity to SEQ ID NO: 50.

The terms T cell, T-cell, t cell, t-cell, and T lymphocyte can be used interchangeably in the present disclosure.

In some embodiments, the extracellular domain comprises a protein or a portion thereof that binds to a target to induce activation and/or proliferation of an immune cell. In some embodiments, the extracellular domain comprises any one of: a) a component of a T cell Receptor (TCR) complex; b) a component of a chimeric antigen receptor (CAR); c) a component of a T cell co-receptor, wherein the T cell co-receptor is a T cell co-stimulatory protein or T cell inhibitory protein; d) a ligand that binds to a cell surface receptor or a component thereof; e) a component of a cytokine receptor; e) a component of a chemokine receptor; g) a component of an integrin receptor; h) a component of an endothelial cell surface protein receptor or a fragment thereof; i) a component of a neuronal guidance protein receptor; and f) a component of a complement receptor. In some embodiments, the component of the T cell co-receptor or the CAR is a component of PD-1, CD28, CD2, OX-40, ICOS, CTLA-4, CD28, CD4, CD8, CD40L, Lag-3, Tim-3, or TIGIT, or a combination thereof. In some embodiments, the ligand or component of the T cell co-receptor or CAR binds to CD19, B cell maturation Ag (BCMA), PD-L1, PD-L2, IL-10, a proliferation-inducing ligand (APRIL), BAFF, OX-40L, ICOS-L, B7-1, B7-2, CD40, CD58, CD59, nectin, CD155, or CD112, or a combination thereof. In some embodiments, the cytokine receptor binds to IL-10, IL-27, TGF-β, IL-12, IL-1, IL-2, IL-4, IL-5, IFN-γ, or IFN-α/β, or a combination thereof. In some embodiments, the component of the complement receptor is a component of a single C3aR, C5aR, CD46/MCP, CD55, CD97, or DAF, or a combination thereof.

In some embodiments, the extracellular domain comprises an amino acid sequence of a component of any one of: a) a chemokine receptor; b) a cytokine receptor; c) a ligand for a cell surface receptor; d) an integrin receptor; e) a cell adhesion molecule or a receptor thereof; f) an endothelial cell surface protein receptor or a fragment thereof; g) a complement receptor; and h) a neuronal guidance protein receptor. In some embodiments, the extracellular domain comprises an amino acid sequence of a component of any one of epithelial growth factor receptor (EGFR), vascular-endothelial growth factor (VEGFR), chemokine receptor (CCR) 4, CCR5, CCR7, CCR10, Lymphocyte function-associated antigen-1 (LFA-1), leukocyte-specific B2 integrins (aLB2, aMB2, aXB2, aDB2), β7 integrins (α4β7 and αEβ7), extracellular matrix (ECM)-binding β1 integrins (α1-α6β1), L-selectin, or sialyl LewisX.

In some embodiments, the extracellular domain is a protein, a peptide, a glycoprotein, an antibody or a fragment thereof. In some embodiments, the antibody or fragment thereof is a Fab fragment, a F(ab)₂ fragment, a diabody, a nanobody, a sdAb, Fv, a V_{H}H fragment, or a single chain Fv fragment.

In some embodiments, the extracellular domains comprise two or more binding sites for targeting two or more non-identical target antigens. In some embodiments, the extracellular domains comprise two or more binding sites for targeting two or more non-identical sites on a target antigen. In some embodiments, the extracellular domain comprises two antigen binding domains or fragments of a bispecific antibody. In some embodiments, the extracellular domain comprises a F(ab)₂ fragment of a bispecific antibody. In some embodiments, the extracellular domain comprises two or more antigen binding domains or fragments of a multi-specific antibody.

In some embodiments, the extracellular domain binds to a target that is a tumor antigen, a pathogen associated protein, or an antigen associated with the disease or disorder that is a cancer, an autoimmune disease or disorder, an infectious disease, an inflammatory disease, a renal disease or disorder, a lung disease or disorder, a liver disease or disorder, a cardiovascular disease or disorder, a neurodegenerative disorder or disorder, or a metabolic disorder or disorder.

In some embodiments, the tumor antigen is any one of a tumor associated antigen (TAA), a tumor secreted antigen (TSA) or an unconventional antigen (UCA). In some embodiments, the TAA is any one of a cancer germline antigen (CGA), a Human endogenous retroviruses (HERVs), tissue differentiation antigen (TDA) and overexpressed tumor antigen. In some embodiments, the TSA is derived from any one of a mosaic single nucleotide variations (mSNVs), a insertion-deletion mutations (INDELs), gene fusions and viral oncoproteins. In some embodiments, the UCA is derived from non-coding regions of the genome or from coding regions of the genome. In some embodiments, the UCA is derived from aberrant transcription, translation, or post-translational modifications.

In some embodiments, the TAA is associated with a solid tumor or cancer or a hematologic cancer. In some embodiments, the TAA is associated with a solid tumor or cancer is selected from a sarcoma, a carcinoma or a lymphoma that manifests as, leads to, or is associated with a solid tumor.

In some embodiments, the TAA is associated with a sarcoma that is a soft tissue sarcoma or a bone sarcoma (osteosarcoma). In some embodiments, the TAA is associated with a sarcoma selected from vesicular rhabdomyosarcoma, vesicular soft tissue sarcoma, ameloblastoma, angiosarcoma, chondrosarcoma, chordoma, bright tissue sarcoma, dedifferentiated liposarcoma, Hyperplastic small round cell tumor of connective tissue, embryonic rhabdomyosarcoma, epithelioid fibrosarcoma, epithelioid hemangioendothelioma, epithelioid sarcoma; sensitive neuroblastoma (esthesioneuroblastoma), Ewing sarcoma, extrarenal rhabdomyosarcoma, extraosseous myxoid chondrosarcoma, extraosseous osteosarcoma, fibrosarcoma, giant cell tumor, hemangiopericytoma, infantile fibrosarcoma, inflammatory myofibroblastoma, Kaposi sarcoma, bone smooth muscle sarcoma, liposarcoma, osteosarcoma, malignant fibrous histiocytoma (MFH), malignant fibrous histiocytoma (MFH), malignant mesenchymal tumor, malignant peripheral nerve sheath tumor, mesenchymal chondrosarcoma, myxoid liposarcoma, myxoid inflammatory fibroblastic sarcoma, multiple tumors with perivascular epithelioid cell differentiation, osteosarcoma, extraperiosteal osteosarcoma, tumors with perivascular epithelial cell differentiation, periosteum osteosarcoma, polymorphic liposarcoma, polymorphic rhabdomyosarcoma, PNET/extraosseous Ewing's tumor, rhabdomyosarcoma, small cell osteosarcoma, single fibroids, synovial sarcoma or capillary dilated osteosarcoma.

In some embodiments, the TAA is associated with a carcinoma selected from basal cell carcinoma, squamous cell carcinoma, renal cell carcinoma, ductal carcinoma in situ (DCIS), invasive ductal carcinoma or adenocarcinoma. In some embodiments, the TAA is associated with a carcinoma selected from adenosquamous carcinoma, anaplastic carcinoma, large cell carcinoma, colorectal carcinoma, pancreatic carcinoma, nasopharyngeal carcinoma or small cell carcinoma.

In some embodiments, the TAA is associated with a solid tumor or cancer selected from anal cancer, appendix cancer; cholangiocarcinoma (i.e., biliary tract cancer), breast cancer, bladder cancer, brain tumor, breast cancer, cervical cancer, colon cancer, colorectal cancer, colon polyp, unidentified primary cancer (cup), esophagus cancer, eye cancer, tubal cancer, kidney cancer, liver cancer, lung cancer, medulloblastoma, melanoma, oral cancer, ovarian cancer, prostate cancer, pancreatic cancer, gastric cancer, testicular cancer, laryngeal cancer, thyroid cancer, uterine cancer, vaginal cancer, or vulvar cancer.

In some embodiments, the breast cancer is an invasive breast duct cancer, carcinoma in situ of the duct, invasive lobular carcinoma or lobular carcinoma in situ. In some embodiments, the pancreatic cancer is adenocarcinoma or islet cell carcinoma. In some embodiments, the colorectal cancer is adenocarcinoma. In some embodiments, colonic polyps are associated with familial adenomatous polyposis. In some embodiments, the bladder cancer is transitional cell bladder cancer, squamous cell bladder cancer, or adenocarcinoma. In some embodiments, the lung cancer is non-small cell lung cancer. In some embodiments, the non-small cell lung cancer is adenocarcinoma, squamous cell lung cancer, or large cell lung cancer. In some embodiments, the non-small cell lung cancer is large cell lung cancer. In some embodiments, the lung cancer is small cell lung cancer. In some embodiments, the prostate cancer is adenocarcinoma or small cell carcinoma. In some embodiments, the ovarian cancer is epithelial ovarian cancer. In some embodiments, the cholangiocarcinoma is proximal cholangiocarcinoma or distal cholangiocarcinoma.

In some embodiments, the TAA is associated with any one of the hematological cancer selected from a leukemia, a myeloma or a lymphoma. In some embodiments, the TAA is associated with a leukemia selected from acute leukemia, acute lymphoblastic leukemia (ALL), acute lymphocytic leukemia, a B cell, T cell or FAB ALL, acute myeloid leukemia (AML), acute myelogenous leukemia, chronic myelocytic leukemia (CML), chronic lymphocytic leukemia (CLL), hairy cell leukemia, acute promyelocytic leukemia (APL), mixed-lineage leukemia (MLL) or myelodysplastic syndrome (MDS).

In some embodiments, the TAA is associated with a myeloma that is a multiple myeloma. In some embodiments, the TAA is associated with a multiple myeloma selected from the hyperdiploid (HMM) or the non-hyperdiploid or hypodiploid subtypes of multiple myeloma. In some embodiments, the TAA is associated with a multiple myeloma selected from light chain myeloma, non-secretory myeloma, solitary plasmacytoma, extramedullary plasmacytoma, monoclonal gammopathy of undetermined significance (MGUS), smoldering multiple myeloma (SMM), immunoglobulin D (IgD) myeloma or, immunoglobulin E (IgE) myeloma.

In some embodiments, the TAA is associated with a lymphoma that is a Hodgkin's lymphoma or a non-Hodgkin's lymphoma. In some embodiments, the TAA is associated with a non-Hodgkin's lymphoma. In some embodiments, the TAA is associated with a non-Hodgkin's lymphoma selected from a Small lymphocytic lymphoma (SLL), Lymphoplasmacytic lymphoma, Diffuse large cell lymphoma, Follicle center cell lymphoma, Burkitt's lymphoma, Burkitt-like lymphoma, Mantle cell lymphoma or Marginal zone B-cell lymphoma. In some embodiments, the TAA is associated with a lymphoma that is a Hodgkin's lymphoma. In some embodiments, the TAA is associated with a Hodgkin's lymphoma selected from nodular sclerosis classical Hodgkin lymphoma, lymphocyte-rich classical Hodgkin lymphoma or lymphocyte-depleted classical Hodgkin lymphoma.

In some embodiments, the TAA is associated with a cancer that is any one of acute leukemia, acute lymphoblastic leukemia (ALL), acute lymphocytic leukemia, B cell, T cell or FAB ALL, acute myeloid leukemia (AML), acute myelogenous leukemia, chronic myelocytic leukemia (CML), chronic lymphocytic leukemia (CLL), hairy cell leukemia, myelodysplastic syndrome (MDS), Hodgkin's lymphoma, Hodgkin's disease, non-Hodgkin's lymphoma, multiple myeloma, colorectal carcinoma, pancreatic carcinoma, nasopharyngeal carcinoma, malignant histiocytosis, paraneoplastic syndrome/hypercalcemia of malignancy, bladder cancer, breast cancer, colorectal cancer, endometrial cancer, head cancer, neck cancer, hereditary nonpolyposis cancer, liver cancer, lung cancer, non-small cell lung cancer, ovarian cancer, pancreatic cancer, prostate cancer, renal cell carcinoma, testicular cancer, adenocarcinomas, sarcomas, malignant melanoma, and hemangioma.

In some embodiments, the extracellular domain binds to a TAA selected from kallikrein 4, papillomavirus binding factor (PBF), preferentially expressed antigen of melanoma (PRAME), Wilms' tumor-I (WTI), Hydroxysteroid Dehydrogenase Like I (HSDLI), mesothelin, cancer testis antigen (NY-ESO-1), carcinoembryonic antigen (CEA), p53, human epidermal growth factor receptor 2/neuro receptor tyrosine kinase (Her2/Neu), carcinoma-associated epithelial cell adhesion molecule (EpCAM), ovarian and uterine carcinoma antigen (CAI25), folate receptor a, sperm protein 17, tumor-associated differentially expressed gene-12 (TADG-12), mucin-16 (MUC-16), LI cell adhesion molecule (LICAM), mannan-MUC-1, Human endogenous retrovirus K (HERV-K-MEL), Kita-kyushu lung cancer antigen-I (KK-LC-1), human cancer/testis antigen (KM-HN-1), cancer testis antigen (LAGE-I), melanoma antigen-A1 (MAGE-A1), Sperm surface zona pellucida binding protein (Spl 7), Synovial Sarcoma, X Breakpoint 4 (SSX-4), Transient axonal glycoprotein-1 (TAG-I), Transient axonal glycoprotein-2 (TAG-2), Enabled Homolog (ENAH), mammoglobin-A, NY-BR-I, Breast Cancer Antigen, (BAGE-1), B melanoma antigen, melanoma antigen-A1 (MAGE-A1), melanoma antigen-A2 (MAGE-A2), mucin k, synovial sarcoma, X breakpoint 2 (SSX-2), Taxol-resistance-associated gene-3 (TRAG-3), Avian Myelocytomatosis Viral Oncogene (c-myc), cyclin B 1, mucin I (MUC I), p62, survivin, lymphocyte common antigen (CD45), DickkopfWNT Signaling Pathway Inhibitor I (DKKI), telomerase, Kirsten rat sarcoma viral oncogene homolog (K-ras), G250, intestinal carboxyl esterase, alpha-fetoprotein, Macrophage Colony-Stimulating Factor (M-CSF), Prostate-specific membrane antigen (PSMA), caspase 5 (CASP-5), Cytochrome C Oxidase Assembly Factor I Homolog (COA-1), O-linked β-N-acetylglucosamine transferase (OGT), Osteosarcoma Amplified 9, Endoplasmic Reticulum Lectin (OS-9), Transforming Growth Factor Beta Receptor 2 (TGF-betaRII), murine leukemia glycoprotein 70 (gp70), Calcitonin Related Polypeptide Alpha (CALCA), Programmed cell death 1 ligand 1 (CD274), Mouse Double Minute 2Homolog (mdm-2), alpha-actinin-4, elongation factor 2, Malic Enzyme 1 (MEI), Nuclear Transcription Factor Y Subunit C (NFYC), G Antigen 1,3 (GAGE-1,3), melanoma antigen-A6 (MAGE-A6), cancer testis antigen XAGE-lb, six transmembrane epithelial antigen of the prostate 1 (STEAPI), PAP, prostate specific antigen (PSA), Fibroblast Growth Factor 5 (FGF5), heat shock protein hsp70-2, melanoma antigen-A9 (MAGE-A9), Arg-specific ADP-ribosyltransferase family C (ARTCI), B-Raf Proto-Oncogene (B-RAF), Serine/Threonine Kinase, beta-catenin, Cell Division Cycle 27 homolog (Cdc27), cyclin dependent kinase 4 (CDK4), cyclin dependent kinase 12 (CDK12), Cyclin Dependent Kinase Inhibitor 2A (CDKN2A), Casein Kinase 1 Alpha 1 (CSNKIAI), Fibronectin 1 (FNI), Gruwih Anest Specific 7 (GAS7), Glycoprotein nonmetastatic melanoma protein B (GPNMB), HAUS Augmin Like Complex Subunit 3 (HAUS3), LDLR-fucosyltransferase, Melanoma Antigen Recognized By T cells 2 (MART2), myostatin (MSTN), Melanoma Associated Antigen (Mutated) 1 (MUM-1-2-3), Poly (A) polymerase gamma (neo-PAP), myosin class I, Protein phosphatase 1 regulatory subunit 3B (PPP1R3B), Peroxiredoxin-5 (PRDX5), Receptor-type tyrosine-protein phosphatase kappa (PTPRK), Transforming protein N-Ras (N-ras), retinoblastoma-associated factor 600 (RBAF600), sirtuin-2 (SIRT2), SNRPDI, triosephosphate isomerase, Ocular Albinism Type 1 Protein (OAI), member RAS oncogene family (RAB38), Tyrosinase related protein 1-2 (TRP-1-2), Melanoma Antigen Gp75 (gp75), tyrosinase, Melan-A (MART-1), Glycoprotein 100 melanoma antigen (gplOO), N-acetylglucosaminyltransferase V gene (GnTVf), Lymphocyte Antigen 6 Complex Locus K (LY6K), melanoma antigen-AlO (MAGE-AlO), melanoma antigen-A12 (MAGE-A12), melanoma antigen-C2 (MAGE-C2), melanoma antigen NA88-A, Taxol-resistant-associated protein 3 (TRAG-3), BDZ binding kinase (pbk), caspase 8 (CASP-8), sarcoma antigen 1 (SAGE), Breakpoint Cluster Region-Abelson oncogene (BCR-ABL), fusion protein in leukemia, dek-can, Elongation Factor Tu GTP Binding Domain Containing 2 (EFTUD2), ETS Variant gene 6/acute myeloid leukemia fusion protein (ETV6-AML1), FMS-like tyrosine kinase-3 internal tandem duplications (FLT3-ITD), cyclin-A1, Fibronectin Type III Domain Containing 3B (FDNC3B) promyelocytic leukemia/retinoic acid receptor alpha fusion protein (pml-RARalpha), melanoma antigen-Cl (MAGE-Cl), membrane protein alternative spliced isoform (D393-CD20), melanoma antigen-A4 (MAGE-A4), or melanoma antigen-A3 (MAGE-A3).

In some embodiments, the autoimmune condition or disorder is any one of Type 1 Diabetes, rheumatoid arthritis (RA), systemic lupus erythematosis (SLE), multiple sclerosis (MS), celiac disease, sjögren syndrome, polymyalgia rheumatica, ankylosing spondylitis, alopecia areata, vasculitis and temporal arteritis. In some embodiments, the tumor associated antigen (TAA) associated with the autoimmune condition or disorder is derived from any one of Carboxypeptidase H, Chromogranin A, Glutamate decarboxylase, Imogen-38, Insulin, Insulinoma antigen-2 and 2B, Islet-specific glucose-6-phosphatase catalytic subunit related protein (IGRP), Proinsulin, α-enolase, Aquaporin-4, β-arrestin, Myelin basic protein, Myelin oligodendrocytic glycoprotein, Proteolipid protein, S100-β, Citrullinated protein, Collagen II, Heat shock proteins, Human cartilage glycoprotein, Double-stranded DNA, La antigen, Nucleosomal histones and ribonucleoproteins (snRNP), Phospholipid-β-2 glycoprotein I complex, Poly (ADP-ribose) polymerase, and Sm antigens of U-1 small ribonucleoprotein complex.

In some embodiments, the pathogen associated antigen is an antigen from a bacterial, a fungal or a parasitic protein or fragment thereof. In some embodiments, the pathogen associated antigen is associated with HIV infection, human Cytomegalovirus infection, Hepatitis B infection, Hepatitis C infection, Ebola virus infection, Dengue, Yellow fever, Listeriosis, Tuberculosis, Cholera, Malaria, Leishmaniasis, or Trypanosoma infection, or a combination thereof.

In some embodiments, the neurodegenerative disorder or condition is any one of Alzheimer's disease (AD) and other dementias, Parkinson's disease (PD) and PD-related disorders, Prion disease, Motor neurone diseases (MND), Huntington's disease (HD), Spinocerebellar ataxia (SCA) or Spinal muscular atrophy (SMA). In some embodiments, the antigen associated with the neurodegenerative disorder or condition is any one of Amyloid β (Ab), tau, alpha-synuclein (α-syn), mHTT or prion PrPSC or a combination thereof.

In some embodiments, the extracellular domain binds to a target with a binding affinity of 1 fM to 100 μM. In some embodiments, the extracellular domain binds to a target with a binding affinity of 1 pM to 100 μM. In some embodiments, the extracellular domain binds to a target with a binding affinity of 1 pM to 10 pM. In some embodiments, the extracellular domain binds to a target with a binding affinity of 10 pM to 50 pM. In some embodiments, the extracellular domain binds to a target with a binding affinity of 10 pM to 100 pM. In some embodiments, the extracellular domain binds to a target with a binding affinity of 100 pM to 500 pM. In some embodiments, the extracellular domain binds to a target with a binding affinity of 500 pM to 1 nM. In some embodiments, the extracellular domain binds to a target with a binding affinity of 1 nM to 10 nM. In some embodiments, the extracellular domain binds to a target with a binding affinity of 10 nM to 100 nM. In some embodiments, the extracellular domain binds to a target with a binding affinity of 100 nM to 500 nM. In some embodiments, the extracellular domain binds to a target with a binding affinity of 500 nM to 1 μM. In some embodiments, the extracellular domain binds to a target with a binding affinity of 1 μM to 10 μM. In some embodiments, the extracellular domain binds to a target with a binding affinity of 1 μM to 5 μM. In some embodiments, the extracellular domain binds to a target with a binding affinity of 5 μM to 7.5 μM. In some embodiments, the extracellular domain binds to a target with a binding affinity of 7.5 μM to 10 μM.

In some embodiments, the extracellular domain comprises a signal peptide at the N-terminus. In some embodiments, the signal peptide can be derived from a surface expressing protein or a secretory protein. In some embodiments, the signal peptide can be derived from Preprolactin, HIV pre-Env, HCV polyprotein, CB virus polyprotein, Pestivirus polyprotein, Precalreticulin, pre-VSV-G, HLA class I histocompatibility antigen or PD-1 signal peptide (PD-1 SP), interleukin 12 (IL12), GM-CSF or CD8 alpha chain (CD8a). In some embodiments, the signal peptide is PD-1 signal peptide (PD-1 SP). In some embodiments, the signal peptide is a HLA class I histocompatibility antigen or a portion thereof. In some embodiments, the extracellular domain is derived from PD-1. In some embodiments, the extracellular domain comprises the amino acid sequence from position 1 to 163 of the amino acid sequence according to any one of SEQ ID NOs: 19-21. In some embodiments, the extracellular domain comprises the amino acid sequence from position 1 to 163 of the amino acid sequence according to SEQ ID NO: 19. In some embodiments, the extracellular domain comprises the amino acid sequence from position 1 to 163 of the amino acid sequence according to SEQ ID NO: 20. In some embodiments, the extracellular domain comprises the amino acid sequence from position 1 to 163 of the amino acid sequence according to SEQ ID NO: 21.

In some embodiments, the extracellular domain comprises the amino acid sequence according to any one of SEQ ID NOs: 22-23. In some embodiments, the extracellular domain comprises the amino acid sequence according to SEQ ID NO: 22. In some embodiments, the extracellular domain comprises the amino acid sequence having at least 50%, 55%, 60%, 65%, 70%, 75%, 80%, 85%, 90%, 95%, 97% or 99% identity to SEQ ID NO: 22. In some embodiments, the extracellular domain comprises the amino acid sequence according to SEQ ID NO: 23. In some embodiments, the extracellular domain comprises the amino acid sequence having at least 50%, 55%, 60%, 65%, 70%, 75%, 80%, 85%, 90%, 95%, 97% or 99% identity to SEQ ID NO: 23.

TABLE 4
Amino acid sequences of domains of RTCR.

PD-1 (PD-1 Signal Peptide_PD-1 Extracellular_PD-1 Transmembrane_PD-1 Intracellular)
(other names: PD1_wt (human-wild type); PD1: WT; PD-1; PD-1 wt; PD-1 wild type; PD1;
PD1wt or PD1 wild type) (SEQ ID NO: 19): Signal Peptide (italicized), Extracellular
domain (IG-like V domain in bold and stalk in bold and underlined), Trans Membrane
(underlined) and Intracellular domain in double underline
MQIPQAPWPVVWAVLQLGWRPGWFLDSPDRPWNPPTFSPALLVVTEGDNATFTCSFSNTSE
SFVLNWYRMSPSNQTDKLAAFPEDRSQPGQDCRFRVTQLPNGRDFHMSVVRARRNDSGTYL
CGAISLAPKAQIKESLRAELRVTERRAEVPTAHPSPSPRPAGQFQTLVVGVVGGLLGSLVL
LVWVLAVICSRAARGTIGARRTGOPLKEDPSAVPVESVDYGELDFOWREKTPEPPVPCVPE
QTEYATIVFPSGMGTSSPARRGSADGPRSAOPLRPEDGHCSWPL
PD-1 Signal Peptide_PD-1 Extracellular_PD-1 Transmembrane (Other name: PD-1
truncated) (SEQ ID NO: 20): PD1 Signal Peptide (italicized), Extracellular domain (IG-
like V domain in bold and stalk in bold and underlined), Trans Membrane (underlined) and
Intracellular tail in double underline
MQIPQAPWPVVWAVLQLGWRPGWFLDSPDRPWNPPTFSPALLVVTEGDNATFTCSFSNTSE
SFVLNWYRMSPSNQTDKLAAFPEDRSQPGQDCRFRVTQLPNGRDFHMSVVRARRNDSGTYL
CGAISLAPKAQIKESLRAELRVTERRAEVPTAHPSPSPRPAGQFQTLVVGVVGGLLGSLVL
LVWVLAVICSR
HLA-A2 Signal Peptide_PD1 Extracellular_PD1 Transmembrane (PD1_TLs; HLASP-
Truncated, PD1-TLs, PD1: TLs) (SEQ ID NO: 21): HLA-A2 Signal Peptide (italicized);
Extracellular domain (IG-like V domain in bold and stalk in bold and underlined), Trans
Membrane (underlined) and Intracellular tail in double underline
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPAGQFQTLVVGVVGGLLGSLV
LLVWVLAVICSR
PD-1 (extracellular domain) (SEQ ID NO: 22): Signal Peptide (italicized), Extracellular
domain (IG-like V domain in bold and stalk in bold and underlined)
MQIPQAPWPVVWAVLQLGWRPGWFLDSPDRPWNPPTFSPALLVVTEGDNATFTCSFSNTSE
SFVLNWYRMSPSNQTDKLAAFPEDRSQPGQDCRFRVTQLPNGRDFHMSVVRARRNDSGTYL
CGAISLAPKAQIKESLRAELRVTERRAEVPTAHPSPSPRPA
PD-1 (HLA A2-Signal Peptide extracellular domain) (other name: PD-1 (HLA A2-SP
extracellular domain) (SEQ ID NO: 23): Signal Peptide (italicized), Extracellular domain
(IG-like V domain in bold and stalk in bold and underlined)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRP
Programed Death Receptor 1/PD-1 (Mature Sequence) (SEQ ID NO: 229)
FLDSPDRPWNPPTFSPALLVVTEGDNATFTCSFSNTSESFVLNWYRMSPSNQTDKLAAFPE
DRSQPGQDCRFRVTQLPNGRDFHMSVVRARRNDSGTYLCGAISLAPKAQIKESLRAELRVT
ERRAEVPTAHPSPSPRPAGQFQTLVVGVVGGLLGSLVLLVWVLAVICSRAARGTIGARRTG
QPLKEDPSAVPVFSVDYGELDFQWREKTPEPPVPCVPEQTEYATIVFPSGMGTSSPARRGS
ADGPRSAQPLRPEDGHCSWPL
CD28 (Mature Sequence) (SEQ ID NO: 242)
MLRLLLALNLFPSIQVTGNKILVKQSPMLVAYDNAVNLSCKYSYNLFSREFRASLHKGLDS
AVEVCVVYGNYSQQLQVYSKTGFNCDGKLGNESVTFYLQNLYVNQTDIYFCKIEVMYPPPY
LDNEKSNGTIIHVKGKHLCPSPLFPGPSKPFWVLVVVGGVLACYSLLVTVAFIIFWVRSKR
SRLLHSDYMNMTPRRPGPTRKHYQPYAPPRDFAAYRS
ICOS (Mature Sequence) (SEQ ID NO: 243)
MKSGLWYFFLFCLRIKVLTGEINGSANYEMFIFHNGGVQILCKYPDIVQQFKMQLLKGGQI
LCDLTKTKGSGNTVSIKSLKFCHSQLSNNSVSFFLYNLDHSHANYYFCNLSIFDPPPFKVT
LTGGYLHIYESQLCCQLKFWLPIGCAAFVVVCILGCILICWLTKKKYSSSVHDPNGEYMFM
RAVNTAKKSRLTDVTL
TNFRSF4/OX40/CD134 (Mature Sequence) (SEQ ID NO: 244)
MCVGARRLGRGPCAALLLLGLGLSTVTGLHCVGDTYPSNDRCCHECRPGNGMVSRCSRSQN
TVCRPCGPGFYNDVVSSKPCKPCTWCNLRSGSERKQLCTATQDTVCRCRAGTQPLDSYKPG
VDCAPCPPGHFSPGDNQACKPWTNCTLAGKHTLQPASNSSDAICEDRDPPATQPQETQGPP
ARPITVQPTEAWPRTSQGPSTRPVEVPGGRAVAAILGLGLVLGLLGPLAILLALYLLRRDQ
RLPPDAHKPPGGGSFRTPIQEEQADAHSTLAKI
TNFRSF5/CD40 (Mature Sequence) (SEQ ID NO: 245)
MVRLPLQCVLWGCLLTAVHPEPPTACREKQYLINSQCCSLCQPGQKLVSDCTEFTETECLP
CGESEFLDTWNRETHCHQHKYCDPNLGLRVQQKGTSETDTICTCEEGWHCTSEACESCVLH
RSCSPGFGVKQIATGVSDTICEPCPVGFFSNVSSAFEKCHPWTSCETKDLVVQQAGTNKTD
VVCGPQDRLRALVVIPIIFGILFAILLVLVFIKKVAKKPTNKAPHPKQEPQEINFPDDLPG
SNTAAPVQETLHGCQPVTQEDGKESRISVQERQ
TNFRSF7 (CD27) (SEQ ID NO: 246)
MARPHPWWLCVLGTLVGLSATPAPKSCPERHYWAQGKLCCQMCEPGTFLVKDCDQHRKAAQ
CDPCIPGVSFSPDHHTRPHCESCRHCNSGLLVRNCTITANAECACRNGWQCRDKECTECDP
LPNPSLTARSSQALSPHPQPTHLPYVSEMLEARTAGHMQTLADFRQLPARTLSTHWPPQRS
LCSSDFIRILVIFSGMFLVFTLAGALFLHQRRKYRSNKGESPVEPAEPCHYSCPREEEGST
IPIQEDYRKPEPACSP
TNFRSF9 (4-1BB) (SEQ ID NO: 247)
MGNSCYNIVATLLLVLNFERTRSLQDPCSNCPAGTFCDNNRNQICSPCPPNSFSSAGGQRT
CDICRQCKGVFRTRKECSSTSNAECDCTPGFHCLGAGCSMCEQDCKQGQELTKKGCKDCCF
GTFNDQKRGICRPWTNCSLDGKSVLVNGTKERDVVCGPSPADLSPGASSVTPPAPAREPGH
SPQIISFFLALTSTALLFLLFFLTLRFSVVKRGRKKLLYIFKQPFMRPVQTTQEEDGCSCR
FPEEEEGGCEL

In some embodiments, the RTCR disclosed herein comprises an amino acid sequence from Table 4.

In some embodiments, the RTCR disclosed herein comprises the amino acid sequence according to any one of SEQ ID NOs: 24-44 and 130-132.

In some embodiments, the RTCR disclosed herein comprises the amino acid sequence according to SEQ ID NO: 24. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 24.

In some embodiments, the RTCR disclosed herein comprises the amino acid sequence according to SEQ ID NO: 25. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 25.

In some embodiments, the RTCR disclosed herein comprises the amino acid sequence according to SEQ ID NO: 26. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 26.

In some embodiments, the RTCR disclosed herein comprises the amino acid sequence according to SEQ ID NO: 27. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 27.

In some embodiments, the RTCR disclosed herein comprises the amino acid sequence according to SEQ ID NO: 28. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 28.

In some embodiments, the RTCR disclosed herein comprises the amino acid sequence according to SEQ ID NO: 29. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 29.

In some embodiments, the RTCR disclosed herein comprises the amino acid sequence according to SEQ ID NO: 30. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 30.

In some embodiments, the extracellular domain of the RTCR disclosed herein comprises the amino acid sequence according to SEQ ID NO: 31. In some embodiments, the extracellular domain of the RTCR disclosed herein comprises the amino acid sequence having at least 50%, 55%, 60%, 65%, 70%, 75%, 80%, 85%, 90%, 95%, 97% or 99% identity to SEQ ID NO: 31.

In some embodiments, the RTCR disclosed herein comprises the amino acid sequence according to SEQ ID NO: 32. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 32.

In some embodiments, the RTCR disclosed herein comprises the amino acid sequence according to SEQ ID NO: 33. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 33.

In some embodiments, the RTCR disclosed herein comprises the amino acid sequence according to SEQ ID NO: 34. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 34.

In some embodiments, the RTCR disclosed herein comprises the amino acid sequence according to SEQ ID NO: 35. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 35.

In some embodiments, the RTCR disclosed herein comprises the amino acid sequence according to SEQ ID NO: 36. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 36.

In some embodiments, the RTCR disclosed herein comprises the amino acid sequence according to SEQ ID NO: 37. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 37.

In some embodiments, the RTCR disclosed herein comprises the amino acid sequence according to SEQ ID NO: 38. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 38.

In some embodiments, the RTCR disclosed herein comprises the amino acid sequence according to SEQ ID NO: 39. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 39.

In some embodiments, the RTCR disclosed herein comprises the amino acid sequence according to SEQ ID NO: 40. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 40.

In some embodiments, the RTCR disclosed herein comprises the amino acid sequence according to SEQ ID NO: 41. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 41.

In some embodiments, the RTCR disclosed herein comprises the amino acid sequence according to SEQ ID NO: 42. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 42.

In some embodiments, the RTCR disclosed herein comprises the amino acid sequence according to SEQ ID NO: 43. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 43.

In some embodiments, the RTCR disclosed herein comprises the amino acid sequence according to SEQ ID NO: 44. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 44.

In some embodiments, the RTCR disclosed herein comprises the amino acid sequence according to SEQ ID NO: 130. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 130.

In some embodiments, the RTCR disclosed herein comprises the amino acid sequence according to SEQ ID NO: 131. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 131.

In some embodiments, the RTCR disclosed herein comprises the amino acid sequence according to SEQ ID NO: 132. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 132.

```
TABLE 5
Example amino acid sequences of PD-1 based recombinant T cell
co-stimulatory receptor (RTCR).
HLA-A2 Signal Peptide_PD-1 Extracellular_CD28 Transmembrane_CD28
Signaling Domain (Other names: PD-1-CD28 Domain Swap; HLA A2-SP-
PD-1_28; HLA A2-SP-PD-1_CD28 DS; HLA A2-SP-PD-1_CD28; PD1_CD28
or PD1:CD28 or PD1_28) (SEQ ID NO: 24): HLA-A2 Signal Peptide
(italicized), PD 1 extracellular domain (IG-like V domain in
bold and stalk in bold and underlined), CD28 Transmembrane
(underlined) and Intracellular domain in double underline; and
CD28 signal domain: Stalk (underlined and italicized),
transmembrane domain (double underlined), intracellular domain
(IC) (dashed underlined)

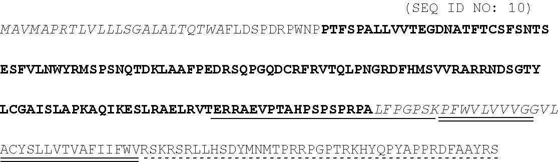

HLA-A2 Signal Peptide_PD-1 Extracellular_ICOS Transmembrane_ICOS
Signaling Domain (Other names: PD-1-ICOS Domain Swap; HLASP-PD-
1-ICOS DS; HLA A2-SP-PD-1_ICOS; PD-1-ICOS; PD-1:ICOS; PD1_ICOS)
HLA-A2 Signal Peptide (italicized), PD 1 extracellular domain
(IG-like V domain in bold and stalk in bold and underlined),
ICOS Transmembrane (underlined) and Intracellular domain in
double underline; and ICOS domain: Stalk (underlined and
italicized), transmembrane domain (double underlined),
intracellular domain (IC) (dashed underlined)

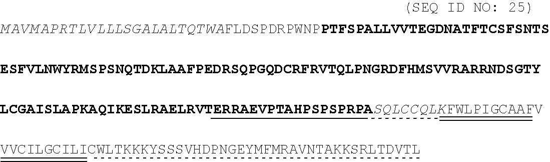

HLA-A2 Signal Peptide_PD-1 Extracellular_ICOS Transmembrane_ICOS
Signaling Domain_4-1BB Signaling Domain (other names: PD1 HLASP-
ICOS-4-1BB; HLA A2-SP-PD-1_ICOS_BBwt; HLA A2-SP-PD-1_ICOS_BB;
HLA A2-SP-PD-1_ICOS_CD137; HLA A2-SP-PD-1_ICOS_CD137 wt;
PD1_ICOS_BBwt; PD1:ICOSBBwt; PD1:ICOSBB) PD 1 extracellular
domain in regular font; ICOS domain underlined; and 4-1BB
domain (CD137 signaling domain, wild type) in bold
                                                                                            (SEQ ID NO: 26)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPASQLCCQLKFWLPIGCAAFV
VVCILGCILICWLTKKKYSSSVHDPNGEYMFMRAVNTAKKSRLTDVTLKRGRKKLLYIFKQ
PFMRPVQTTQEEDGCSCRFPEEEEGGCEL
HLA-A2 Signal Peptide_PD-1 Extracellular_ICOS Transmembrane_ICOS
Signaling Domain_Truncated 4-1BB Signaling Domain (Other names:
PD1 HLASP-ICOS-truncated 4-1BB; HLA A2-SP-PD-1-ICOS_BBt; HLA
A2-SP-PD-1_ICOS_BBt; HLA A2-SP-PD-1_ICOS_truncated CD137; HLA
A2-SP-PD-1_ICOS_truncated CD137 wt; PD1_ICOS_BBt; PD1:ICOSBBt)
PD 1 extracellular domain in regular font; ICOS domain
underlined; and truncated 4-1BB (truncated CD137 signaling
domain in bold
                                                                                            (SEQ ID NO: 27)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPASQLCCQLKFWLPIGCAAFV
VVCILGCILICWLTKKKYSSSVHDPNGEYMFMRAVNTAKKSRLTDVTLQPFMRPVQTTQEE
DGCSCRFPEEEEGGCEL
HLA-A2 Signal Peptide_PD-1 Extracellular_ICOS Transmembrane_ICOS
Signaling Domain_Truncated OX-40 Signaling Domain (Other names:
PD1 HLASP-ICOS-truncated OX40; HLA A2-SP-PD-1_ICOS_OX40t,
HLA A2-SP-PD-1_ICOS 40t; HLA A2-SP-PD-1_ICOS_truncated CD134;
PD1_ICOS_OX40t; PD1:ICOS40t) PD 1 extracellular domain in
regular font; ICOS domain underlined; and truncated OX40
(truncated CD134 signaling domain) in bold
                                                                                            (SEQ ID NO: 28)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPASQLCCQLKFWLPIGCAAFV
VVCILGCILICWLTKKKYSSSVHDPNGEYMFMRAVNTAKKSRLTDVTLGGGSFRTPIQEEQ
ADAHSTLA
HLA-A2 Signal Peptide_PD-1 Extracellular_ICOS Transmembrane_ICOS
Signaling Domain (mini-CD28)_Truncated 4-1BB Signaling Domain
(Other names: PD1_ICOS(28)_BBt; PD1_ICOS(28)_truncated CD137;
PD1:ICOS(28)-BBt or PD1:ICOS(28)BBt) PD 1 extracellular domain
in regular font; ICOS(28) domain: ICOS portions are underlined
and inserted CD28 portion bold and underline; and truncated
CD137 domain in bold
                                                                                            (SEQ ID NO: 29)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPASQLCCQLKFWLPIGCAAFV
VVCILGCILICWLTKKKYSSSVHDPNGEYMFMTPRRPGPTRKHYQPYAPPRAVNTAKKSRL
TDVTLQPFMRPVQTTQEEDGCSCRFPEEEEGGCEL
HLA-A2 Signal Peptide_PD-1 Extracellular_ICOS Transmembrane_ICOS
Signaling Domain (mini-CD28)_Truncated OX-40 Signaling Domain
(Other names: HLASP-ICOS DS-CD28(PRRP)-mutated CD134; PD1_ICOS
(28)_OX40t; PD1_ICOS(28)_40t; PD1_ICOS(28)_truncated CD134;
PD1:ICOS(28)-OX40t; PD1:ICOS(28)OX40t; PD1:ICOS(28)-40t or
PD1:ICOS(28)40t) PD 1 extracellular domain in regular font;
ICOS (28) domain: ICOS portions are underlined and inserted
CD28 portion bold and underline; and truncated CD134 domain
in bold
                                                                                            (SEQ ID NO: 30)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPASQLCCQLKFWLPIGCAAFV
VVCILGCILICWLTKKKYSSSVHDPNGEYMFMTPRRPGPTRKHYQPYAPPRAVNTAKKSRL
TDVTLGGGSFRTPIQEEQADAHSTLA
PD-1 Extracellular (without HLA A2 Signal Peptide
                                                                                            (SEQ ID NO: 31)
FLDSPDRPWNPPTFSPALLVVTEGDNATFTCSFSNTSESFVLNWYRMSPSNQTDKLAAFPE
DRSQPGQDCRFRVTQLPNGRDFHMSVVRARRNDSGTYLCGAISLAPKAQIKESLRAELRVT
ERRAEVPTAHPSPSPRPA
PD-1 extracellular domain without signal peptide-CD28 domain
swap (DS) (Other name: PD1_28)
                                                                                            (SEQ ID NO: 32)
FLDSPDRPWNPPTFSPALLVVTEGDNATFTCSFSNTSESFVLNWYRMSPSNQTDKLAAFPE
DRSQPGQDCRFRVTQLPNGRDFHMSVVRARRNDSGTYLCGAISLAPKAQIKESLRAELRVT
ERRAEVPTAHPSPSPRPALFPGPSKPFWVLVVVGGVLACYSLLVTVAFIIFWVRSKRSRLL
HSDYMNMTPRRPGPTRKHYQPYAPPRDFAAYRS
PD-1 extracellular domain without signal peptide-CD28 DS-CD137
domain (Other name: PD1_28_BBwt)
                                                                                            (SEQ ID NO: 33)
FLDSPDRPWNPPTFSPALLVVTEGDNATFTCSFSNTSESFVLNWYRMSPSNQTDKLAAFPE
DRSQPGQDCRFRVTQLPNGRDFHMSVVRARRNDSGTYLCGAISLAPKAQIKESLRAELRVT
ERRAEVPTAHPSPSPRPALFPGPSKPFWVLVVVGGVLACYSLLVTVAFIIFWVRSKRSRLL
HSDYMNMTPRRPGPTRKHYQPYAPPRDFAAYRSKRGRKKLLYIFKQPFMRPVQTTQEEDGC
SCRFPEEEEGGCEL
PD-1 extracellular domain without signal peptide-CD28 DS-
truncated CD137 domain (Other name: PD1_28_BBt)
                                                                                            (SEQ ID NO: 34)
FLDSPDRPWNPPTFSPALLVVTEGDNATFTCSFSNTSESFVLNWYRMSPSNQTDKLAAFPE
DRSQPGQDCRFRVTQLPNGRDFHMSVVRARRNDSGTYLCGAISLAPKAQIKESLRAELRVT
ERRAEVPTAHPSPSPRPALFPGPSKPFWVLVVVGGVLACYSLLVTVAFIIFWVRSKRSRLL
HSDYMNMTPRRPGPTRKHYQPYAPPRDFAAYRSQPFMRPVQTTQEEDGCSCRFPEEEEGGC
EL
PD-1 extracellular domain without signal peptide-CD28 DS-
truncated CD134 domain (Other name: PD1_28_OX40t)
                                                                                            (SEQ ID NO: 35)
FLDSPDRPWNPPTFSPALLVVTEGDNATFTCSFSNTSESFVLNWYRMSPSNQTDKLAAFPE
DRSQPGQDCRFRVTQLPNGRDFHMSVVRARRNDSGTYLCGAISLAPKAQIKESLRAELRVT
ERRAEVPTAHPSPSPRPALFPGPSKPFWVLVVVGGVLACYSLLVTVAFIIFWVRSKRSRLL
HSDYMNMTPRRPGPTRKHYQPYAPPRDFAAYRSGGGSFRTPIQEEQADAHSTLA
PD-1 extracellular domain without signal peptide-ICOS DS
(Other name: PD1_ICOS)
                                                                                            (SEQ ID NO: 36)
FLDSPDRPWNPPTFSPALLVVTEGDNATFTCSFSNTSESFVLNWYRMSPSNQTDKLAAFPE
DRSQPGQDCRFRVTQLPNGRDFHMSVVRARRNDSGTYLCGAISLAPKAQIKESLRAELRVT
ERRAEVPTAHPSPSPRPASQLCCQLKFWLPIGCAAFVVVCILGCILICWLTKKKYSSSVHD
PNGEYMFMRAVNTAKKSRLTDVTL
PD-1 extracellular domain without signal peptide-ICOS DS-CD137
(Other name: PD1_ICOS_BBwt)
                                                                                            (SEQ ID NO: 37)
FLDSPDRPWNPPTFSPALLVVTEGDNATFTCSFSNTSESFVLNWYRMSPSNQTDKLAAFPE
DRSQPGQDCRFRVTQLPNGRDFHMSVVRARRNDSGTYLCGAISLAPKAQIKESLRAELRVT
ERRAEVPTAHPSPSPRPASQLCCQLKFWLPIGCAAFVVVCILGCILICWLTKKKYSSSVHD
PNGEYMFMRAVNTAKKSRLTDVTLKRGRKKLLYIFKQPFMRPVQTTQEEDGCSCRFPEEEE
GGCEL
PD-1 extracellular domain without signal peptide-ICOS DS-
truncated CD137 (Other name: PD1_ICOS_BBt)
                                                                                            (SEQ ID NO: 38)
FLDSPDRPWNPPTFSPALLVVTEGDNATFTCSFSNTSESFVLNWYRMSPSNQTDKLAAFPE
DRSQPGQDCRFRVTQLPNGRDFHMSVVRARRNDSGTYLCGAISLAPKAQIKESLRAELRVT
ERRAEVPTAHPSPSPRPASQLCCQLKFWLPIGCAAFVVVCILGCILICWLTKKKYSSSVHD
PNGEYMFMRAVNTAKKSRLTDVTLQPFMRPVQTTQEEDGCSCRFPEEEEGGCEL
PD-1 extracellular domain without signal peptide-ICOS DS-
truncated CD134 (Other name: PD1_ICOS_OX40t
                                                                                            (SEQ ID NO: 39)
FLDSPDRPWNPPTFSPALLVVTEGDNATFTCSFSNTSESFVLNWYRMSPSNQTDKLAAFPE
DRSQPGQDCRFRVTQLPNGRDFHMSVVRARRNDSGTYLCGAISLAPKAQIKESLRAELRVT
ERRAEVPTAHPSPSPRPASQLCCQLKFWLPIGCAAFVVVCILGCILICWLTKKKYSSSVHD
PNGEYMFMRAVNTAKKSRLTDVTLGGGSFRTPIQEEQADAHSTLA
PD-1 extracellular domain without signal peptide-ICOS DS-CD28
(PRRP)-truncated CD137)(Other name: PD1_ICOS(28)_BBt
                                                                                            (SEQ ID NO: 40)
FLDSPDRPWNPPTFSPALLVVTEGDNATFTCSFSNTSESFVLNWYRMSPSNQTDKLAAFPE
DRSQPGQDCRFRVTQLPNGRDFHMSVVRARRNDSGTYLCGAISLAPKAQIKESLRAELRVT
ERRAEVPTAHPSPSPRPASQLCCQLKFWLPIGCAAFVVVCILGCILICWLTKKKYSSSVHD
PNGEYMFMTPRRPGPTRKHYQPYAPPRAVNTAKKSRLTDVTLQPFMRPVQTTQEEDGCSCR
FPEEEEGGCEL
PD-1 extracellular domain without signal peptide-ICOS DS-CD28
(PRRP)-truncated CD134 (Other name: PD1_ICOS(28)_OX40t)
                                                                                            (SEQ ID NO: 41)
FLDSPDRPWNPPTFSPALLVVTEGDNATFTCSFSNTSESFVLNWYRMSPSNQTDKLAAFPE
DRSQPGQDCRFRVTQLPNGRDFHMSVVRARRNDSGTYLCGAISLAPKAQIKESLRAELRVT
ERRAEVPTAHPSPSPRPASQLCCQLKFWLPIGCAAFVVVCILGCILICWLTKKKYSSSVHD
PNGEYMFMTPRRPGPTRKHYQPYAPPRAVNTAKKSRLTDVTLGGGSFRTPIQEEQADAHST
LA
PD-1 extracellular domain without signal peptide-ICOS DS-
truncated CD137 domain-truncated CD2 signaling domain (Other
name: PD1_ICOS_BBt:CD2t)
                                                                                            (SEQ ID NO: 42)
FLDSPDRPWNPPTFSPALLVVTEGDNATFTCSFSNTSESFVLNWYRMSPSNQTDKLAAFPE
DRSQPGQDCRFRVTQLPNGRDFHMSVVRARRNDSGTYLCGAISLAPKAQIKESLRAELRVT
ERRAEVPTAHPSPSPRPASQLCCQLKFWLPIGCAAFVVVCILGCILICWLTKKKYSSSVHD
PNGEYMFMRAVNTAKKSRLTDVTLQPFMRPVQTTQEEDGCSCRFPEEEEGGCELQNPATSQ
HPPPPPGHRSQAPSHRPPPPGHRVQHQPQKRPPAPSGTQVHQQKGPPLPRPRVQPKPPHGA
AENSLSPSSN
PD-1 extracellular domain without signal peptide-ICOS DS-
truncated CD137 domain-truncated CD2 signaling domain (Other
name: PD1_ICOS_BBt:IL2RB(YLRQ))
                                                                                            (SEQ ID NO: 43)
FLDSPDRPWNPPTESPALLVVTEGDNATFTCSFSNTSESFVLNWYRMSPSNQTDKLAAFPE
DRSQPGQDCRFRVTQLPNGRDFHMSVVRARRNDSGTYLCGAISLAPKAQIKESLRAELRVT
ERRAEVPTAHPSPSPRPASQLCCQLKFWLPIGCAAFVVVCILGCILICWLTKKKYSSSVHD
PNGEYMFMRAVNTAKKSRLTDVTLQPFMRPVQTTQEEDGCSCRFPEEEEGGCELNCRNTGP
WLKKVLKCNTPDPSKFFSQLSSEHGGDVQKWLSSPFPSSSFSPGGLAPEISPLEVLERDKV
TQLLPLNTDAYLSLQELQGQDPTHLVSYLRQWVVIPPPLSSPGPQAS
PD-1 extracellular domain without signal peptide-ICOS DS-
truncated CD137 domain-truncated CD2 signaling domain- IL-2
receptor binding (IL2RB)(YLRQ) protein (Other name:
PD1_ICOS_BBt_CD2t_IL2RB(YLRQ)
                                                                                            (SEQ ID NO: 44)
FLDSPDRPWNPPTFSPALLVVTEGDNATFTCSFSNTSESFVLNWYRMSPSNQTDKLAAFPE
DRSQPGQDCRFRVTQLPNGRDFHMSVVRARRNDSGTYLCGAISLAPKAQIKESLRAELRVT
ERRAEVPTAHPSPSPRPASQLCCQLKFWLPIGCAAFVVVCILGCILICWLTKKKYSSSVHD
PNGEYMFMRAVNTAKKSRLTDVTLQPFMRPVQTTQEEDGCSCRFPEEEEGGCELQNPATSQ
HPPPPPGHRSQAPSHRPPPPGHRVQHQPQKRPPAPSGTQVHQQKGPPLPRPRVQPKPPHGA
AENSLSPSSNNCRNTGPWLKKVLKCNTPDPSKFFSQLSSEHGGDVQKWLSSPFPSSSFSPG
GLAPEISPLEVLERDKVTQLLPLNTDAYLSLQELQGQDPTHLVSYLRQWVVIPPPLSSPGP
QAS
HLA-A2 Signal Peptide_PD-1 Extracellular_CD28 Transmembrane_CD28
Signaling Domain_4-1BB Signaling Domain (Other names:
PD1_28_BBwt; PD1_28_BB; PD1_CD28_BB; PD1_CD28_BB wt;
PD1_CD28_CD137; PD1:28BB; PD1:28BB wt; PD1:28-BB or
PD1:28-BB wt)
                                                                                          (SEQ ID NO: 130)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPALFPGPSKPFWVLVVVGGVL
ACYSLLVTVAFIIFWVRSKRSRLLHSDYMNMTPRRPGPTRKHYQPYAPPRDFAAYRSKRGR
KKLLYIFKQPFMRPVQTTQEEDGCSCRFPEEEEGGCEL
HLA-A2 Signal Peptide_PD1 Extracellular_CD28 Transmembrane_CD28
Signaling Domain_Truncated 4-1BB Signaling Domain (Other names:
PD1_28_BBt; PD1_CD28_BBt; PD1_CD28_truncated CD137; PD1:28BBt;
or PD1:28-BBt)
                                                                                          (SEQ ID NO: 131)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPALFPGPSKPFWVLVVVGGVL
ACYSLLVTVAFIIFWVRSKRSRLLHSDYMNMTPRRPGPTRKHYQPYAPPRDFAAYRSQPFM
RPVQTTQEEDGCSCRFPEEEEGGCEL
HLA-A2 Signal Peptide_PD-1 Extracellular_CD28 Transmembrane_CD28
Signaling Domain_Truncated OX-40 Signaling Domain (Other names:
PD1_28_OX40t; PD1_28_40t; PD1_CD28_OX40t; PD1_CD28_40t;
PD-1_CD28_truncated CD134; PD1:2840t; PD1:280X40t; PD1:20-OX40t
or PD1:28-40t) PD1 Extracellular (with HLA-A2 Signal
Peptide)_CD28 Transmembrane_CD28 Signaling Domain_Truncated
OX-40 Signaling Domain
                                                                                          (SEQ ID NO: 132)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPALFPGPSKPFWVLVVVGGVL
ACYSLLVTVAFIIFWVRSKRSRLLHSDYMNMTPRRPGPTRKHYQPYAPPRDFAAYRSGGGS
FRTPIQEEQADAHSTLA
HLA-A2 Signal Peptide_PD-1 Extracellular_ICOS (Stalk,
Transmembrane, and Signaling Domain)_TNFRSF4/OX40/CD134
Signaling Domain (Other name: PD1-ICOS-TNR4(OX40))
                                                                                            (SEQ ID NO: 45)
MAVMAPRTLVLLLSGALALTQTWAFLDSPDRPWNPPTFSPALLVVTEGDNATFTCSESNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPASQLCCQLKFWLPIGCAAFV
VVCILGCILICWLTKKKYSSSVHDPNGEYMFMRAVNTAKKSRLTDVTLALYLLRRDQRLPP
DAHKPPGGGSFRTPIQEEQADAHSTLAKI
HLA-A2 Signal Peptide_PD-1 Extracellular_ICOS (Stalk,
Transmembrane, and Signaling Domain)_TNFRSF5 Signaling
Domain (Other name: PD1-ICOS-TNR5)
                                                                                            (SEQ ID NO: 46)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPASQLCCQLKEWLPIGCAAFV
VVCILGCILICWLTKKKYSSSVHDPNGEYMFMRAVNTAKKSRLTDVTLKKVAKKPTNKAPH
PKQEPQEINFPDDLPGSNTAAPVQETLHGCQPVTQEDGKESRISVQERQ
HLA-A2 Signal Peptide_PD-1 Extracellular_ICOS (Stalk,
Transmembrane, and Signaling Domain)_TNFRSF7 (CD27) Signaling
Domain (Other name: PD1-ICOS-TNR7)
                                                                                            (SEQ ID NO: 47)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPASQLCCQLKFWLPIGCAAFV
VVCILGCILICWLTKKKYSSSVHDPNGEYMFMRAVNTAKKSRLTDVTLQRRKYRSNKGESP
VEPAEPCHYSCPREEEGSTIPIQEDYRKPEPACSP
HLA-A2 Signal Peptide_PD-1 Extracellular_ICOS (Stalk,
Transmembrane, and Signaling Domain)_TNFRSF9 (4-1BB)
Signaling Domain (Other name: PD1-ICOS-TNR9(41BB))
                                                                                            (SEQ ID NO: 48)
MAVMAPRTLVLLLSGALALTQTWAFLDSPDRPWNPPTESPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPASQLCCQLKFWLPIGCAAFV
VVCILGCILICWLTKKKYSSSVHDPNGEYMFMRAVNTAKKSRLTDVTLKRGRKKLLYIFKQ
PFMRPVQTTQEEDGCSCRFPEEEEGGCEL
HLA-A2 Signal Peptide_PD-1 Extracellular_ICOS (Stalk,
Transmembrane, and Signaling Domain)_Truncated TNFRSF9
Signaling Domain (41BBt) (Other name: PD1-ICOS-TNR9(41BBt))
                                                                                            (SEQ ID NO: 52)
MAVMAPRTLVLLLSGALALTOTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPASQLCCQLKEWLPIGCAAFV
VVCILGCILICWLTKKKYSSSVHDPNGEYMFMRAVNTAKKSRLTDVTLQPFMRPVQTTQEE
DGCSCRFPEEEEGGCEL
HLA-A2 Signal Peptide_PD-1 Extracellular_ICOS (Stalk,
Transmembrane, and Signaling Domain)_Truncated TNFRSF4
Signaling Domain (OX40t) (Other name: PD1-ICOS-TNR4(OX40t))
                                                                                            (SEQ ID NO: 53)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPASQLCCQLKEWLPIGCAAFV
VVCILGCILICWLTKKKYSSSVHDPNGEYMFMRAVNTAKKSRLTDVTLGGGSFRTPIQEEQ
ADAHSTLA
HLA-A2 Signal Peptide_PD-1 Extracellular_ICOS (Stalk,
Transmembrane, and Signaling Domain)_Truncated TNFRSF5
Signaling Domain (TNFRSF5t) (Other name: PD1-ICOS-TNR5t)
                                                                                            (SEQ ID NO: 54)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPASQLCCQLKFWLPIGCAAFV
VVCILGCILICWLTKKKYSSSVHDPNGEYMFMRAVNTAKKSRLTDVTLAPHPKQEPQEINF
PDDLPGSNTAAPVQETLHGCQPVTQEDGKESRISVQERQ
HLA-A2 Signal Peptide_PD-1 Extracellular_ICOS (Stalk,
Transmembrane, and Signaling Domain)_Truncated TNFRSF7 (CD27)
Signaling Domain (TNFRSF7t) (Other name: PD1-ICOS-TNR7t)
                                                                                            (SEQ ID NO: 55)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPASQLCCQLKFWLPIGCAAFV
VVCILGCILICWLTKKKYSSSVHDPNGEYMFMRAVNTAKKSRLTDVTLGESPVEPAEPCHY
SCPREEEGSTIPIQEDYRKPEPACSP
HLA-A2 Signal Peptide_PD-1 Extracellular_CD28 (Stalk,
Transmembrane, and Signaling Domain)_TNFRSF4/OX40/CD134
Signaling Domain (Other name: PD1-28-TNR4(OX40))
                                                                                            (SEQ ID NO: 56)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPALFPGPSKPFWVLVVVGGVL
ACYSLLVTVAFIIFWVRSKRSRLLHSDYMNMTPRRPGPTRKHYQPYAPPRDFAAYRSALYL
LRRDQRLPPDAHKPPGGGSFRTPIQEEQADAHSTLAKI
HLA-A2 Signal Peptide_PD-1 Extracellular_CD28 (Stalk,
Transmembrane, and Signaling Domain)_TNFRSF5 Signaling
Domain (Other name: PD1-28-TNR5)
                                                                                            (SEQ ID NO: 57)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPALFPGPSKPFWVLVVVGGVL
ACYSLLVTVAFIIFWVRSKRSRLLHSDYMNMTPRRPGPTRKHYQPYAPPRDFAAYRSKKVA
KKPTNKAPHPKQEPQEINFPDDLPGSNTAAPVQETLHGCQPVTQEDGKESRISVQERQ
HLA-A2 Signal Peptide_PD-1 Extracellular_CD28 (Stalk,
Transmembrane, and Signaling Domain)_TNFRSF7 (CD27) Signaling
Domain (Other name: PD1-28-TNR7)
                                                                                            (SEQ ID NO: 58)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPALFPGPSKPFWVLVVVGGVL
ACYSLLVTVAFIIFWVRSKRSRLLHSDYMNMTPRRPGPTRKHYQPYAPPRDFAAYRSQRRK
YRSNKGESPVEPAEPCHYSCPREEEGSTIPIQEDYRKPEPACSP
HLA-A2 Signal Peptide_PD-1 Extracellular_CD28 (Stalk,
Transmembrane, and Signaling Domain)_ TNFRSF9 (4-1BB)
Signaling Domain (Other name: PD1-28-TNR9(41BB))
                                                                                            (SEQ ID NO: 59)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPALFPGPSKPFWVLVVVGGVL
ACYSLLVTVAFIIFWVRSKRSRLLHSDYMNMTPRRPGPTRKHYQPYAPPRDFAAYRSKRGR
KKLLYIFKQPFMRPVQTTQEEDGCSCRFPEEEEGGCEL
HLA-A2 Signal Peptide_PD-1 Extracellular_CD28 (Stalk,
Transmembrane, and Signaling Domain)_Truncated TNFRSF9
Signaling Domain (41BBt) (Other name: PD1-28-TNR9(41BBt))
                                                                                            (SEQ ID NO: 60)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPALFPGPSKPFWVLVVVGGVL
ACYSLLVTVAFIIFWVRSKRSRLLHSDYMNMTPRRPGPTRKHYQPYAPPRDFAAYRSQPFM
RPVQTTQEEDGCSCRFPEEEEGGCEL
HLA-A2 Signal Peptide_PD-1 Extracellular_CD28 (Stalk,
Transmembrane, and Signaling Domain)_Truncated TNFRSF4
Signaling Domain (OX40t) (Other name: PD1-28-TNR4(OX40t))
                                                                                            (SEQ ID NO: 61)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPALFPGPSKPFWVLVVVGGVL
ACYSLLVTVAFIIFWVRSKRSRLLHSDYMNMTPRRPGPTRKHYQPYAPPRDFAAYRSGGGS
FRTPIQEEQADAHSTLA
HLA-A2 Signal Peptide_PD-1 Extracellular_CD28 (Stalk,
Transmembrane, and Signaling Domain)_Truncated TNFRSF5
Signaling Domain (TNFRSF5t) (Other name: PD1-28-TNR5t)
                                                                                            (SEQ ID NO: 62)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPALFPGPSKPFWVLVVVGGVL
ACYSLLVTVAFIIFWVRSKRSRLLHSDYMNMTPRRPGPTRKHYQPYAPPRDFAAYRSAPHP
KQEPQEINFPDDLPGSNTAAPVQETLHGCQPVTQEDGKESRISVQERQ
HLA-A2 Signal Peptide_PD-1 Extracellular_CD28 (Stalk,
Transmembrane, and Signaling Domain)_Truncated TNFRSF7 (CD27)
Signaling Domain (TNFRSF7t)(Other name: PD1-28-TNR7t)
                                                                                            (SEQ ID NO: 63)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPALFPGPSKPFWVLVVVGGVL
ACYSLLVTVAFIIFWVRSKRSRLLHSDYMNMTPRRPGPTRKHYQPYAPPRDFAAYRSGESP
VEPAEPCHYSCPREEEGSTIPIQEDYRKPEPACSP
HLA-A2 Signal Peptide_PD-1 Extracellular_CD28 (Stalk,
Transmembrane, and Signaling Domain)_TNFRSF9 (4-1BB)
Transmembrane and Signaling Domain (Other name: PD1-28-
TNR9(41BB))
                                                                                            (SEQ ID NO: 64)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPALFPGPSKPFWVLVVVGGVL
ACYSLLVTVAFIIFWVRSKRSRLLHSDYMNMTPRRPGPTRKHYQPYAPPRDFAAYRSKRGR
KKLLYIFKOPFMRPVQTTQEEDGCCRFPEEEEGGCEL
HLA-A2 Signal Peptide_PD-1 Extracellular_TNFRSF4/OX40/CD134
Transmembrane and Signaling Domain (Other name: PD1-TNR4(OX40))
                                                                                            (SEQ ID NO: 65)
MAVMAPRTLVLLLSGALALTQTWAFLDSPDRPWNPPTESPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPAVAAILGLGLVLGLLGPLAI
LLALYLLRRDQRLPPDAHKPPGGGSFRTPIQEEQADAHSTLAKI
HLA-A2 Signal Peptide PD-1 Extracellular_TNFRSF9 (4-1BB)
Transmembrane and Signaling Domain (Other name: PD1-TNR9(41BB))
                                                                                            (SEQ ID NO: 66)
MAVMAPRTLVLLLSGALALTQTWAFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGTY
LCGAISLAPKAQIKESLRAELRVTERRAEVPTAHPSPSPRPAIISFFLALTSTALLFLLFF
LTLRFSVVKRGRKKLLYIFKQPFMRPVQTTQEEDGCSCRFPEEEEGGCEL
```

TABLE 6
Example amino acid sequences of third and fourth signaling domains of RTCR.

CD2 truncated Signaling Domain (Other name: CD2 or 2) (SEQ ID NO: 49)
QNPATSQHPPPPPGHRSQAPSHRPPPPGHRVQHQPQKRPPAPSGTQVHQQKGPPLPRP
RVQPKPPHGAAENSLSPSSN
IL-2 receptor binding (IL2RB) protein Signaling Domain (YLRQ shown in bold) (Other
name: IL2RB(YLRQ) (SEQ ID NO: 50)
NCRNTGPWLKKVLKCNTPDPSKFFSQLSSEHGGDVQKWLSSPFPSSSFSPGGLAPEISPLE
VLERDKVTQLLPLNTDAYLSLQELQGQDPTHLVSYLRQWVVIPPPLSSPGPQAS

In some embodiments, the extracellular domain is derived from CD19 binding protein. In some embodiments, the CD19 binding protein is a CD19 binding chimeric antigen receptor (CAR). In some embodiments, the extracellular domain comprises the amino acid sequence according to SEQ ID NO: 51. In some embodiments, the extracellular domain comprises the amino acid sequence having at least 50%, 55%, 60%, 65%, 70%, 75%, 80%, 85%, 90%, 95%, 97% or 99% identity to SEQ ID NO: 51.

TABLE 7
Example amino acid sequences related to CD19 CAR based RTCR.

CD19 binding extracellular domain, FMC63scFV (Other name: CD19) (SEQ ID NO: 51):
CD8a leader/signal peptide (bold, SEQ ID NO: 117) and CD8a Hinge (underlined, SEQ ID
NO: 118) [FMC63 scFV (CD8a Leader_Light Chain_Linker_Heavy Chain_CD8a Hinge)]
MALPVTALLLPLALLLHAARPDIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGG
TKLEITGGGGSGGGGSGGGGSEVKLQESGPGLVAPSQSLSVTCTVSGVSLPDYGVSWIRQP
PRKGLEWLGVIWGSETTYYNSALKSRLTIIKDNSKSQVFLKMNSLQTDDTAIYYCAKHYYY
GGSYAMDYWGQGTSVTVSSTTTPAPRPPTPAPTIASQPLSLRPEACRPAAGGAVHTRGLDF
AC

In some embodiments, the extracellular domain comprises a hinge region. In some embodiments, the hinge region is derived from CD8, PD-1, CD28, ICOS, or IgG. In some embodiments, the transmembrane domain of the RTCR disclosed herein, is derived from CD8, PD-1, CD28, ICOS, CD134, TNFRSF5, TNFRSF7, 4-1BB, or IgG.

The present disclosure also provides a nucleic acid encoding the RTCR disclosed herein. In some embodiments, the nucleic acid encoding the RTCR disclosed herein is according to SEQ ID NO: 75-86. In some embodiments, the nucleic acid disclosed herein comprises a nucleic acid sequence encoding a chimeric intracellular domain. In some embodiments, the RTCR disclosed herein is for expression in a T cell, wherein the T cell co-expresses at least one of the endogenous co-stimulatory molecules CD28, CD2, OX-40, ICOS, CD28, CD4, CD8, CD134, TNFRSF5, TNFRSF7, 4-1BB, and CD40L or a combination thereof.

In some embodiments, an RTCR of the disclosure, or a nucleic acid encoding the same, comprises a transmembrane and signaling domain. In some embodiments, the transmembrane and signaling domain are selected from CD8, PD-1, CD28, ICOS, CD134, TNFRSF5, TNFRSF7, and 4-1BB. In some embodiments, the transmembrane and signaling domain are selected from Table 8.

TABLE 8
Example transmembrane and signaling domains for use in RTCR of the disclosure

| SEQ ID NO | Sequence |
|---|---|
| 240 | TNFRSF4/OX40/CD134 Transmembrane and Signaling Domain |
| | VAAILGLGLVLGLLGPLAILLALYLLRRDQRLPPDAHKPPGGGSFRTPIQEEQADAHSTLAKI |
| 241 | TNFRSF5 (CD40) Transmembrane and Signaling Domain |
| | ALVVIPIIFGILFAILLVLVFIKKVAKKPTNKAPHPKQEPQEINFPDDLPGSNTAAPVQETLH |
| | GCQPVTQEDGKESRISVQERQ |
| 242 | TNFRSF7 (CD27) Transmembrane and Signaling Domain |
| | ILVIFSGMFLVFTLAGALFLHQRRKYRSNKGESPVEPAEPCHYSCPREEEGSTIPIQEDYRKP |
| | EPACSP |
| 243 | TNFRSF9 (4-1BB) Transmembrane and Signaling Domain |
| | IISFFLALTSTALLFLLFFLTLRFSVVKRGRKKLLYIFKQPFMRPVQTTQEEDGCSCRFPEEE |
| | EGGCEL |

The present disclosure also provides a vector comprising the nucleic acid disclosed herein. In some embodiments, the vector disclosed herein is any one of a viral vector, a plasmid, a cosmid, a yeast artificial chromosome, a bacterial artificial chromosome or a transposon/transposase system. In some embodiments, the viral vector is an adeno-viral vector or a lentiviral vector. In some embodiments, the vector is a lentiviral vector.

The present disclosure also provides a cell comprising the nucleic acid or the vector disclosed herein. In some embodiments, the cell disclosed herein is a modified T cell. In some embodiments, the modified T cell is an allogenic T cell. In some embodiments, the modified T cell is an autologous T cell. In some embodiments, the modified T cell is any one of a naïve T cell, an early memory T cell, a stem cell-like T cell, a stem memory T cell (T_{SCM}), a central memory T cell (T_{CM}) and a regulatory T cell (T_{reg}).

In some embodiments, the extracellular domain is a B cell maturation Ag (BCMA) binding protein. In some embodiments, the BCMA binding protein is a BCMA specific T cell receptor (TCR). In some embodiments, the BCMA binding protein is a BCMA specific chimeric antigen receptor (CAR).

In some embodiments, the BCMA binding chimeric antigen receptor comprises the amino acid sequence according to SEQ ID NO: 137. In some embodiments, the BCMA binding chimeric antigen receptor comprises the amino acid sequence having at least 50%, 55%, 60%, 65%, 70%, 75%, 80%, 85%, 90%, 95%, 97% or 99% identity to SEQ ID NO: 137.

In some embodiments, the extracellular domain is a B cell maturation Ag (BCMA) binding protein. In some embodiments, the BCMA binding protein is a BCMA specific T cell receptor (TCR). In some embodiments, the BCMA binding protein is a BCMA specific chimeric antigen receptor (CAR).

TABLE 9
Example amino acid sequences of BCMA specific chimeric antigen receptors (CAR)
and BCMA specific CAR-based RTCR.

11-D5-3 scFv (CD8a Signal Peptide_11-D5-3 scFv, mouse_CD8a Hinge_BB_Z) (SEQ ID
NO: 133): CD8a Signal Peptide (bold, SEQ ID NO: 117)_11-D5-3 scFv, mouse
(italics)_CD8a Hinge (bold, SEQ ID NO: 118)_BB_Z)
MALPVTALLLPLALLLHAARPDIVLTQSPPSLAMSLGKRATISCRASESVTILGSHLIHWY
QQKPGQPPTLLIQLASNVQTGVPARFSGSGSRTDFTLTIDPVEEDDVAVYYCLQSRTIPRT
FGGGTKLEIKGSTSGSGKPGSGEGSTKGQIQLVQSGPELKKPGETVKISCKASGYTFTDYS
INWVKRAPGKGLKWMGWINTETREPAYAYDFRGRFAFSLETSASTAYLQINNLKYEDTATY
FCALDYSYAMDYWGQGTSVTVSSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSC
RFPEEEEGGCELRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATKDTYDALHMQAL
PPR
FHVH33 HVV (CD8a Signal Peptide_FHVH33 HVV_CD8a Hinge_BB_Z) (SEQ ID NO:
134): (CD8a Signal Peptide (bold, SEQ ID NO: 117)_FHVH33 HVV (italicized)_CD8a
Hinge (bold, SEQ ID NO: 118)_BB_Z)
MALPVTALLLPLALLLHAARPEVQLLESGGGLVQPGGSLRLSCAASGFTFSSYAMSWVRQA
PGKGLEWVSSISGSGDYIYYADSVKGRFTISRDISKNTLYLQMNSLRAEDTAVYYCAKEGT
GANSSLADYRGQGTLVTVSSFVPVFLPAKPTTTPAPRPPTPAPTIASQPLSLRPEACRPAA
GGAVHTRGLDFACDIYIWAPLAGTCGVLLLSLVITLYCNHRNKRGRKKLLYIFKQPFMRPV
QTTQEEDGCSCRFPEEEEGGCELRVKFSRSADAPAYQQGQNQLYNELNLGRREEYDVLDKR
RGRDPEMGGKPQRRKNPQEGLYNELQKDKMAEAYSEIGMKGERRRGKGHDGLYQGLSTATK
DTYDALHMQALPPR
BCAR003 HVV (CD8a Signal Peptide_BCAR003 HVV_CD8a Hinge_BB_Z) (SEQ ID
NO: 135): CD8a Signal Peptide (Bold)_BCAR003 HVV (italicized)_CD8a Hinge
(Bold)_BB_Z
MALPVTALLLPLALLLHAARPQVKLEESGGGLVQAGRSLRLSCAASEHTFSSHVMGWFRQA
PGKERESVAVIGWRDISTSYADSVKGRFTISRDNAKKTLYLQMNSLKPEDTAVYYCAARRI
DAADFDSWGQGTQVTVSSGGGGSGGGGSGGGGSGGGGSGGGGSAVQLVESGGGLVQAGDSL
RLTCTASGRAFSTYFMAWFRQAPGKEREFVAGIAWSGGSTAYADSVKGRFTISRDNAKNTV
YLQMNSLKSEDTAVYYCASRGIEVEEFGAWGQGTQVTVSSTSTTTPAPRPPTPAPTIASQP
LSLRPEACRPAAGGAVHTRGLDFACDIYIWAPLAGTCGVLLLSLVITLYCKRGRKKLLYIF
KQPFMRPVQTTQEEDGCSCRFPEEEEGGCELRVKFSRSADAPAYQQGQNQLYNELNLGRRE
EYDVLDKRRGRDPEMGGKPQRRKNPQEGLYNELQKDKMAEAYSEIGMKGERRRGKGHDGLY
QGLSTATKDTYDALHMQALPPR
GSI5022 HVV (CD8a Signal Peptide_GSI5022 HVV_CD8a Hinge_BB_Z) (SEQ ID NO:
136): CD8a Signal Peptide (bold, SEQ ID NO: 117)_GSI5022 HVV (italicized)_CD8a
Hinge (bold, SEQ ID NO: 118)_BB_Z
MALPVTALLLPLALLLHAARPQVKLEESGGGLVQAGRSLRLSCAASEHTFSSHVMGWFRQA
PGKERESVAVIGWRDISTSYADSVKGRFTISRDNAKKTLYLQMNSLKPEDTAVYYCAARRI
DAADFDSWGQGTQVTVSSGGGGSGGGGSGGGGSEVQLVESGGGLVQAGGSLRLSCAASGRT
FTMGWFRQAPGKEREFVAAISLSPTLAYYAESVKGRFTISRDNAKNTVVLQMNSLKPEDTA
LYYCAADRKSVMSIRPDYWGQGTQVTVSSTSTTTPAPRPPTPAPTIASQPLSLRPEACRPA
AGGAVHTRGLDFACDIYIWAPLAGTCGVLLLSLVITLYCKRGRKKLLYIFKQPFMRPVQTT
QEEDGCSCRFPEEEEGGCELRVKFSRSADAPAYQQGQNQLYNELNLGRREEYDVLDKRRGR
DPEMGGKPQRRKNPQEGLYNELQKDKMAEAYSEIGMKGERRRGKGHDGLYQGLSTATKDTY
DALHMQALPPR
BCMAsdAb1 HVV (CD8a Signal Peptide_anti-BCMAsdAB1 HHV_CD8a Hinge) (SEQ
ID NO: 137): CD8a Signal Peptide (bold, SEQ ID NO: 117)_anti-BCMAsdAB1 HHV
(italicized)_CD8a Hinge (bold, SEQ ID NO: 118)
MALPVTALLLPLALLLHAARPEVQLQASGGGLAQPGGSLRLSCAASGRTFSTYFMAWFRQP
PGKGLEYVGGIRWSDGVPHYADSVKGRFTISRDNAKNTVYLQMNSLRAEDTAVYFCASRGI
ADGSDFGSYGQGTQVTVSSTTTPAPRPPTPAPTIASQPLSLRPEACRPAAGGAVHTRGLDF
AC

TABLE 10
Nucleic acid sequences of intracellular signaling domains,
extracellular domains of RTCRs and RTCRs.

CD2 truncated Signaling Domain (SEQ ID NO: 70)
CAGAATCCTGCCACCTCTCAGCACCCTCCACCTCCACCTGGACACAGATCTCAGGCCCCAT
CTCACAGACCTCCACCACCTGGTCATCGGGTGCAGCATCAGCCCCAGAAAAGACCTCCTGC
TCCTAGCGGAACACAGGTGCACCAGCAAAAGGGACCTCCACTGCCTAGACCTAGAGTGCAG
CCTAAGCCTCCTCATGGCGCTGCCGAGAATAGCCTGTCTCCTAGCAGCAAC
IL2RB(YLRQ) Signaling Domain (SEQ ID NO: 71)
AATTGCAGAAACACAGGCCCCTGGCTGAAGAAAGTGCTGAAGTGCAACACCCCTGATCCGA
GCAAGTTCTTTAGCCAGCTGAGCAGCGAGCATGGCGGCGACGTTCAGAAATGGCTGTCTAG
CCCATTTCCTAGCAGCAGCTTCAGCCCTGGTGGACTGGCCCCTGAGATTAGCCCTCTGGAA
GTGCTGGAACGGGACAAAGTGACCCAGCTGCTGCCCCTGAATACCGACGCTTACCTGAGCC
TGCAAGAGCTGCAAGGACAGGACCCTACACACCTGGTGTCCTACCTGAGACAGTGGGTCGT
GATCCCTCCACCTCTCTCTAGTCCTGGACCTCAGGCCTCT
PD-1 (PD-1 Signal Peptide_PD1 Extracellular_PD1
Transmembrane_PD1 Intracellular)
PD-1 (Other name: PD1-wt or PD1 (SEQ ID NO: 72)
ATGCAGATTCCTCAAGCTCCTTGGCCTGTCGTGTGGGCCGTTCTGCAACTTGGATGGCGGC
CTGGCTGGTTCCTGGACTCTCCTGACAGACCCTGGAATCCTCCAACATTCAGCCCCGCTCT
GCTGGTGGTTACCGAGGGCGATAATGCCACCTTCACCTGTAGCTTCAGCAACACCAGCGAG
AGCTTCGTGCTGAACTGGTACAGAATGAGCCCCAGCAACCAGACCGACAAGCTGGCCGCCT
TTCCTGAGGATAGATCTCAGCCCGGCCAGGACTGCCGGTTCAGAGTTACACAGCTGCCCAA
CGGCCGGGACTTCCACATGTCTGTCGTCCGGGCCAGAAGAAACGACAGCGGCACATATCTG
TGCGGCGCCATTTCTCTGGCCCCTAAGGCTCAGATCAAAGAGAGCCTGAGAGCCGAGCTGA
GAGTGACAGAAAGACGGGCCGAAGTGCCCACAGCTCACCCTTCACCTTCTCCAAGACCTGC
CGGCCAGTTCCAGACACTGGTCGTGGGAGTTGTTGGCGGACTGCTGGGATCTCTGGTGCTG
CTTGTTTGGGTGCTCGCCGTGATCTGTAGCAGAGCCGCCAGAGGAACAATCGGCGCCAGAA
GGACAGGCCAGCCTCTGAAAGAGGATCCCTCTGCTGTCCCCGTGTTCAGCGTGGACTATGG
CGAGCTGGATTTCCAGTGGCGGGAAAAGACACCCGAGCCTCCAGTGCCTTGTGTGCCTGAG
CAGACAGAGTACGCCACCATCGTGTTCCCTAGCGGCATGGGCACATCTAGCCCTGCCAGAA
GAGGATCTGCCGACGGACCTAGATCTGCCCAGCCTCTCAGACCTGAGGATGGCCACTGTTC
TTGGCCTCTT
PD-1 Extracellular (Other name: PD-1) (SEQ ID NO: 73)
TTTCTGGACAGCCCCGACAGACCCTGGAATCCTCCTACATTCAGCCCCGCTCTGCTGGTGG
TTACCGAGGGCGATAATGCCACCTTCACCTGTAGCTTCAGCAACACCAGCGAGAGCTTCGT
GCTGAACTGGTACAGAATGAGCCCCAGCAACCAGACCGACAAGCTGGCCGCCTTTCCTGAG
GATAGATCTCAGCCCGGCCAGGACTGCCGGTTCAGAGTTACACAGCTGCCCAACGGCCGGG
ACTTCCACATGTCTGTCGTTCGGGCCAGAAGAAACGACAGCGGCACATATCTGTGCGGCGC
CATTTCTCTGGCCCCTAAGGCTCAGATCAAAGAGAGCCTGAGAGCCGAGCTGAGAGTGACA
GAAAGACGGGCCGAAGTGCCCACAGCTCACCCTTCACCTTCTCCAAGACCTGCT
PD-1 (extracellular domain without signal peptide)-CD28 domain
swap (DS) (SEQ ID NO: 74)
TTTCTGGACAGCCCCGACAGACCCTGGAATCCTCCTACATTCAGCCCCGCTCTGCTGGTGG
TTACCGAGGGCGATAATGCCACCTTCACCTGTAGCTTCAGCAACACCAGCGAGAGCTTCGT
GCTGAACTGGTACAGAATGAGCCCCAGCAACCAGACCGACAAGCTGGCCGCCTTTCCTGAG
GATAGATCTCAGCCCGGCCAGGACTGCCGGTTCAGAGTTACACAGCTGCCCAACGGCCGGG
ACTTCCACATGTCTGTCGTTCGGGCCAGAAGAAACGACAGCGGCACATATCTGTGCGGCGC
CATTTCTCTGGCCCCTAAGGCTCAGATCAAAGAGAGCCTGAGAGCCGAGCTGAGAGTGACA
GAAAGACGGGCCGAAGTGCCCACAGCTCACCCTTCACCTTCTCCAAGACCTGCTCTGTTCC
CCGGACCTAGCAAGCCCTTTTGGGTGCTCGTTGTTGTTGGCGGCGTGCTGGCCTGTTATAG
CCTGCTGGTTACCGTGGCCTTCATCATCTTTTGGGTCCGAAGCAAGCGGAGCCGGCTGCTG
CACAGCGACTACATGAACATGACCCCTAGACGGCCCGGACCAACCAGAAAGCACTACCAGC
CTTACGCTCCTCCTAGAGACTTCGCCGCCTACAGATCT
PD-1 (extracellular domain without signal peptide)-CD28
DS-CD137 domain (SEQ ID NO: 75)
TTTCTGGACAGCCCCGACAGACCCTGGAATCCTCCTACATTCAGCCCCGCTCTGCTGGTGG
TTACCGAGGGCGATAATGCCACCTTCACCTGTAGCTTCAGCAACACCAGCGAGAGCTTCGT
GCTGAACTGGTACAGAATGAGCCCCAGCAACCAGACCGACAAGCTGGCCGCCTTTCCTGAG
GATAGATCTCAGCCCGGCCAGGACTGCCGGTTCAGAGTTACACAGCTGCCCAACGGCCGGG
ACTTCCACATGTCTGTCGTTCGGGCCAGAAGAAACGACAGCGGCACATATCTGTGCGGCGC
CATTTCTCTGGCCCCTAAGGCTCAGATCAAAGAGAGCCTGAGAGCCGAGCTGAGAGTGACA
GAAAGACGGGCCGAAGTGCCCACAGCTCACCCTTCACCTTCTCCAAGACCTGCTCTGTTCC
CCGGACCTAGCAAGCCCTTTTGGGTGCTCGTTGTTGTTGGCGGCGTGCTGGCCTGTTATAG
CCTGCTGGTTACCGTGGCCTTCATCATCTTTTGGGTCCGAAGCAAGCGGAGCCGGCTGCTG
CACAGCGACTACATGAACATGACCCCTAGACGGCCCGGACCAACCAGAAAGCACTACCAGC
CTTACGCTCCTCCTAGAGACTTCGCCGCCTACAGATCCAAGCGGGGCAGAAAGAAGCTGCT
GTACATCTTCAAGCAGCCCTTCATGCGGCCCGTGCAGACCACACAAGAGGAAGATGGCTGC
TCCTGTCGGTTCCCCGAGGAAGAAGAAGGCGGTTGCGAACTG
PD-1 (extracellular domain without signal peptide)-CD28 DS-
truncated CD137 domain (SEQ ID NO: 76)
TTTCTGGACAGCCCCGACAGACCCTGGAATCCTCCTACATTCAGCCCCGCTCTGCTGGTGG
TTACCGAGGGCGATAATGCCACCTTCACCTGTAGCTTCAGCAACACCAGCGAGAGCTTCGT
GCTGAACTGGTACAGAATGAGCCCCAGCAACCAGACCGACAAGCTGGCCGCCTTTCCTGAG
GATAGATCTCAGCCCGGCCAGGACTGCCGGTTCAGAGTTACACAGCTGCCCAACGGCCGGG
ACTTCCACATGTCTGTCGTTCGGGCCAGAAGAAACGACAGCGGCACATATCTGTGCGGCGC
CATTTCTCTGGCCCCTAAGGCTCAGATCAAAGAGAGCCTGAGAGCCGAGCTGAGAGTGACA
GAAAGACGGGCCGAAGTGCCCACAGCTCACCCTTCACCTTCTCCAAGACCTGCTCTGTTCC
CCGGACCTAGCAAGCCCTTTTGGGTGCTCGTTGTTGTTGGCGGCGTGCTGGCCTGTTATAG
CCTGCTGGTTACCGTGGCCTTCATCATCTTTTGGGTCCGAAGCAAGCGGAGCCGGCTGCTG
CACAGCGACTACATGAACATGACCCCTAGACGGCCCGGACCAACCAGAAAGCACTACCAGC
CTTACGCTCCTCCTAGAGACTTCGCCGCCTACAGATCCCAGCCTTTCATGAGGCCTGTGCA
GACCACACAAGAAGAGGACGGCTGCTCCTGTCGGTTCCCCGAGGAAGAGGAAGGCGGTTGC
GAACTT
PD-1 (extracellular domain without signal peptide)-CD28 DS-
truncated CD134 domain (SEQ ID NO: 77)
TTTCTGGACAGCCCCGACAGACCCTGGAATCCTCCTACATTCAGCCCCGCTCTGCTGGTGG
TTACCGAGGGCGATAATGCCACCTTCACCTGTAGCTTCAGCAACACCAGCGAGAGCTTCGT
GCTGAACTGGTACAGAATGAGCCCCAGCAACCAGACCGACAAGCTGGCCGCCTTTCCTGAG
GATAGATCTCAGCCCGGCCAGGACTGCCGGTTCAGAGTTACACAGCTGCCCAACGGCCGGG
ACTTCCACATGTCTGTCGTTCGGGCCAGAAGAAACGACAGCGGCACATATCTGTGCGGCGC
CATTTCTCTGGCCCCTAAGGCTCAGATCAAAGAGAGCCTGAGAGCCGAGCTGAGAGTGACA
GAAAGACGGGCCGAAGTGCCCACAGCTCACCCTTCACCTTCTCCAAGACCTGCTCTGTTCC
CCGGACCTAGCAAGCCCTTTTGGGTGCTCGTTGTTGTTGGCGGCGTGCTGGCCTGTTATAG
CCTGCTGGTTACCGTGGCCTTCATCATCTTTTGGGTCCGAAGCAAGCGGAGCCGGCTGCTG
CACAGCGACTACATGAACATGACCCCTAGACGGCCCGGACCAACCAGAAAGCACTACCAGC
CTTACGCTCCTCCTAGAGACTTCGCCGCCTACAGATCTGGCGGCGGAAGCTTCAGAACCCC
TATCCAAGAGGAACAGGCCGACGCTCACTCTACACTGGCT
PD-1 (extracellular domain without signal peptide)-ICOS DS
(SEQ ID NO: 78)
TTTCTGGACAGCCCCGACAGACCCTGGAATCCTCCTACATTCAGCCCCGCTCTGCTGGTGG
TTACCGAGGGCGATAATGCCACCTTCACCTGTAGCTTCAGCAACACCAGCGAGAGCTTCGT
GCTGAACTGGTACAGAATGAGCCCCAGCAACCAGACCGACAAGCTGGCCGCCTTTCCTGAG
GATAGATCTCAGCCCGGCCAGGACTGCCGGTTCAGAGTTACACAGCTGCCCAACGGCCGGG
ACTTCCACATGTCTGTCGTTCGGGCCAGAAGAAACGACAGCGGCACATATCTGTGCGGCGC
CATTTCTCTGGCCCCTAAGGCTCAGATCAAAGAGAGCCTGAGAGCCGAGCTGAGAGTGACA
GAAAGACGGGCCGAAGTGCCCACAGCTCACCCTTCACCTTCTCCAAGACCTGCCAGCCAGC
TGTGCTGCCAGCTGAAGTTTTGGCTGCCTATCGGCTGTGCCGCCTTCGTGGTTGTGTGTAT
CCTGGGCTGCATCCTGATCTGCTGGCTGACCAAGAAAAAGTACAGCAGCTCCGTGCACGAC
CCCAACGGCGAGTACATGTTCATGAGAGCCGTGAACACCGCCAAGAAGTCCAGACTGACCG
ACGTGACACTT
PD-1 (extracellular domain without signal peptide)-ICOS
DS-truncated CD137 (SEQ ID NO: 79)
TTTCTGGACAGCCCCGACAGACCCTGGAATCCTCCTACATTCAGCCCCGCTCTGCTGGTGG
TTACCGAGGGCGATAATGCCACCTTCACCTGTAGCTTCAGCAACACCAGCGAGAGCTTCGT
GCTGAACTGGTACAGAATGAGCCCCAGCAACCAGACCGACAAGCTGGCCGCCTTTCCTGAG
GATAGATCTCAGCCCGGCCAGGACTGCCGGTTCAGAGTTACACAGCTGCCCAACGGCCGGG
ACTTCCACATGTCTGTCGTTCGGGCCAGAAGAAACGACAGCGGCACATATCTGTGCGGCGC
CATTTCTCTGGCCCCTAAGGCTCAGATCAAAGAGAGCCTGAGAGCCGAGCTGAGAGTGACA
GAAAGACGGGCCGAAGTGCCCACAGCTCACCCTTCACCTTCTCCAAGACCTGCCAGCCAGC
TGTGCTGCCAGCTGAAGTTTTGGCTGCCTATCGGCTGTGCCGCCTTCGTGGTTGTGTGTAT
CCTGGGCTGCATCCTGATCTGCTGGCTGACCAAGAAAAAGTACAGCAGCTCCGTGCACGAC
CCCAACGGCGAGTACATGTTCATGAGAGCCGTGAACACCGCCAAGAAGTCCAGACTGACCG
ACGTGACCCTGAAGCGGGGCAGAAAGAAACTGCTGTACATCTTCAAGCAGCCCTTCATGCG
GCCCGTGCAGACCACACAAGAGGAAGATGGCTGCTCCTGCAGATTCCCCGAGGAAGAAGAA
GGCGGCTGCGAACTT
PD-1 (extracellular domain without signal peptide)-ICOS
DS-truncated CD137 (SEQ ID NO: 80)
TTTCTGGACAGCCCCGACAGACCCTGGAATCCTCCTACATTCAGCCCCGCTCTGCTGGTGG
TTACCGAGGGCGATAATGCCACCTTCACCTGTAGCTTCAGCAACACCAGCGAGAGCTTCGT
GCTGAACTGGTACAGAATGAGCCCCAGCAACCAGACCGACAAGCTGGCCGCCTTTCCTGAG
GATAGATCTCAGCCCGGCCAGGACTGCCGGTTCAGAGTTACACAGCTGCCCAACGGCCGGG
ACTTCCACATGTCTGTCGTTCGGGCCAGAAGAAACGACAGCGGCACATATCTGTGCGGCGC
CATTTCTCTGGCCCCTAAGGCTCAGATCAAAGAGAGCCTGAGAGCCGAGCTGAGAGTGACA
GAAAGACGGGCCGAAGTGCCCACAGCTCACCCTTCACCTTCTCCAAGACCTGCCAGCCAGC
TGTGCTGCCAGCTGAAGTTTTGGCTGCCTATCGGCTGTGCCGCCTTCGTGGTTGTGTGTAT
CCTGGGCTGCATCCTGATCTGCTGGCTGACCAAGAAAAAGTACAGCAGCTCCGTGCACGAC
CCCAACGGCGAGTACATGTTCATGAGAGCCGTGAACACCGCCAAGAAGTCCAGACTGACCG
ACGTGACACTTCAGCCTTTCATGAGGCCCGTGCAGACCACACAAGAAGAGGACGGCTGCTC
CTGCAGATTCCCCGAGGAAGAGGAAGGCGGTTGCGAACTT
PD-1 (extracellular domain without signal peptide)-ICOS
DS-truncated CD134 (SEQ ID NO: 81)
TTTCTGGACAGCCCCGACAGACCCTGGAATCCTCCTACATTCAGCCCCGCTCTGCTGGTGG
TTACCGAGGGCGATAATGCCACCTTCACCTGTAGCTTCAGCAACACCAGCGAGAGCTTCGT
GCTGAACTGGTACAGAATGAGCCCCAGCAACCAGACCGACAAGCTGGCCGCCTTTCCTGAG
GATAGATCTCAGCCCGGCCAGGACTGCCGGTTCAGAGTTACACAGCTGCCCAACGGCCGGG
ACTTCCACATGTCTGTCGTTCGGGCCAGAAGAAACGACAGCGGCACATATCTGTGCGGCGC
CATTTCTCTGGCCCCTAAGGCTCAGATCAAAGAGAGCCTGAGAGCCGAGCTGAGAGTGACA
GAAAGACGGGCCGAAGTGCCCACAGCTCACCCTTCACCTTCTCCAAGACCTGCCAGCCAGC
TGTGCTGCCAGCTGAAGTTTTGGCTGCCTATCGGCTGTGCCGCCTTCGTGGTTGTGTGTAT
CCTGGGCTGCATCCTGATCTGCTGGCTGACCAAGAAAAAGTACAGCAGCTCCGTGCACGAC
CCCAACGGCGAGTACATGTTCATGAGAGCCGTGAACACCGCCAAGAAGTCCAGACTGACCG
ACGTGACACTTGGCGGCGGAAGCTTTAGAACCCCTATCCAAGAGGAACAGGCCGACGCTCA
CTCTACACTGGCT
PD-1 (extracellular domain without signal peptide)-ICOS
DS-CD28(PRRP)-truncated CD137) (SEQ ID NO: 82)
TTTCTGGACAGCCCCGACAGACCCTGGAATCCTCCTACATTCAGCCCCGCTCTGCTGGTGG
TTACCGAGGGCGATAATGCCACCTTCACCTGTAGCTTCAGCAACACCAGCGAGAGCTTCGT
GCTGAACTGGTACAGAATGAGCCCCAGCAACCAGACCGACAAGCTGGCCGCCTTTCCTGAG
GATAGATCTCAGCCCGGCCAGGACTGCCGGTTCAGAGTTACACAGCTGCCCAACGGCCGGG
ACTTCCACATGTCTGTCGTTCGGGCCAGAAGAAACGACAGCGGCACATATCTGTGCGGCGC
CATTTCTCTGGCCCCTAAGGCTCAGATCAAAGAGAGCCTGAGAGCCGAGCTGAGAGTGACA
GAAAGACGGGCCGAAGTGCCCACAGCTCACCCTTCACCTTCTCCAAGACCTGCCAGCCAGC
TGTGCTGCCAGCTGAAGTTTTGGCTGCCTATCGGCTGTGCCGCCTTCGTGGTTGTGTGTAT
CCTGGGCTGCATCCTGATCTGCTGGCTGACCAAGAAAAAGTACAGCAGCTCCGTGCACGAC
CCCAACGGCGAGTACATGTTCATGACCCCTAGAAGGCCTGGACCTACCAGAAAGCACTACC
AGCCTTACGCTCCTCCTAGAGCCGTGAACACCGCCAAGAAGTCCAGACTGACCGACGTGAC
ACTTCAGCCTTTCATGAGGCCCGTGCAGACCACACAAGAAGAGGACGGCTGCTCCTGCAGA
TTCCCCGAGGAAGAGGAAGGCGGTTGCGAACTT
PD-1 (extracellular domain without signal peptide)-ICOS
DS-CD28(PRRP)-truncated CD134) (SEQ ID NO: 83)
TTTCTGGACAGCCCCGACAGACCCTGGAATCCTCCTACATTCAGCCCCGCTCTGCTGGTGG
TTACCGAGGGCGATAATGCCACCTTCACCTGTAGCTTCAGCAACACCAGCGAGAGCTTCGT
GCTGAACTGGTACAGAATGAGCCCCAGCAACCAGACCGACAAGCTGGCCGCCTTTCCTGAG
GATAGATCTCAGCCCGGCCAGGACTGCCGGTTCAGAGTTACACAGCTGCCCAACGGCCGGG
ACTTCCACATGTCTGTCGTTCGGGCCAGAAGAAACGACAGCGGCACATATCTGTGCGGCGC
CATTTCTCTGGCCCCTAAGGCTCAGATCAAAGAGAGCCTGAGAGCCGAGCTGAGAGTGACA
GAAAGACGGGCCGAAGTGCCCACAGCTCACCCTTCACCTTCTCCAAGACCTGCCAGCCAGC
TGTGCTGCCAGCTGAAGTTTTGGCTGCCTATCGGCTGTGCCGCCTTCGTGGTTGTGTGTAT
CCTGGGCTGCATCCTGATCTGCTGGCTGACCAAGAAAAAGTACAGCAGCTCCGTGCACGAC
CCCAACGGCGAGTACATGTTCATGACCCCTAGAAGGCCTGGACCTACCAGAAAGCACTACC
AGCCTTACGCTCCTCCTAGAGCCGTGAACACCGCCAAGAAGTCCAGACTGACCGACGTGAC
ACTTGGCGGCGGAAGCTTTAGAACCCCTATCCAAGAGGAACAGGCCGACGCTCACTCTACA
CTGGCT
PD-1:ICOSBBt:CD2t (PD-1 extracellular domain without signal
peptide)-ICOS DS-truncated CD137 domain-truncated CD2
signaling domain) (SEQ ID NO: 84)
TTTCTGGACAGCCCCGACAGACCCTGGAATCCTCCTACATTCAGCCCCGCTCTGCTGGTGG
TTACCGAGGGCGATAATGCCACCTTCACCTGTAGCTTCAGCAACACCAGCGAGAGCTTCGT
GCTGAACTGGTACAGAATGAGCCCCAGCAACCAGACCGACAAGCTGGCCGCCTTTCCTGAG
GATAGATCTCAGCCCGGCCAGGACTGCCGGTTCAGAGTTACACAGCTGCCCAACGGCCGGG
ACTTCCACATGTCTGTCGTTCGGGCCAGAAGAAACGACAGCGGCACATATCTGTGCGGCGC
CATTTCTCTGGCCCCTAAGGCTCAGATCAAAGAGAGCCTGAGAGCCGAGCTGAGAGTGACA
GAAAGACGGGCCGAAGTGCCCACAGCTCACCCTTCACCTTCTCCAAGACCTGCCAGCCAGC
TGTGCTGCCAGCTGAAGTTTTGGCTGCCTATCGGCTGTGCCGCCTTCGTGGTTGTGTGTAT
CCTGGGCTGCATCCTGATCTGCTGGCTGACCAAGAAAAAGTACAGCAGCTCCGTGCACGAC
CCCAACGGCGAGTACATGTTCATGAGAGCCGTGAACACCGCCAAGAAGTCCAGACTGACCG
ACGTGACCCTCCAGCCTTTCATGAGGCCTGTGCAGACCACACAAGAAGAGGACGGCTGCTC
CTGTCGGTTCCCCGAGGAAGAGGAAGGCGGTTGCGAACTCCAGAATCCTGCCACCTCTCAG
CACCCTCCACCTCCACCTGGACACAGATCTCAGGCCCCATCTCACAGACCTCCACCACCTG
GTCATCGGGTGCAGCATCAGCCCCAGAAAAGACCTCCTGCTCCTAGCGGAACACAGGTGCA
CCAGCAAAAGGGACCTCCACTGCCTAGACCTAGAGTGCAGCCTAAGCCTCCTCATGGCGCT
GCCGAGAATAGCCTGTCTCCTAGCAGCAAC
PD-1 extracellular domain without signal peptide-ICOS
DS-truncated CD134 domain-truncated CD2 signaling domain
(SEQ ID NO: 85)
TTTCTGGACAGCCCCGACAGACCCTGGAATCCTCCTACATTCAGCCCCGCTCTGCTGGTGG
TTACCGAGGGCGATAATGCCACCTTCACCTGTAGCTTCAGCAACACCAGCGAGAGCTTCGT
GCTGAACTGGTACAGAATGAGCCCCAGCAACCAGACCGACAAGCTGGCCGCCTTTCCTGAG
GATAGATCTCAGCCCGGCCAGGACTGCCGGTTCAGAGTTACACAGCTGCCCAACGGCCGGG
ACTTCCACATGTCTGTCGTTCGGGCCAGAAGAAACGACAGCGGCACATATCTGTGCGGCGC
CATTTCTCTGGCCCCTAAGGCTCAGATCAAAGAGAGCCTGAGAGCCGAGCTGAGAGTGACA
GAAAGACGGGCCGAAGTGCCCACAGCTCACCCTTCACCTTCTCCAAGACCTGCCAGCCAGC
TGTGCTGCCAGCTGAAGTTTTGGCTGCCTATCGGCTGTGCCGCCTTCGTGGTTGTGTGTAT
CCTGGGCTGCATCCTGATCTGCTGGCTGACCAAGAAAAAGTACAGCAGCTCCGTGCACGAC
CCCAACGGCGAGTACATGTTCATGAGAGCCGTGAACACCGCCAAGAAGTCCAGACTGACCG
ACGTGACCCTCCAGCCTTTCATGAGGCCTGTGCAGACCACACAAGAAGAGGACGGCTGCTC
CTGTCGGTTCCCCGAGGAAGAGGAAGGCGGCTGCGAACTGAATTGCAGAAACACAGGCCCC
TGGCTGAAGAAAGTGCTGAAGTGCAACACCCCTGATCCGAGCAAGTTCTTTAGCCAGCTGA
GCAGCGAGCATGGCGGCGACGTTCAGAAATGGCTGTCTAGCCCATTTCCTAGCAGCAGCTT
CAGCCCTGGTGGACTGGCCCCTGAGATTAGCCCTCTGGAAGTGCTGGAACGGGACAAAGTG
ACCCAGCTGCTGCCCCTGAATACCGACGCTTACCTGAGCCTGCAAGAGCTGCAAGGACAGG
ACCCTACACACCTGGTGTCCTACCTGAGACAGTGGGTCGTGATCCCTCCACCTCTCTCTAG
TCCTGGACCTCAGGCCTCT
PD-1 extracellular domain without signal peptide)-ICOS
DS-truncated CD137 domain-truncated CD2 signaling
domain- IL-2 receptor binding (IL2RB)(YLRQ) (SEQ ID NO: 86)
TTTCTGGACAGCCCCGACAGACCCTGGAATCCTCCTACATTCAGCCCCGCTCTGCTGGTGG
TTACCGAGGGCGATAATGCCACCTTCACCTGTAGCTTCAGCAACACCAGCGAGAGCTTCGT
GCTGAACTGGTACAGAATGAGCCCCAGCAACCAGACCGACAAGCTGGCCGCCTTTCCTGAG
GATAGATCTCAGCCCGGCCAGGACTGCCGGTTCAGAGTTACACAGCTGCCCAACGGCCGGG
ACTTCCACATGTCTGTCGTTCGGGCCAGAAGAAACGACAGCGGCACATATCTGTGCGGCGC
CATTTCTCTGGCCCCTAAGGCTCAGATCAAAGAGAGCCTGAGAGCCGAGCTGAGAGTGACA
GAAAGACGGGCCGAAGTGCCCACAGCTCACCCTTCACCTTCTCCAAGACCTGCCAGCCAGC
TGTGCTGCCAGCTGAAGTTTTGGCTGCCTATCGGCTGTGCCGCCTTCGTGGTTGTGTGTAT
CCTGGGCTGCATCCTGATCTGCTGGCTGACCAAGAAAAAGTACAGCAGCTCCGTGCACGAC
CCCAACGGCGAGTACATGTTCATGAGAGCCGTGAACACCGCCAAGAAGTCCAGACTGACCG
ACGTGACCCTCCAGCCTTTCATGAGGCCTGTGCAGACCACACAAGAAGAGGACGGCTGCTC
CTGTCGGTTCCCCGAGGAAGAGGAAGGCGGTTGCGAACTCCAGAATCCTGCCACCTCTCAG
CACCCTCCACCTCCACCTGGACACAGATCTCAGGCCCCATCTCACAGACCTCCACCACCTG
GTCATCGGGTGCAGCATCAGCCCCAGAAAAGACCTCCTGCTCCTAGCGGAACACAGGTGCA
CCAGCAAAAGGGACCTCCACTGCCTAGACCTAGAGTGCAGCCTAAGCCTCCTCATGGCGCT
GCCGAGAATAGCCTGTCTCCTAGCAGCAACAACTGCCGCAACACAGGCCCCTGGCTGAAGA
AAGTGCTGAAGTGCAACACCCCTGATCCGAGCAAGTTCTTTAGCCAGCTGAGCAGCGAGCA
TGGCGGCGACGTTCAGAAATGGCTGTCTAGCCCATTTCCAAGCAGCAGCTTCAGCCCTGGT
GGACTGGCCCCTGAGATTAGCCCTCTGGAAGTGCTGGAACGGGACAAAGTGACCCAGCTGC
TGCCCCTGAATACCGACGCTTACCTGAGCCTGCAAGAGCTGCAAGGACAGGACCCTACACA
CCTGGTGTCCTACCTGAGACAGTGGGTCGTGATCCCACCTCCTTTGAGCAGTCCAGGACCT
CAGGCCTCT
FMC63 scFV (CD8a Leader_Light Chain_Linker_Heavy
Chain_CD8a Hinge) (CD19 binding extracellular domain)
(SEQ ID NO: 91)
ATGGCTCTGCCTGTGACAGCTCTGCTGCTGCCTCTGGCTCTGCTTCTGCATGCCGCCAGAC
CTGACATCCAGATGACCCAGACAACCAGCAGCCTGTCTGCCAGCCTGGGCGATAGAGTGAC
CATCAGCTGTAGAGCCAGCCAGGACATCAGCAAGTACCTGAACTGGTATCAGCAGAAACCC
GACGGCACCGTGAAGCTGCTGATCTACCACACCAGCAGACTGCACAGCGGCGTGCCAAGCA
GATTTTCTGGCAGCGGCTCTGGCACCGACTACAGCCTGACCATCTCCAACCTGGAACAAGA
GGATATCGCTACCTACTTCTGCCAGCAAGGCAACACCCTGCCTTACACCTTTGGCGGAGGC
ACCAAGCTGGAAATCACAGGCGGCGGAGGAAGCGGAGGCGGAGGATCTGGTGGTGGTGGAT
CTGAAGTGAAACTGCAAGAGTCTGGCCCTGGCCTGGTGGCCCCATCTCAATCTCTGAGCGT
GACCTGTACCGTCAGCGGAGTGTCCCTGCCTGATTATGGCGTGTCCTGGATCCGGCAGCCT
CCTAGAAAAGGCCTGGAATGGCTGGGCGTGATCTGGGGCAGCGAGACAACCTACTACAACA
GCGCCCTGAAGTCCCGGCTGACCATCATCAAGGACAACTCCAAGAGCCAGGTGTTCCTGAA
GATGAACAGCCTCCAGACCGACGACACCGCCATCTACTATTGCGCCAAGCACTACTACTAC
GGCGGCAGCTACGCCATGGATTATTGGGGCCAGGGCACCAGCGTGACCGTGTCTAGTACAA
CAACCCCTGCTCCTCGGCCTCCTACACCAGCTCCTACAATTGCCAGCCAGCCACTGTCTCT
GAGGCCCGAAGCTTGTAGACCTGCTGCTGGCGGAGCCGTGCATACAAGAGGACTGGATTTC
GCCTGC
CD28_BB signaling domain: CD28 signaling domain_4-1BB
Signaling Domain (SEQ NO: 151)
CTGTTCCCCGGACCTAGCAAGCCCTTTTGGGTGCTCGTTGTTGTTGGCGGCGTGCTGGCCT
GTTATAGCCTGCTGGTTACCGTGGCCTTCATCATCTTTTGGGTCCGAAGCAAGCGGAGCCG
GCTGCTGCACAGCGACTACATGAACATGACCCCTAGACGGCCCGGACCAACCAGAAAGCAC
TACCAGCCTTACGCTCCTCCTAGAGACTTCGCCGCCTACAGATCCAAGCGGGGCAGAAAGA
AGCTGCTGTACATCTTCAAGCAGCCCTTCATGCGGCCCGTGCAGACCACACAAGAGGAAGA
TGGCTGCTCCTGTCGGTTCCCCGAGGAAGAAGAAGGCGGTTGCGAACTG
CD28 Signaling Domain_truncated 4-1BB Signaling Domain
(Other name: CD28_BBt): (SEQ ID NO: 152)
CTGTTCCCCGGACCTAGCAAGCCCTTTTGGGTGCTCGTTGTTGTTGGCGGCGTGCTGGCCT
GTTATAGCCTGCTGGTTACCGTGGCCTTCATCATCTTTTGGGTCCGAAGCAAGCGGAGCCG
GCTGCTGCACAGCGACTACATGAACATGACCCCTAGACGGCCCGGACCAACCAGAAAGCAC
TACCAGCCTTACGCTCCTCCTAGAGACTTCGCCGCCTACAGATCCCAGCCTTTCATGAGGC
CTGTGCAGACCACACAAGAAGAGGACGGCTGCTCCTGTCGGTTCCCCGAGGAAGAGGAAGG
CGGTTGCGAACTT
CD28 Signaling Domain_truncated OX-40 Signaling Domain
(Other name: CD28_OX40t) (SEQ ID NO: 153)
CTGTTCCCCGGACCTAGCAAGCCCTTTTGGGTGCTCGTTGTTGTTGGCGGCGTGCTGGCCT
GTTATAGCCTGCTGGTTACCGTGGCCTTCATCATCTTTTGGGTCCGAAGCAAGCGGAGCCG
GCTGCTGCACAGCGACTACATGAACATGACCCCTAGACGGCCCGGACCAACCAGAAAGCAC
TACCAGCCTTACGCTCCTCCTAGAGACTTCGCCGCCTACAGATCTGGCGGCGGAAGCTTCA
GAACCCCTATCCAAGAGGAACAGGCCGACGCTCACTCTACACTGGCT
ICOS Transmembrane_ICOS Signaling Domain (SEQ ID NO: 154)
AGCCAGCTGTGCTGCCAGCTGAAGTTTTGGCTGCCTATCGGCTGTGCCGCCTTCGTGGTTG
TGTGTATCCTGGGCTGCATCCTGATCTGCTGGCTGACCAAGAAAAAGTACAGCAGCTCCGT
GCACGACCCCAACGGCGAGTACATGTTCATGAGAGCCGTGAACACCGCCAAGAAGTCCAGA
CTGACCGACGTGACACTT
ICOS Transmembrane_ICOS Signaling Domain_4-1BB Signaling
Domain (SEQ ID NO: 155)
AGCCAGCTGTGCTGCCAGCTGAAGTTTTGGCTGCCTATCGGCTGTGCCGCCTTCGTGGTTG
TGTGTATCCTGGGCTGCATCCTGATCTGCTGGCTGACCAAGAAAAAGTACAGCAGCTCCGT
GCACGACCCCAACGGCGAGTACATGTTCATGAGAGCCGTGAACACCGCCAAGAAGTCCAGA
CTGACCGACGTGACCCTGAAGCGGGGCAGAAAGAAACTGCTGTACATCTTCAAGCAGCCCT
TCATGCGGCCCGTGCAGACCACACAAGAGGAAGATGGCTGCTCCTGCAGATTCCCCGAGGA
AGAAGAAGGCGGCTGCGAACTT
ICOS Transmembrane_ICOS Signaling Domain_Truncated 4-1BB
Signaling Domain (SEQ ID NO: 156)
AGCCAGCTGTGCTGCCAGCTGAAGTTTTGGCTGCCTATCGGCTGTGCCGCCTTCGTGGTTG
TGTGTATCCTGGGCTGCATCCTGATCTGCTGGCTGACCAAGAAAAAGTACAGCAGCTCCGT
GCACGACCCCAACGGCGAGTACATGTTCATGAGAGCCGTGAACACCGCCAAGAAGTCCAGA
CTGACCGACGTGACACTTCAGCCTTTCATGAGGCCCGTGCAGACCACACAAGAAGAGGACG
GCTGCTCCTGCAGATTCCCCGAGGAAGAGGAAGGCGGTTGCGAACTT
ICOS Transmembrane_ICOS Signaling Domain_Truncated OX-40
Signaling Domain (ICOS_OX40t) (SEQ ID NO: 157)
AGCCAGCTGTGCTGCCAGCTGAAGTTTTGGCTGCCTATCGGCTGTGCCGCCTTCGTGGTTG
TGTGTATCCTGGGCTGCATCCTGATCTGCTGGCTGACCAAGAAAAAGTACAGCAGCTCCGT
GCACGACCCCAACGGCGAGTACATGTTCATGAGAGCCGTGAACACCGCCAAGAAGTCCAGA
CTGACCGACGTGACACTTGGCGGCGGAAGCTTTAGAACCCCTATCCAAGAGGAACAGGCCG
ACGCTCACTCTACACTGGCT
ICOS Transmembrane_ICOS Signaling Domain (mini-CD28)_Truncated
4-1BB Signaling Domain (ICOS(28)_BBt) (SEQ ID NO: 158)
AGCCAGCTGTGCTGCCAGCTGAAGTTTTGGCTGCCTATCGGCTGTGCCGCCTTCGTGGTTG
TGTGTATCCTGGGCTGCATCCTGATCTGCTGGCTGACCAAGAAAAAGTACAGCAGCTCCGT
GCACGACCCCAACGGCGAGTACATGTTCATGACCCCTAGAAGGCCTGGACCTACCAGAAAG
CACTACCAGCCTTACGCTCCTCCTAGAGCCGTGAACACCGCCAAGAAGTCCAGACTGACCG
ACGTGACACTTCAGCCTTTCATGAGGCCCGTGCAGACCACACAAGAAGAGGACGGCTGCTC
CTGCAGATTCCCCGAGGAAGAGGAAGGCGGTTGCGAACTT
ICOS(28)_truncated OX-40 (CD134) intracellular domain
(ICOS(28)_OX40t) (SEQ ID NO: 159)
AGCCAGCTGTGCTGCCAGCTGAAGTTTTGGCTGCCTATCGGCTGTGCCGCCTTCGTGGTTG
TGTGTATCCTGGGCTGCATCCTGATCTGCTGGCTGACCAAGAAAAAGTACAGCAGCTCCGT
GCACGACCCCAACGGCGAGTACATGTTCATGACCCCTAGAAGGCCTGGACCTACCAGAAAG
CACTACCAGCCTTACGCTCCTCCTAGAGCCGTGAACACCGCCAAGAAGTCCAGACTGACCG
ACGTGACACTTGGCGGCGGAAGCTTTAGAACCCCTATCCAAGAGGAACAGGCCGACGCTCA
CTCTACACTGGCT
ICOS Transmembrane_ICOS Signaling Domain_4-1BB Signaling
Domain with mutated polybasic region (ICOS_BB(xPB))
(SEQ ID NO: 160)
AGCCAGCTGTGCTGCCAGCTGAAGTTTTGGCTGCCTATCGGCTGTGCCGCCTTCGTGGTTG
TGTGTATCCTGGGCTGCATCCTGATCTGCTGGCTGACCAAGAAAAAGTACAGCAGCTCCGT
GCACGACCCCAACGGCGAGTACATGTTCATGAGAGCCGTGAACACCGCCAAGAAGTCCAGA
CTGACCGACGTGACCCTGGCTGCCGGCGCTGCAGCTCTGCTGTACATCTTCAAGCAGCCCT
TCATGCGGCCCGTGCAGACCACACAAGAGGAAGATGGCTGCTCCTGCAGATTCCCCGAGGA
AGAAGAAGGCGGCTGCGAACTT
ICOS Transmembrane_ICOS Signaling Domain_4-1BB Signaling
Domain with mutated lysines (ICOS_BB(xUb)) (SEQ ID NO: 161)
AGCCAGCTGTGCTGCCAGCTGAAGTTTTGGCTGCCTATCGGCTGTGCCGCCTTCGTGGTTG
TGTGTATCCTGGGCTGCATCCTGATCTGCTGGCTGACCAAGAAAAAGTACAGCAGCTCCGT
GCACGACCCCAACGGCGAGTACATGTTCATGAGAGCCGTGAACACCGCCAAGAAGTCCAGA
CTGACCGACGTGACCCTGAAGCGGGGCAGAAAGAAACTGCTGTACATCTTCGCACAGCCCT
TCATGCGGCCCGTGCAGACCACACAAGAGGAAGATGGCTGCTCCTGCAGATTCCCCGAGGA
AGAAGAAGGCGGCTGCGAACTT
ICOS Transmembrane_ICOS Signaling Domain_4-1BB Signaling
Domain with mutated lysines and polybasic regions
(ICOS_BB(xPBxUb)) (SEQ ID NO: 162)
AGCCAGCTGTGCTGCCAGCTGAAGTTTTGGCTGCCTATCGGCTGTGCCGCCTTCGTGGTTG
TGTGTATCCTGGGCTGCATCCTGATCTGCTGGCTGACCAAGAAAAAGTACAGCAGCTCCGT
GCACGACCCCAACGGCGAGTACATGTTCATGAGAGCCGTGAACACCGCCAAGAAGTCCAGA
CTGACCGACGTGACCCTGGCTGCCGGCGCTGCAGCTCTGCTGTACATCTTCGCACAGCCCT
TCATGCGGCCCGTGCAGACCACACAAGAGGAAGATGGCTGCTCCTGCAGATTCCCCGAGGA
AGAAGAAGGCGGCTGCGAACTT
ICOS Transmembrane_ICOS Signaling Domain_Wild Type OX-40
Signaling Domain (ICOS_40) (SEQ ID NO: 163)
AGCCAGCTGTGCTGCCAGCTGAAGTTTTGGCTGCCTATCGGCTGTGCCGCCTTCGTGGTTG
TGTGTATCCTGGGCTGCATCCTGATCTGCTGGCTGACCAAGAAAAAGTACAGCAGCTCCGT
GCACGACCCCAACGGCGAGTACATGTTCATGAGAGCCGTGAACACCGCCAAGAAGTCCAGA
CTGACCGACGTGACACTTAGGAGAGACCAGCGTCTGCCACCAGATGCACATAAGCCACCTG
GCGGCGGAAGCTTTAGAACCCCTATCCAAGAGGAACAGGCCGACGCTCACTCTACACTGGC
TAAAATC
ICOS Transmembrane_ICOS Signaling Domain_OX-40 Signaling
Domain with mutated polybasic region (ICOS_40 (xPB)
(SEQ ID NO: 164)
AGCCAGCTGTGCTGCCAGCTGAAGTTTTGGCTGCCTATCGGCTGTGCCGCCTTCGTGGTTG
TGTGTATCCTGGGCTGCATCCTGATCTGCTGGCTGACCAAGAAAAAGTACAGCAGCTCCGT
GCACGACCCCAACGGCGAGTACATGTTCATGAGAGCCGTGAACACCGCCAAGAAGTCCAGA
CTGACCGACGTGACACTTGCCGCGGACCAGGCCCTGCCACCAGATGCACATAAGCCACCTG
GCGGCGGAAGCTTTAGAACCCCTATCCAAGAGGAACAGGCCGACGCTCACTCTACACTGGC
TAAAATC
ICOS Transmembrane_ICOS Signaling Domain_OX-40 Signaling
Domain with mutated lysine residues (ICOS_40 (xUb)):
(SEQ ID NO: 165)
AGCCAGCTGTGCTGCCAGCTGAAGTTTTGGCTGCCTATCGGCTGTGCCGCCTTCGTGGTTG
TGTGTATCCTGGGCTGCATCCTGATCTGCTGGCTGACCAAGAAAAAGTACAGCAGCTCCGT
GCACGACCCCAACGGCGAGTACATGTTCATGAGAGCCGTGAACACCGCCAAGAAGTCCAGA
CTGACCGACGTGACACTTAGGAGAGACCAGCGTCTGCCACCAGATGCACATGCACCACCTG
GCGGCGGAAGCTTTAGAACCCCTATCCAAGAGGAACAGGCCGACGCTCACTCTACACTGGC
TGCAATC
ICOS Transmembrane_ICOS Signaling Domain_OX-40 Signaling
Domain with mutated lysine residues and polybasic regions
(ICOS_40 (xPBxUb)): (SEQ ID NO: 166)
AGCCAGCTGTGCTGCCAGCTGAAGTTTTGGCTGCCTATCGGCTGTGCCGCCTTCGTGGTTG
TGTGTATCCTGGGCTGCATCCTGATCTGCTGGCTGACCAAGAAAAAGTACAGCAGCTCCGT
GCACGACCCCAACGGCGAGTACATGTTCATGAGAGCCGTGAACACCGCCAAGAAGTCCAGA
CTGACCGACGTGACACTTGCCGCGGACCAGGCCCTGCCACCAGATGCACATGCACCACCTG
GCGGCGGAAGCTTTAGAACCCCTATCCAAGAGGAACAGGCCGACGCTCACTCTACACTGGC
TGCAATC
PD-1 Extracellular (SEQ ID NO: 167)
TTCCTGGACTCTCCTGACAGACCCTGGAATCCTCCAACATTCAGCCCCGCTCTGCTGGTGG
TTACCGAGGGCGATAATGCCACCTTCACCTGTAGCTTCAGCAACACCAGCGAGAGCTTCGT
GCTGAACTGGTACAGAATGAGCCCCAGCAACCAGACCGACAAGCTGGCCGCCTTTCCTGAG
GATAGATCTCAGCCCGGCCAGGACTGCCGGTTCAGAGTTACACAGCTGCCCAACGGCCGGG
ACTTCCACATGTCTGTCGTCCGGGCCAGAAGAAACGACAGCGGCACATATCTGTGCGGCGC
CATTTCTCTGGCCCCTAAGGCTCAGATCAAAGAGAGCCTGAGAGCCGAGCTGAGAGTGACA
GAAAGACGGGCCGAAGTGCCCACAGCTCACCCTTCACCTTCTCCAAGACCTGCC
HLA-A2 Signal Peptide_PD-1 Extracellular_PD-1 Transmembrane
(PD-1 (HLASP-Truncated; PD-1_TLs) (SEQ ID NO: 168)
ATGGCTGTGATGGCTCCTAGAACACTGGTGCTGCTGCTGTCTGGTGCCCTGGCTCTGACTC
AGACATGGGCCTTTCTGGACAGCCCCGACAGACCCTGGAATCCTCCTACATTCAGCCCCGC
TCTGCTGGTGGTTACCGAGGGCGATAATGCCACCTTCACCTGTAGCTTCAGCAACACCAGC
GAGAGCTTCGTGCTGAACTGGTACAGAATGAGCCCCAGCAACCAGACCGACAAGCTGGCCG
CCTTTCCTGAGGATAGATCTCAGCCCGGCCAGGACTGCCGGTTCAGAGTTACACAGCTGCC
CAACGGCCGGGACTTCCACATGTCTGTCGTTCGGGCCAGAAGAAACGACAGCGGCACATAT
CTGTGCGGCGCCATTTCTCTGGCCCCTAAGGCTCAGATCAAAGAGAGCCTGAGAGCCGAGC
TGAGAGTGACAGAAAGACGGGCCGAAGTGCCCACAGCTCACCCTTCACCTTCTCCAAGACC
TGCCGGCCAGTTCCAGACACTGGTCGTGGGAGTTGTTGGCGGCCTGCTGGGATCTCTGGTT
CTGCTTGTTTGGGTGCTCGCCGTGATCTGCTCTAGA
HLA-A2 Signal Peptide_PD-1 Extracellular_CD28
Transmembrane_CD28 Signaling Domain (HLASP CD28 DS or
PD-1_CD28 or PD-1_28) (SEQ ID NO: 169)
ATGGCTGTGATGGCTCCTAGAACACTGGTGCTGCTGCTGTCTGGTGCCCTGGCTCTGACTC
AGACATGGGCCTTTCTGGACAGCCCCGACAGACCCTGGAATCCTCCTACATTCAGCCCCGC
TCTGCTGGTGGTTACCGAGGGCGATAATGCCACCTTCACCTGTAGCTTCAGCAACACCAGC
GAGAGCTTCGTGCTGAACTGGTACAGAATGAGCCCCAGCAACCAGACCGACAAGCTGGCCG
CCTTTCCTGAGGATAGATCTCAGCCCGGCCAGGACTGCCGGTTCAGAGTTACACAGCTGCC
CAACGGCCGGGACTTCCACATGTCTGTCGTTCGGGCCAGAAGAAACGACAGCGGCACATAT
CTGTGCGGCGCCATTTCTCTGGCCCCTAAGGCTCAGATCAAAGAGAGCCTGAGAGCCGAGC
TGAGAGTGACAGAAAGACGGGCCGAAGTGCCCACAGCTCACCCTTCACCTTCTCCAAGACC
TGCTCTGTTCCCCGGACCTAGCAAGCCCTTTTGGGTGCTCGTTGTTGTTGGCGGCGTGCTG
GCCTGTTATAGCCTGCTGGTTACCGTGGCCTTCATCATCTTTTGGGTCCGAAGCAAGCGGA
GCCGGCTGCTGCACAGCGACTACATGAACATGACCCCTAGACGGCCCGGACCAACCAGAAA
GCACTACCAGCCTTACGCTCCTCCTAGAGACTTCGCCGCCTACAGATCT
HLA-A2 Signal Peptide_PD-1 Extracellular_CD28
Transmembrane_CD28 Signaling Domain_4-1BB Signaling Domain
(PD-1_28_BBwt) (SEQ ID NO: 170)
ATGGCTGTGATGGCCCCTAGAACACTGGTGCTGCTGCTGTCTGGTGCCCTGGCTCTGACTC
AGACATGGGCCTTTCTGGACAGCCCCGACAGACCCTGGAATCCTCCTACATTCAGCCCCGC
TCTGCTGGTGGTTACCGAGGGCGATAATGCCACCTTCACCTGTAGCTTCAGCAACACCAGC
GAGAGCTTCGTGCTGAACTGGTACAGAATGAGCCCCAGCAACCAGACCGACAAGCTGGCCG
CCTTTCCTGAGGATAGATCTCAGCCCGGCCAGGACTGCCGGTTCAGAGTTACACAGCTGCC
CAACGGCCGGGACTTCCACATGTCTGTCGTTCGGGCCAGAAGAAACGACAGCGGCACATAT
CTGTGCGGCGCCATTTCTCTGGCCCCTAAGGCTCAGATCAAAGAGAGCCTGAGAGCCGAGC
TGAGAGTGACAGAAAGACGGGCCGAAGTGCCCACAGCTCACCCTTCACCTTCTCCAAGACC
TGCTCTGTTCCCCGGACCTAGCAAGCCCTTTTGGGTGCTCGTTGTTGTTGGCGGCGTGCTG
GCCTGTTATAGCCTGCTGGTTACCGTGGCCTTCATCATCTTTTGGGTCCGAAGCAAGCGGA
GCCGGCTGCTGCACAGCGACTACATGAACATGACCCCTAGACGGCCCGGACCAACCAGAAA
GCACTACCAGCCTTACGCTCCTCCTAGAGACTTCGCCGCCTACAGATCCAAGCGGGGCAGA
AAGAAGCTGCTGTACATCTTCAAGCAGCCCTTCATGCGGCCCGTGCAGACCACACAAGAGG
AAGATGGCTGCTCCTGTCGGTTCCCCGAGGAAGAAGAAGGCGGTTGCGAACTG
HLA-A2 Signal Peptide_PD-1 Extracellular_CD28
Transmembrane_CD28 Signaling Domain_Truncated 4-1BB
Signaling Domain (PD-1_28_BBt) (SEQ ID NO: 171)
ATGGCTGTGATGGCCCCTAGAACACTGGTGCTGCTGCTGTCTGGTGCCCTGGCTCTGACTC
AGACATGGGCCTTTCTGGACAGCCCCGACAGACCCTGGAATCCTCCTACATTCAGCCCCGC
TCTGCTGGTGGTTACCGAGGGCGATAATGCCACCTTCACCTGTAGCTTCAGCAACACCAGC
GAGAGCTTCGTGCTGAACTGGTACAGAATGAGCCCCAGCAACCAGACCGACAAGCTGGCCG
CCTTTCCTGAGGATAGATCTCAGCCCGGCCAGGACTGCCGGTTCAGAGTTACACAGCTGCC
CAACGGCCGGGACTTCCACATGTCTGTCGTTCGGGCCAGAAGAAACGACAGCGGCACATAT
CTGTGCGGCGCCATTTCTCTGGCCCCTAAGGCTCAGATCAAAGAGAGCCTGAGAGCCGAGC
TGAGAGTGACAGAAAGACGGGCCGAAGTGCCCACAGCTCACCCTTCACCTTCTCCAAGACC
TGCTCTGTTCCCCGGACCTAGCAAGCCCTTTTGGGTGCTCGTTGTTGTTGGCGGCGTGCTG
GCCTGTTATAGCCTGCTGGTTACCGTGGCCTTCATCATCTTTTGGGTCCGAAGCAAGCGGA
GCCGGCTGCTGCACAGCGACTACATGAACATGACCCCTAGACGGCCCGGACCAACCAGAAA
GCACTACCAGCCTTACGCTCCTCCTAGAGACTTCGCCGCCTACAGATCCCAGCCTTTCATG
AGGCCTGTGCAGACCACACAAGAAGAGGACGGCTGCTCCTGTCGGTTCCCCGAGGAAGAGG
AAGGCGGTTGCGAACTT
HLA-A2 Signal Peptide_PD-1 Extracellular_CD28
Transmembrane_CD28 Signaling Domain_Truncated OX-40
Signaling Domain (PD-1_28_OX40t) (SEQ ID NO: 172)
ATGGCTGTGATGGCCCCTAGAACACTGGTGCTGCTGCTGTCTGGTGCCCTGGCTCTGACTC
AGACATGGGCCTTTCTGGACAGCCCCGACAGACCCTGGAATCCTCCTACATTCAGCCCCGC
TCTGCTGGTGGTTACCGAGGGCGATAATGCCACCTTCACCTGTAGCTTCAGCAACACCAGC
GAGAGCTTCGTGCTGAACTGGTACAGAATGAGCCCCAGCAACCAGACCGACAAGCTGGCCG
CCTTTCCTGAGGATAGATCTCAGCCCGGCCAGGACTGCCGGTTCAGAGTTACACAGCTGCC
CAACGGCCGGGACTTCCACATGTCTGTCGTTCGGGCCAGAAGAAACGACAGCGGCACATAT
CTGTGCGGCGCCATTTCTCTGGCCCCTAAGGCTCAGATCAAAGAGAGCCTGAGAGCCGAGC
TGAGAGTGACAGAAAGACGGGCCGAAGTGCCCACAGCTCACCCTTCACCTTCTCCAAGACC
TGCTCTGTTCCCCGGACCTAGCAAGCCCTTTTGGGTGCTCGTTGTTGTTGGCGGCGTGCTG
GCCTGTTATAGCCTGCTGGTTACCGTGGCCTTCATCATCTTTTGGGTCCGAAGCAAGCGGA
GCCGGCTGCTGCACAGCGACTACATGAACATGACCCCTAGACGGCCCGGACCAACCAGAAA
GCACTACCAGCCTTACGCTCCTCCTAGAGACTTCGCCGCCTACAGATCTGGCGGCGGAAGC
TTCAGAACCCCTATCCAAGAGGAACAGGCCGACGCTCACTCTACACTGGCT
HLA-A2 Signal Peptide_PD-1 Extracellular_ICOS intracellular
domain (PD-1_ICOS) (SEQ ID NO: 173)
ATGGCTGTGATGGCTCCTAGAACACTGGTGCTGCTGCTGTCTGGTGCCCTGGCTCTGACTC
AGACATGGGCCTTTCTGGACAGCCCCGACAGACCCTGGAATCCTCCTACATTCAGCCCCGC
TCTGCTGGTGGTTACCGAGGGCGATAATGCCACCTTCACCTGTAGCTTCAGCAACACCAGC
GAGAGCTTCGTGCTGAACTGGTACAGAATGAGCCCCAGCAACCAGACCGACAAGCTGGCCG
CCTTTCCTGAGGATAGATCTCAGCCCGGCCAGGACTGCCGGTTCAGAGTTACACAGCTGCC
CAACGGCCGGGACTTCCACATGTCTGTCGTTCGGGCCAGAAGAAACGACAGCGGCACATAT
CTGTGCGGCGCCATTTCTCTGGCCCCTAAGGCTCAGATCAAAGAGAGCCTGAGAGCCGAGC
TGAGAGTGACAGAAAGACGGGCCGAAGTGCCCACAGCTCACCCTTCACCTTCTCCAAGACC
TGCCAGCCAGCTGTGCTGCCAGCTGAAGTTTTGGCTGCCTATCGGCTGTGCCGCCTTCGTG
GTTGTGTGTATCCTGGGCTGCATCCTGATCTGCTGGCTGACCAAGAAAAAGTACAGCAGCT
CCGTGCACGACCCCAACGGCGAGTACATGTTCATGAGAGCCGTGAACACCGCCAAGAAGTC
CAGACTGACCGACGTGACACTT
HLA-A2 Signal Peptide_PD-1 Extracellular_ICOS DS_CD137
(PD-1_ICOS_BBwt) (SEQ ID NO: 174)
ATGGCTGTGATGGCTCCTAGAACACTGGTGCTGCTGCTGTCTGGTGCCCTGGCTCTGACTC
AGACATGGGCCTTTCTGGACAGCCCCGACAGACCCTGGAATCCTCCTACATTCAGCCCCGC
TCTGCTGGTGGTTACCGAGGGCGATAATGCCACCTTCACCTGTAGCTTCAGCAACACCAGC
GAGAGCTTCGTGCTGAACTGGTACAGAATGAGCCCCAGCAACCAGACCGACAAGCTGGCCG
CCTTTCCTGAGGATAGATCTCAGCCCGGCCAGGACTGCCGGTTCAGAGTTACACAGCTGCC
CAACGGCCGGGACTTCCACATGTCTGTCGTTCGGGCCAGAAGAAACGACAGCGGCACATAT
CTGTGCGGCGCCATTTCTCTGGCCCCTAAGGCTCAGATCAAAGAGAGCCTGAGAGCCGAGC
TGAGAGTGACAGAAAGACGGGCCGAAGTGCCCACAGCTCACCCTTCACCTTCTCCAAGACC
TGCCAGCCAGCTGTGCTGCCAGCTGAAGTTTTGGCTGCCTATCGGCTGTGCCGCCTTCGTG
GTTGTGTGTATCCTGGGCTGCATCCTGATCTGCTGGCTGACCAAGAAAAAGTACAGCAGCT
CCGTGCACGACCCCAACGGCGAGTACATGTTCATGAGAGCCGTGAACACCGCCAAGAAGTC
CAGACTGACCGACGTGACCCTGAAGCGGGGCAGAAAGAAACTGCTGTACATCTTCAAGCAG
CCCTTCATGCGGCCCGTGCAGACCACACAAGAGGAAGATGGCTGCTCCTGCAGATTCCCCG
AGGAAGAAGAAGGCGGCTGCGAACTT
HLA-A2 Signal Peptide_PD-1 Extracellular_ICOS_truncated
CD137 (PD-1_ICOS_BBt) (SEQ ID NO: 175)
ATGGCTGTGATGGCTCCTAGAACACTGGTGCTGCTGCTGTCTGGTGCCCTGGCTCTGACTC
AGACATGGGCCTTTCTGGACAGCCCCGACAGACCCTGGAATCCTCCTACATTCAGCCCCGC
TCTGCTGGTGGTTACCGAGGGCGATAATGCCACCTTCACCTGTAGCTTCAGCAACACCAGC
GAGAGCTTCGTGCTGAACTGGTACAGAATGAGCCCCAGCAACCAGACCGACAAGCTGGCCG
CCTTTCCTGAGGATAGATCTCAGCCCGGCCAGGACTGCCGGTTCAGAGTTACACAGCTGCC
CAACGGCCGGGACTTCCACATGTCTGTCGTTCGGGCCAGAAGAAACGACAGCGGCACATAT
CTGTGCGGCGCCATTTCTCTGGCCCCTAAGGCTCAGATCAAAGAGAGCCTGAGAGCCGAGC
TGAGAGTGACAGAAAGACGGGCCGAAGTGCCCACAGCTCACCCTTCACCTTCTCCAAGACC
TGCCAGCCAGCTGTGCTGCCAGCTGAAGTTTTGGCTGCCTATCGGCTGTGCCGCCTTCGTG
GTTGTGTGTATCCTGGGCTGCATCCTGATCTGCTGGCTGACCAAGAAAAAGTACAGCAGCT
CCGTGCACGACCCCAACGGCGAGTACATGTTCATGAGAGCCGTGAACACCGCCAAGAAGTC
CAGACTGACCGACGTGACACTTCAGCCTTTCATGAGGCCCGTGCAGACCACACAAGAAGAG
GACGGCTGCTCCTGCAGATTCCCCGAGGAAGAGGAAGGCGGTTGCGAACTT
HLA-A2 Signal Peptide_PD1 Extracellular_ICOS_truncated
CD134 (PD1_ICOS_OX40t) (SEQ ID NO: 176)
ATGGCTGTGATGGCTCCTAGAACACTGGTGCTGCTGCTGTCTGGTGCCCTGGCTCTGACTC
AGACATGGGCCTTTCTGGACAGCCCCGACAGACCCTGGAATCCTCCTACATTCAGCCCCGC
TCTGCTGGTGGTTACCGAGGGCGATAATGCCACCTTCACCTGTAGCTTCAGCAACACCAGC
GAGAGCTTCGTGCTGAACTGGTACAGAATGAGCCCCAGCAACCAGACCGACAAGCTGGCCG
CCTTTCCTGAGGATAGATCTCAGCCCGGCCAGGACTGCCGGTTCAGAGTTACACAGCTGCC
CAACGGCCGGGACTTCCACATGTCTGTCGTTCGGGCCAGAAGAAACGACAGCGGCACATAT
CTGTGCGGCGCCATTTCTCTGGCCCCTAAGGCTCAGATCAAAGAGAGCCTGAGAGCCGAGC
TGAGAGTGACAGAAAGACGGGCCGAAGTGCCCACAGCTCACCCTTCACCTTCTCCAAGACC
TGCCAGCCAGCTGTGCTGCCAGCTGAAGTTTTGGCTGCCTATCGGCTGTGCCGCCTTCGTG
GTTGTGTGTATCCTGGGCTGCATCCTGATCTGCTGGCTGACCAAGAAAAAGTACAGCAGCT
CCGTGCACGACCCCAACGGCGAGTACATGTTCATGAGAGCCGTGAACACCGCCAAGAAGTC
CAGACTGACCGACGTGACACTTGGCGGCGGAAGCTTTAGAACCCCTATCCAAGAGGAACAG
GCCGACGCTCACTCTACACTGGCT
HLA-A2 Signal Peptide_PD-1 Extracellular_ICOS
Transmembrane_ICOS Signaling Domain (mini-CD28)_Truncated
4-1BB Signaling (PD-1_ICOS(28)_BBt) (SEQ ID NO: 177)
ATGGCTGTGATGGCTCCTAGAACACTGGTGCTGCTGCTGTCTGGTGCCCTGGCTCTGACTC
AGACATGGGCCTTTCTGGACAGCCCCGACAGACCCTGGAATCCTCCTACATTCAGCCCCGC
TCTGCTGGTGGTTACCGAGGGCGATAATGCCACCTTCACCTGTAGCTTCAGCAACACCAGC
GAGAGCTTCGTGCTGAACTGGTACAGAATGAGCCCCAGCAACCAGACCGACAAGCTGGCCG
CCTTTCCTGAGGATAGATCTCAGCCCGGCCAGGACTGCCGGTTCAGAGTTACACAGCTGCC
CAACGGCCGGGACTTCCACATGTCTGTCGTTCGGGCCAGAAGAAACGACAGCGGCACATAT
CTGTGCGGCGCCATTTCTCTGGCCCCTAAGGCTCAGATCAAAGAGAGCCTGAGAGCCGAGC
TGAGAGTGACAGAAAGACGGGCCGAAGTGCCCACAGCTCACCCTTCACCTTCTCCAAGACC
TGCCAGCCAGCTGTGCTGCCAGCTGAAGTTTTGGCTGCCTATCGGCTGTGCCGCCTTCGTG
GTTGTGTGTATCCTGGGCTGCATCCTGATCTGCTGGCTGACCAAGAAAAAGTACAGCAGCT
CCGTGCACGACCCCAACGGCGAGTACATGTTCATGACCCCTAGAAGGCCTGGACCTACCAG
AAAGCACTACCAGCCTTACGCTCCTCCTAGAGCCGTGAACACCGCCAAGAAGTCCAGACTG
ACCGACGTGACACTTCAGCCTTTCATGAGGCCCGTGCAGACCACACAAGAAGAGGACGGCT
GCTCCTGCAGATTCCCCGAGGAAGAGGAAGGCGGTTGCGAACTT
HLA-A2 Signal Peptide_PD-1 Extracellular_ICOS
Transmembrane_ICOS Signaling Domain (mini-CD28)_Truncated
OX-40 Signaling Domain (PD-1 (HLASP-ICOS DS-CD28(PRRP)-
mutated CD134) (PD-1_ICOS_OX40t, PD-1:ICOS40t) (SEQ ID NO: 178)
ATGGCTGTGATGGCTCCTAGAACACTGGTGCTGCTGCTGTCTGGTGCCCTGGCTCTGACTC
AGACATGGGCCTTTCTGGACAGCCCCGACAGACCCTGGAATCCTCCTACATTCAGCCCCGC
TCTGCTGGTGGTTACCGAGGGCGATAATGCCACCTTCACCTGTAGCTTCAGCAACACCAGC
GAGAGCTTCGTGCTGAACTGGTACAGAATGAGCCCCAGCAACCAGACCGACAAGCTGGCCG
CCTTTCCTGAGGATAGATCTCAGCCCGGCCAGGACTGCCGGTTCAGAGTTACACAGCTGCC
CAACGGCCGGGACTTCCACATGTCTGTCGTTCGGGCCAGAAGAAACGACAGCGGCACATAT
CTGTGCGGCGCCATTTCTCTGGCCCCTAAGGCTCAGATCAAAGAGAGCCTGAGAGCCGAGC
TGAGAGTGACAGAAAGACGGGCCGAAGTGCCCACAGCTCACCCTTCACCTTCTCCAAGACC
TGCCAGCCAGCTGTGCTGCCAGCTGAAGTTTTGGCTGCCTATCGGCTGTGCCGCCTTCGTG
GTTGTGTGTATCCTGGGCTGCATCCTGATCTGCTGGCTGACCAAGAAAAAGTACAGCAGCT
CCGTGCACGACCCCAACGGCGAGTACATGTTCATGACCCCTAGAAGGCCTGGACCTACCAG
AAAGCACTACCAGCCTTACGCTCCTCCTAGAGCCGTGAACACCGCCAAGAAGTCCAGACTG
ACCGACGTGACACTTGGCGGCGGAAGCTTTAGAACCCCTATCCAAGAGGAACAGGCCGACG
CTCACTCTACACTGGCT
11-D5-3 scFv (CD8a Signal Peptide_11-D5-3 scFv,
mouse_CD8a Hinge_BBZ) (SEQ ID NO: 179)
ATGGCTCTGCCTGTGACAGCTCTGCTGCTGCCTCTGGCTCTGCTTCTGCATGCCGCCAGAC
CTGACATCGTGCTGACACAGTCTCCACCTAGCCTGGCCATGAGCCTGGGAAAGAGAGCCAC
CATCAGCTGTAGAGCCAGCGAGAGCGTGACAATCCTGGGCTCTCACCTGATCCACTGGTAT
CAGCAGAAGCCCGGCCAGCCTCCTACACTGCTGATTCAGCTGGCCTCCAATGTGCAGACAG
GCGTGCCAGCCAGATTTTCTGGCAGCGGCAGCAGAACCGACTTCACCCTGACAATCGACCC
CGTGGAAGAGGACGATGTGGCCGTGTACTACTGCCTCCAGAGCCGGACAATCCCCAGAACA
TTTGGCGGAGGCACCAAGCTGGAAATCAAGGGCAGCACAAGCGGCTCTGGCAAGCCTGGAT
CTGGCGAGGGATCTACCAAGGGACAGATCCAGCTGGTGCAGTCTGGCCCCGAGCTGAAGAA
ACCTGGCGAGACAGTGAAGATCAGCTGCAAGGCCAGCGGCTACACCTTCACCGACTACAGC
ATCAACTGGGTCAAGAGAGCCCCTGGCAAGGGCCTGAAATGGATGGGCTGGATCAACACCG
AAACCAGAGAGCCCGCCTACGCCTACGACTTCAGAGGCAGATTCGCCTTCAGCCTGGAAAC
CAGCGCCAGCACAGCCTACCTCCAGATCAACAACCTGAAGTACGAGGACACCGCCACCTAC
TTTTGCGCCCTGGATTACAGCTACGCCATGGACTATTGGGGCCAGGGCACAAGCGTGACCG
TGTCCTCTACAACAACCCCTGCTCCTCGGCCTCCAACACCAGCTCCTACAATTGCCTCTCA
GCCCCTGTCTCTGAGGCCCGAAGCTTGTAGACCTGCTGCTGGCGGAGCCGTGCATACAAGA
GGACTGGATTTCGCCTGCGACATCTACATCTGGGCCCCTCTGGCTGGAACATGTGGCGTGC
TGCTGCTGAGCCTGGTCATCACCCTGTACTGCAAGCGGGGCAGAAAGAAGCTGCTGTACAT
CTTCAAGCAGCCCTTCATGCGGCCCGTGCAGACCACACAAGAGGAAGATGGCTGCTCCTGT
CGGTTCCCTGAGGAAGAAGAAGGCGGCTGCGAGCTGAGAGTGAAGTTCAGCAGATCCGCCG
ACGCTCCTGCCTATCAGCAGGGACAGAATCAGCTCTACAACGAGCTGAACCTGGGGCGCAG
AGAAGAGTACGACGTGCTGGACAAGAGAAGAGGCAGGGACCCTGAGATGGGCGGAAAGCCC
CAGAGAAGAAAGAACCCTCAAGAGGGCCTGTACAATGAGCTGCAAAAGGACAAGATGGCCG
AGGCCTACAGCGAGATCGGAATGAAGGGCGAACGCAGAAGAGGAAAGGGCCACGACGGACT
GTATCAGGGCCTGAGCACAGCCACCAAGGACACCTATGATGCCCTGCACATGCAGGCCCTG
CCTCCAAGA
FHVH33 HVV (CD8a Signal Peptide_FHVH33 HVV_CD8a Hinge_BBZ)
(SEQ ID NO: 180)
ATGGCTCTGCCTGTGACAGCTCTGCTGCTGCCTCTGGCTCTGCTTCTGCATGCCGCTAGAC
CTGAGGTGCAGCTGCTTGAATCTGGCGGAGGACTGGTTCAGCCTGGCGGATCTCTGAGACT
GTCTTGTGCCGCCAGCGGCTTCACCTTTAGCAGCTACGCCATGAGCTGGGTCCGACAGGCT
CCTGGCAAAGGCCTTGAATGGGTGTCCAGCATCAGCGGCAGCGGCGACTACATCTACTACG
CCGATAGCGTGAAGGGCAGATTCACCATCAGCCGGGACATCAGCAAGAACACCCTGTACCT
CCAGATGAACAGCCTGAGAGCCGAGGACACCGCCGTGTACTACTGTGCCAAAGAAGGCACC
GGCGCCAATAGCAGCCTGGCCGATTATAGAGGCCAGGGCACACTGGTCACCGTGTCCAGTT
TCGTGCCTGTGTTCCTGCCTGCCAAGCCTACCACAACACCCGCTCCTAGACCTCCAACACC
AGCTCCAACAATCGCCAGCCAGCCTCTGTCTCTGAGGCCAGAAGCTTGTAGACCTGCTGCT
GGCGGAGCCGTGCATACAAGAGGACTGGATTTCGCCTGCGACATCTACATCTGGGCCCCTC
TGGCTGGAACATGTGGCGTGCTGCTGCTGAGCCTGGTCATCACACTGTACTGCAACCACCG
GAACAAGCGGGGCAGAAAGAAGCTGCTGTACATCTTTAAGCAGCCCTTCATGCGGCCCGTG
CAGACCACACAAGAGGAAGATGGCTGCTCCTGTCGGTTCCCCGAGGAAGAAGAAGGCGGCT
GCGAGCTGAGAGTGAAGTTCAGCAGATCCGCAGACGCCCCTGCCTATCAGCAGGGACAGAA
CCAGCTGTACAACGAGCTGAACCTGGGGAGAAGAGAAGAGTACGACGTGCTGGACAAGCGG
AGAGGCAGAGATCCTGAGATGGGCGGAAAGCCCCAGCGGAGAAAGAATCCTCAAGAGGGCC
TGTATAATGAGCTGCAAAAGGACAAGATGGCCGAGGCCTACAGCGAGATCGGAATGAAGGG
CGAGCGCAGAAGAGGCAAGGGACACGATGGACTGTACCAGGGCCTGAGCACCGCCACCAAG
GATACCTATGATGCCCTGCACATGCAGGCCCTGCCTCCAAGA
BCAR003 HVV (CD8a Signal Peptide_BCAR003 HVV_CD8a Hinge_BBZ)
(SEQ ID NO: 181)
ATGGCTCTGCCTGTGACAGCTCTGCTGCTGCCTCTGGCTCTGCTTCTGCATGCCGCTAGAC
CCCAAGTGAAGCTGGAAGAGTCTGGCGGAGGACTGGTGCAGGCTGGCAGATCTCTGAGACT
GTCTTGTGCCGCCAGCGAGCACACCTTTAGCTCTCACGTGATGGGCTGGTTCAGACAGGCC
CCTGGCAAAGAAAGGGAAAGCGTGGCCGTTATCGGCTGGCGGGATATCAGCACAAGCTACG
CCGATAGCGTGAAGGGCAGATTCACCATCAGCCGGGACAACGCCAAGAAAACCCTGTACCT
CCAGATGAACAGCCTGAAGCCTGAGGACACCGCCGTGTACTACTGCGCCGCCAGAAGAATT
GACGCCGCCGACTTTGATTCTTGGGGCCAGGGAACCCAAGTGACCGTTTCTAGCGGAGGCG
GTGGAAGTGGCGGCGGTGGATCAGGTGGTGGTGGATCTGGTGGCGGAGGAAGCGGCGGAGG
CGGATCTGCTGTTCAGCTTGTTGAATCAGGTGGCGGCCTGGTTCAGGCCGGGGATTCTCTT
AGACTGACCTGCACAGCCAGCGGCAGAGCCTTCAGCACCTACTTCATGGCCTGGTTTCGGC
AGGCTCCCGGAAAAGAACGGGAATTTGTGGCCGGAATCGCTTGGAGCGGAGGCTCTACAGC
CTATGCCGATTCCGTGAAAGGCCGGTTTACCATCAGCAGAGATAATGCCAAAAACACGGTG
TACCTGCAAATGAACTCTCTGAAGTCCGAGGATACGGCCGTCTATTACTGTGCCAGCAGAG
GCATCGAGGTGGAAGAGTTTGGAGCCTGGGGACAGGGCACACAAGTCACAGTGTCTAGCAC
CAGCACCACCACACCAGCTCCTAGACCTCCAACTCCTGCTCCTACAATCGCCAGCCAGCCT
CTGTCTCTGAGGCCAGAAGCTTGTAGACCTGCTGCTGGCGGAGCCGTGCATACAAGAGGAC
TGGATTTCGCCTGCGACATCTACATCTGGGCCCCTCTGGCTGGAACATGTGGCGTGCTGCT
GCTGAGCCTGGTCATCACCCTGTACTGCAAGCGGGGCAGAAAGAAGCTGCTGTACATCTTC
AAGCAGCCCTTCATGCGGCCCGTGCAGACCACACAAGAGGAAGATGGCTGCTCCTGTCGGT
TCCCCGAGGAAGAAGAAGGCGGCTGCGAGCTGAGAGTGAAGTTCAGCAGAAGTGCCGACGC
TCCCGCCTATCAGCAGGGACAGAACCAGCTGTACAACGAGCTGAACCTGGGGAGAAGAGAA
GAGTACGACGTGCTGGACAAGCGGAGAGGCAGAGATCCTGAGATGGGCGGAAAGCCCCAGC
GGAGAAAGAATCCTCAAGAGGGCCTGTATAATGAGCTGCAAAAGGACAAGATGGCCGAGGC
CTACAGCGAGATCGGAATGAAGGGCGAGCGCAGAAGAGGCAAGGGACACGATGGACTGTAT
CAGGGCCTGAGCACCGCCACCAAGGATACCTATGATGCCCTGCACATGCAGGCCCTGCCTC
CAAGA
GSI5022 HVV (CD8a Signal Peptide_GSI5022 HVV_CD8a Hinge_BBZ)
(SEQ ID NO: 182)
ATGGCTCTGCCTGTGACAGCTCTGCTGCTGCCTCTGGCTCTGCTTCTGCATGCCGCTAGAC
CCCAAGTGAAGCTGGAAGAGTCTGGCGGAGGACTGGTGCAGGCTGGCAGATCTCTGAGACT
GTCTTGTGCCGCCAGCGAGCACACCTTTAGCTCTCACGTGATGGGCTGGTTCAGACAGGCC
CCTGGCAAAGAAAGGGAAAGCGTGGCCGTTATCGGCTGGCGGGATATCAGCACAAGCTACG
CCGATAGCGTGAAGGGCAGATTCACCATCAGCCGGGACAACGCCAAGAAAACCCTGTACCT
CCAGATGAACAGCCTGAAGCCTGAGGACACCGCCGTGTACTACTGCGCCGCCAGAAGAATT
GACGCCGCCGACTTTGATTCTTGGGGCCAGGGAACCCAAGTGACCGTTTCTAGCGGAGGCG
GTGGAAGTGGCGGCGGTGGATCAGGTGGTGGTGGATCTGAAGTGCAGCTGGTGGAATCAGG
CGGCGGTCTTGTTCAAGCCGGTGGTTCACTGAGACTGAGCTGTGCCGCTTCCGGCAGAACC
TTTACCATGGGATGGTTTAGGCAGGCTCCAGGGAAAGAACGCGAGTTCGTGGCCGCCATTT
CTCTGTCTCCAACACTGGCCTACTACGCCGAGTCCGTGAAAGGCCGGTTCACAATCTCCAG
AGATAATGCCAAGAACACCGTGGTGCTGCAAATGAACTCCCTGAAGCCAGAAGATACAGCC
CTGTATTACTGTGCCGCCGACCGGAAGTCCGTGATGAGCATCAGACCTGACTACTGGGGAC
AGGGCACACAAGTCACAGTGTCCAGCACCAGCACCACAACACCCGCTCCTAGACCTCCAAC
ACCAGCTCCAACAATCGCCAGCCAGCCTCTGTCTCTGAGGCCAGAAGCTTGTAGACCTGCT
GCTGGCGGAGCCGTGCATACAAGAGGACTGGATTTCGCCTGCGACATCTACATCTGGGCCC
CTCTGGCTGGAACATGTGGCGTGCTGCTGCTGAGCCTGGTCATCACCCTGTACTGCAAGCG
GGGCAGAAAGAAGCTGCTGTACATCTTCAAGCAGCCCTTCATGCGGCCCGTGCAGACCACA
CAAGAGGAAGATGGCTGCTCCTGTCGGTTCCCCGAGGAAGAAGAAGGCGGCTGCGAGCTGA
GAGTGAAGTTCAGCAGATCCGCCGACGCTCCTGCCTATCAGCAGGGACAGAACCAGCTGTA
CAACGAGCTGAACCTGGGGAGAAGAGAAGAGTACGACGTGCTGGATAAGCGGAGAGGCAGA
GATCCTGAGATGGGCGGAAAGCCCCAGCGGAGAAAGAATCCTCAAGAGGGCCTGTATAATG
AGCTGCAAAAGGACAAGATGGCCGAGGCCTACAGCGAGATCGGAATGAAGGGCGAGCGCAG
AAGAGGCAAGGGACACGATGGACTGTATCAGGGCCTGAGCACCGCCACCAAGGATACCTAT
GATGCCCTGCACATGCAGGCCCTGCCTCCAAGA
BCMAsdAb1 HHV (CD8a Signal Peptide_anti-BCMAsdAb1 HHV_CD8a
Hinge) (SEQ ID NO: 183)
ATGGCTCTGCCTGTGACAGCTCTGCTGCTGCCTCTGGCTCTGCTTCTGCATGCCGCTAGAC
CTGAAGTGCAGTTGCAGGCTTCTGGCGGAGGACTTGCTCAACCTGGCGGAAGCCTGAGACT
GTCTTGTGCCGCCTCTGGCAGAACCTTCAGCACCTACTTCATGGCCTGGTTCAGACAGCCT
CCTGGCAAAGGCCTGGAATACGTTGGCGGAATCCGTTGGAGTGATGGCGTGCCACACTACG
CCGATAGCGTGAAGGGCAGATTCACCATCAGCCGGGACAACGCCAAGAACACCGTGTACCT
CCAGATGAACAGCCTGAGAGCCGAGGATACCGCCGTGTACTTCTGTGCCAGCAGAGGAATC
GCCGACGGCAGCGATTTTGGCTCTTATGGCCAGGGCACCCAAGTGACCGTGTCCAGCACAA
CAACCCCTGCTCCTAGACCTCCTACACCAGCTCCTACAATCGCCAGCCAGCCTCTGTCTCT
GAGGCCAGAGGCTTGTAGACCTGCTGCTGGCGGAGCCGTGCATACAAGAGGACTGGATTTC
GCCTGC
CD28 signaling domain: CD28 transmembrane_CD28 signaling
domain (SEQ ID NO: 194)
CTGTTCCCCGGACCTAGCAAGCCCTTTTGGGTGCTCGTTGTTGTTGGCGGCGTGCTGGCCT
GTTATAGCCTGCTGGTTACCGTGGCCTTCATCATCTTTTGGGTCCGAAGCAAGCGGAGCCG
GCTGCTGCACAGCGACTACATGAACATGACCCCTAGACGGCCCGGACCAACCAGAAAGCAC
TACCAGCCTTACGCTCCTCCTAGAGACTTCGCCGCCTACAGATCT
Human CD137/4-1BB (SEQ ID NO: 195)
AAGCGGGGCAGAAAGAAACTGCTGTACATCTTCAAGCAGCCCTTCATGCGGCCCGTGCAGA
CCACACAAGAGGAAGATGGCTGCTCCTGCAGATTCCCCGAGGAAGAAGAAGGCGGCTGCGA
ACTT
Human CD134/OX40 (SEQ ID NO: 196)
AGGAGAGACCAGCGTCTGCCACCAGATGCACATAAGCCACCTGGCGGCGGAAGCTTTAGAA
CCCCTATCCAAGAGGAACAGGCCGACGCTCACTCTACACTGGCTAAAATC
truncated/mutated CD137/4-1BB (SEQ ID NO: 197)
CAGCCTTTCATGAGGCCCGTGCAGACCACACAAGAAGAGGACGGCTGCTCCTGCAGATTCC
CCGAGGAAGAGGAAGGCGGTTGCGAACTT
Truncated/mutated CD134/OX40 (SEQ ID NO: 198)
GGCGGCGGAAGCTTTAGAACCCCTATCCAAGAGGAACAGGCCGACGCTCACTCTACACTGG
CT
CD28 (Mature Sequence) (SEQ ID NO: 249)
ATGTTGCGATTGCTTTTGGCCTTGAATTTGTTCCCCTCGATTCAGGTTACCGGAAATAAGA
TACTTGTAAAGCAGTCGCCAATGCTCGTGGCGTATGATAATGCTGTGAACTTATCGTGTAA
GTACTCTTACAACCTTTTTTCGAGGGAATTTCGCGCTAGTCTACACAAGGGTCTAGACTCG
GCTGTGGAGGTGTGCGTGGTCTATGGTAATTATTCGCAGCAGCTTCAGGTTTACAGCAAGA
CTGGCTTCAACTGTGACGGTAAACTAGGAAACGAGTCAGTGACCTTTTACCTCCAAAACCT
ATATGTAAACCAAACAGACATTTATTTTTGCAAGATAGAGGTGATGTACCCTCCTCCCTAT
CTAGACAACGAGAAGTCCAATGGAACAATAATCCACGTTAAGGGGAAACACCTTTGCCCCT
CGCCACTGTTCCCAGGTCCCTCAAAGCCCTTTTGGGTCCTAGTTGTCGTGGGGGGGGTATT
GGCATGCTATTCGTTGTTGGTCACAGTCGCCTTTATAATCTTTTGGGTTAGAAGTAAGCGC
AGTCGTCTACTCCACAGCGATTATATGAATATGACACCAAGGCGTCCCGGCCCTACGCGAA
AACATTACCAGCCCTACGCGCCGCCTCGAGATTTCGCGGCTTATCGAAGC
ICOS (Mature Sequence) (SEQ ID NO: 250)
ATGAAGAGCGGTCTATGGTACTTCTTCCTATTCTGTCTGAGAATCAAAGTGTTAACCGGAG
AAATTAATGGATCGGCCAATTATGAGATGTTTATTTTCCATAACGGCGGAGTACAAATACT
CTGTAAGTACCCTGACATAGTACAGCAGTTTAAGATGCAGCTTTTGAAAGGGGGCCAAATT
CTCTGCGACTTAACCAAGACAAAGGGATCTGGAAACACTGTATCGATAAAGTCCCTCAAGT
TCTGTCATTCCCAATTATCGAATAACAGCGTATCTTTCTTCTTATACAATCTGGACCACTC
CCACGCCAATTATTACTTCTGTAACCTCAGTATTTTCGACCCGCCACCTTTCAAGGTTACT
TTGACAGGAGGTTATCTGCATATCTACGAGTCGCAGCTTTGTTGTCAGCTCAAGTTCTGGC
TGCCCATAGGTTGCGCCGCGTTTGTAGTAGTTTGTATTTTGGGATGTATCTTGATTTGTTG
GCTTACCAAGAAGAAGTACAGCTCGTCGGTCCATGATCCTAATGGCGAGTACATGTTTATG
AGGGCCGTGAATACAGCCAAAAAATCCCGCTTAACGGACGTTACCTTA
CD28 (Stalk, Transmembrane, and Signaling Domain)
(SEQ ID NO: 251)
CTGTTCCCAGGTCCCTCAAAGCCCTTTTGGGTCCTAGTTGTCGTGGGGGGGGTATTGGCAT
GCTATTCGTTGTTGGTCACAGTCGCCTTTATAATCTTTTGGGTTAGAAGTAAGCGCAGTCG
TCTACTCCACAGCGATTATATGAATATGACACCAAGGCGTCCCGGCCCTACGCGAAAACAT
TACCAGCCCTACGCGCCGCCTCGAGATTTCGCGGCTTATCGAAGC
ICOS (Stalk, Transmembrane, and Signaling Domain)
(SEQ ID NO: 252)
TCGCAGCTTTGTTGTCAGCTCAAGTTCTGGCTGCCCATAGGTTGCGCCGCGTTTGTAGTAG
TTTGTATTTTGGGATGTATCTTGATTTGTTGGCTTACCAAGAAGAAGTACAGCTCGTCGGT
CCATGATCCTAATGGCGAGTACATGTTTATGAGGGCCGTGAATACAGCCAAAAAATCCCGC
TTAACGGACGTTACCTTA
TNFRSF4/OX40/CD134 (Mature Sequence) (SEQ ID NO: 253)
ATGTGCGTTGGGGCCCGTCGCCTTGGCCGCGGTCCGTGTGCAGCTTTATTGCTACTTGGCC
TAGGGTTGTCGACAGTGACCGGCCTGCACTGTGTGGGGGACACCTATCCATCGAATGACCG
GTGTTGCCATGAATGCAGGCCGGGAAATGGCATGGTGTCGCGGTGTAGCCGCTCCCAGAAT
ACTGTGTGTCGTCCTTGCGGCCCCGGCTTCTATAATGACGTAGTATCGAGTAAACCTTGTA
AGCCGTGCACGTGGTGCAATCTTAGGTCCGGAAGTGAACGTAAGCAATTATGCACAGCTAC
TCAGGACACCGTGTGTCGTTGCCGAGCAGGGACACAGCCCCTCGATTCATATAAACCTGGA
GTTGATTGCGCACCGTGTCCCCCAGGGCATTTTTCACCTGGCGACAATCAGGCTTGCAAAC
CATGGACTAACTGTACTCTAGCAGGGAAGCATACGCTCCAACCGGCTTCTAATAGCTCTGA
TGCGATATGCGAAGATCGTGATCCCCCCGCAACCCAGCCACAGGAGACACAGGGACCCCCC
GCCCGTCCGATCACCGTGCAGCCGACCGAAGCATGGCCGCGTACTTCGCAGGGGCCTTCGA
CTCGACCCGTCGAGGTTCCCGGTGGTAGAGCAGTTGCAGCTATTTTAGGCCTGGGTCTTGT
GCTTGGGTTGTTGGGTCCCCTCGCCATACTACTAGCTTTGTATTTGTTGCGCCGCGATCAA
CGCTTGCCTCCCGATGCGCACAAGCCACCTGGAGGCGGGTCTTTCCGGACACCTATACAAG
AAGAACAGGCGGACGCCCATAGCACACTCGCTAAGATT
TNFRSF4/OX40/CD134 Transmembrane and Signaling Domain
(SEQ ID NO: 254)
GTTGCAGCTATTTTAGGCCTGGGTCTTGTGCTTGGGTTGTTGGGTCCCCTCGCCATACTAC
TAGCTTTGTATTTGTTGCGCCGCGATCAACGCTTGCCTCCCGATGCGCACAAGCCACCTGG
AGGCGGGTCTTTCCGGACACCTATACAAGAAGAACAGGCGGACGCCCATAGCACACTCGCT
AAGATT
TNFRSF4/OX40/CD134 Signaling Domain (SEQ ID NO: 255)
GCTTTGTATTTGTTGCGCCGCGATCAACGCTTGCCTCCCGATGCGCACAAGCCACCTGGAG
GCGGGTCTTTCCGGACACCTATACAAGAAGAACAGGCGGACGCCCATAGCACACTCGCTAA
GATT
Truncated TNFRSF4 Signaling Domain (OX40t) (SEQ ID NO: 256)
GGAGGCGGGTCTTTCCGGACACCTATACAAGAAGAACAGGCGGACGCCCATAGCACACTCG
CT
TNFRSF5/CD40 (Mature Sequence) (SEQ ID NO: 257)
ATGGTGCGACTCCCGCTACAATGTGTGTTGTGGGGGTGCTTACTAACGGCGGTCCATCCAG
AACCACCTACAGCCTGTCGGGAGAAGCAGTACCTAATTAATAGTCAGTGCTGCAGTCTATG
TCAGCCGGGACAGAAACTAGTATCCGACTGTACCGAATTTACCGAGACCGAGTGCCTACCG
TGTGGTGAATCAGAATTCCTCGATACGTGGAATAGAGAGACCCACTGTCACCAACACAAGT
ATTGCGACCCTAACTTAGGACTGAGGGTGCAGCAAAAGGGGACATCGGAGACGGATACAAT
TTGTACATGCGAGGAAGGGTGGCATTGTACATCCGAAGCATGTGAGAGTTGCGTTCTTCAC
CGTAGCTGTAGTCCGGGATTTGGGGTGAAACAAATTGCTACTGGCGTCTCGGATACGATCT
GCGAACCCTGTCCTGTAGGATTTTTCTCAAATGTTTCTTCAGCGTTCGAGAAATGTCATCC
ATGGACCTCGTGCGAGACGAAGGATTTAGTAGTGCAGCAAGCTGGAACGAATAAAACCGAT
GTAGTCTGTGGGCCGCAAGATCGCTTACGAGCGTTGGTTGTTATTCCTATCATCTTCGGAA
TCCTATTCGCAATTCTCCTGGTATTGGTTTTCATAAAAAAGGTAGCGAAGAAACCGACAAA
CAAGGCCCCCCACCCGAAACAGGAGCCACAGGAGATTAATTTCCCTGACGATCTTCCGGGC
AGTAACACTGCTGCACCGGTGCAGGAAACTTTGCACGGATGTCAACCGGTCACGCAAGAAG
ATGGAAAAGAAAGTCGAATCTCCGTGCAAGAACGCCAG
TNFRSF5 Transmembrane and Signaling Domain (SEQ ID NO: 258)
GCGTTGGTTGTTATTCCTATCATCTTCGGAATCCTATTCGCAATTCTCCTGGTATTGGTTT
TCATAAAAAAGGTAGCGAAGAAACCGACAAACAAGGCCCCCCACCCGAAACAGGAGCCACA
GGAGATTAATTTCCCTGACGATCTTCCGGGCAGTAACACTGCTGCACCGGTGCAGGAAACT
TTGCACGGATGTCAACCGGTCACGCAAGAAGATGGAAAAGAAAGTCGAATCTCCGTGCAAG
AACGCCAG
TNFRSF5 Signaling Domain (SEQ ID NO: 259)
AAAAAGGTAGCGAAGAAACCGACAAACAAGGCCCCCCACCCGAAACAGGAGCCACAGGAGA
TTAATTTCCCTGACGATCTTCCGGGCAGTAACACTGCTGCACCGGTGCAGGAAACTTTGCA
CGGATGTCAACCGGTCACGCAAGAAGATGGAAAAGAAAGTCGAATCTCCGTGCAAGAACGC
CAG
Truncated TNFRSF5 Signaling Domain (TNFRSF5t) (SEQ ID NO: 260)
GCCCCCCACCCGAAACAGGAGCCACAGGAGATTAATTTCCCTGACGATCTTCCGGGCAGTA
ACACTGCTGCACCGGTGCAGGAAACTTTGCACGGATGTCAACCGGTCACGCAAGAAGATGG
AAAAGAAAGTCGAATCTCCGTGCAAGAACGCCAG
TNFRSF7 (CD27) (SEQ ID NO: 261)
ATGGCGCGCCCCCACCCTTGGTGGCTTTGTGTGCTGGGGACACTGGTGGGACTGTCCGCAA
CGCCCGCTCCAAAGAGTTGCCCCGAACGACATTATTGGGCACAAGGTAAACTCTGTTGTCA
AATGTGTGAGCCTGGAACGTTTCTCGTCAAGGATTGCGACCAACATAGAAAAGCGGCTCAG
TGCGATCCGTGTATTCCTGGAGTAAGTTTCAGCCCGGATCACCATACAAGACCTCACTGCG
AGTCTTGTCGTCATTGTAACTCAGGGTTGTTGGTGAGAAACTGCACAATTACAGCCAACGC
TGAATGCGCATGCAGAAACGGTTGGCAATGCCGAGACAAAGAATGCACGGAGTGCGATCCC
CTCCCGAATCCTTCTTTGACGGCCCGTTCCTCCCAAGCACTAAGTCCACATCCCCAACCAA
CGCACCTTCCTTACGTATCTGAAATGTTAGAGGCACGTACGGCTGGGCATATGCAAACCCT
TGCCGATTTTCGACAGTTACCCGCTAGGACTTTGTCAACACACTGGCCGCCCCAGAGATCG
CTGTGCTCGTCGGATTTTATTAGGATCCTGGTCATTTTTTCTGGGATGTTTCTCGTGTTTA
CCTTAGCGGGAGCATTATTCCTACATCAACGCAGGAAGTATCGCTCCAACAAGGGCGAAAG
TCCAGTTGAACCTGCCGAACCATGCCACTATTCGTGCCCCCGAGAGGAGGAGGGAAGTACC
ATACCTATCCAAGAAGACTATCGCAAGCCCGAGCCAGCATGCAGCCCT
TNFRSF7 (CD27) Transmembrane and Signaling Domain
(SEQ ID NO: 262)
ATCCTGGTCATTTTTTCTGGGATGTTTCTCGTGTTTACCTTAGCGGGAGCATTATTCCTAC
ATCAACGCAGGAAGTATCGCTCCAACAAGGGCGAAAGTCCAGTTGAACCTGCCGAACCATG
CCACTATTCGTGCCCCCGAGAGGAGGAGGGAAGTACCATACCTATCCAAGAAGACTATCGC
AAGCCCGAGCCAGCATGCAGCCCT
TNFRSF7 (CD27) Signaling Domain (SEQ ID NO: 263)
CAACGCAGGAAGTATCGCTCCAACAAGGGCGAAAGTCCAGTTGAACCTGCCGAACCATGCC
ACTATTCGTGCCCCCGAGAGGAGGAGGGAAGTACCATACCTATCCAAGAAGACTATCGCAA
GCCCGAGCCAGCATGCAGCCCT
Truncated TNFRSF7 (CD27) Signaling Domain (TNFRSF7t)
(SEQ ID NO: 264)
GGCGAGAGCCCCGTGGAACCTGCCGAGCCTTGTCACTACAGCTGCCCCAGAGAGGAAGAGG
GCAGCACAATCCCCATCCAAGAGGACTACAGAAAGCCCGAGCCTGCCTGCTCTCCT
TNFRSF9 (4-1BB) (SEQ ID NO: 265)
ATGGGGAACAGTTGCTACAACATAGTCGCGACGCTACTACTAGTGTTGAATTTTGAGCGTA
CCAGATCGCTGCAGGATCCGTGTTCGAATTGCCCTGCAGGAACGTTTTGTGACAACAACAG
AAATCAAATATGTTCCCCATGCCCGCCGAACTCCTTTTCTTCGGCCGGAGGCCAGCGAACT
TGCGACATCTGCCGCCAATGCAAGGGAGTCTTCCGGACTCGGAAAGAATGCTCCTCCACGA
GTAACGCCGAATGCGATTGCACCCCTGGGTTTCACTGCCTGGGCGCAGGGTGCAGCATGTG
CGAGCAGGATTGCAAGCAAGGTCAGGAGCTGACGAAAAAAGGATGCAAAGACTGTTGTTTT
GGAACGTTCAATGATCAAAAACGCGGTATCTGTAGGCCTTGGACAAACTGCAGTCTAGACG
GAAAATCGGTGCTCGTTAATGGCACGAAGGAACGCGATGTCGTCTGCGGTCCCAGCCCGGC
TGATCTATCACCAGGTGCATCATCCGTAACACCCCCCGCTCCTGCAAGAGAACCGGGCCAC
TCCCCTCAAATTATAAGTTTCTTCTTGGCCTTGACTTCGACGGCGCTATTGTTTTTGCTCT
TCTTTCTTACGTTACGGTTTTCCGTTGTCAAAAGAGGGCGAAAAAAGTTGCTTTATATTTT
CAAACAACCATTTATGCGACCGGTTCAAACCACACAAGAGGAAGATGGATGCTCCTGCCGG
TTCCCAGAAGAGGAGGAGGGCGGTTGCGAGCTC
TNFRSF9 (4-1BB) Transmembrane and Signaling Domain
(SEQ ID NO: 266)
ATTATAAGTTTCTTCTTGGCCTTGACTTCGACGGCGCTATTGTTTTTGCTCTTCTTTCTTA
CGTTACGGTTTTCCGTTGTCAAAAGAGGGCGAAAAAAGTTGCTTTATATTTTCAAACAACC
ATTTATGCGACCGGTTCAAACCACACAAGAGGAAGATGGATGCTCCTGCCGGTTCCCAGAA
GAGGAGGAGGGCGGTTGCGAGCTC
TNFRSF9 (4-1BB) Signaling Domain (SEQ ID NO: 267)
CAGCCTTTCATGAGGCCTGTGCAGACCACACAAGAAGAGGACGGCTGCTCCTGTCGGTTCC
CCGAGGAAGAGGAAGGCGGTTGCGAACTT
TNFRSF6/FAS/CD95 (Mature Sequence) (SEQ ID NO: 268)
ATGTTGGGCATATGGACCCTACTACCCTTGGTCCTGACTTCGGTGGCGCGGCTATCGAGCA
AATCTGTCAACGCGCAAGTGACTGACATTAACTCCAAGGGTCTCGAGCTACGAAAGACGGT
CACTACCGTTGAAACGCAGAATCTTGAAGGCCTCCACCACGACGGTCAATTTTGCCACAAA
CCCTGTCCGCCCGGTGAGCGCAAGGCCCGTGATTGCACAGTTAACGGTGACGAACCTGACT
GCGTTCCATGCCAAGAGGGGAAGGAATACACAGACAAGGCCCATTTCTCTAGCAAATGTAG
GCGGTGCCGTCTCTGTGACGAGGGACATGGTCTTGAGGTTGAAATAAACTGTACTCGGACA
CAAAACACCAAGTGCAGGTGTAAGCCGAATTTTTTTTGTAACTCTACCGTGTGTGAACATT
GTGATCCTTGTACGAAATGTGAGCACGGAATTATAAAGGAATGTACTTTAACTTCCAACAC
AAAGTGCAAGGAAGAGGGCTCTCGGAGCAATCTAGGATGGTTATGCTTGTTACTCCTTCCG
ATTCCACTTATTGTCTGGGTTAAGCGTAAAGAAGTGCAGAAGACATGTCGAAAGCATAGGA
AAGAGAACCAAGGTAGCCATGAGTCCCCTACCCTCAATCCCGAAACCGTGGCCATTAATCT
GTCCGATGTGGACTTGTCGAAATATATAACCACTATTGCAGGCGTCATGACGTTAAGTCAA
GTGAAAGGATTCGTTCGCAAAAACGGGGTGAACGAAGCGAAGATCGATGAGATCAAGAACG
ATAATGTCCAAGACACGGCTGAACAAAAGGTGCAGCTTCTCAGGAACTGGCATCAATTACA
TGGTAAAAAGGAAGCGTACGACACGTTAATTAAGGACCTCAAAAAAGCCAATTTGTGCACC
CTAGCGGAGAAAATCCAAACCATTATACTCAAAGATATAACTTCAGACTCGGAAAACTCTA
ACTTCCGTAACGAGATTCAGTCGCTGGTT
Programed Death Receptor 1/PD-1 (Mature Sequence)
(SEQ ID NO: 269)
TTCCTGGACTCTCCTGACAGACCCTGGAATCCTCCAACATTCAGCCCCGCTCTGCTGGTGG
TTACCGAGGGCGATAATGCCACCTTCACCTGTAGCTTCAGCAACACCAGCGAGAGCTTCGT
GCTGAACTGGTACAGAATGAGCCCCAGCAACCAGACCGACAAGCTGGCCGCCTTTCCTGAG
GATAGATCTCAGCCCGGCCAGGACTGCCGGTTCAGAGTTACACAGCTGCCCAACGGCCGGG
ACTTCCACATGTCTGTCGTCCGGGCCAGAAGAAACGACAGCGGCACATATCTGTGCGGCGC
CATTTCTCTGGCCCCTAAGGCTCAGATCAAAGAGAGCCTGAGAGCCGAGCTGAGAGTGACA
GAAAGACGGGCCGAAGTGCCCACAGCTCACCCTTCACCTTCTCCAAGACCTGCCGGCCAGT
TCCAGACACTGGTCGTGGGAGTTGTTGGCGGACTGCTGGGATCTCTGGTGCTGCTTGTTTG
GGTGCTCGCCGTGATCTGTAGCAGAGCCGCCAGAGGAACAATCGGCGCCAGAAGGACAGGC
CAGCCTCTGAAAGAGGATCCCTCTGCTGTCCCCGTGTTCAGCGTGGACTATGGCGAGCTGG
ATTTCCAGTGGCGGGAAAAGACACCCGAGCCTCCAGTGCCTTGTGTGCCTGAGCAGACAGA
GTACGCCACCATCGTGTTCCCTAGCGGCATGGGCACATCTAGCCCTGCCAGAAGAGGATCT
GCCGACGGACCTAGATCTGCCCAGCCTCTCAGACCTGAGGATGGCCACTGTTCTTGGCCTC
TT

The present disclosure provides, in some aspects, a chimeric co-stimulatory intracellular protein (CIP) comprising a first and at least a second signal transduction domains, wherein the first and the at least second signal transduction domains are non-identical; and wherein the at least second signal transduction domain comprises a mutant intracellular signaling domain of a tumor necrosis factor receptor (TNFR) family protein.

The present disclosure also provides a chimeric co-stimulatory intracellular protein (CIP) comprising a first and at least a second signal transduction domains, wherein the first and the at least second signal transduction domains are non-identical; and wherein the at least second signal transduction domain comprises a mutant CD137 (4-1BB) intracellular domain, a wildtype or mutant CD134 (OX-40) intracellular domain, a wildtype or mutant CD27 intracellular domain, or a wildtype or mutant CD40 intracellular domain.

In some embodiments of the CIP disclosed herein, the mutant intracellular signaling domain of a TNFR family protein is any one of a mutant CD137 (4-1BB) intracellular domain a wildtype or mutant CD134 (OX-40) intracellular domain, a wildtype or mutant CD27 intracellular domain, a wildtype or mutant CD40 intracellular domain. In some embodiments, the CIP further comprises a transmembrane domain. In some embodiments of the CIP disclosed herein, the mutant CD137 intracellular domain is a truncated CD137 intracellular domain.

In some embodiments of the CIP disclosed herein, the truncated CD137 intracellular domain comprises an amino acid sequence according to amino acid position 13 to amino acid position 42 of the CD137 intracellular domain, of the present disclosure. In some embodiments of the CIP disclosed herein, the truncated CD137 intracellular domain comprises a deletion of a continuous stretch of one, two, three, four, five, six, seven, eight, nine, ten or more amino acids from the N-terminus the CD137 intracellular domain, of the present disclosure. In some embodiments of the CIP disclosed herein, the truncated CD137 intracellular domain comprises a deletion of one, two, three, four, five, six, seven, eight, nine, ten or more amino acids from amino acid position 1 to amino acid position 12 of the N-terminus of the CD137 intracellular domain, of the present disclosure. In some embodiments of the CIP disclosed herein, the CD137 intracellular domain of the present disclosure comprises an amino acid sequence according to SEQ ID NO: 1.

In some embodiments of the CIP disclosed herein, the truncated CD137 intracellular domain comprises an amino acid sequence according to SEQ ID NO: 3.

In some embodiments of the CIP disclosed herein, the mutant CD137 intracellular domain comprises a deletion of one, two, three or four lysine residue(s) from amino acid position 1 to amino acid position 12 of the N-terminus of the CD137 intracellular domain, of the present disclosure. In some embodiments of the CIP disclosed herein, the mutant CD137 intracellular domain comprises one or more lysine mutation(s) from amino acid position 1 to amino acid position 12 of the N-terminus of the CD137 intracellular domain, of the present disclosure. In some embodiments of the CIP disclosed herein, the mutant CD137 intracellular domain comprises one or more lysine mutation(s) at amino acid positions selected from amino acid positions 1, 5, 6 and 12 of the N-terminus of the CD137 intracellular domain, of the present disclosure.

In some embodiments of the CIP disclosed herein, the mutant CD137 intracellular domain comprises a deletion of one or more proximal basic amino acids from amino acid position 1 to amino acid position 12 of the N-terminus of the CD137 intracellular domain, of the present disclosure. In some embodiments of the CIP disclosed herein, the mutant CD137 intracellular domain comprises one or more proximal basic amino acid mutation(s) from amino acid position 1 to amino acid position 12 of the N-terminus of the CD137 intracellular domain, of the present disclosure. In some embodiments of the CIP disclosed herein, the mutant CD137 intracellular domain comprises one or more proximal basic amino acid mutation(s) at amino acid positions selected from amino acid positions 1, 2, 3, 4, 5 and 6 of the N-terminus of the CD137 intracellular domain, of the present disclosure. In some embodiments of the CIP disclosed herein, the mutant CD137 intracellular domain further comprises a lysine mutation at amino acid position 12 of the N-terminus of the CD137 intracellular domain, of the present disclosure.

In some embodiments of the CIP disclosed herein, the mutant CD134 intracellular domain is a truncated CD134 intracellular domain. In some embodiments of the CIP disclosed herein, the truncated CD134 intracellular domain comprises an amino acid sequence according to amino acid position 15 to amino acid position 37 of the CD134 intracellular domain, of the present disclosure. In some embodiments of the CIP disclosed herein, the truncated CD134 intracellular domain comprise a deletion of a continuous stretch of one, two, three, four, five, six, seven, eight, nine, ten, eleven, twelve, thirteen or more amino acids from the N-terminus of the CD134 intracellular domain, of the present disclosure. In some embodiments of the CIP disclosed herein, the truncated CD134 intracellular domain comprises a deletion of one, two, three, four, five, six, seven, eight, nine, ten, eleven, twelve, thirteen or more amino acids from amino acid position 1 to amino acid position 14 of the N-terminus of the CD134 intracellular domain, of the present disclosure.

In some embodiments of the CIP disclosed herein, the truncated CD134 intracellular domain comprises an amino acid sequence according to SEQ ID NO: 6.

In some embodiments of the CIP disclosed herein, the mutant CD134 intracellular domain comprises a deletion of a lysine residue from amino acid position 1 to amino acid position 14 of the N-terminus of the CD134 intracellular domain, of the present disclosure. In some embodiments of the CIP disclosed herein, the mutant CD134 intracellular domain comprises a lysine mutation at amino acid position 12 of the N-terminus of the CD134 intracellular domain, of the present disclosure.

In some embodiments of the CIP disclosed herein, the mutant CD134 intracellular domain comprises a deletion of one or more proximal basic amino acids from amino acid position 1 to amino acid position 14 of the N-terminus of the CD134 intracellular domain, of the present disclosure. In some embodiments of the CIP disclosed herein, the mutant CD134 intracellular domain comprises one or more proximal basic amino acid mutation(s) from amino acid position 1 to amino acid position 14 of the N-terminus of the CD134 intracellular domain. In some embodiments of the CIP disclosed herein, the mutant CD134 intracellular domain comprises one or more proximal basic amino acid mutation(s) at amino acid positions selected from amino acid positions 1, 2, and 5 of the N-terminus of the CD134 intracellular domain. In some embodiments of the CIP disclosed herein, the mutant CD134 intracellular domain further comprises a lysine mutation at amino acid position 12 of the N-terminus of the CD134 intracellular domain

In some embodiments, the CIP disclosed herein comprises a first signal transduction domain derived from a protein of the CD28 family. In some embodiments, the CIP disclosed herein comprises a first signal transduction domain derived from any one of CD28, CD28H, ICOS or a combination thereof.

In some embodiments, the CIP disclosed herein comprises a first signal transduction domain derived from ICOS. In some embodiments, the first signal transduction domain derived from ICOS comprises an amino acid sequence according to SEQ ID NO: 9.

In some embodiments, the CIP disclosed herein comprises a first signal transduction domain comprising a portion of a CD28 intracellular domain combined with an ICOS domain according to SEQ ID NO: 9. In some embodiments of the CIP disclosed herein, the first signal transduction domain comprises an amino acid sequence according to any one of SEQ ID NOs: 12 or 109. In some embodiments, the CIP disclosed herein comprises a first signal transduction domain derived from CD28. In some embodiments of the CIP disclosed herein, the first signal transduction domain derived from CD28 comprises an amino acid sequence according to SEQ ID NO: 10. In some embodiments of the CIP disclosed herein, the first signal transduction domain derived from CD28 comprises an amino acid sequence according to any one of SEQ ID NOs: 121-122. In some embodiments, the CIP comprises an amino acid sequence according to any one of SEQ ID NOs: 14-17.

In some embodiments, the third signal transduction domain of the CIP disclosed herein is a CD2 signaling domain. In some embodiments, the third signal transduction domain of the CIP disclosed herein is a mutant CD2 signaling domain. In some embodiments, the mutant CD2 signaling domain is a truncated CD2 signaling domain. In some embodiments, the third signal transduction domain of the CIP disclosed herein is a CD2 signaling domain comprising an amino acid sequence according to SEQ ID NO: 49. In some embodiments, the third signal transduction domain of the CIP disclosed herein is a CD2 signaling domain comprising an amino acid sequence having at least 50%, 55%, 60%, 65%, 70%, 75%, 80%, 85%, 90%, 95%, 97% or 99% identity to SEQ ID NO: 49.

In some embodiments, the third signal transduction domain of the CIP disclosed herein, is an IL-2RB protein signaling domain comprising an amino acid sequence according to SEQ ID NO: 50. In some embodiments, the third signal transduction domain of the CIP disclosed herein is an IL-2RB protein signaling domain comprising an amino acid sequence having at least 50%, 55%, 60%, 65%, 70%, 75%, 80%, 85%, 90%, 95%, 97% or 99% identity to SEQ ID NO: 50.

In some embodiments, the fourth signal transduction domain of the CIP disclosed herein is a CD2 signaling domain. In some embodiments, the fourth signal transduction domain of the CIP disclosed herein is a mutant CD2 signaling domain. In some embodiments, the mutant CD2 signaling domain is a truncated CD2 signaling domain. In some embodiments, the fourth signal transduction domain of the CIP disclosed herein is a CD2 signaling domain comprising an amino acid sequence according to SEQ ID NO: 49. In some embodiments, the fourth signal transduction domain of the CIP disclosed herein is a CD2 signaling domain comprising an amino acid sequence having at least 50%, 55%, 60%, 65%, 70%, 75%, 80%, 85%, 90%, 95%, 97% or 99% identity to SEQ ID NO: 49.

In some embodiments, the fourth signal transduction domain of the CIP disclosed herein, is an IL-2RB protein signaling domain comprising an amino acid sequence according to SEQ ID NO: 50. In some embodiments, the fourth signal transduction domain of the CIP disclosed herein is an IL-2RB protein signaling domain comprising an amino acid sequence having at least 50%, 55%, 60%, 65%, 70%, 75%, 80%, 85%, 90%, 95%, 97% or 99% identity to SEQ ID NO: 50.

In some embodiments, the CIP disclosed herein is for expression in a T cell, wherein the T cell co-expresses at least one of the endogenous co-stimulatory molecules CD28, CD2, OX-40, ICOS, CD28, CD4, CD8 and CD40L or a combination thereof.

In some embodiments, the CIP disclosed herein, is co-expressed with a T cell receptor (TCR) in a T cell. In some embodiments, the TCR is an endogenous TCR. In some embodiments, the TCR is an artificial TCR. In some embodiments, the artificial TCR is an affinity enhanced TCR. In some embodiments, the CIP when co-expressed with a TCR in a T cell provides a second activation signal for inducing activation and proliferation of the T cell, wherein the first activation signal is provided by antigen binding by the TCR.

In some embodiments, the CIP disclosed herein, is expressed in a T cell as a component of an artificial receptor for a target. In some embodiments, the artificial receptor is a chimeric antigen receptor (CAR), a receptor for a ligand or a component thereof, an antibody or a fragment thereof. In some embodiments, the CIP disclosed herein, is expressed as a component of a CAR. In some embodiments, the CIP disclosed herein, is expressed as a component of an antibody or a fragment thereof. In some embodiments, the antibody or a fragment thereof is a Fab fragment, a F(ab)2 fragment, a diabody, a nanobody, a sdAb, a Fv, a V_{H}H fragment, or a single chain Fv fragment. In some embodiments, the CIP expressed as a component of an artificial receptor in a T cell, as disclosed herein induces activation and proliferation of the T cell upon target binding by the artificial receptor.

### Fas Dominant Negative Receptor (“FASDD”)

Fas cell surface death receptor (Fas) is also known as APT1; CD95; FAS1; APO-1; FAS™; ALPS1A; TNFRSF6. GenBank ID: 355 (human), 14102 (mouse), 246097 (rat), 282488 (cattle), 486469 (dog). The protein product of Fas includes, but is not limited to, NCBI Reference Sequences NP_000034.1, NP_001307548.1, NP_690610.1 and NP_69061 1. 1.

Fas is a member of the TNF-receptor superfamily and contains a death domain. It is involved in the regulation of programmed cell death, and has been implicated in the pathogenesis of various malignancies and diseases of the immune system. The interaction of Fas with its ligand allows the formation of a death-inducing signaling complex with other components, e.g., Fas-associated protein with death domain (FADD), which can induce programmed cell death.

In some embodiments, a Fas polypeptide is a human Fas polypeptide. In some embodiments, a human Fas polypeptide comprises or has the amino acid sequence of NCBI Reference No.: NP_000034.1 (SEQ ID NO: 200), which is provided below. In some embodiments, a human Fas polypeptide comprises or has an amino acid sequence that is at least about 80%, at least about 85%, at least about 90%, at least about 95%, at least about 99%, or at least about 100% homologous or identical to the sequence set forth in SEQ ID NO: 200.

(SEQ ID NO: 200)
MLGIWTLLPLVLTSVARLSSKSWAQVTDINSK
GLELRKTVTTVETQNLEGLHHDGQFCHKPCPP
GERKARDCTWGDEPDCVPCQEGKEYTDKAHFS
SKCRRCRLCDEGHGLEVEINCTRTQNTKCRCK
PNFFCNSTVCEHCDPCTKCEHGIIKECTLTSN
TKCKEEGSRSNLGWLCLLLLPIPLIVWVKRKE
VQKTCRKHRKENQGSHESPTLNPETVAINLSD
VDLSKYITTIAGVMTLSQVKGFVRKNGWEAKI
DEIKNDNVQDTAEQKVQLLRNWHQLHGKKEAY
DTLIKDLKKANLCTLAEKIQTIILKDITSDSE
NSNFRNEIQSLV

An exemplary nucleotide sequence encoding the amino acid sequence of SEQ ID NO: 200 is set forth in SEQ ID NO: 201, which is provided below:

(SEQ ID NO: 201)
ATGCTGGGCATCTGGACCCTCCTACCTCTGGT
TCTTACGTCTGTTGCTAGATTATCGTCCAAAA
GTGTTAATGCCCAAGTGACTGACATCAACTCC
AAGGGATTGGAATTGAGGAAGACTGTTACTAC
AGTTGAGACTCAGAACTTGGAAGGCCTGCATC
ATGATGGCCAATTCTGCCATAAGCCCTGTCCT
CCAGGTGAAAGGAAAGCTAGGGACTGCACAGT
CAATGGGGATGAACCAGACTGCGTGCCCTGCC
AAGAAGGGAAGGAGTACACAGACAAAGCCCAT
TTTTCTTCCAAATGCAGAAGATGTAGATTGTG
TGATGAAGGACATGGCTTAGAAGTGGAAATAA
ACTGCACCCGGACCCAGAATACCAAGTGCAGA
TGTAAACCAAACTTTTTTTGTAACTCTACTGT
ATGTGAACACTGTGACCCTTGCACCAAATGTG
AACATGGAATCATCAAGGAATGCACACTCACC
AGCAACACCAAGTGCAAAGAGGAAGGATCCAG
ATCTAACTTGGGGTGGCTTTGTCTTCTTCTTT
TGCCAATTCCACTAATTGTTTGGGTGAAGAGA
AAGGAAGTACAGAAAACATGCAGAAAGCACAG
AAAGGAAAACCAAGGTTCTCATGAATCTCCAA
CCTTAAATCCTGAAACAGTGGCAATAAATTTA
TCTGATGTTGACTTGAGTAAATATATCACCAC
TATTGCTGGAGTCATGACACTAAGTCAAGTTA
AAGGCTTTGTTCGAAAGAATGGTGTCAATGAA
GCCAAAATAGATGAGATCAAGAATGACAATGT
CCAAGACACAGCAGAACAGAAAGTTCAACTGC
TTCGTAATTGGCATCAACTTCATGGAAAGAAA
GAAGCGTATGACACATTGATTAAAGATCTCAA
AAAAGCCAATCTTTGTACTCTTGCAGAGAAAA
TTCAGACTATCATCCTCAAGGACATTACTAGT
GACTCAGAAAATTCAAACTTCAGAAATGAAAT
CCAAAGCTTGGTC

In some embodiments, the term “dominant negative FAS polypeptide” (or “FASDD”) refers to the dominant negative form of a FAS polypeptide, which is a gene product of a dominant negative mutation of a FAS gene. In some embodiments, a dominant negative mutation (also called “antimorphic mutations”) has an altered gene product that acts antagonistically to the wild-type allele. In some embodiments, a dominant negative FAS polypeptide adversely affects the normal, wild-type FAS polypeptide within the same cell. In some embodiments, the dominant negative FAS polypeptide interacts with a wild-type Fas polypeptide, but blocks its signal transduction to downstream molecules, e.g., FADD.

In some embodiments, the dominant negative FAS polypeptide comprises a heterologous signal peptide, for example, an IL-2 signal peptide, a kappa leader sequence, a CD8 leader sequence or a peptide with essentially equivalent activity.

In some embodiments, the dominant negative FAS polypeptide comprises at least one modification in the intracellular domain. In some embodiments, the at least one modification prevents the binding of FAS to a FADD polypeptide. In some embodiments, the at least one modification is within the death domain. In some embodiments, the at least one modification is within amino acids about 200 to about 320 of SEQ ID NO: 200. In some embodiments, the at least one modification is within amino acids about 200 to about 319 of SEQ ID NO: 200. In some embodiments, the at least one modification is within amino acids about 202 to about 319 of SEQ ID NO: 200. In some embodiments, the at least one modification is within amino acids about 226 to about 319 of SEQ ID NO: 200. Death domains of Fas protein are disclosed in Tartaglia L A et al. Cell. (1993); 74 (5): 845-53; Itoh and Nagata. J Biol Chem. (1993); 268 (15): 10932; Boldin M P et al. J Biol Chem. (1995); 270 (14): 7795-8; and Huang B et al. Nature (1996); 384 (6610): 638-41, all of which are incorporated by reference herein. In some embodiments, the at least one modification is within the extracellular binding domain.

In some embodiments, the modification is selected from the group consisting of mutations, deletions, and insertions. In some embodiments, the mutation is a point mutation.

In some embodiments, the modification is a deletion. In some embodiments, the dominant negative FAS polypeptide comprises a partial or complete deletion of the death domain. In some embodiments, the dominant negative FAS polypeptide comprises or has a deletion of amino acid residues 230-314 of a human wild-type FAS polypeptide (e.g., one having the amino acid sequence set forth in SEQ ID NO: 200). In some embodiments, the FASDD has the amino acid sequence set forth in SEQ ID NO: 201. SEQ ID NO: 201 is provided below.

(SEQ ID NO: 201)
MLGIWTLLPLVLTSVARLSSKSWAQVTDINSKGLELRKTVTTVETQNLEG
LHHDGQFCHKPCPPGERKARDCTWGDEPDCVPCQEGKEYTDKAHFSSKCR
RCRLCDEGHGLEVEINCTRTQNTKCRCKPNFFCNSTVCEHCDPCTKCEHG
IIKECTLTSNTKCKEEGSRSNLGWLCLLLLPIPLIVWVKRKEVQKTCRKH
RKENQGSHESPTLNPETVAINLSDVDLLKDITSDSENSNERNEIQSLV

An exemplary nucleotide sequence encoding the amino acid sequence of SEQ ID NO: 151 is set forth in SEQ ID NO: 202, which is provided below.

(SEQ ID NO: 202)
ATGCTGGGCATCTGGACCCTCCTACCTCTGGTTCTTACGTCTGTTGCTAG
ATTATCGTCCAAAAGTGTTAATGCCCAAGTGACTGACATCAACTCCAAGG
GATTGGAATTGAGGAAGACTGTTACTACAGTTGAGACTCAGAACTTGGAA
GGCCTGCATCATGATGGCCAATTCTGCCATAAGCCCTGTCCTCCAGGTGA
AAGGAAAGCTAGGGACTGCACAGTCAATGGGGATGAACCAGACTGCGTGC
CCTGCCAAGAAGGGAAGGAGTACACAGACAAAGCCCATTTTTCTTCCAAA
TGCAGAAGATGTAGATTGTGTGATGAAGGACATGGCTTAGAAGTGGAAAT
AAACTGCACCCGGACCCAGAATACCAAGTGCAGATGTAAACCAAACTTTT
TTTGTAACTCTACTGTATGTGAACACTGTGACCCTTGCACCAAATGTGAA
CATGGAATCATCAAGGAATGCACACTCACCAGCAACACCAAGTGCAAAGA
GGAAGGTTCCAGATCTAACTTGGGGTGGCTTTGTCTTCTTCTTTTGCCAA
TTCCACTAATTGTTTGGGTGAAGAGAAAGGAAGTACAGAAAACATGCAGA
AAGCACAGAAAGGAAAACCAAGGTTCTCATGAATCTCCAACCTTAAATCC
TGAAACAGTGGCAATAAATTTATCTGATGTTGACTTGCTCAAGGACATTA
CTAGTGACTCAGAAAATTCAAACTTCAGAAATGAAATCCAAAGCTTGGTC

In some embodiments, the dominant negative Fas polypeptide comprises or has an amino acid sequence that is at least about 80%, at least about 85%, at least about 90%, at least about 95%, at least about 99%, or at least about 100% homologous or identical to the amino acid sequence set forth in SEQ ID NO: 201. In some embodiments, the dominant negative Fas polypeptide having an amino acid sequence that is at least about 80%, at least about 85%, at least about 90%, at least about 95%, at least about 99%, or at least about 100% homologous or identical to the amino acid sequence set forth in SEQ ID NO: 201 comprises or has deletion of amino acid residues 230-314 of a human FAS polypeptide (e.g., one having the amino acid sequence set forth in SEQ ID NO: 200).

In some embodiments, the modification is a point mutation. In some embodiments, the dominant negative FAS polypeptide comprises or has a point mutation at position 260 of a human FAS polypeptide (e.g., one having the amino acid sequence set forth in SEQ ID NO: 200). In some embodiments, the point mutation is D260V. In some embodiments, the dominant negative FAS polypeptide having the point mutation D260V of a human wild-type FAS polypeptide is designated as “hFasD260V” hFasD260V has the amino acid sequence set forth in SEQ ID NO: 203. SEQ ID NO: 203 is provided below.

(SEQ ID NO: 203)
MLGIWTLLPLVLTSVARLSSKSWAQVTDINSKGLELRKTVTTVETQNLEG
LHHDGQFCHKPCPPGERKARDCTWGDEPDCVPCQEGKEYTDKAHFSSKCR
RCRLCDEGHGLEVEINCTRTQNTKCRCKPNFFCNSTVCEHCDPCTKCEHG
IIKECTLTSNTKCKEEGSRSNLGWLCLLLLPIPLIVWVKRKEVQKTCRKH
RKENQGSHESPTLNPETVAINLSDVDLSKYITTIAGVMTLSQVKGFVRKN
GWEAKIVEIKNDNVQDTAEQKVQLLRNWHQLHGKKEAYDTLIKDLKKANL
CTLAEKIQTIILKDITSDSENSNERNEIQSLV

An exemplary nucleotide sequence encoding the amino acid sequence of SEQ ID NO: 203 is set forth in SEQ ID NO: 204, which is provided below.

(SEQ ID NO: 204)
ATGCTGGGCATCTGGACCCTCCTACCTCTGGTTCTTACGTCTGTTGCTAG
ATTATCGTCCAAAAGTGTTAATGCCCAAGTGACTGACATCAACTCCAAGG
GATTGGAATTGAGGAAGACTGTTACTACAGTTGAGACTCAGAACTTGGAA
GGCCTGCATCATGATGGCCAATTCTGCCATAAGCCCTGTCCTCCAGGTGA
AAGGAAAGCTAGGGACTGCACAGTCAATGGGGATGAACCAGACTGCGTGC
CCTGCCAAGAAGGGAAGGAGTACACAGACAAAGCCCATTTTTCTTCCAAA
TGCAGAAGATGTAGATTGTGTGATGAAGGACATGGCTTAGAAGTGGAAAT
AAACTGCACCCGGACCCAGAATACCAAGTGCAGATGTAAACCAAACTTTT
TTTGTAACTCTACTGTATGTGAACACTGTGACCCTTGCACCAAATGTGAA
CATGGAATCATCAAGGAATGCACACTCACCAGCAACACCAAGTGCAAAGA
GGAAGGATCCAGATCTAACTTGGGGTGGCTTTGTCTTCTTCTTTTGCCAA
TTCCACTAATTGTTTGGGTGAAGAGAAAGGAAGTACAGAAAACATGCAGA
AAGCACAGAAAGGAAAACCAAGGTTCTCATGAATCTCCAACCTTAAATCC
TGAAACAGTGGCAATAAATTTATCTGATGTTGACTTGAGTAAATATATCA
CCACTATTGCTGGAGTCATGACACTAAGTCAAGTTAAAGGCTTTGTTCGA
AAGAATGGTGTCAATGAAGCCAAAATAGTTGAGATCAAGAATGACAATGT
CCAAGACACAGCAGAACAGAAAGTTCAACTGCTTCGTAATTGGCATCAAC
TTCATGGAAAGAAAGAAGCGTATGACACATTGATTAAAGATCTCAAAAAA
GCCAATCTTTGTACTCTTGCAGAGAAAATTCAGACTATCATCCTCAAGGA
CATTACTAGTGACTCAGAAAATTCAAACTTCAGAAATGAAATCCAAAGCT
TGGTC

In some embodiments, the dominant negative FAS polypeptide comprises or has an amino acid sequence that is at least about 80%, at least about 85%, at least about 90%, at least about 95%, at least about 99%, or at least about 100% homologous or identical to the amino acid sequence set forth in SEQ ID NO: 203. In some embodiments, the dominant negative FAS polypeptide having an amino acid sequence that is at least about 80%, at least about 85%, at least about 90%, at least about 95%, at least about 99%, or at least about 100% homologous or identical to the amino acid sequence set forth in SEQ ID NO: 203 comprises or has the point mutation D260V of a human FAS polypeptide (e.g., one having the amino acid sequence set forth in SEQ ID NO: 200). In certain non-limiting embodiments, the dominant negative FAS polypeptide comprises a heterologous signal peptide, for example, an IL-2 signal peptide, a kappa leader sequence, a CD8 leader sequence or a peptide with essentially equivalent activity.

In some embodiments, the FASDD comprises an amino acid sequence from Table 11.

TABLE 11

| SEQ ID NO: | Amino acid sequence |
|---|---|
| 230 | TNFRSF6/FAS/CD95 (Signal Peptide-Mature Sequence) |
| | MLGIWTLLPLVLTSVARLSSKSVNAQVTDINSKGLELRKTVTTVETQNLEGL |
| | HHDGQFCHKPCPPGERKARDCTVNGDEPDCVPCQEGKEYTDKAHFSSKCRRC |
| | RLCDEGHGLEVEINCTRTQNTKCRCKPNFFCNSTVCEHCDPCTKCEHGIIKE |
| | CTLTSNTKCKEEGSRSNLGWLCLLLLPIPLIVWVKRKEVQKTCRKHRKENQG |
| | SHESPTLNPETVAINLSDVDLSKYITTIAGVMTLSQVKGFVRKNGVNEAKID |
| | EIKNDNVQDTAEQKVQLLRNWHQLHGKKEAYDTLIKDLKKANLCTLAEKIQT |
| | IILKDITSDSENSNFRNEIQSLV |
| 231 | Tumor necrosis factor receptor superfamily member |
| | 6/FAS/CD95 (Extracellular) |
| | QVTDINSKGLELRKTVTTVETQNLEGLHHDGQFCHKPCPPGERKARDCTVNG |
| | DEPDCVPCQEGKEYTDKAHFSSKCRRCRLCDEGHGLEVEINCTRTQNTKCRC |
| | KPNFFCNSTVCEHCDPCTKCEHGIIKECTLTSNTKCKEEGSRSN |
| 232 | Tumor necrosis factor receptor superfamily member |
| | 6/FAS/CD95 (Transmembrane) |
| | LGWLCLLLLPIPLIVWV |
| 233 | Tumor necrosis factor receptor superfamily member |
| | 6/FAS/CD95 (Intracellular domain) |
| | KRKEVQKTCRKHRKENQGSHESPTLNPETVAINLSDVDLSKYITTIAGVMTL |
| | SQVKGFVRKNGVNEAKIDE I KNDNVQDTAEQKVQLLRNWHQLHGKKEAYDTL |
| | IKDLKKANLCTLAEKIQTIILKDITSDSENSNFRNEIQSLV |
| 234 | Tumor necrosis factor receptor superfamily member |
| | 6/FAS/CD95 Death Domain (230-314) |
| | SKYITTIAGVMTLSQVKGFVRKNGVNEAKIDE I KNDNVQDTAEQKVQLLRNW |
| | HQLHGKKEAYDTLIKDLKKANLCTLAEKIQTII |
| 235 | Tumor necrosis factor receptor superfamily member |
| | 6/FAS/CD95 Truncated (Signal Peptide-Mature Sequence) |
| | MLGIWTLLPLVLTSVARLSSKSVNAQVTDINSKGLELRKTVTTVETQNLEGL |
| | HHDGQFCHKPCPPGERKARDCTVNGDEPDCVPCQEGKEYTDKAHFSSKCRRC |
| | RLCDEGHGLEVEINCTRTQNTKCRCKPNFFCNSTVCEHCDPCTKCEHGIIKE |
| | CTLTSNTKCKEEGSRSNLGWLCLLLLPIPLIVWVKRKEVQK |
| 236 | Tumor necrosis factor receptor superfamily member |
| | 6/FAS/CD95 Truncated (Mature Sequence) |
| | QVTDINSKGLELRKTVTTVETQNLEGLHHDGQFCHKPCPPGERKARDCTVNG |
| | DEPDCVPCQEGKEYTDKAHFSSKCRRCRLCDEGHGLEVEINCTRTQNTKCRC |
| | KPNFFCNSTVCEHCDPCTKCEHGIIKECTLTSNTKCKEEGSRSNLGWLCLLL |
| | LPIPLIVWVKRKEVQK |
| 237 | Tumor necrosis factor receptor superfamily member |
| | 6/FAS/CD95 Deleted Death Domain (230-314) |
| | (Signal Peptide-Mature Sequence) |
| | MLGIWTLLPLVLTSVARLSSKSVNAQVTDINSKGLELRKTVTTVETQNLEGL |
| | HHDGQFCHKPCPPGERKARDCTVNGDEPDCVPCQEGKEYTDKAHFSSKCRRC |
| | RLCDEGHGLEVEINCTRTQNTKCRCKPNFFCNSTVCEHCDPCTKCEHGIIKE |
| | CTLTSNTKCKEEGSRSNLGWLCLLLLPIPLIVWVKRKEVQKTCRKHRKENQG |
| | SHESPTLNPETVAINLSDVDLLKDITSDSENSNFRNEIQSLV |
| 238 | Tumor necrosis factor receptor superfamily member |
| | 6/FAS/CD95 (R250Q D260V) (Signal Peptide-Mature |
| | Sequence) |
| | MLGIWTLLPLVLTSVARLSSKSVNAQVTDINSKGLELRKTVTTVETQNLEGL |
| | HHDGQFCHKPCPPGERKARDCTVNGDEPDCVPCQEGKEYTDKAHFSSKCRRC |
| | RLCDEGHGLEVEINCTRTQNTKCRCKPNFFCNSTVCEHCDPCTKCEHGIIKE |
| | CTLTSNTKCKEEGSRSNLGWLCLLLLPIPLIVWVKRKEVQKTCRKHRKENQG |
| | SHESPTLNPETVAINLSDVDLSKYITTIAGVMTLSQVKGFVQKNGVNEAKIV |
| | EIKNDNVQDTAEQKVQLLRNWHQLHGKKEAYDTLIKDLKKANLCTLAEKIQT |
| | IILKDITSDSENSNFRNEIQSLV |

### Compositions Comprising Further Molecules

In some embodiments, a composition, a cell, one or more isolated nucleic acids, and one or more isolated oligopeptides, of the present disclosure further comprises one or more additional molecules. In some embodiments, the composition, the cell, isolated oligopeptides, and isolated nucleic acids further comprise a safety switch. In some embodiments, the safety switch comprises a truncated EGFR (“EGFRt”), an RQR8 protein, or an inducible Caspase-9.

In some embodiments, the safety switch comprises a signal peptide. In some embodiments, the signal peptide comprises a sequence selected from Table 12.

TABLE 12
Example Signal Peptide Sequences

| SEQ ID | |
|---|---|
| NO: | Sequence |
| 216 | Signal Peptide Derived from HLA (amino acid) |
| | MAVMAPRTLVLLLSGALALTQTWA |
| 217 | Signal Peptide Derived from HLA (nucleotide) |
| | ATGGCTGTGATGGCCCCTAGAACACTGGTGCTGCTGCTGTCTGGT |
| | GCCCTGGCTCTGACTCAGACATGGGCC |
| 218 | Signal Peptide Derived from huGMCSF (amino acid) |
| | MLLLVTSLLLCELPHPAFLLIP |
| 219 | Signal Peptide Derived from huGMCSF (nucleotide) |
| | ATGCTGCTGCTGGTTACATCTCTGCTGCTGTGCGAGCTGCCCCATC |
| | CTGCCTTTCTGCTGATCCCC |
| 220 | Signal Peptide Derived from CD8a (amino acid) |
| | MALPVTALLLPLALLLHAARP |
| 221 | Signal Peptide Derived from CD8a (nucleotide) |
| | ATGGCTCTGCCTGTGACAGCTCTGCTGCTGCCTCTGGCTCTGCTTC |
| | TGCATGCCGCTAGACCT |
| 222 | Signal Peptide Derived from PD-1 (amino acid) |
| | MQIPQAPWPVVWAVLQLGWRPGW |
| 223 | Signal Peptide Derived from PD-1 (nucleotide) |
| | ATGCAGATTCCTCAAGCTCCTTGGCCTGTCGTGTGGGCCGTTCTGC |
| | AACTTGGATGGCGGCCTGGCTGG |
| 224 | Signal Peptide Derived from FAS (amino acid) |
| | MLGIWTLLPLVLTSVARLSSKSVNA |
| 225 | Signal Peptide Derived from FAS (nucleotide) |
| | ATGTTGGGCATATGGACCCTACTACCCTTGGTCCTGACTTCGGTGGC |
| | GCGGCTATCGAGCAAATCTGTCAACGCG |

In some embodiments, the EGFRt safety switch comprises an amino acid sequence of SEQ ID NO:205.

(SEQ ID NO: 205)
MLLLVTSLLLCELPHPAFLLIPRKVCNGIGIGEFKDSLSINATNIKHFKN
CTSISGDLHILPVAFRGDSFTHTPPLDPQELDILKTVKEITGELLIQAWP
ENRTDLHAFENLEIIRGRTKQHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFM

In some embodiments, the nucleic acid encoding SEQ ID NO:205 comprises SEQ ID NO: 270.

(SEQ ID NO: 270)
ATGCTGCTGCTGGTTACATCTCTGCTGCTGTGCGAGCTGCCCCATCCTGC
CTTTCTGCTGATCCCCAGAAAAGTGTGCAACGGCATCGGCATCGGAGAGT
TCAAGGACAGCCTGAGCATCAACGCCACCAACATCAAGCACTTCAAGAAC
TGCACCAGCATCAGCGGCGACCTGCACATTCTGCCTGTGGCCTTTAGAGG
CGACAGCTTCACCCACACACCTCCACTGGACCCTCAAGAGCTGGACATCC
TGAAAACCGTGAAAGAGATCACCGGATTTCTGTTGATCCAGGCTTGGCCC
GAGAACCGGACAGATCTGCACGCCTTCGAGAACCTGGAAATCATCAGAGG
CCGGACCAAGCAGCACGGCCAGTTTTCTCTGGCTGTGGTGTCCCTGAACA
TCACCAGCCTGGGCCTGAGAAGCCTGAAAGAAATCAGCGACGGCGACGTG
ATCATCTCCGGCAACAAGAACCTGTGCTACGCCAACACCATCAACTGGAA
GAAGCTGTTCGGCACCAGCGGCCAGAAAACAAAGATCATCAGCAACCGGG
GCGAGAACAGCTGCAAGGCTACAGGCCAAGTGTGCCACGCTCTGTGTAGC
CCTGAAGGCTGTTGGGGACCCGAGCCTAGAGATTGCGTGTCCTGTCGGAA
TGTGTCCCGGGGCAGAGAATGCGTGGACAAGTGCAATCTGCTGGAAGGCG
AGCCCCGCGAGTTCGTGGAAAACAGCGAGTGCATCCAGTGTCACCCCGAG
TGTCTGCCCCAGGCCATGAACATTACCTGTACCGGCAGAGGCCCCGACAA
CTGTATTCAGTGCGCCCACTACATCGACGGCCCTCACTGCGTGAAAACAT
GTCCTGCTGGCGTGATGGGAGAGAACAACACCCTCGTGTGGAAGTATGCC
GACGCCGGACATGTGTGCCACCTGTGTCACCCTAATTGCACCTACGGCTG
TACAGGCCCTGGCCTGGAAGGCTGTCCAACAAACGGACCTAAGATCCCCT
CTATCGCCACCGGCATGGTTGGAGCCCTGCTGCTTCTGCTGGTGGTGGCC
CTTGGAATCGGCCTGTTCATG

In some embodiments, the EGFRt safety switch comprises an amino acid sequence of SEQ ID NO:206.

(SEQ ID NO: 206)
RKVCNGIGIGEFKDSLSINATNIKHFKNCTSISGDLHILPVAFRGDSFTH
TPPLDPQELDILKTVKEITGELLIQAWPENRTDLHAFENLEIIRGRTKQH
GQFSLAVVSLNITSLGLRSLKEISDGDVIISGNKNLCYANTINWKKLFGT
SGQKTKIISNRGENSCKATGQVCHALCSPEGCWGPEPRDCVSCRNVSRGR
ECVDKCNLLEGEPREFVENSECIQCHPECLPQAMNITCTGRGPDNCIQCA
HYIDGPHCVKTCPAGVMGENNTLVWKYADAGHVCHLCHPNCTYGCTGPGL
EGCPTNGPKIPSIATGMVGALLLLLVVALGIGLEM

In some embodiments, the nucleic acid encoding SEQ ID NO:206 comprises SEQ ID NO: 207.

(SEQ ID NO: 207)
AGAAAAGTGTGCAACGGCATCGGCATCGGAGAGTTCAAGGACAGCCTGAG
CATCAACGCCACCAACATCAAGCACTTCAAGAACTGCACCAGCATCAGCG
GCGACCTGCACATTCTGCCTGTGGCCTTTAGAGGCGACAGCTTCACCCAC
ACACCTCCACTGGACCCTCAAGAGCTGGACATCCTGAAAACCGTGAAAGA
GATCACCGGATTTCTGTTGATCCAGGCTTGGCCCGAGAACCGGACAGATC
TGCACGCCTTCGAGAACCTGGAAATCATCAGAGGCCGGACCAAGCAGCAC
GGCCAGTTTTCTCTGGCTGTGGTGTCCCTGAACATCACCAGCCTGGGCCT
GAGAAGCCTGAAAGAAATCAGCGACGGCGACGTGATCATCTCCGGCAACA
AGAACCTGTGCTACGCCAACACCATCAACTGGAAGAAGCTGTTCGGCACC
AGCGGCCAGAAAACAAAGATCATCAGCAACCGGGGCGAGAACAGCTGCAA
GGCTACAGGCCAAGTGTGCCACGCTCTGTGTAGCCCTGAAGGCTGTTGGG
GACCCGAGCCTAGAGATTGCGTGTCCTGTCGGAATGTGTCCCGGGGCAGA
GAATGCGTGGACAAGTGCAATCTGCTGGAAGGCGAGCCCCGCGAGTTCGT
GGAAAACAGCGAGTGCATCCAGTGTCACCCCGAGTGTCTGCCCCAGGCCA
TGAACATTACCTGTACCGGCAGAGGCCCCGACAACTGTATTCAGTGCGCC
CACTACATCGACGGCCCTCACTGCGTGAAAACATGTCCTGCTGGCGTGAT
GGGAGAGAACAACACCCTCGTGTGGAAGTATGCCGACGCCGGACATGTGT
GCCACCTGTGTCACCCTAATTGCACCTACGGCTGTACAGGCCCTGGCCTG
GAAGGCTGTCCAACAAACGGACCTAAGATCCCCTCTATCGCCACCGGCAT
GGTTGGAGCCCTGCTGCTTCTGCTGGTGGTGGCCCTTGGAATCGGCCTGT
TCATG

In some embodiments, the cell disclosed herein further comprises a sequence encoding an artificial antigen receptor, a therapeutic polypeptide, an immune cell modulatory protein, or a combination thereof. In some embodiments, the artificial antigen receptor comprises a chimeric antigen receptor (CAR). In some embodiments, the artificial antigen receptor comprises a recombinant T cell receptor (rTCR). In some embodiments, the artificial antigen receptor comprises an enhanced affinity TCR. In some embodiments, the artificial antigen receptor binds to a tumor associated antigen (TAA), a pathogen associated protein, or an antigen associated with the disease or disorder is a cancer, an autoimmune disease or disorder, an infectious disease, an inflammatory disease, a renal disease or disorder, a lung disease or disorder, a liver disease or disorder a neurodegenerative disorder or disorder, or a metabolic disorder or disorder.

In some embodiments, the artificial antigen receptor binds to a TAA associated with a solid tumor or a hematologic cancer. In some embodiments, artificial antigen receptor binds to a TAA associated with a cancer selected from any one of leukemia, acute leukemia, acute lymphoblastic leukemia (ALL), acute lymphocytic leukemia, B cell, T cell or FAB ALL, acute myeloid leukemia (AML), acute myelogenous leukemia, chronic myelocytic leukemia (CML), chronic lymphocytic leukemia (CLL), hairy cell leukemia, myelodysplastic syndrome (MDS), a lymphoma, Hodgkin's disease, a malignant lymphoma, non-Hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, bladder cancer, breast cancer, colorectal cancer, endometrial cancer, head cancer, neck cancer, hereditary nonpolyposis cancer, Hodgkin's lymphoma, liver cancer, lung cancer, non-small cell lung cancer, ovarian cancer, pancreatic cancer, prostate cancer, renal cell carcinoma, testicular cancer, adenocarcinomas, sarcomas, malignant melanoma, and hemangioma.

In some embodiments, the artificial antigen receptor binds to a TAA selected from kallikrein 4, papillomavirus binding factor (PBF), preferentially expressed antigen of melanoma (PRAME), Wilms' tumor-I (WTI), Hydroxysteroid Dehydrogenase Like I (HSDLI), mesothelin, cancer testis antigen (NY-ES0-1), carcinoembryonic antigen (CEA), p53, human epidermal growth factor receptor 2/neuro receptor tyrosine kinase (Her2/Neu), carcinoma-associated epithelial cell adhesion molecule (EpCAM), ovarian and uterine carcinoma antigen (CAI25), folate receptor a, sperm protein 17, tumor-associated differentially expressed gene-12 (TADG-12), mucin-16 (MUC-16), LI cell adhesion molecule (LICAM), mannan-MUC-1, Human endogenous retrovirus K (HERV-K-MEL), Kita-kyushu lung cancer antigen-I (KK-LC-1), human cancer/testis antigen (KM-HN-1), cancer testis antigen (LAGE-I), melanoma antigen-A1 (MAGE-AI), Sperm surface zona pellucida binding protein (Spl 7), Synovial Sarcoma, X Breakpoint 4 (SSX-4), Transient axonal glycoprotein-1 (TAG-I), Transient axonal glycoprotein-2 (TAG-2), Enabled Homolog (ENAH), mammoglobin-A, NY-BR-I, Breast Cancer Antigen, (BAGE-1), B melanoma antigen, melanoma antigen-A1 (MAGE-A1), melanoma antigen-A2 (MAGE-A2), mucin k, synovial sarcoma, X breakpoint 2 (SSX-2), Taxol-resistance-associated gene-3 (TRAG-3), Avian Myelocytomatosis Viral Oncogene (c-myc), cyclin B 1, mucin I (MUC I), p62, survivin, lymphocyte common antigen (CD45), DickkopfWNT Signaling Pathway Inhibitor I (DKKI), telomerase, Kirsten rat sarcoma viral oncogene homolog (K-ras), G250, intestinal carboxyl esterase, alpha-fetoprotein, Macrophage Colony-Stimulating Factor (M-CSF), Prostate-specific membrane antigen (PSMA), caspase 5 (CASP-5), Cytochrome C Oxidase Assembly Factor I Homolog (COA-1), O-linked β-N-acetylglucosamine transferase (OGT), Osteosarcoma Amplified 9, Endoplasmic Reticulum Lectin (OS-9), Transforming Growth Factor Beta Receptor 2 (TGF-betaRII), murine leukemia glycoprotein 70 (gp70), Calcitonin Related Polypeptide Alpha (CALCA), Programmed cell death 1 ligand 1 (CD274), Mouse Double Minute 2Homolog (mdm-2), alpha-actinin-4, elongation factor 2, Malic Enzyme 1 (MEI), Nuclear Transcription Factor Y Subunit C (NFYC), G Antigen 1,3 (GAGE-1,3), melanoma antigen-A6 (MAGE-A6), cancer testis antigen XAGE-lb, six transmembrane epithelial antigen of the prostate 1 (STEAPI), PAP, prostate specific antigen (PSA), Fibroblast Growth Factor 5 (FGF5), heat shock protein hsp70-2, melanoma antigen-A9 (MAGE-A9), Arg-specific ADP-ribosyltransferase family C (ARTCI), B-Raf Proto-Oncogene (B-RAF), Serine/Threonine Kinase, beta-catenin, Cell Division Cycle 27 homolog (Cdc27), cyclin dependent kinase 4 (CDK4), cyclin dependent kinase 12 (CDK12), Cyclin Dependent Kinase Inhibitor 2A (CDKN2A), Casein Kinase 1 Alpha 1 (CSNKIAI), Fibronectin 1 (FN1), Gruwih Anest Specific 7 (GAS7), Glycoprotein nonmetastatic melanoma protein B (GPNMB), HAUS Augmin Like Complex Subunit 3 (HAUS3), LDLR-fucosyltransferase, Melanoma Antigen Recognized By T cells 2 (MART2), myostatin (MSTN), Melanoma Associated Antigen (Mutated) 1 (MUM-1-2-3), Poly (A) polymerase gamma (neo-PAP), myosin class I, Protein phosphatase 1 regulatory subunit 3B (PPP1R3B), Peroxiredoxin-5 (PRDX5), Receptor-type tyrosine-protein phosphatase kappa (PTPRK), Transforming protein N-Ras (N-ras), retinoblastoma-associated factor 600 (RBAF600), sirtuin-2 (SIRT2), SNRPDI, triosephosphate isomerase, Ocular Albinism Type 1 Protein (OAI), member RAS oncogene family (RAB38), Tyrosinase related protein 1-2 (TRP-1-2), Melanoma Antigen Gp75 (gp75), tyrosinase, Melan-A (MART-1), Glycoprotein 100 melanoma antigen (gplOO), N-acetylglucosaminyltransferase V gene (GnTVf), Lymphocyte Antigen 6 Complex Locus K (LY6K), melanoma antigen-AlO (MAGE-AlO), melanoma antigen-A12 (MAGE-A12), melanoma antigen-C2 (MAGE-C2), melanoma antigen NA88-A, Taxol-resistant-associated protein 3 (TRAG-3), BDZ binding kinase (pbk), caspase 8 (CASP-8), sarcoma antigen 1 (SAGE), Breakpoint Cluster Region-Abelson oncogene (BCR-ABL), fusion protein in leukemia, dek-can, Elongation Factor Tu GTP Binding Domain Containing 2 (EFTUD2), ETS Variant gene 6/acute myeloid leukemia fusion protein (ETV6-AML1), FMS-like tyrosine kinase-3 internal tandem duplications (FLT3-ITD), cyclin-A1, Fibronectin Type III Domain Containing 3B (FDNC3B) promyelocytic leukemia/retinoic acid receptor alpha fusion protein (pml-RARalpha), melanoma antigen-Cl (MAGE-Cl), membrane protein alternative spliced isoform (D393-CD20), melanoma antigen-A4 (MAGE-A4), and melanoma antigen-A3 (MAGE-A3).

In some embodiments, the artificial antigen receptor binds to an antigen associated with an autoimmune condition or disorder selected from any one of Type 1 Diabetes, rheumatoid arthritis (RA), systemic lupus erythematosis (SLE), or multiple sclerosis (MS). In some embodiments, the artificial antigen receptor binds to an antigen associated with an autoimmune condition or disorder selected from any one of Carboxypeptidase H, Chromogranin A, Glutamate decarboxylase, Imogen-38, Insulin, Insulinoma antigen-2 and 2B, Islet-specific glucose-6-phosphatase catalytic subunit related protein (IGRP), Proinsulin, α-enolase, Aquaporin-4, β-arrestin, Myelin basic protein, Myelin oligodendrocytic glycoprotein, Proteolipid protein, S100-β, Citrullinated protein, Collagen II, Heat shock proteins, Human cartilage glycoprotein, Double-stranded DNA, La antigen, Nucleosomal histones and ribonucleoproteins (snRNP), Phospholipid-β-2 glycoprotein I complex, Poly (ADP-ribose) polymerase, Sm antigens of U-1 small ribonucleoprotein complex.

In some embodiments, the artificial antigen receptor binds to a pathogen associated antigen from a bacterial, a fungal or a parasitic protein or fragment thereof. In some embodiments, the artificial antigen receptor binds to an antigen associated with HIV infection, human Cytomegalovirus infection, Hepatitis B infection, Hepatitis C infection, Ebola virus infection, Dengue, Yellow fever, Listeriosis, Tuberculosis, Cholera, Malaria, Leishmaniasis, or Trypanosoma infection, or a combination thereof.

In some embodiments, the artificial antigen receptor binds to an antigen associated with a neurodegenerative disorder or condition selected from Alzheimer's disease (AD) and other dementias, Parkinson's disease (PD) and PD-related disorders, Prion disease, Motor neurone diseases (MND), Huntington's disease (HD), Spinocerebellar ataxia (SCA) or Spinal muscular atrophy (SMA). In some embodiments, the antigen associated with the neurodegenerative disorder or condition is any one of Amyloid β (AB), tau, alpha-synuclein (α-syn), mHTT or prion PrPsc or a combination thereof.

In some embodiments, the therapeutic polypeptide is a cytokine, a cytokine receptor, a chemokine, a chemokine receptor, an immunogenic polypeptide, or a cell surface protein that binds to a target on the surface of another cell. In some embodiments, the immune cell modulatory protein is a cytokine, a chemokine, a transcription factor, a protein kinase, a protease, a component or an adaptor protein of a cell signaling pathway.

### Methods of Making

In some embodiments, the cell disclosed herein expresses the RTCR and FASDD disclosed herein. In some embodiments, the cell disclosed herein expresses the RTCR disclosed herein stably or transiently. In some embodiments, the cell disclosed herein expresses the RTCR disclosed herein stably. In some embodiments, the cell disclosed herein expresses the RTCR disclosed herein transiently. In some embodiments, the cell disclosed herein expresses the FASS disclosed herein stably or transiently. In some embodiments, the cell disclosed herein expresses the FASDD disclosed herein stably. In some embodiments, the cell disclosed herein expresses the FASDD disclosed herein transiently.

In some embodiments, the cell disclosed herein further expresses a safety switch, e.g., a truncated EGFR.

In some embodiments, the cell disclosed herein co-expresses at least one of the endogenous co-stimulatory molecules CD28, CD2, OX-40, ICOS, CD28, CD4, CD8 and CD40L or a combination thereof.

The present disclosure also provides a modified T lymphocyte (T cell), comprising: (a) a modification of an endogenous sequence encoding a T cell Receptor (TCR), wherein the modification reduces or eliminates a level of expression or activity of the TCR; and (b) a recombinant T cell co-stimulatory receptor (RTCR) disclosed herein. In some embodiments, the modification of an endogenous sequence encoding a T cell Receptor (TCR) is done using a nucleic acid modifying system. In some embodiments, the nucleic acid modifying system is one or more of a CRISPR/Cas protein, a Transcription Activator-Like Effector Nuclease (TALEN), a Zinc Finger Nuclease (ZFN), and an endonuclease. In some embodiments, the modification of an endogenous sequence encoding a T cell Receptor (TCR) is done by nonhomologous end joining repair. In some embodiments, the nonhomologous end joining repair is generated by zinc finger nuclease, introduced into the cell by physical means, viral vector, or non-viral vector. In some embodiments, the nonhomologous end joining repair is generated by TALE nuclease, introduced into the cell by physical means, viral vector, or non-viral vector. In some embodiments, the modification of an endogenous sequence encoding a T cell Receptor (TCR) reduces or eliminates a level of expression of the alpha chain of the TCR. In some embodiments, the modification of an endogenous sequence encoding a T cell Receptor (TCR) reduces or eliminates a level of expression of beta chain of the TCR. In some embodiments, the modification of an endogenous sequence encoding a T cell Receptor (TCR) reduces or eliminates a level of expression of both the alpha chain and the beta chain TCR alpha chain.

A skilled person in the art would appreciate that during production and/or storage of proteins, N-terminal glutamate (E) or glutamine (Q) can be cyclized to form a lactam (e.g., spontaneously or catalyzed by an enzyme present during production and/or storage). Accordingly, in some embodiments where the N-terminal residue of an amino acid sequence of a polypeptide is E or Q, a corresponding amino acid sequence with the E or Q replaced with pyroglutamate is also contemplated herein.

A skilled person in the art would also appreciate that during protein production and/or storage, the C-terminal lysine (K) of a protein can be removed (e.g., spontaneously or catalyzed by an enzyme present during production and/or storage). Such removal of K is often observed with proteins that comprise an Fc domain at its C-terminus. Accordingly, in some embodiments where the C-terminal residue of an amino acid sequence of a polypeptide is K, a corresponding amino acid sequence with the K removed is also contemplated herein.

In some embodiments, the modified T cell disclosed herein co-expresses at least one of the endogenous co-stimulatory molecules CD28, CD2, OX-40, ICOS, CD28, CD4, CD8 and CD40L or a combination thereof.

In some embodiments, the method disclosed herein further comprises a modification of an endogenous sequence encoding a component of major histocompatibility complex (MHC) class I (MHC-I), wherein the modification reduces or eliminates a level of expression or activity of the MHC-I. In some embodiments, the modification reduces or eliminates the expression or activity of β2-macroglobulin.

The present disclosure also provides a composition comprising the RTCR and FASDD disclosed herein. The present disclosure also provides a composition comprising the nucleic acid encoding the RTCR and FASDD disclosed herein. The present disclosure also provides a composition comprising the vector comprising the nucleic acid disclosed herein. The present disclosure also provides a composition comprising the cell disclosed herein. The present disclosure also provides a composition comprising the modified T cell disclosed herein.

In some embodiments, the vector comprises a truncated MSCV promoter, e.g., comprising the nucleic acid sequence of SEQ ID NO:208.

(SEQ ID NO: 208)
GGAATGAAAGACCCCACCTGTAGGTTTGGCAAGCTAGCTTAAGTAACGCC
ATTTTGCAAGGCATGGAAAATACATAACTGAGAATAGAGAAGTTCAGATC
AAGGTTAGGAACAGAGAGACAGCAGAATATGGGCCAAACAGGATATCTGT
GGTAAGCAGTTCCTGCCCCGGCTCAGGGCCAAGAACAGATGGTCCCCAGA
TGCGGTCCCGCCCTCAGCAGTTTCTAGAGAACCATCAGATGTTTCCAGGG
TGCCCCAAGGACCTGAAATGACCCTGTGCCTTATTTGAACTAACCAATCA
GTTCGCTTCTCGCTTCTGTTCGCGCGCTTCTGCTCCCCGAGCTCAATAAA
AGAGCCCACAACCCCTCACTCGGCGCGCCAGTCC

In some embodiments, the vector comprises a MND promoter, e.g., comprising the nucleic acid sequence of SEQ ID NO:209.

(SEQ ID NO: 209)
GGTTAGGAACAGAGAGACAGCAGAATATGGGCCAAACAGGATATCTGTGG
TAAGCAGTTCCTGCCCCGGCTCAGGGCCAAGAACAGTTGGAACAGCAGAA
TATGGGCCAAACAGGATATCTGTGGTAAGCAGTTCCTGCCCCGGCTCAGG
GCCAAGAACAGATGGTCCCCAGATGCGGTCCCGCCCTCAGCAGTTTCTAG
AGAACCATCAGATGTTTCCAGGGTGCCCCAAGGACCTGAAATGACCCTGT
GCCTTATTTGAACTAACCAATCAGTTCGCTTCTCGCTTCTGTTCGCGCGC
TTCTGCTCCCCGAGCTCAATAAAAG

In some embodiments, the vector comprises a WPRE regulatory element, e.g., comprising the nucleic acid sequence of SEQ ID NO:210.

(SEQ ID NO: 210)
CGATAATCAACCTCTGGATTACAAAATTTGTGAAAGATTGACTGGTATTC
TTAACTATGTTGCTCCTTTTACGCTATGTGGATACGCTGCTTTAATGCCT
TTGTATCATGCTATTGCTTCCCGTATGGCTTTCATTTTCTCCTCCTTGTA
TAAATCCTGGTTGCTGTCTCTTTATGAGGAGTTGTGGCCCGTTGTCAGGC
AACGTGGCGTGGTGTGCACTGTGTTTGCTGACGCAACCCCCACTGGTTGG
GGCATTGCCACCACCTGTCAGCTCCTTTCCGGGACTTTCGCTTTCCCCCT
CCCTATTGCCACGGCGGAACTCATCGCCGCCTGCCTTGCCCGCTGCTGGA
CAGGGGCTCGGCTGTTGGGCACTGACAATTCCGTGGTGTTGTCGGGGAAA
TCATCGTCCTTTCCTTGGCTGCTCGCCTGTGTTGCCACCTGGATTCTGCG
CGGGACGTCCTTCTGCTACGTCCCTTCGGCCCTCAATCCAGCGGACCTTC
CTTCCCGCGGCCTGCTGCCGGCTCTGCGGCCTCTTCCGCGTCTTCGCCTT
CGCCCTCAGACGAGTCGGATCTCCCTTTGGGCCGCCTCCCCGCATCGG

In some embodiments, the ICS transgene is cloned into a 3^{rd} generation lentiviral vector (e.g., pRRLSIN) under the control of an MSCV promoter and transduced into autologous T cells from leukapheresis or tumor infiltrating lymphocytes (TIL) of cancer patients. The transduced T cells (CD4+, CD8+) can then be ex vivo expanded into ICS T cells, e.g., of desired dose to re-infuse as a cancer immunotherapy.

In some embodiments, ICS T cells are ICS lentiviral gene-engineered polyclonal T cells made from circulating T cells isolated from PBMCs of cancer patients for autologous cell therapy. It is contemplated herein that, unlike the re-directed CAR T and TCR T cell therapy, the polyclonal endogenous TCRs are preserved in a prepared final product. After lentiviral vector transduction, the final composition can consist of, consist essentially of, or comprise a cryopreserved mixture of ICS-engineered polyclonal tumor targeting cells, unmodified polyclonal tumor targeting cells, ICS-engineered bystander cells, and unmodified bystander cells. In some embodiments, bystander cells will be limited by the positive selection of CD4/CD8 cells at the start of the manufacturing process.

In some embodiments, ICS TILs are ICS lentiviral gene-engineered polyclonal T cells made from Tumor Infiltrating Lymphocytes sourced from a patient's solid tumor following surgical resection and ex vivo expansion and transduction of the TIL population. The TIL population can be enriched for tumor reactive cells, i.e., more enriched compared to what is present in the circulation of a cancer patient.

In some embodiments, ICS Polyclonal TCR-T cells are ICS lentiviral gene-engineered poly-clonal T cells made from circulating T cells isolated from PBMCs of cancer patients for autologous cell therapy. In some embodiments, in addition to the ICS transgene cassette, these cells also receive exogenous TCRs (one or more than one TCR) derived from TILs of the same patient.

In some embodiments, ICS Autologous TCR-T (Antigen X) cells are ICS lentiviral gene-engineered poly-clonal T Cells made from the circulating T cells isolated from PBMCs of cancer patients for autologous cell therapy. In some embodiments, in addition to the ICS transgene cassette, these cells also receive exogenous a TCR directed against a common Tumor-Associated Antigen/HLA combination (e.g., NY-ESO-1 for HLA-A2).

In some embodiments, ICS Autologous CAR-T (Antigen X) cells are ICS lentivirally gene-engineered polyclonal T cells made from circulating T cells isolated from PBMCs of cancer patients, e.g., for autologous cell therapy. In some embodiments, in addition to the ICS Transgene cassette, these cells also receive a CAR construct re-directing T cell activity against a solid-tumor antigen target.

In some embodiments, Allogenic Personalized ICS TCR-T are cells from healthy donors that have been genetically modified (e.g., by TCR knockout) to prevent an allogenic T cell response, and that have been modified to express the ICS Transgene and TCRs derived from patient TIL populations.

In some embodiments, Allogenic Common ICS TCR-T are cells from healthy donors that have been genetically modified (e.g., by TCR knockout) to prevent an allogenic T cell response, and that have been modified to express the ICS Transgene and a TCR directed against a common tumor antigen/MHC combination.

In some embodiments, Allogenic CAR ICS-T are cells from healthy donors that have been genetically modified (e.g., by TCR knockout) to prevent an allogenic T cell response, and that have been modified to express the ICS Transgene and a CAR construct targeting T cells to tumor antigens.

The present disclosure also provides a composition comprising a population of cells, wherein the population comprises a plurality of the cell comprising the nucleic acid encoding or a vector comprising the nucleic acid encoding the RTCR and FASDD disclosed herein. The present disclosure also provides a composition comprising a population of cells, wherein the population comprises a plurality of the modified T cell disclosed herein.

The present disclosure also provides a method of producing a plurality of modified T cells, wherein the method comprises: a) providing a plurality of primary T cells disclosed herein; b) providing a composition comprising the RTCR and FASDD disclosed herein, the nucleic acid encoding the RTCR and FASDD disclosed herein, or the vector comprising the nucleic acid encoding the RTCR and FASDD disclosed herein; and c) introducing into the plurality of primary T cells of (a) the composition of (b), to produce a plurality of modified T cells under conditions that stably express the RTCR and FASDD within the plurality of modified T cells. In some embodiments, the method of producing a plurality of modified T cells disclosed herein, further comprises a step of modifying an endogenous sequence encoding an endogenous T cell Receptor (TCR), wherein the modification reduces or eliminates a level of expression or activity of the endogenous TCR. In some embodiments, the method of producing a plurality of modified T cells disclosed herein, further comprises a step of modifying an endogenous sequence, wherein the modification reduces or eliminates a level of expression or activity of a major histocompatibility complex (MHC) class I (MHC-I).

In some embodiments, the RTCR and FASDD are produced in a single polypeptide that is then processed into one or more discrete polypeptide, e.g., into a polypeptide comprising the RTCR and a polypeptide comprising the FASDD. In some embodiments, vector encodes a self-cleaving peptide, e.g., a ribosome skipping sequence, e.g., a P2A, T2A, E2A, F2A, or functional variant thereof. In some embodiments, a single polypeptide encodes a self-cleaving peptide, e.g., a ribosome skipping sequence, e.g., a P2A, T2A, E2A, F2A, or functional variant thereof, the RTCR, and the FASDD.

In some embodiments, the vector or single polypeptide further comprises a safety switch, e.g., truncated EGFR, thereby producing three discrete polypeptides.

In some embodiments, the P2A self-cleaving peptide comprises an amino acid sequence of SEQ ID NO:211. In some embodiments, SEQ ID NO:211 is encoded by a nucleic acid sequence comprising SEQ ID NO:212.

(SEQ ID NO: 211)
GSGATNFSLLKQAGDVEENPGP
(SEQ ID NO: 212)
GGATCCGGCGCCACCAATTTCAGCCTGCTGAAACAGGCTGGCGACGTGGA
AGAGAACCCTGGACCT

In some embodiments, the P2A self-cleaving peptide comprises an amino acid sequence of SEQ ID NO:213. In some embodiments, SEQ ID NO:213 is encoded by a nucleic acid sequence comprising SEQ ID NO:214.

(SEQ ID NO: 213)
GSGEGRGSLLTCGDVEENPGP
(SEQ ID NO: 214)
GGGTCTGGGGAAGGACGGGGTTCTCTTTTAACGTGTGGTGACGTGGAGGA
AAATCCGGGACCA

In some embodiments, the modifying an endogenous sequence encoding a T cell Receptor (TCR) is done using a nucleic acid modifying system. In some embodiments, the modifying an endogenous sequence that reduces or eliminates a level of expression or activity of is done using a nucleic acid modifying system. In some embodiments, the nucleic acid modifying system is a one or more of a CRISPR/Cas protein, a Transcription Activator-Like Effector Nuclease (TALEN), a Zinc Finger Nuclease (ZFN), and an endonuclease. In some embodiments, the modifying an endogenous sequence is done by nonhomologous end joining repair. In some embodiments, the nonhomologous end joining repair is generated by zinc finger nuclease, introduced into the cell by physical means, viral vector, or non-viral vector. In some embodiments, the nonhomologous end joining repair is generated by TALE nuclease, introduced into the cell by physical means, viral vector, or non-viral vector. In some embodiments, the modifying an endogenous sequence encoding a T cell Receptor (TCR) reduces or eliminates a level of expression of the alpha chain of the TCR. In some embodiments, the modifying an endogenous sequence encoding a T cell Receptor (TCR) reduces or eliminates a level of expression of beta chain of the TCR. In some embodiments, the modifying an endogenous sequence encoding a T cell Receptor (TCR) reduces or eliminates a level of expression of both the alpha chain and the beta chain TCR alpha chain.

In some embodiments, the modifying an endogenous sequence that reduces or eliminates a level of expression or activity of a major histocompatibility complex (MHC) class I (MHC-I), wherein the modifying of an endogenous sequence reduces or eliminates a level of expression or activity of the MHC-I. In some embodiments, the modifying of an endogenous sequence reduces or eliminates the expression or activity of β2-macroglobulin.

In some embodiments, the method of producing a plurality of modified T cells disclosed herein, further comprises d) maintaining or expanding the plurality of modified T cells in a suitable cell culture media; and e) either: i) cryopreserving the plurality of modified T cells in a suitable cell freezing media; or ii) preparing the plurality of modified T cells for administering to a subject suffering from a disease or disorder.

The compositions comprising the cells or modified T cells of the disclosure, and the plurality of modified T cells produced by the methods of the disclosure, intended for administration to a subject may be required to meet one or more “release criteria” that indicate that the composition is safe and efficacious for formulation as a pharmaceutical product and/or administration to a subject. Release criteria may include a requirement that a composition of the disclosure (e.g., a cell or modified T cell of the disclosure) comprises a particular percentage of cells or modified T cells expressing the RTCR of the disclosure on their cell surface. The expansion process should be continued until a specific criterion has been met (e.g., achieving a certain total number of cells or modified T cells of the disclosure or a certain percentage of total number of cells or modified T cells expressing the RTCR of the disclosure).

Certain criteria signal a point at which the expansion process should end. For example, cells should generally be formulated, reactivated, or cryopreserved once they reach a cell size of 300 fL (otherwise, cells reaching a size above this threshold may start to die). Cryopreservation immediately once a population of cells reaches an average cell size of less than 300 fL may yield better cell recovery upon thawing and culture because the cells have not yet reached a fully quiescent state prior to cryopreservation (a fully quiescent size is approximately 180 fL). Prior to expansion, T cells of the disclosure may have a cell size of about 180 fL, but may more than quadruple their cell size to approximately 900 fL at 3 days post-expansion. Over the next 6-12 days, the population of T cells will slowly decrease cell size to full quiescence at 180 fL.

A process for preparing a cell population for formulation may include, but is not limited to the steps of, concentrating the cells of the cell population, washing the cells, and/or further selection of the cells via drug resistance or magnetic bead sorting against a particular surface-expressed marker. A process for preparing a cell population for formulation may further include a sorting step to ensure the safety and purity of the final product. For example, if a tumor cell from a patient has been used to stimulate a modified T cell of the disclosure or that have been modified in order to stimulate a modified T cell of the disclosure that is being prepared for formulation, it is critical that no tumor cells from the patient are included in the final product.

In some embodiments, the cell disclosed herein, or the modified T cell disclosed herein, expresses on the cell surface the RTCR comprising a mutant CD137, a mutant CD134, a mutant CD40, or a mutant CD27 intracellular signaling domain disclosed herein, at a level that is at least about 2×, 3×, 4×, 5×, 6×, 7×, 8×, 9×, 10× or 20×, more as compared to the level of expression of a co-stimulatory molecule comprising a wild type CD137 or a wild type CD134 intracellular domain, respectively.

### Pharmaceutical Compositions or Formulation

In some embodiments, the compositions disclosed herein, and the population of modified T cells produced using the methods disclosed herein, is in the form of a pharmaceutical formulation (or composition). In some embodiments, the pharmaceutical formulation disclosed herein comprises a pharmaceutically acceptable carrier. A pharmaceutical formulation of the disclosure may be distributed into bags for infusion, cryopreservation, and/or storage.

A pharmaceutical formulation of the disclosure may be cryopreserved using a standard protocol and, optionally, an infusible cryopreservation medium. For example, a DMSO free cryopreservant (e.g., CryoSOfree™ DMSO-free Cryopreservation Medium) may be used to reduce freezing-related toxicity. A cryopreserved pharmaceutical formulation of the disclosure may be stored for infusion to a patient at a later date. An effective treatment may require multiple administrations of a pharmaceutical formulation of the disclosure and, therefore, pharmaceutical formulations may be packaged in pre-aliquoted “doses” that may be stored frozen but separated for thawing of individual doses.

A pharmaceutical formulation of the disclosure may be stored at room temperature. An effective treatment may require multiple administrations of a pharmaceutical formulation of the disclosure and, therefore, pharmaceutical formulations may be packaged in pre-aliquoted “doses” that may be stored together but separated for administration of individual doses.

A pharmaceutical formulation of the disclosure may be archived for subsequent re-expansion and/or selection for generation of additional doses to the same patient in the case of an allogenic therapy who may need an administration at a future date following, for example, a remission and relapse of a condition.

As noted above, the disclosure provides for stable formulations, which preferably comprise a phosphate buffer with saline or a chosen salt, as well as preserved solutions and formulations containing a preservative as well as multi-use preserved formulations suitable for pharmaceutical or veterinary use, comprising at least one modified cell in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, polymers, or mixtures thereof in an aqueous diluent. Any suitable concentration or mixture can be used as known in the art, such as about 0.0015%, or any range, value, or fraction therein. Non-limiting examples include, no preservative, about 0.1-2% m-cresol (e.g., 0.2, 0.3. 0.4, 0.5, 0.9, or 1.0%), about 0.1-3% benzyl alcohol (e.g., 0.5, 0.9, 1.1, 1.5, 1.9, 2.0, or 2.5%), about 0.001-0.5% thimerosal (e.g., 0.005, 0.01), about 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, or 1.0%), and the like.

As noted above, the disclosure provides an article of manufacture, comprising packaging material and at least one vial comprising a solution of at least one modified cell with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, or 72 hours or greater.

The articles of manufacture of the present disclosure are useful for administration over a period ranging from immediate to twenty-four hours or greater. Accordingly, the presently claimed articles of manufacture offer significant advantages to the patient. Formulations of the disclosure can optionally be safely stored at temperatures of from about 2° C. to about 40° C. and retain the biological activity of the protein for extended periods of time, thus allowing a package label indicating that the solution can be held and/or used over a period of 6, 12, 18, 24, 36, 48, 72, or 96 hours or greater.

The products of the present disclosure include packaging material. The packaging material provides, in addition to the information required by the regulatory agencies, the conditions under which the product can be used.

### Therapeutic Applications

In some aspects, the present disclosure also provides a method of treating a disease or disorder, comprising administering to a subject in need thereof a therapeutically effective number of the cell comprising the nucleic acid encoding or the vector comprising the nucleic acid encoding the RTCR and FASDD disclosed herein, a therapeutically effective number of any one of the modified T cell disclosed herein, a therapeutically effective amount of any one of the compositions disclosed herein, or a therapeutically effective number of the plurality of modified T cells produced by the method disclosed herein.

The methods of the present disclosure can improve a variety of clinical endpoints. For example, in some embodiments, the method increases overall survival in the subject relative to individuals not receiving treatment. In some embodiments, the method increases progression free survival in the subject relative to individuals not receiving treatment. In some embodiments, the method increases overall survival and progression free survival in the subject relative to individuals not receiving treatment.

In some embodiments, the subject is a mammal. In some embodiments, the mammal is any one of a human, a primate, a rodent, a canine, a feline, an ungulate, an equine and a porcine. In some embodiments, the mammal is a human. In some embodiments, the mammal is a non-human primate.

In some embodiments, the disease or disorder is any one of a cancer, an autoimmune disorder, an infectious disease, an inflammatory disease or condition, a renal disease or disorder, a lung disease or disorder, a liver disease or disorder, a cardiovascular system disease or disorder, a neurodegenerative disorder or condition, or a metabolic disorder or condition.

In some embodiments, the cancer is a solid tumor or a hematologic cancer.

In some embodiments, the infectious disease is caused by a bacterium, a virus, a fungi, a protozoa, or a parasite. The method can comprise administering an effective amount of the presently disclosed cells or a composition comprising thereof to a subject having a pathogen infection. Exemplary, non-limiting viral infections susceptible to treatment may include Cytomegalovirus (CMV), Epstein Barr Virus (EBV), Human Immunodeficiency Virus (HIV), and influenza virus infections.

In some embodiments, the neurodegenerative disorder or condition is any one of Alzheimer's disease (AD) and other dementias, Parkinson's disease (PD) and PD-related disorders, Prion disease, Motor neuron diseases (MND), Huntington's disease (HD), Spinocerebellar ataxia (SCA) or Spinal muscular atrophy (SMA).

### Kits

In some aspects, provided herein are kits for inducing and/or enhancing an immune response and/or treating and/or preventing a neoplasm or a pathogen infection in a subject. In some embodiments, the kit comprises an effective amount of presently disclosed cells or a pharmaceutical composition comprising thereof. In some embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding an antigen-recognizing receptor (e.g., a CAR or a TCR) directed toward an antigen of interest and an isolated nucleic acid molecule encoding a dominant negative Fas polypeptide in expressible form, which may optionally be comprised in the same or different vectors.

If desired, the cells and/or nucleic acid molecules are provided together with instructions for administering the cells or nucleic acid molecules to a subject having or at risk of developing a neoplasm or pathogen or immune disorder. The instructions generally include information about the use of the composition for the treatment and/or prevention of neoplasia or a pathogen infection. In some embodiments, the instructions include at least one of the following: description of the therapeutic agent; dosage schedule and administration for treatment or prevention of a neoplasia, a pathogen infection, or immune disorder or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

The following examples are provided to better illustrate the present disclosure and are not to be interpreted as limiting the scope of the disclosure. To the extent that specific materials are mentioned, it is merely for purposes of illustration and is not intended to limit the disclosure. One skilled in the art may develop equivalent means or reactants without the exercise of inventive capacity and without departing from the scope of the disclosure.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1—Materials and Methods

### Media

DMEM was supplemented with antibiotics (“Pen/Strep”), Glutamax, 10 mM HEPES, 10 μg/mL Gentamycin and 10% FBS to make complete DMEM.

RPMI was supplemented with Pen/Strep, Glutamax, 10 μg/mL Gentamycin, 10% FBS, to make complete RPMI.

T Cell growth media was made by supplementing CTS AIM-V media (Invitrogen) with 100 I.U./mL IL-2, IL-7, and IL-15 (Miltneyi) and 1% human serum (Gemini Bio). Cytokine was omitted from the media for T cell activation assays.

TIL Phase 1 (“pre-REP”) media was made by supplementing RPMI-1640 media with 10% heat-shocked human AB serum (Gemini Bio), Pen/Strep, Glutamax, 10 mM HEPES, 10 μg/mL Gentamicin, and 6000 I.U. IL-2 (gift from Amoytop Biotech). Media was prepared fresh on the day of use.

TIL Phase 2 (“REP”) was made by mixing TIL Phase 1 media in a 50/50 ratio with CTS-AIM V media.

### Cells and Cell Lines

Leukopacks were purchased from either Discovery Life Sciences or Stem Express (CGT global) and PBMCs were isolated by density gradient centrifugation over Ficoll-Paque Plus (GE Healthcare). Cells were frozen in 90% FBS/10% DMSO for viable cell storage and stored long-term in liquid nitrogen. Cells were thawed prior to use in experiments.

A549, K562, and A375 cells were purchased from ATCC and cultured in complete DMEM.

K562-GFP cells were prepared by transducing K562 parental cells with a lentivirus encoding GFP and Puromycin, followed by limiting dilution and clonal selection to create a stable cell line.

K562-GFP/PDL1 cells were created by dual transduction of a K562 parental cell line with a GFP/Puromycin encoding lentivirus and a PD-L1 encoding lentivirus. Limiting dilution was performed followed by screening of clones for GFP and PD-L1 expression. A single clone was selected for high GFP and PD-L1 expression.

K562-GFP/PDL1/FASL cells were created by transducing the K562-GFP/PDL1 clone with a lentivirus encoding FAS Ligand (FASL) followed by two rounds of cell sorting to enrich FAS-L expressing cells to >95% expressing.

A375-GFP cells were created by transducing A375 parental cells with a GFP expression construct to >98% efficiency and maintained as a polyclonal line.

A375-GFP/PDL1 cells were created by transducing A375 parental cells with a lentivirus encoding PDL1_P2A_huEGFRt under a PGK promoter and GFP under an MSCV promoter.

A549-HLA-A2 and A549-HLA-A2/PDL1 target cell lines were created by transducing parental cells with lentivirus encoding HLA-A2 w/wo an additional transduction containing a PDL1 and were subcloned by limiting dilution. Single cell clones were screened and selected based on HLA-A2 and PD-L1 expression.

### Peptides

NY-ESO-1 peptide (157-165, SLLMWITQV (SEQ ID NO:148)) was ordered from Anaspec.

### Plasmids and Cloning

A lentiviral plasmid containing the PGK promoter driving a truncated human EGFR receptor (huEGFRt) followed by the MSCV promoter driving GFP and a subsequent WPRE sequence was ordered from vector builder.

To generate PD-1/TCR vectors; PD-1CCR sequences followed by a P2A sequence were designed in SnapGene then sequence optimized and ordered as a single gene block from Invitrogen. Gene blocks placed in-frame with the huEGFRt sequence using NEB builder homology-based recombination. PD-1/FASDD constructs were ordered as a single gene block and cloned downstream of the PGK promoter following excision of the huEGFRt sequence by enzymatic digestion. TCR sequences were constructed from 2 or 3 gene block fragments (Invitrogen) and cloned with NEB builder downstream of the MSCV promoter following GFP excision.

To generate constructs driving PD-1 switch receptors alone, or in combination with a dominant negative FAS receptor, or huEGFRt (separated by P2A and T2A self-cleaving peptides) a single promoter construct was created with the MSCV promoter driving PD-1 CCR_P2A_huEGFRt or PD-1 CCR_P2A_FASDD_T2A_huEGFRt. All constructs were synthesized as 2 or 3 gene blocks and cloned using NEBuilder reactions.

To PD-L1_P2A and HLA-A2 were cloned in-frame with the huEGFRt and in place of GFP, respectively.

All plasmids were sequence confirmed by Sanger Sequencing (GeneWiz/Azenta).

### Lentiviral Production

VSVG pseudo-typed lentivirus was produced according to manufacturer's protocols for 293 FT cells (Invitrogen) and Lipofectamine 3000 (Invitrogen). In brief, 293 FT were seeded the night before or the day of at 0.9×10⁶ or 1.4×10⁶ cells/well, respectively. Once the cells had adhered and reached at least 80% confluency a mix of lentiviral plasmid, packaging vector (psPAX2) and VSVG envelope expressing plasmid (PMD2.G) were transfected using lipofectamine 3000 (Invitrogen), according to the manufacturer's protocol. After 18 hrs the media was replaced with 3 mLs of fresh DMEM. Viral supernatants were harvested 24, 48, and 72 hrs following changing the media and spun down at 1500 RPM to remove 293 FT cell/debris. Lentivirus was concentrated using Lenti-X Concentrator (Takara) and aliquoted/stored at −80C in single use aliquots.

### T Cell Culture, Transduction, and Isolation

Human T cells were isolated from frozen PBMCs using CD4/CD8 microbeads (Miltenyi) and activated in T cell growth media with T Cell TransAct (Miltenyi) in T Cell growth media, according to manufacturer's protocols. 24 hrs after activation, activated T cells were transferred to 24 well plates and lentivirus was added prior to a spinfection (800G, 2 hrs, 32 C) and continued culture. 72 hours after activation cells were expanded in T Cell Growth media and cultured for 8-20 days.

After day 5 in culture, cell transduction was determined by flow cytometry and transduced cells were enriched based on huEGFRt expression. To isolate cells based on EGFR expression, T cells cultures were collected and washed in MACS buffer. Cells were then stained in 1:100 anti-EGFR-APC antibody in MACS buffer at 4C for 30 minutes. Cells were then washed and incubated with anti-APC microbeads (Miltenyi) for 15-30 minutes at 4 C. Unbound microbeads were then removed by centrifugation and huEGFRt cells were isolated by positive selection on mini-macs columns. Cells were eluted from the mini-MACS columns and put back into culture in T cell growth media and used within 2 weeks for experiments. To create stable cell lines, cells were collected and transduced as with primary T cells. EGFR selection was performed twice, two weeks apart.

### TIL Culture and Transduction

Human tumor samples were procured through the cooperative human tissue network (CHTN) and digested with Collagenase and DNAse I (Invitrogen) with mechanical disruption using C-Tubes and a gentleMACS instrument (Miltenyi). Single cell digests were either cryopreserved in 10% DMSO or used fresh to initiate TIL culture.

Fresh or thawed disassociated tumor cells (DTCs) were cultured in Phase 1, pre-rapid expansion protocol (REP) media for 12 days in 24-well tissue-culture treated plates, with media exchanges and expansions as needed. Cells were transduced by adding lentiviral particles on day 1 and day 2 of Pre-REP culture. On day 12, cells were collected, counted, and transduction efficiency along with T cell composition was determined. To initiate the REP, 50,000 TILs were activated in a single well of a 24 well plate with 4×10⁶ irradiated feeder cells, 30 ng/mL OKT3 (Biolegend), and 2 mLs of REP media. Cells were expanded on day 5 or 6 of REP and expanded with freshly prepared REP media, as required. On day 12 of the REP cells were harvested and used to set up stimulation assays. No EGFR selection was done on transduced cells.

### Evaluation of T Cell and CCR/ICS Function

For K562 stimulation K562 cells, or PDL1/FAS expressing versions were collected and resuspended in CTS-AIMV media with 1% Human serum and aliquoted to flat-bottom plates (25,000 cells/well). T cells were collected, labeled with Tag-IT violet (Biolegend) and resuspended to 1×10⁶ cells/mL and 25,000 cells were added to each well. Anti-CD3 stimulatory antibodies (clone OKT3 or HIT3a, Biolegend) was added to the final indicated concentration and a total volume of 200 μLs. At 18 hrs post assay initiation 100 μL of culture supernatant was collected and frozen for cytokine analysis and replaced with 100 μL fresh media. If necessary, a 150 μL media exchange was done on day 3 post stimulation. Cells were harvested on day 4 to evaluate T cell expansion and target cell killing.

For allogenic A375 T cell reactivity assays, 10,000 A375 or A375-PDL1 cells were plated with the indicated number of T cells in 96-well U-bottom plates. Cells were briefly centrifuged (300×G, 2 minutes) and returned to culture for 120 hours. Media was exchanged with fresh media as required (day 4 or 5). On day 6 cells were collected and analyzed by flow cytometry.

To evaluate NY-ESO-1 TCR function, A549 or A375 cells were plated 1 day prior to the addition of T cells in DMEM in 96 well flat-bottom plates. The media was exchanged prior to the addition of cognate T cells and for A549 cells, NY-ESO-1 peptide was added as indicated for a 2 hr pre-incubation prior to media exchange and T cell addition. Following EGFR+ selection, T cells were collected, labeled with Tag-IT violet, counted, and resuspended at the appropriate concentration in media and distributed to APC-bearing wells. Supernatant was collected at 18 hr post stimulation to assess cytokine secretion and proliferation/T cell killing was assessed following 96 hrs of stimulation.

### Cytokine Multiplex Assay

Following collection of T cell supernatants cytokines were measured with the Legendplex Multi-Analyte Flow Assay Kit for human cytokines (Biolegend). Secreted cytokines were measured by flow cytometry and the values were normalized to the maximal response of the control group in order to combine and analyze multiple experiments and normalize for variability between experiments and donors. Alternatively, analyzed cytokines were normalized by standard curve and expressed as ng/ml.

### Flow Cytometry

Cells were collected and washed in MACS Buffer (PBS−/−, 1% FBS, 1 mM EDTA) before being stained in MACS buffer containing relevant antibodies. Anti-EGFR-APC, anti-mouse TCRbeta-FITC, anti-human PD-1-PE, anti-CD3 APC-Cy7, anti-CD8 PE-Cy7, FAS were all purchased from Biolegend. Following addition of antibodies, cells were stained for 30-60 minutes at 4° C. Cells were then washed 2× in MACS buffer and analyzed on an ACEA novocyte flow cytometer. Cells were collected at constant volume, allowing for accurate cell counts to be obtained.

### Example 2—Development of ICS Gene-Engineered T Cells

### Expression of Multi-Component Synthetic ICS Gene Under Strong MSCV Promoter

The ICS transgene containing multiple functional components is developed to modify T cells for adoptive cell therapy. Since PD-1 is the principal immune checkpoint receptor against which the most effective cancer immunotherapy has been developed, one of major goals of the ICS transgene aims to modify T cells to subvert negative signaling from PD-1 receptor, using cancer-associated PD-1 ligands to the advantage of the adoptively transferred cells. In T cells, the chimeric costimulatory receptor (CCR) expresses on cell surface and binds PD-L1, switching the antagonistic PD-1 signaling into an agonistic co-stimulatory signal, thereby increasing T cell effector functions. Additionally, FAS can limit the antigen-dependent survival and proliferation of T cells by inducing pro-apoptotic signaling following TCR stimulation. Overexpression of a FAS dominant negative receptor (FASDD) can protect cells from this pathway. The combined use of a PD-1 CCR and dominant negative FAS mutant should provide dual protection against two of the immunosuppressive mechanisms operating within the TME and provide synergistic benefit to the function of adoptively transferred engineered T cells. As both transgene constructs may increase T cell functionality and result in on or off-tumor toxicity a truncated human EGFR (huEGFRt) transgene is included as a safety switch alongside the PD-1 CCR and the FASDD.

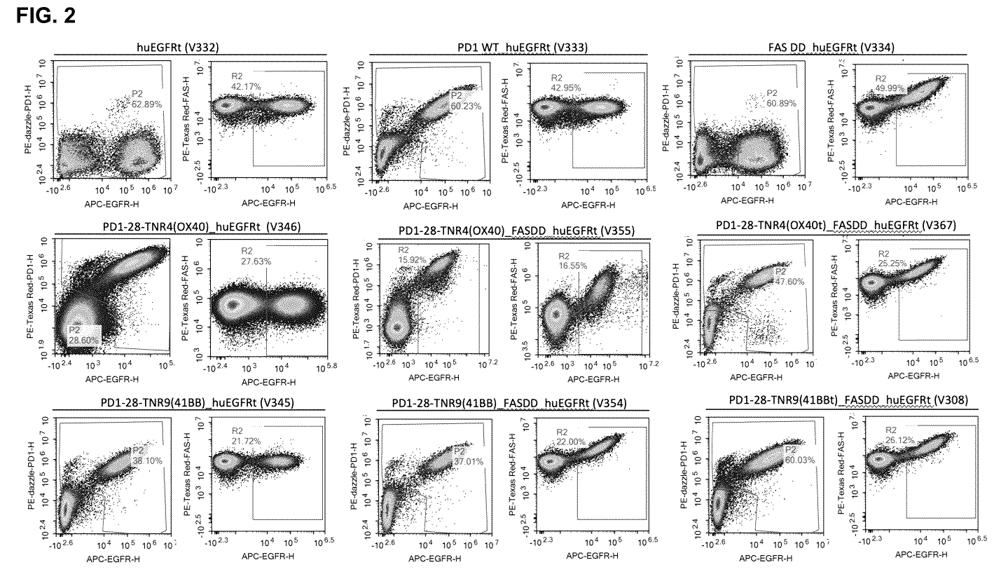

The ICS transgene containing the three components is expressed under regulation of an MSCV promoter in a lentiviral vector (FIG. 1A). The gene encodes one polypeptide that is processed by ribosomal skipping at P2A and T2A sites, resulting in three membrane proteins PD-1CCR, FASDD, and huEGFRt (FIG. 1B). The three proteins are concurrently and stably expressed in T cells (schematically depicted in FIG. 1C). When transduced into primary T cells, all three proteins are expressed in a concerted manner, as demonstrated by the ICS transgenes of PD1-28-TNR4 (OX40)_FASDD_huEGFRt and PD1-28TNR9 (41BB)_FASDD_huEGFRt (FIG. 2)

PD-1 Chimeric Proteins are TCR Co-Stimulatory Receptors (PD-1CCR) Activated by PD-L1 that Function with Variable Strength.

Other groups have identified a PD1-CD28 fusion protein to function as a chimeric switch receptor, but no concerted effort has been made to improve upon this construct, and it is unknown if the CD28-based 2^{nd} generation construct is sufficient to overcome inhibitory signaling within the TME. Therefore, a library of constructs was generated comprising various 2^{nd} generation (CD28 family or TNFRSF), and 3^{rd} generation (CD28 family linked to TNFRSF family) with or without FASDD and huEGFRt and transduced into primary T cells to evaluate functionality and in vitro tumor-killing efficacy in the presence of increasing PD-L1 levels.

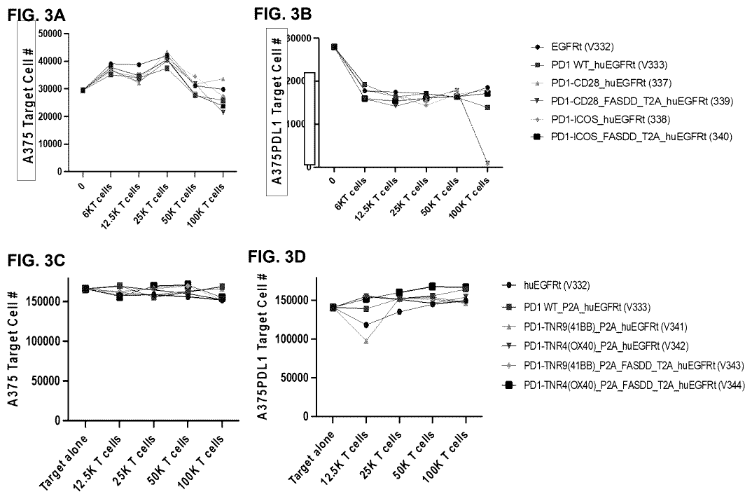

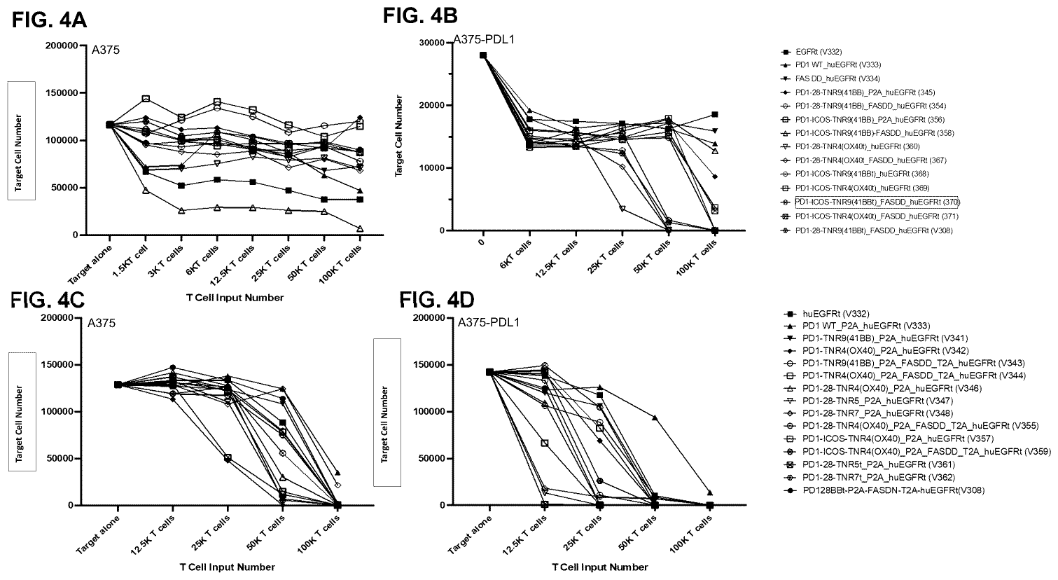

To first evaluate functionality, an HLA-mismatch reactivity assay was used in which transduced and selected T cells were incubated with A375 cells with or without engineered PD-L1 overexpression. After six days the number of T cells and target cells in culture were evaluated by flow cytometry. FIGS. 3A-3D and 4D demonstrate minimal target cell killing in all 2^{nd} generation constructs tested, except at the highest T cell:Target cell ratio for the PD1-CD28 construct, indicating that under these conditions, the 2^{nd} generation receptors confer minimal benefit to T cell functional avidity (FIGS. 3A-3D). This is in stark contrast to the dramatic increase in cytotoxicity of A375-PDL1 cells observed with T cells expressing various 3^{rd} generation constructs (FIGS. 4A-4D), though there was a range of efficacy observed for 3^{rd} generation constructs. In general, the 3^{rd} generation constructs incorporating a truncated TNFRSF9 (4-1BBt), a WT or truncated TNFRSF4 (CD134/OX-40), a WT or truncated TNFRSF5 (CD40), or a WT or Truncated TNFRSF7 (CD27) demonstrated significant ability to kill PD-L1 expressing A375 cells and the CD28-based constructs out-performed the ICOS-based constructs.

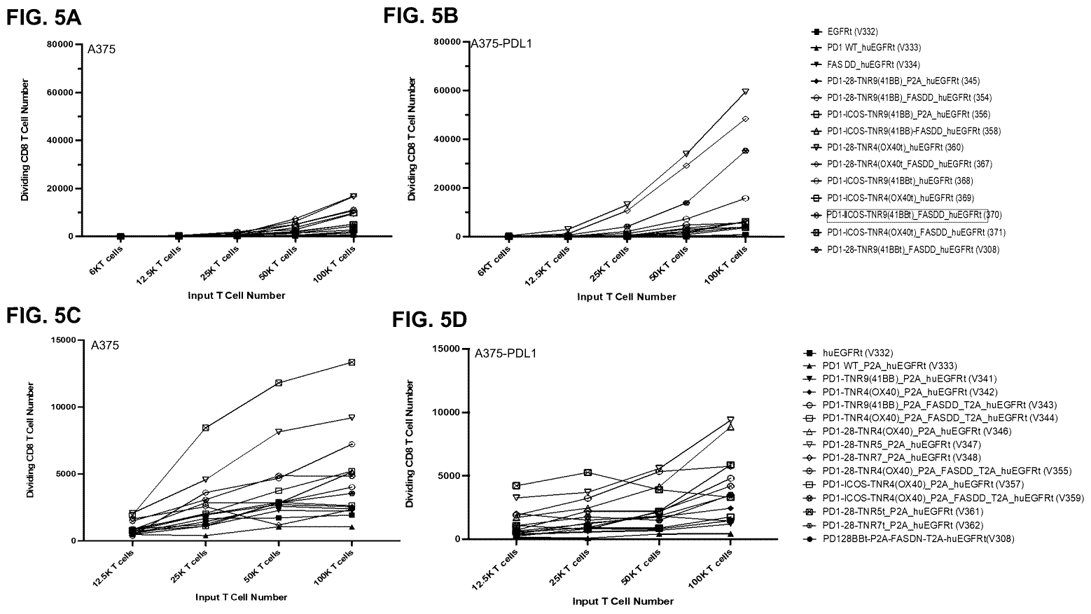

Alongside the target cell killing, number of CD8+ T cells in culture that had undergone at least one division was assessed (FIGS. 5A-5D). It was found that the PD-1 switch receptors could mediate increased T cell proliferation in the presence of PD-L1 expressing target cells. Again, the constructs incorporating a truncated TNFRSF9 (4-1BBt), a WT or truncated TNFRSF4 (CD134/OX-40), a WT or truncated TNFRSF5 (CD40), or a WT or truncated TNFRSF7 (CD27) demonstrated significant ability to proliferate in response to PDL1 expressing A375 cells and the CD28-based constructs generally out-performed the ICOS-based constructs. Surprisingly, the TNFRSF5-based PD1-CCRs proliferated in the absence of exogenous PD-L1, possibly suggesting the presence of tonic signaling in these constructs (FIG. 5C).

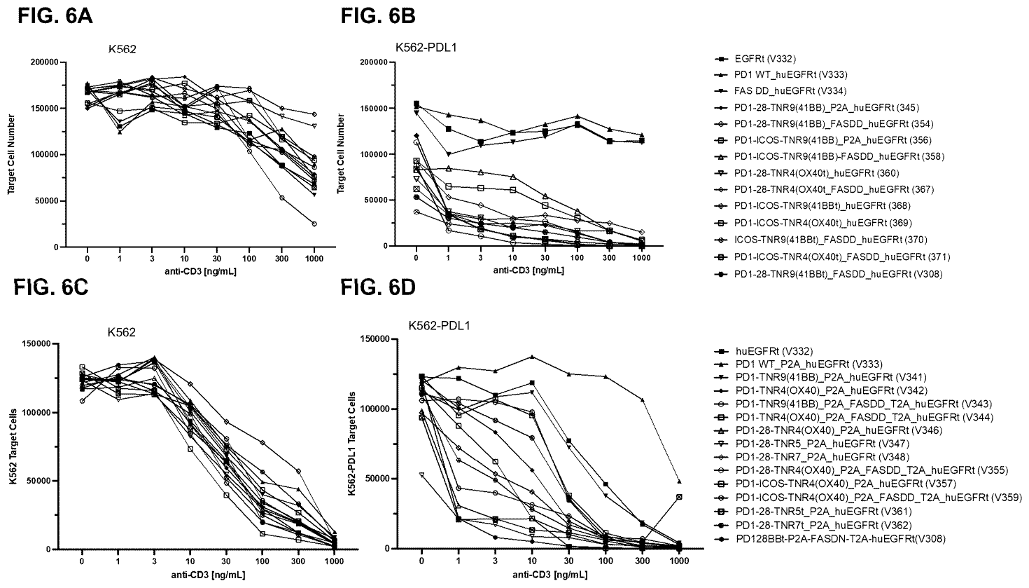

To determine if the PD-1CCR functioned as intended to turn the signal transmitted by PD-1 ligand into a co-stimulatory signal under conditions of varying TCR stimulation, a system utilizing the HLA-negative K562 cell line engineered to express PD-L1 and stimulated T cells transduced with various PD-1 constructs (Wild-Type PD-1 (WT), 2nd Generation PD-1-CD28, 3^{rd} Generation PD-1-28BBt, or ICS transgene cassette) with anti-CD3 in the presence of parental or modified K562 cells was used. Target cell killing, T cell proliferation, and T cell cytokine production were evaluated as functional readouts (FIGS. 6A-6D, 7A-7D, and 8A-8D).

FIGS. 6A-6D demonstrate that the various ICS transgenes or constructs containing the PD1CCR component have minimal effect on K562 cell killing in the absence of TCR stimulation and PD-L1 expression, and track with cells transduced with huEGFRt across multiple anti-CD3 concentrations (FIGS. 6A and 6C). However, the ICS transgenes mediated robust K562 cell cytotoxicity in the presence of PD-L1, even at low concentrations of anti-CD3. Though some constructs out-performed others, it was difficult to assign a particular order to construct functionality, though again the PD1-28-TNFRSF4/5/7/9t constructs were amongst the best performers in the presence of PD-L1 (FIGS. 6B and 6D).

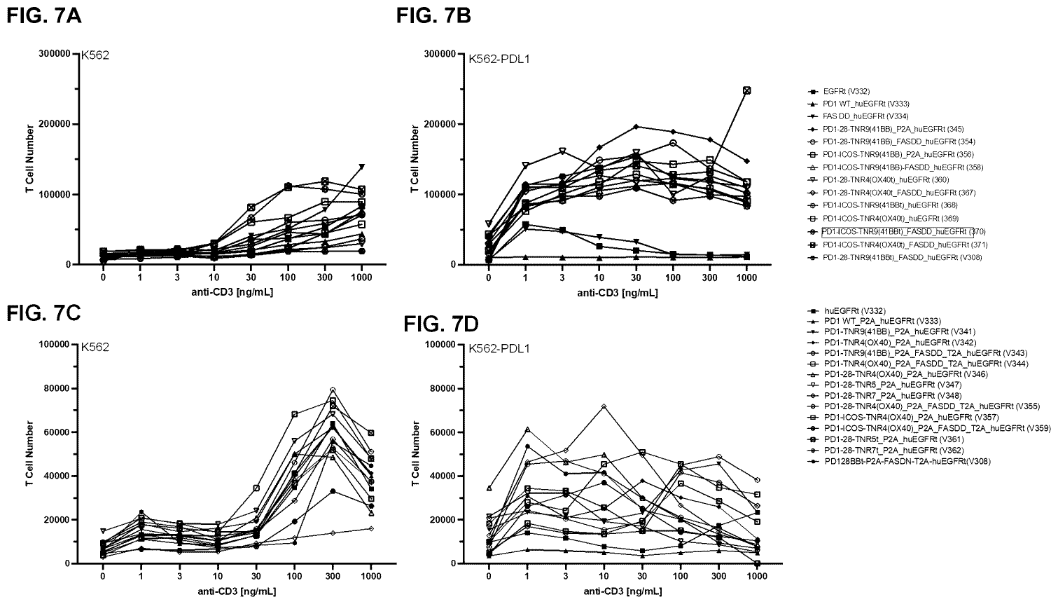

Similarly to K562 killing, T cells transduced with the ICS transgenes or individual components mediated robust T cell proliferation in the presence of PD-L1, particularly at low concentrations of anti-CD3, when compared to the huEGFRt control T cells (FIG. 7A-7D). The PD-1-CCRs are capable of causing T cell proliferation at anti-CD3 concentrations of as little as 1 ng/mL in the presence of PD-L1, while untransduced cells begin proliferating between 30 and 100 ng/mL. Importantly, the transgenes containing the PD-1-CCR required the concurrent expression of PD-L1 on target cells and stimulation through the TCR to initiate proliferation.

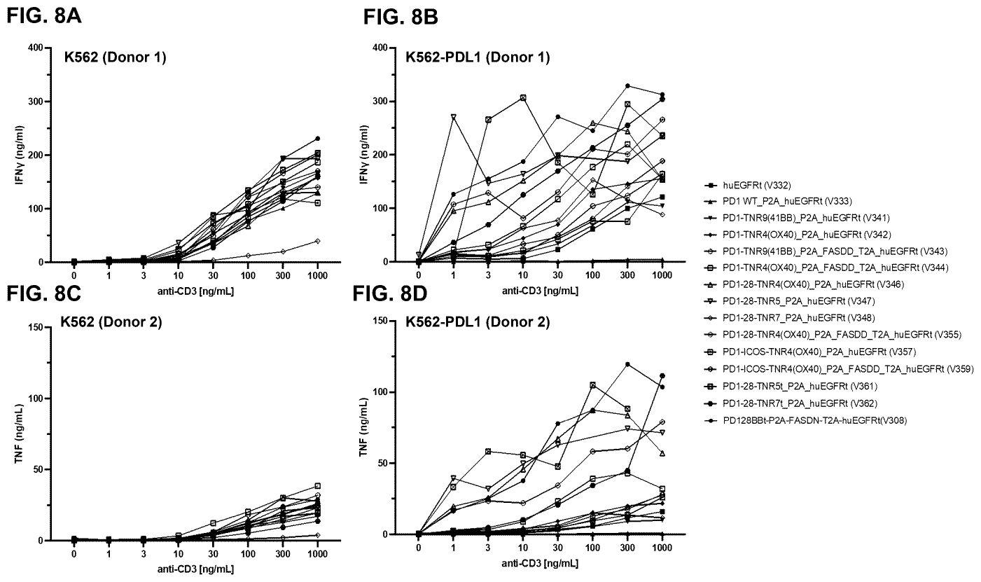

When activated by increasing anti-CD3 in co-culture with parental K562 cells, T cells transduced by various chimeric constructs and controls did not produce noticeably different levels of the effector cytokines IFNγ or TNFα (FIGS. 8A and 8B). However, in the presence of K562-PD-L1 cells, the inclusion of all switch receptors led to more cytokine production than the untransduced and wildtype-expressing control T cells (FIGS. 8B and 8D). The presence of the transgene mediates cytokine production in the presence of PD-L1 with as little as 1 ng/ml of anti-CD3, while cells in the absence of PD-L1 required over 30 ng/ml anti-CD3 to start measuring a response. This indicates a 30-fold increase in antigen sensitivity in the presence of PD-L1 by ICS transgene-expressing cells. Furthermore, the data identify PD1-28-TNFRSF4/4t/5/5t/7t/9t constructs as particularly efficacious (FIGS. 8B and 8D), while the other constructs showed moderate or minimal benefit to cytokine production.

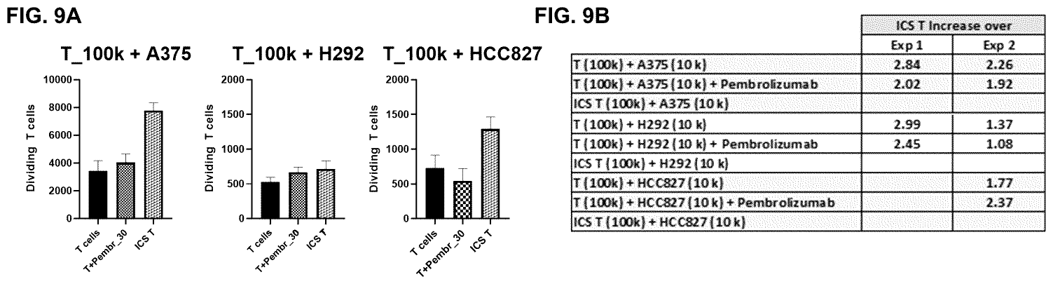

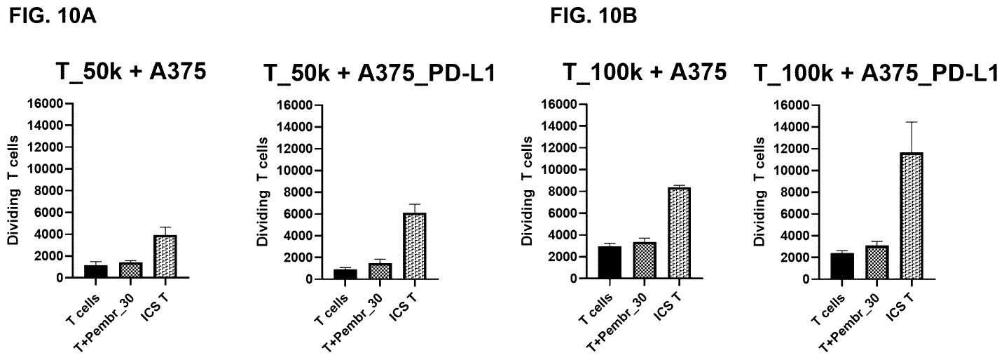

The increased functionality of the PD1-CCR/ICS transgene (V308) was tested across three cell types and was found to increase T cell proliferation in response to HLA-Mismatched target cells by approximately two-fold in the absence of exogenous PD-L1 (FIGS. 9A and 9B). This outperformed 30 μg/mL pembrolizumab, especially in the presence of exogenously expressed PD-L1 (FIGS. 10A and 10B).

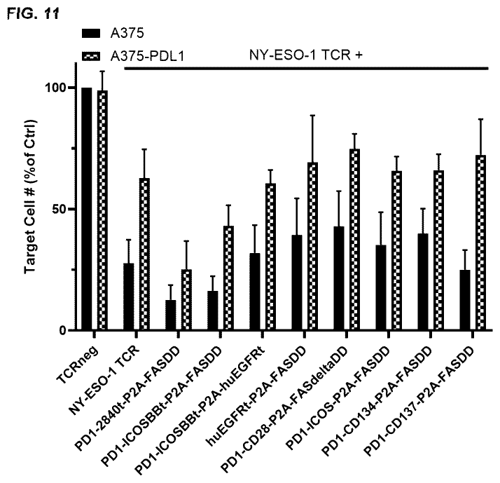

Additionally, evaluation of selected second and third generation constructs against second generation switch receptor constructs in the presence of a high-affinity TCR reacting against endogenous pMHC ligand was performed. To do this, the PD1-CCR and the FASDD proteins were expressed alongside a high affinity TCR directed against a peptide derived from the tumor associated antigen NY-ESO-1 in the context of HLA-A2. Cells transduced with the TCR and indicated constructs were incubated with antigen positive A375 or A375-PDL1 target cells and evaluated for target cell killing (FIG. 11). These data again demonstrated the lack of efficacy for 2^{nd} generation switch receptor constructs, but showed that 3^{rd} generation constructs, particularly CD28-based constructs, can dramatically enhance killing of A375 targets, especially in the presence of PD-L1.

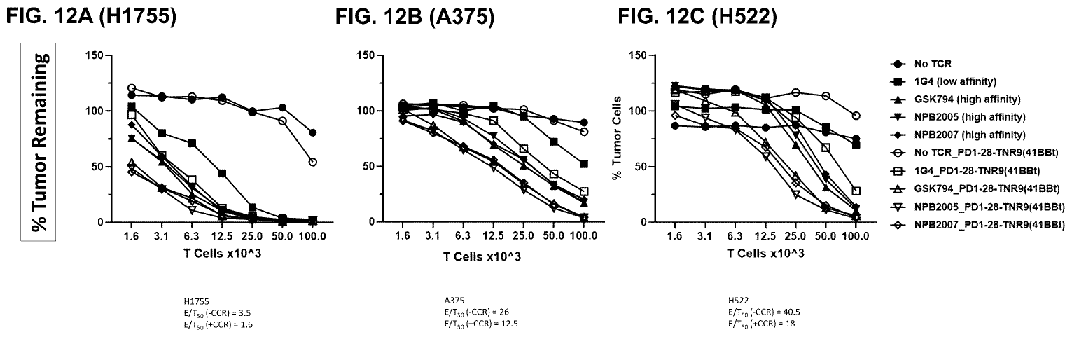

To broaden these observations to multiple cell lines and both low and high affinity TCRs, the PD1-28-BBt construct was expressed with the parental low affinity 1G4 TCR or 1 of 2 high affinity modified versions and expressed in T cells. Transduced T cells were then incubated with H1755 Targets (FIG. 12A) A375 Targets (FIG. 12B) or H522 Targets (FIG. 12C). In each case, inclusion of the PD1-CCR increased target cell killing as demonstrated by enhanced tumor cell killing at lower T Cell:Target Cell ratios.

Taken together, these data demonstrate the feasibility of the PD1-CCR constructs and identify the 3^{rd}-generation constructs PD1-28-TNFRSF4/4t/5/5t/7t/9t as mediating robust cytotoxicity, proliferation, and cytokine production. This amounts to a roughly 30-fold increase in sensitivity for high-affinity TCR interactions (K562 assay) and can increase killing in low-affinity interactions (HLA-mismatch A375 assay) at lower effector to target ratios. Additionally, the increased proliferation in the HLA-Mismatch reactivity assay by T cells expressing the ICS transgene construct V308 was not recreated by simple blockade of the PD-1/PDL1 ligand interaction, as mediated by Pembrolizumab, and is not limited to a single target cell (FIGS. 10A-10B). This indicates a true and broadly applicable costimulatory signal is being delivered by the PD-1 CCR, rather than simple removal of the negative signal conveyed by endogenous PD-1, to increase T cell reactivity. This strategy should increase T cell functionality in the immunosuppressive TME and increase T cell sensitivity to rare tumor associated antigens, above and beyond what PD-1 blockade can achieve.

### Example 3—FASDD Supports T Cell Survival and Proliferation Under Potent TCR Activation

Recurrent antigen stimulation can trigger restimulation-induced T cell death (RICD), a homeostatic mechanism that could potentially reduce T cell persistence and weaken anti-tumor efficacy of cell therapy products. FAS/FASL-induced apoptosis are a major mechanism of restimulation-induced cell death (“RICD”), and preliminary evidence in animal models has demonstrated the utility of including a FAS dominant receptor to protect adoptively transferred T cells from RICD in various tumor models for both TCR-T and CAR-T.

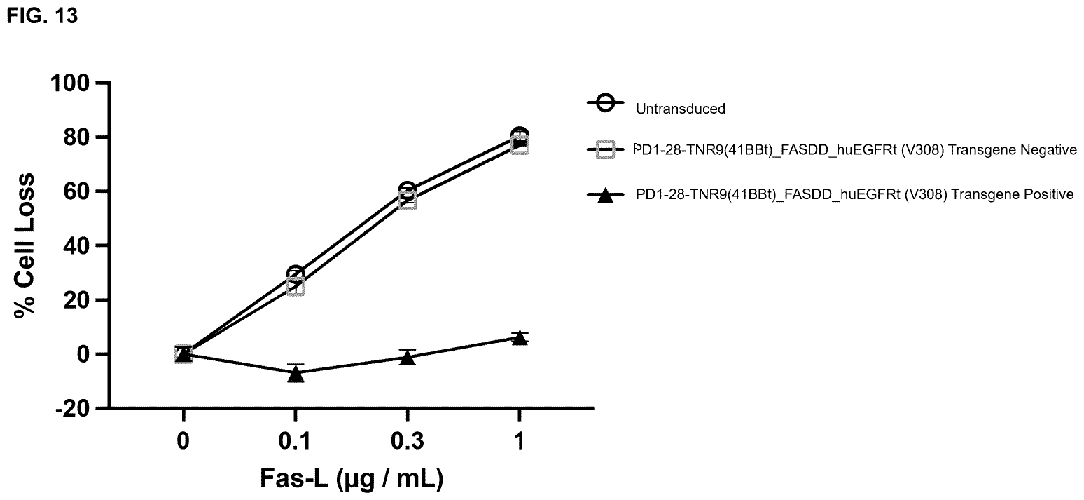

Primary human T cells expressing the ICS transgene cassette demonstrated almost complete protection from FASL-induced apoptosis in vitro (as shown in FIG. 13). Importantly, in a mixed cell population protection was mediated in-cis and not in-trans, indicating that only the cells that were expressing FASDD bearing constructs were protected from FAS-L mediated apoptosis.

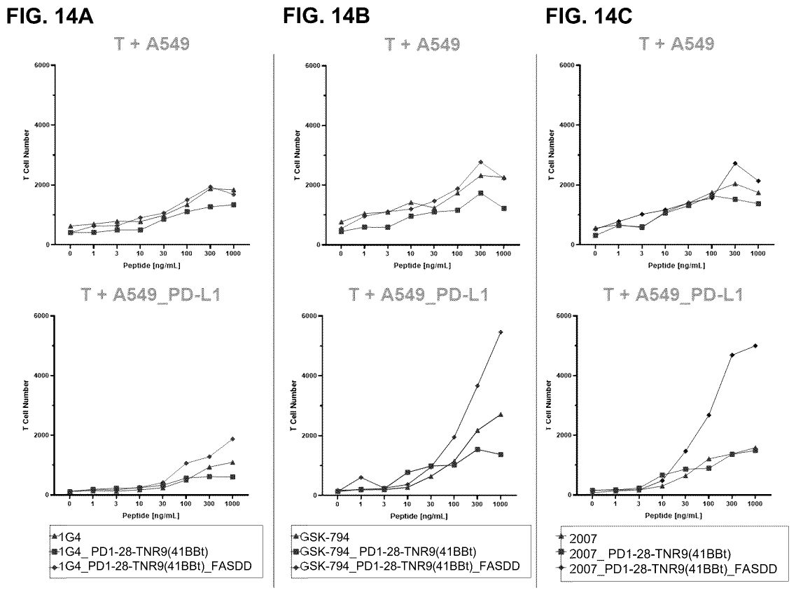

To further demonstrate the importance of the FASDD transgene in the ICS construct, T cells were transduced with either a low affinity (1G4) or two high affinity (GSK-794 or 2007) NY-ESO-1 specific TCR by itself, or along with PD1-28-TNR9 (41BBt) or PD1-28-TNR9 (41BBt)-FASDD. These cells were then stimulated on HLA-A2 expressing A549 cells co-incubated with the indicated amount of NY-ESO-1 stimulatory peptide (FIGS. 14A-14C). T cells expressing TCRs, and the ICS transgene consisting of the PD1-28-TNR9 (41BBt) PD-1CCR and FASDD demonstrated increased proliferation/survival than cells bearing either TCR or TCR and PD-1-CCR alone in the presence of PD-1. This is consistent with FASDD providing protection from T cell apoptosis following strong TCR stimulation. Strikingly, in the absence of FASDD, T cells expressing TCRs alone generally proliferated equivalent or better than cells expressing TCRs and PD-1CCR, suggesting that the additional costimulatory signal delivered by the PD-1—SR had negative impact on cell survival in the absence of the FASDD. There was a differential effect on high-affinity TCRs over the low-affinity TCR. T cells expressing high-affinity TCRs, PD-1CCR and FASDD resulted in a 2-3-fold increase in proliferation upon high PD-L1 stimulation while T cells expressing low-affinity TCR had only about 40% increase in growth (FIGS. 14A-14C).

Taken together, the data demonstrate that FASDD is effective in preventing loss of T cells from RICD and enhances T cell survival and persistence upon TCR stimulation and is highly relevant to increasing the functionality of TCR-T therapies. Importantly, both a PD-1 CCR and the dominant negative FAS construct are required for optimal T cell function and define the core of the ICS transgene cassette.

### Example 4—PD1CCR and FASDD Increase Anti-Tumor Potency by Enhanced Effector Function and T Cell Proliferation in TCR-T Cells

FIGS. 12A-12C demonstrate that the ICS component PD1-28-TNR9 (41BBt) PD1 CCR construct enhances the efficacy of T cells engineered to express both low and high-affinity TCRs in context of a specific antigen-pMHC interaction. FIGS. 14A-14C demonstrate that the ICS transgene component FAS-DD functions to enhance antigen-specific T Cell expansion with PD-1 CCR and that the ICS transgene works to enhance T cell expansion in the presence of PD-L1, only when FAS-DD is co-expressed in the transgene cassette. This likely indicates that the enhanced signaling through the PD-1 CCR causes increased stimulation induced apoptosis that can be countered by the FAS dominant negative transgene. Conversely, the data demonstrate that in the absence of PD-L1 there is minimal benefit to the PD1-CCR transgene. T cell expansion is dependent on the presence of cognate peptide demonstrating that the ICS transgene does not induce T cell activation by itself.

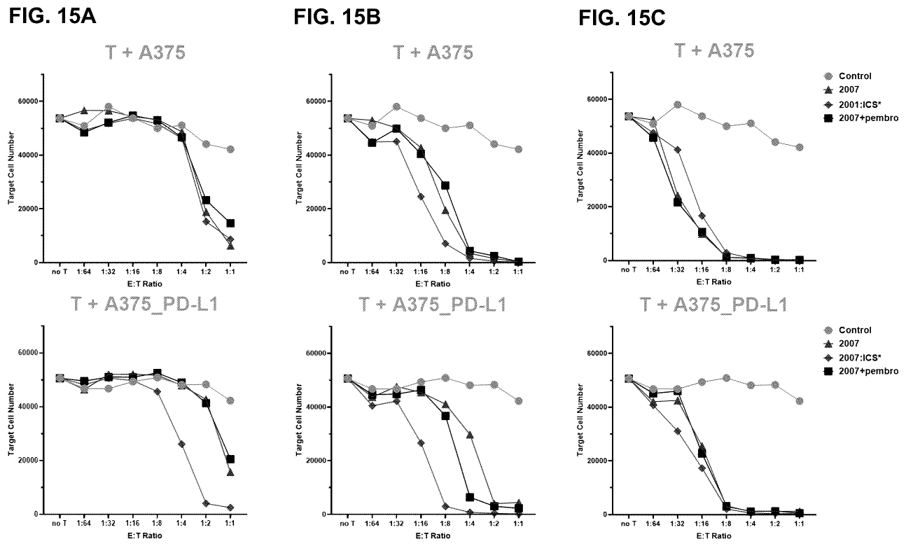

To investigate the effect of increasing PD-L1 on target cells naturally expressing a defined pMHC ligand for cognate T cells, ICS-engineered T cells co-expressing low affinity (IG4) or high affinity (GSK-794 and 2007) TCRs were co-cultured with A375 stably expressing PD-L1, pembrolizumab again served as a control for blocking PD-1/PD-L1 interactions. Inclusion of the ICS transgene PD-1-28-TNR9 (41BBt)_FASDD resulted in increased T cell-mediated killing of PD-L1 bearing target cells expressing endogenous pMHC complex, above PD-1 blockade alone. FIGS. 15A-15C demonstrate that the ICS transgene cassette can enhance the TCR-dependent target cell killing for both low affinity (IG4) or high affinity (GSK-794 and 2007) TCRs. Furthermore, the data demonstrate that the ICS transgene functions better than the PD-1 blocking antibody Pembrolizumab in increasing T cell functionality in the presence of PD-1 ligands, confirming the observations in FIGS. 9A-9B and 10A-10B.

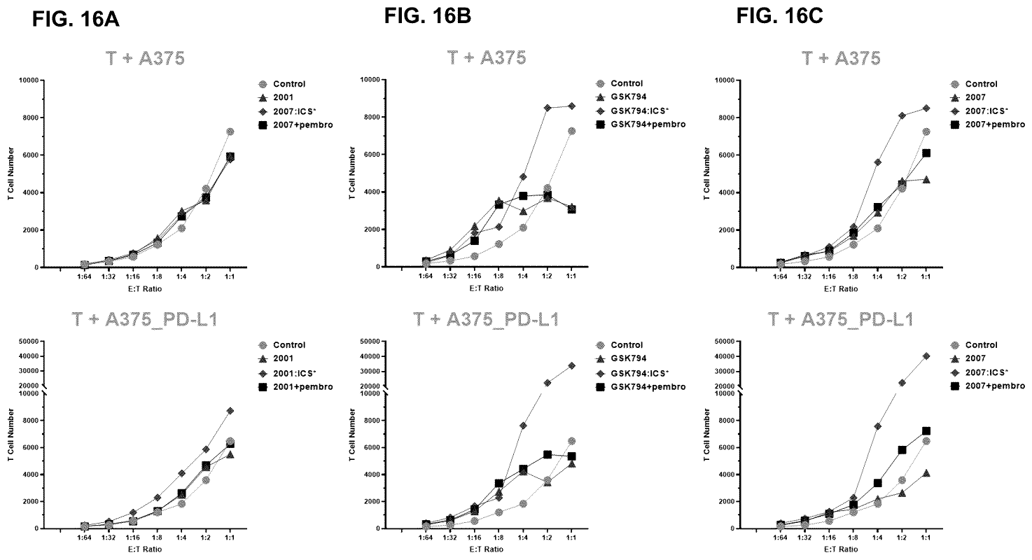

Inclusion of the ICS transgene PD-1-28-TNR9 (41BBt)_FASDD resulted in increased T cell proliferation in response to A375 and A375-PDL1 target cells expressing endogenous pMHC complex above PD-1 blockade alone. FIGS. 16A-16C demonstrate that the ICS transgene increases T cell proliferation and survival in response to cognate antigen. There was a moderate effect in the absence of PD-L1 overexpression (top row) that may demonstrate PD-1-CCR signaling in response to endogenous PD-L1 or the effect of the FAS-DN receptor. Strikingly, the ICS transgene mediated at least a 60-fold increased T cell expansion of the GSK-794 and 2007 high affinity TCRs in the presence of PD-L1. This can be compared to the modest increase in T cell number present in the pembrolizumab treated samples. These data demonstrate the advantage of arming effector T cells with the ICS transgene over use of PD-1 blocking antibodies.

### Example 5—In Vivo Evaluation of Polyclonal ICS T Cells

### Anti-Tumor Efficacy by ICS T Cells

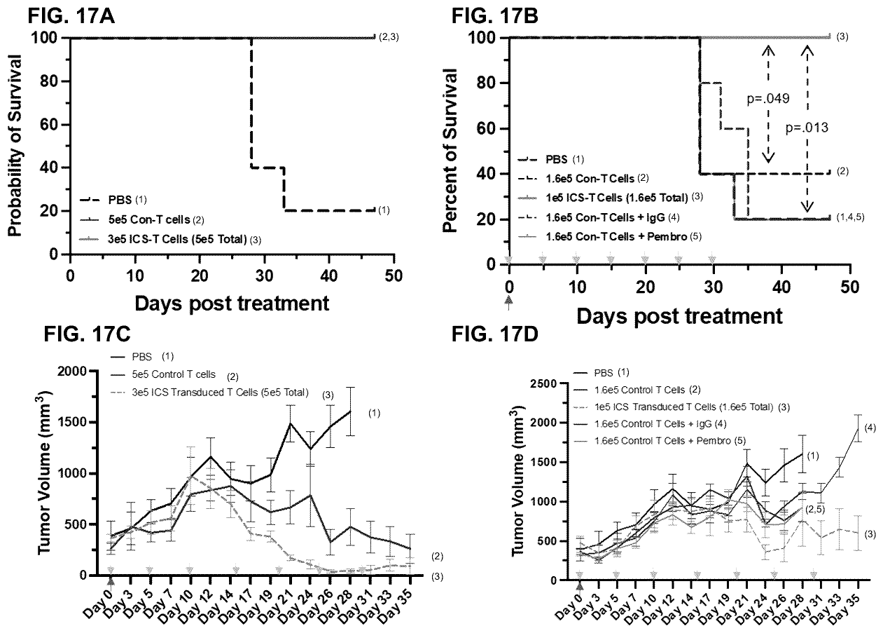

To evaluate the in vivo efficacy of the ICS-transduced T cells, HCC827 tumor cells were implanted subcutaneously in NSG mice to establish xenograft tumors. ICS T cells were produced by lentiviral delivery of the PD-1-28-TNR9 (41BBt) FASDD_huEGFRt ICS transgene cassette to about 60% transduction efficiency. When the average tumor volume reached 300 mm³, mice were randomized into treatment groups by tumor volume and were treated with three doses of ICS T cells at 3×10⁵ (high), 1×10⁵ (medium) and 0.3×10⁵ (low) transduced cells/mouse and an equal number of total T cells for matched controls. Survival of treated mice (FIGS. 17A-17B) and associated tumor volumes (FIGS. 17C-17D) demonstrate a dose dependent reduction in tumor volume in the ICS-Transduced groups that resulted in tumor control and mouse survival for the middle and high dose groups. At the high T cell dose, both transduced and untransduced cells were able to control tumor volume and mediate mouse survival, though PD-1-28-TNR9 (41BBt)_FASDD_huEGFRt ICS-transduced cells were able to clear tumor quicker than untransduced cells. At a lower dose, the PD-1-28-TNR9 (41BBt)_FASDD_huEGFRt ICS transgene mediated tumor control and survival while untransduced T cells and untransduced T cells with pembrolizumab treatment had minimal effect on tumor volume and mouse survival. These data demonstrate the efficacy of the ICS transgene in killing an established tumor and to do so with greater efficacy than PD-1 blockade alone.

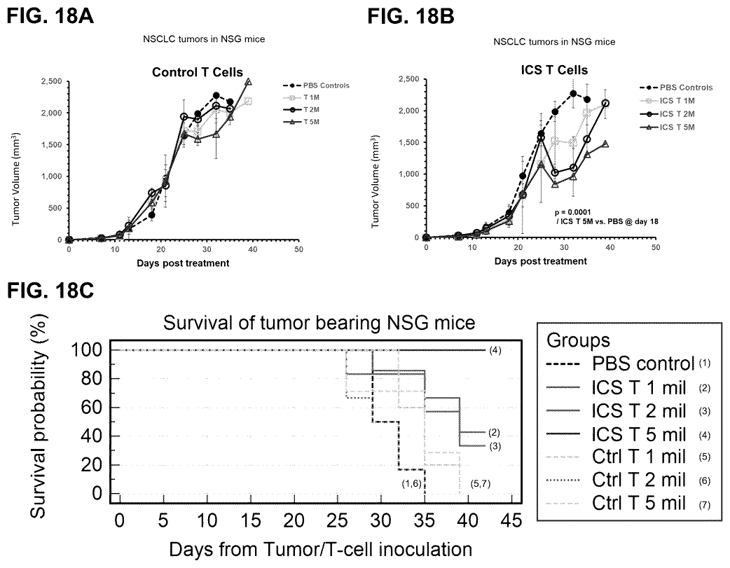

In a separate tumor model (NCI-H292), tumor cells were implanted subcutaneously, and T cells were dosed intravenously at time of tumor implantation (FIGS. 18A-18C). Though the tumor was not prevented from growing, even at the highest dose of ICS transduced T cells, the ICS-Transgene transduced T cells slowed tumor progression at all doses, and in a dose-dependent manner. In contrast, a slight delay in tumor growth was only observed in the highest control T cell dose group. The delayed tumor growth led to a prolonged survival of all three ICS-T treated groups, with all mice in the highest dose group surviving until the end of the experiment.

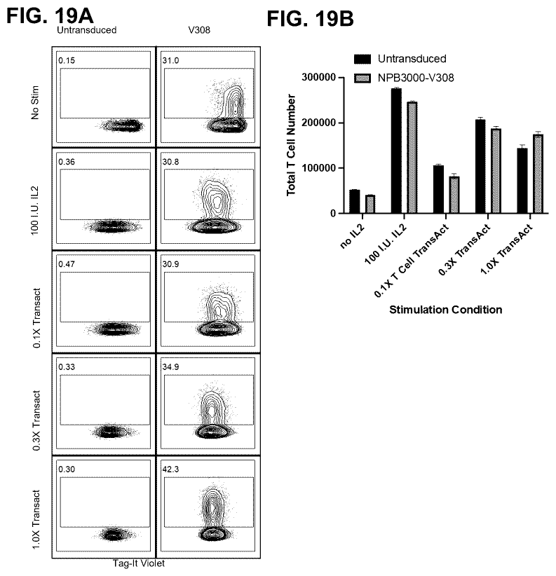

Of concern with CAR Receptors is that the combination of intracellular signaling domains and extracellular domains will result in tonic signaling that can lead to cytokine production and/or proliferation of transduced cells in the absence of external stimulation. Additionally, it is a concern that the presence of the transgene may interfere with, or otherwise alter the signaling and activation downstream of cytokine or TCR Receptors. The ICS transgene PD-1-28-TNR9 (41BBt)_FASDD_huEGFRt (V308) does not induce tonic signaling or alter Cytokine/TCR dependent growth. FIGS. 19A-19B demonstrate that in the absence of PD-1 ligands the ICS transgene does not interfere with IL-2 withdrawal mediated apoptosis, IL-2 dependent cell growth, or TCR-dependent proliferation/survival over a 96-hour period. This indicates that the transgene cassette has little ligand-independent function and should be safe from the concern of transgene-dependent tumorigenicity.

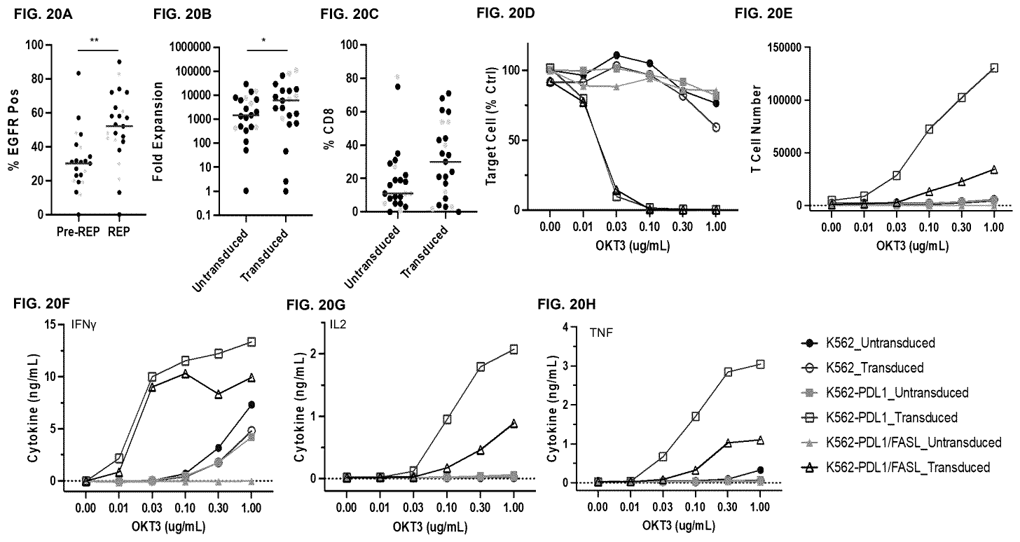

FIGS. 20A-20H further demonstrate the benefit of the ICS transgene PD-1-28-TNR9 (41BBt) FASDD_huEGFRt to the growth and functionality of Tumor Infiltrating Lymphocytes. FIG. 20A shows that TILs are readily transduced by lentivirus containing the ICS transgene during the Pre-REP phase of TIL expansion, and that the % transduced increases following completion of the REP phase. The increase in % transduced is likely due to a cell-intrinsic increase in TIL expansion during the REP resulting in a greater total expansion of the transduced TIL population compared to untransduced TILs (FIG. 20B). ICS transduction also trended towards an increased CD8 positive cell percentage at the end of the REP, possibly enhancing in-vivo functionality of the final product (FIG. 20C). In a K562 assay with target cells engineered to express either PD-L1 or PD-L1 and FAS-L the ICS-Transduced TILs were able to completely clear both PD-L1 expressing target cells (FIG. 20D) and proliferated robustly in response to anti-CD3 (FIG. 20E). This was dependent on the PD-L1/ICS interaction as there was minimal target cell lysis and proliferation in the absence of PD-L1. Further, while the ICS transgene was able to mitigate some FAS-mediated apoptosis, there was less T cell proliferation in the FAS-L expressing cells. This can be contrasted with the near complete loss of the untransduced T cells in the K562-PDL1/FASL target cell co-culture. The efficacy of the ICS transgene in increasing T cell functionality is further demonstrated by the cytokine secretion measured 18 hours after initiating the co-culture. The ICS transgene greatly increased the IFNγ production in response to target cells expressing PD-L1, and largely protected cells from the effects of FAS-L which completely abrogated IFNγ secretion by untransduced T cells (FIG. 20F). Furthermore, the ICS transgene was able to induce both IL-2 and TNF secretion by TILs in response to targets expressing PD-L1. This is remarkable as TILs largely exist in an exhausted state with limited ability to secrete either cytokine, as demonstrated by the minimal/non-existent cytokine secretion by untransduced T cells (FIGS. 20G and 20H).

### High Dose of ICS T in Mice Did not Demonstrate Increased GvHD Toxicity in a T Cell Xenograft Model

The development of GvHD in mice upon injection of human T cells or PBMCs is used to investigate the auto-immune toxicity of T cell-modulating drug candidates. Infusion of 10 million human T cells will lead to the development of auto inflammation and terminal GvHD, usually within 30-40 days post injection. As reported (A1-Khami et al. Mol Cancer Ther (2020) 19:2105-2116), the anti-PD-1 antibody from Pfizer at 0.1 mg/kg, which is one tenth of 10 mg/kg efficacious dose, could induce the acceleration of auto inflammation, leading to body weight loss and animal death. In a preliminary xenograft anti-tumor efficacy study, injection of 12.5 million cells (5 million transduced cells), which is more than 100-fold high than the anti-tumor efficacious dose in this xenograft model, did not result in any acute toxicity (data not shown). Indeed, the development of terminal GvHD and body weight loss in this study occurred with similar kinetics between the transduced and untransduced (control) T Cell injected mouse groups. Importantly, the PD-1CCR was confirmed to bind to and be activated by mouse PD-L1 in vitro (data not shown). Based on these studies and published data on anti-PD-1 antibodies, adoptive cell therapy utilizing the ICS transgenes should be considered safe for clinical evaluation.

### Safety Switch to Precisely and Timely Control Intolerable Side Effects

Incorporated into the design of the ICS transgene cassette is a truncated form of human EGFR, that lacks the two N-Terminal extracellular ligand-binding domains and the entirety of the intracellular domain. Treatment with available anti-EGFR therapeutic antibodies, such as Cetuximab, should selectively deplete cells expressing the ICS transgene cassette.

1. An isolated cell comprising:
a) a chimeric PD-1 switch receptor comprising:
(i) an extracellular domain that binds to PD-L1 or PD-L2;
(ii) a transmembrane domain; and
(iii) an intracellular domain; and
b) a FAS dominant negative receptor (FASDD).

2. The isolated cell of claim 1, wherein the FASDD comprises a dominant negative FAS mutant.

3. The isolated cell of claim 2, wherein the dominant negative FAS mutant comprises at least one modification in a cytoplasmic death domain of FAS or in the extracellular ligand binding domain.

4. The isolated cell of claim 1, wherein the FASDD comprises:
a) a deletion of amino acid residues 230-314 of SEQ ID NO:200 or a portion thereof, optionally wherein the FASDD comprises SEQ ID NO:201;
b) a point mutation at position 260 of SEQ ID NO:200, optionally wherein the point mutation is D260V, and optionally wherein the FASDD comprises SEQ ID NO:203; and/or
c) one or more mutations selected from Thr13fs; Val15_Ala16insTer; Thr28Ala; Lys33Glu; Leu37Ter; His54Arg; Cys59Phe; His60fs; Pro62Arg; Cys73Gly; Asp78fs; Cys82Arg; Val83Met; Pro84Leu; His96Arg; Asp108Gly; Glu116Gly; Arg121Trp; Thr122Ile; Cys135Phe; Thr138Ile; Val139fs; Asp144Glu; Thr160Ala; Thr163Ile; Gly169Val; Gly175Glu; Leu177Arg; Cys178Tyr; Leu179Arg; Ile184Val; Val188fs; Lys193Arg; Glu194Lys; Asn206fs; His210Tyr; Ser212Thr; Thr214Ile, Thr214Asn; Val220fs, Val220Met; Ile222Met; Asn223His; Lys231fs; Tyr232Cys, Tyr232His; Thr234Ala; Thr235del; Thr241Lys, Thr241Pro; Gly247Ala; Val249Leu; Arg250Pro, Arg250Gln; Gly253Asp, Gly253Ser, Gly253Val; Val254Ala; Ala257Asp; Ile259Arg, Ile259Thr; Asp260Gly; Asp260Val, Asp260Tyr; Ile262Ser; Thr270Ile, Thr270Lys; Glu272Gly, Glu272Lys; Gln273Ter, Gln273Arg, Glu289Asp; Ala290Glu; Leu294fs; Ala301Thr; Thr305Ile; Ile310Ser; Leu315Phe; Thr319Ile; Ser320Gly; Asp321Asn; Asn326Asp; Glu330fs; or a combination thereof, wherein the one or more mutations are relative to SEQ ID NO:200.

5. The isolated cell of claim 1, wherein the expression of the endogenous FAS gene in the cell is reduced or inhibited.

6. The isolated cell of claim 1, wherein the extracellular domain of the PD-1 switch receptor is derived from PD-1 and/or comprises SEQ ID NO: 9.

7. (canceled)

8. The isolated cell of claim 1, wherein the PD-1 switch receptor comprises a transmembrane domain derived from CD8, PD-1, CD28, ICOS, or IgG and/or wherein the PD-1 switch receptor comprises an intracellular domain connected to the C-terminus of the transmembrane domain, optionally wherein the intracellular domain comprises a first and at least a second signal transduction domain,
wherein the first and the at least second signal transduction domains are non-identical, and optionally wherein the first signal transduction domain is derived from CD28, CD28H, ICOS, or a combination thereof and the at least second domain is derived from CD137 (4-1BB), CD134 (OX-40), CD40 (TNFRSF5), or CD27 (TNFRSF7).

9. (canceled)

10. The isolated cell of claim 1, wherein the at least second signal transduction domain comprises a mutant CD137 (4-1BB) intracellular domain, a mutant CD134 (OX-40) intracellular domain, a mutant CD40 (TNFRSF5) intracellular domain, or a mutant CD27 (TNFRSF7) intracellular domain, optionally wherein:
a) the mutant CD137 intracellular domain comprises:
(1) a truncated CD137 intracellular domain, optionally wherein the truncated CD137 intracellular domain comprises:
li) an amino acid sequence according to amino acid position 13 to amino acid position 42 of the CD137 intracellular domain;
lii) a deletion of a continuous stretch of one, two, three, four, five, six, seven, eight, nine, ten or more amino acids from the N-terminus the CD137 intracellular domain; or
liii) a deletion of one, two, three, four, five, six, seven, eight, nine, ten or more amino acids from amino acid position 1 to amino acid position 12 of the N-terminus of the CD137 intracellular domain; and/or
(1) an amino acid sequence according to SEQ ID NO:3;
(2) a deletion of one, two, three or four lysine residue(s) from amino acid position 1 to amino acid position 12 of the N-terminus of the CD137 intracellular domain;
(3) one or more lysine mutation(s) from amino acid position 1 to amino acid position 12 of the N-terminus of the CD137 intracellular domain, optionally one or more lysine mutation(s) at amino acid positions selected from amino acid positions 1, 5, 6 and 12 of the N-terminus of the CD137 intracellular domain;
(4) a deletion of one or more proximal basic amino acids from amino acid position 1 to amino acid position 12 of the N-terminus of the CD137 intracellular domain; and/or
(5) one or more proximal basic amino acid mutation(s) from amino acid position 1 to amino acid position 12 of the N-terminus of the CD137 intracellular domain optionally one or more proximal basic amino acid mutation(s) at amino acid positions selected from amino acid positions 1, 2, 3, 4, 5 and 6 of the N-terminus of the CD137 intracellular domain, and
optionally a lysine mutation at amino acid position 12 of the N-terminus of the CD137 intracellular domain;
b) the mutant CD134 intracellular domain comprises:
(1) a truncated CD134 intracellular domain, optionally wherein the truncated CD134 intracellular domain comprises:
li) an amino acid sequence according to amino acid position 15 to amino acid position 37 of the CD134 intracellular domain;
lii) a deletion of a continuous stretch of one, two, three, four, five, six, seven, eight, nine, ten, eleven, twelve, thirteen or more amino acids from the N-terminus of the CD134 intracellular domain; or
liii) a deletion of one, two, three, four, five, six, seven, eight, nine, ten, eleven, twelve, thirteen or more amino acids from amino acid position 1 to amino acid position 14 of the N-terminus of the CD134 intracellular domain; and/or
(1) an amino acid sequence according to SEQ ID NO:6;
(2) a deletion of a lysine residue from amino acid position 1 to amino acid position 14 of the N-terminus of the CD134 intracellular domain;
(3) a lysine mutation at amino acid position 12 of the N-terminus of the CD134 intracellular domain;
(4) a deletion of one or more proximal basic amino acids from amino acid position 1 to amino acid position 14 of the N-terminus of the CD134 intracellular domain; and/or
(5) one or more proximal basic amino acid mutation(s) from amino acid position 1 to amino acid position 14 of the N-terminus of the CD134 intracellular domain, optionally one or more proximal basic amino acid mutation(s) at amino acid positions selected from amino acid positions 1, 2, and 5 of the N-terminus of the CD134 intracellular domain, and
optionally wherein the mutant CD134 intracellular domain further comprises a lysine mutation at amino acid position 12 of the N-terminus of the CD134 intracellular domain;
c) the mutant CD40 intracellular domain comprises:
(1) a truncated CD40 intracellular domain, optionally wherein the truncated CD40 intracellular domain comprises:
li) an amino acid sequence according to amino acid position 11 to amino acid position 62 of the CD40 intracellular domain;
lii) a deletion of a continuous stretch of one, two, three, four, five, six, seven, eight, nine, ten, or more amino acids from the N-terminus of the CD40 intracellular domain;
liii) a deletion of one or more proximal basic amino acids from amino acid position 1 to amino acid position 10 of the N-terminus of the CD40 intracellular domain; and/or
(1) an amino acid sequence according to SEQ ID NO:239;
(2) a deletion of a lysine residue from amino acid position 1 to amino acid position 10 of the N-terminus of the CD40 intracellular domain;
(3) a lysine mutation at amino acid position 10 of the N-terminus of the CD40 intracellular domain;
(4) a deletion of one or more proximal basic amino acids from amino acid position 1 to amino acid position 10 of the N-terminus of the CD40 intracellular domain; and/or
(5) one or more proximal basic amino acid mutation(s) from amino acid position 1 to amino acid position 10 of the N-terminus of the CD40 intracellular domain, optionally one or more proximal basic amino acid mutation(s) at amino acid positions selected from amino acid positions 1, 2, 5, 6, and 10 of the N-terminus of the CD40 intracellular domain; or
d) the mutant CD27 intracellular domain comprises:
(1) a truncated CD27 intracellular domain, optionally wherein the truncated CD27 intracellular domain comprises:
li) an amino acid sequence according to amino acid position 9 to amino acid position 48 of the CD27 intracellular domain;
lii) a deletion of a continuous stretch of one, two, three, four, five, six, seven, eight, nine, or more amino acids from the N-terminus of the CD27 intracellular domain;
liii) a deletion of one or more proximal basic amino acids from amino acid position 1 to amino acid position 9 of the N-terminus of the CD27 intracellular domain; and/or
(1) an amino acid sequence according to SEQ ID NO:226;
(2) a deletion of a lysine residue from amino acid position 1 to amino acid position 9 of the N-terminus of the CD27 intracellular domain;
(3) a lysine mutation at amino acid position 9 of the N-terminus of the CD27 intracellular domain;
(4) a deletion of one or more proximal basic amino acids from amino acid position 1 to amino acid position 9 of the N-terminus of the CD27 intracellular domain; and/or
(5) one or more proximal basic amino acid mutation(s) from amino acid position 1 to amino acid position 9 of the N-terminus of the CD27 intracellular domain, optionally one or more proximal basic amino acid mutation(s) at amino acid positions selected from amino acid positions 4 and 9 of the N-terminus of the CD27 intracellular domain.

11. The isolated cell of claim 1, wherein the transmembrane and intracellular domain comprise any one of SEQ ID NOs: 147, 193, and 240-243.

12. The isolated cell of claim 1, wherein the intracellular domain further comprises a third signal transduction domain, optionally wherein the third signal transduction domain is derived from a CD2 signaling domain, a MYD88 signaling domain, an interleukin 2 receptor binding (IL-2RB) protein signaling domain, or a combination thereof, and/or
wherein the intracellular domain further comprises a fourth signal transduction domain, optionally wherein the fourth signal transduction domain is derived from a CD2 signaling domain, a MYD88 signaling domain, an interleukin 2 receptor binding (IL-2RB) protein signaling domain, or a combination thereof, and wherein the third and the fourth signal transduction domain are not identical.

13. (canceled)

14. The isolated cell of claim 1, further comprising a safety switch, optionally wherein the safety switch is selected from a truncated EGFR, an RQR8 protein, and an inducible Caspase-9.

15. The isolated cell of claim 14, wherein the truncated EGFR comprises SEQ ID NO: 205 or 206.

16. The isolated cell of claim 1, wherein the cell is an immunomodulatory cell.

17. The isolated cell of claim 16, wherein the immunomodulatory cell is a T cell, a natural killer T cell (NK-T cell), or a tumor infiltrating lymphocyte (TIL).

18. The isolated cell of claim 17, wherein the T cell is:
a) an allogenic T cell or an autologous T cell; and/or
b) a naïve T cell, an early memory T cell, a stem cell-like T cell, a stem memory T cell (T_{SCM}), a central memory T cell (T_{CM}), or a regulatory T cell (T_{reg}).

19.-20. (canceled)

21. The isolated cell of claim 1, wherein the cell further comprises a sequence encoding an artificial antigen receptor, a therapeutic polypeptide, or an immune cell modulatory protein, or a combination thereof.

22. The isolated cell of claim 21, wherein the artificial antigen receptor comprises a chimeric antigen receptor (CAR) or an exogenous T cell receptor (TCR).

23.-24. (canceled)

25. A composition comprising one or more isolated nucleic acids, wherein the one or more isolated nucleic acids encode:
a) at least one chimeric PD-1 switch receptor, the chimeric PD-1 switch receptor comprising:
(i) an extracellular domain that binds to PD-L1 or PD-L2;
(ii) a transmembrane domain; and
(iii) an intracellular domain; and
b) a FAS dominant negative receptor (FASDD).

26. An isolated nucleic acid encoding:
a) a chimeric PD-1 switch receptor comprising:
(i) an extracellular domain that binds to PD-L1 or PD-L2;
(ii) a transmembrane domain; and
(iii) an intracellular domain; and
b) a FAS dominant negative receptor (FASDD).

27.-41. (canceled)

42. An isolated cell comprising the composition or the isolated nucleic acid of claim 25.

43.-51. (canceled)

52. A pharmaceutical composition comprising an effective amount of the isolated cell of claim 1, and a pharmaceutically acceptable carrier or excipient.

53. A method of treating a disease or disorder in a subject in need thereof, the method comprising administering an effective amount of the isolated cell of claim 1 to the subject.

54. (canceled)
